SEQUENCE LISTING (245 sequences)

SEQ ID NO: 1 - DNA, 1179 bp, Photobacterium iliopiscarium
  source 1..1179: mol_type = genomic DNA
atgtttctacaaagaccagagccatattcagatgaaagtttagaaagttttttcatcagagttgcgaacaaaaatggctatgaagatatccatcggttcctcattgtaacaaaaaagtatttacaagatgaaaaccacgatagatttaaggctttccctactgatattaaaacaatcaatccttactcgtctaaaaacaatcatggttcaagagtagcagcactgcacaaaatagcccttatgacttttaatgagcaacctgagatactgtctttagcaataaataggacatctctaatatattcgccatcaacaaccgctctagtacgtggtagtgaggttattcctcgctcattattacgaaaaaacattattccatgttgcccaagatgtttacatgaggaaggttttggacattatttatggcatttcaacggttataattactgccacaaacataatactccattaatatataagtgcgcatgtggcgcagattatgattatcgaatagcaggcttaaatggattttgcagagaatgtgatacgtaccttaatccaaaccaacaacatccagatagtgccgaaagcaaaacttccgcttggctttcaggcaagactatcgagcaactgcctgacttgccccagagctatcgttggggtctaattcattggtggactcaccttactcaaaaagagtttgatggtgcttcctttattgacttttggcatcgttggcctgaatcctttcatcatttaatacaacaaaaaattgaatttaatttagagcattcaattattcatcatgataatttgaagctaaaagatctactgggagatttgttctttaactgtattcgattacctgaaaggaatttaaagtacaacattgtgcttaatgagttattgagatttatagatgataatttatggggtaacaatggacggcttgcaaatctaaaaatgaatgctgtagaagtctctattttcctcaattgctcgtgcgatcaaattgtattaatggtcgaacaacggtttttaaaacccagccggaaagctaaaccaaataaaccattaagttttactgattacttgtttcacctaggagatgtttattgcctttggttagctgaatttcaaaccgatgagtttaatcgaacatttatcatttcgaggtag

SEQ ID NO: 2 - DNA, 1911 bp, Photobacterium iliopiscarium
  source 1..1911: mol_type = genomic DNA
atgagtacactagctaaaattattaattcatcgtcatcagatataaatgttgaactcaaaagagcttttcgcccactgagtccaaatattgatatcaccgacaacgaagatatcgctctcatcattttaacgaacctagccgcaaaagtagacgcaaaaaaaaatttgaccgatagagaaaactgtaaaaaaaaattaaaagataacaactggtggtcacgttgtattaatacaatgaaatttcgacaaagccacaatgttaaatttcccgagataaaagcaaccggaataattagagctcacccaatgggtgaaatacccaagtacttactatcttcttcaaaattaaaacaaaaaactttggcttatgcgaatgattcaaaggatgtcagtgtcgctaattttttaatcagtgaatttatcttgcacggcaaaatttgttgcttgggcgtcttgcttacagatgagcagcatcctctctgggatcgtttaacaaaactgggctgttatcaaaaaacacgcaagtacgtagcaaaacaacttaaaaaaattccaaattcttgtattgaagtacagctaggtgctaattctgttcgacagatttcattacctgacgggaataactcttacttatctctatcccctgttacctctcaaacaatgcagagttcatgctatcagtctcttcataaacactattggttaagtaaaattacccgttatagcagagcttcaaatatgggtgtacttccgatgacctgcggtggaacattacgcatgttaagtcatactataagctttgaagatgtcaaacattgccaaatatcgcccaactcatcttggttaactaaagattcaatacaagctttctacgactattttgattcttcaaactggatgatgcctcacattcaaaagcaacaaaagttaaaaatactaaaaatcacaatcacagaaatgatatctcgttggttagcattacaaaaaaacacaatacaacataatcagttagagcttgtacataaactaaatttcgatctatcaaatacaaaatccttaagacaatttgcctacgatccaaaaacaactagatttctatttaccttaattgaaaatagatcaaacacacagcaacctaatgagaactttgataatcaatatcttcttttgcctcaaatttctgtttgtggtgcatctgctatgagttcatccatcactattggaataccctctttaaccgcattttatggctttattcatgcatttgagcgaaacatgcaaaaggtcttttcaaacctaaaagtcgattcatttgctatctgcattcatacttttcacttacaaaaaagagggatgactagggaaagtataataaaattgacaagtaatatatcttcccctgcaactcatgatgaatggcattgtgatctgaaatttagtctggctctcaaagtaaataatcttactcacctcaaccaaagcgaaatattgtatgcattgccaaaacgacttgctggaggctacgcaagaataaatatagatgacataaagacgattcaagtatcttctagctttaaggatttggttaaacaaatacccacttttcaaggacaatggctatccttatacaaggcgaaaattgattcctttgaaagcattattcaacaaataaaagcatcgcaaaatataatcccaacttgtgttggatatcattacctagaaaagccgacagaacgcacttttgccctaagagactacaaacatgcgtttgctgagcctatcattggtctcatcaaaccaataactattaaagaagatacaagtttgcatgatttgttttgggaccaaattaacaagccacactacaccacaatagagacaaggaatatgaataatgaagctccctattag

SEQ ID NO: 3 - DNA, 1050 bp, Photobacterium iliopiscarium
  source 1..1050: mol_type = genomic DNA
atgaagctccctattagtcttgcttatgaacgatcaatccatcccagcgatgtttgcttttttaccatttggccagatggaaataaagcccctttatcttttatgagccgaacagtacttggtcaaatggaaaccgcatcactcgcttacgatactaaaggaaaccctaaacaaacggctactcctgaagttcttgcacaaggaaaccctcatcatatggatttctgtagcgttccctttggcgcagagcatattgaatgcgttttttcagtttcattctcttcagagcttcgcaaaccttacaagtgtagcgagcctgaggttaaaaacactttgattgagctggtcaaattatatgagtcaaaaatcggttgggatgaactcgcaggtcggtttctaaccaatatttgtcaaggaaaatggttgtggaaaaacacgaaaaaaacgtaccaactaaatattgaaattaaaccttggccttggaaaaataaccccgtcattttcgaagatattagaacaaattacagtacttataatgactgccaatctgataccaaatggcaagaattaaaacaactcatcatcaatgctttctcctcaaaaaatggcctcgctattttcgagataaaagcacaattaactctaccaacaaatgctgagctttacccaagccaagcatttattgaaaaagataagaaagaaaaaaaacatcaatctagccgtatatttcaacatacatcaatcaatggacaaaaaagccctatcataggctgctataaagcaggagctggcatttttatgattgatgattggtaccctaatgcagaagaacctattcgtatagggagattcggtgtacaccaacaagatgtaacttgctatcgccccccttcaactcatcaagatctcttttctttattggagcagtctgaaaaatatatagctttattaaaaaatagaaaaaaaacacctcaaaaaacaataaatgaactgcactttttaatggctaaccttatcaaaggcgggttgttccagcataaaggagaataa

SEQ ID NO: 4 - DNA, 600 bp, Photobacterium iliopiscarium
  source 1..600: mol_type = genomic DNA
atggattggtactataaaacgattcgatttattccgactaattgcaataatgaatttctagcagctaagtgtatcaatatattacacggttttagctaccgttatgatactcactctattggtatttcttttccaaactggtgtaatgagaccgtaggagatcggatttcatttgtaagcatcgaaaaaatggcattagattacctactcactcaaaattactttaaagaaatgcaagatcttggttacttcgatatatcaaaaacattgattgttccaattgaatgtaactttgcatgttttaagcgttatcaaaagatcgacaaatcgtcagcggctggttttcatcgaaagataaagaggcttgctaaaagagcgcataatagaggtgaggtttttgataacaagaagtataacatttacaaacaatgcgatattggacactatcattcattagcagaagaaagtaaaagtaaagggtatggatttagattaaacattcaaagaactaatgaaatcagcatcaataatatacctatttttagtagctatggattagcaaacacagcacacgcattacaatctgttcccataatttaa

SEQ ID NO: 5 - DNA, 687 bp, Photobacterium iliopiscarium
  source 1..687: mol_type = genomic DNA
atgcaaccttctcctgcaacgacatatcaaacagactctcctcttgagttcgcatttactcaaccagctagaaagttaacaaaatctcgtgggaaaaatatacatcgttacgttagcattaaaatgaatactatcatttcagttgaatcgacattggaattcgatgcctgttttcattttgattttaataaaaatatctctcgattttgctcacaacctattagatatagttatgtcattgatggaattattcgtacttatgttcctgactttttatgcgaattttattgtggtgagatggttctatacgaagttaaggcaccaaatgctgttaattcaaaacgttttactaaagagtttgaggcaaaaagacagtatgcgagaaaactttttgatgtagatcttgaattgattgaagaacacgatattagacatattcctcttcttaaaaacttaaaacgtatgcacagatatgcatctcattctgaattaacttatgagcaaacaactattcttaagtatttgaataaacatggctcaacaaagctagaaacaataattgatcattttagtaatagtaaaaatataattccgatgatttatgatttattgtctaagtacttgataaatactgaccttagagttcttctgaataaccaaactgaattaaagacaacttatgtctag

SEQ ID NO: 6 - DNA, 1812 bp, Photobacterium iliopiscarium
  source 1..1812: mol_type = genomic DNA
atgtctagaacatcattttctagttttcatggaaccccaacatctgatagcgaaaatgcttccagtaagctcacatttatcgaaaatgatgatttgcaagatttaacatcatttagcaaaaaagctcaaaatgaagctaaactcagattaagtatacttcgaaatatattgaataaaagttcagaaataaacttagttttaattgaacgcttacgggctgatcttcaacaacaaggagagcgatcaataccaagtgcaattactatctatcgatggtggttaacttataaaaaatcaaactttagtattttatctttagtaccaaagactaagcaaaagggaaatagaaacacgaaggtacctaaaacaatatcagtctatattgatcaagctgttgaacaagtaatttctgccaataaaatcaatgttagtacagcttttagacgagtcagaagaaaagtaagacaatataatcttacccataaaacaaagcatacctacccttcatatgaagccataagaaagcgcgtaaaaaaaatcaccccctatgaaaaattagctgcaagcaaaggagatcgaatagcaaaacgagaattccgtcgtatggggaaaaaaatccaaactgagtacttactagaacgagtagaagttgatcatacatggttagatttatttgctgttcatgaagaacatcgagtacctataggtcgtccttatttaactcaactagtagattgtcatagcaaatcagtaattggtttctaccttggctttgaaccaccaagttacatgtctgttgctttagcattaaaaaatgcaatcaaacgcaaagataatctattacaaaactatccatcaattcaaaatgagtggttatgttatggaatccctgacctacttgttacggataatggtaaagagtttttgtctaaagactttactcttgcttgtgacagtttacttataaacattcatcaaaacagagtagaaacacctgataataaaccgcatacagaacggcagtatggaaccactaacactgaattacttaatgatcttcctggtaaaacatttagtaattacttacaaagagagggctacaattccagcgacgaagcctcattaacattaagtgaaatcaaacaaatataccttacatggcttgttgatatttttcatcctagaccaaattcaagagggacaaattgcccaaatagagtatggaaacacgctgaaaaaaattggcctccgaatgaatttcttggtaccgacgaagaacttgatttctattttactaagttagataaacgaatgttaagaaaagaagggattggactagcaacagagcttttttatagcagtgaacgattagcagaataccgaggtaagaaaggtaatcataaagttgcgataaaatataaccgcgaaaatatgggcttcatatgggttcttgatgaagatacagaatcttatttcaaagtccctgctattgattatgaatatgcgagtacagtaactttatggatgcataaaaaaaacctaaaaataaaacaagaactcaatattaccgaacataacttagactctgaaattgatgctgaattaagaattgatgaaattgttgatcaatctattcaaaagaaaaaaacaacaataacaaataaacgtcgtgctgctcgatatcaagaaaacgaacaaatggctgctaatgctcgacaacctaaagaaaaaaaatctgctaataatcaaaagacatctactaaaaatattgaaaaagaagatagtagtggatgggatattgattatgtatag

SEQ ID NO: 7 - DNA, 987 bp, Photobacterium iliopiscarium
  source 1..987: mol_type = genomic DNA
atggatgacaacattacaagaattaccaaagcaaaaaaagcgtttgtatctacgcctgatgtagctgctatcataaacaatatagatcgatgcagaaggctctctaaatttggggcagaaccatcgtgtatgatggtatatggcgcatctggcgttggaaaaacctcaattataagaaaatacctaaaagcgaatgataaagactcaacagcacgacaagatattgtccctgttgtttatatcgaattaccagagaatgcaaaacctgtagatgccgctcgtgaactcctgctctaccttgaagatcccttagctctttatgaatcagacttagctattttaacaaaacgaatagtagatttagtaccaacaatgaaaatagagcttattatcattgatgaatttcaacacttagtagaaaaatcttcaaatagaatactctcaagagttggtgactggttaaagatgttaataaataagacaaaatgtcctgtcattctttttggtatgccctattcaaaagtagtcttggcagcaaacagtcagctgagaagccgcttttcgatacagtttgaattaaaaccatttagttatatcaataattatggcatttatgctggttttctcagacgtttagatgaagccttaccttttgatcaattatcaaaccttgcatcagaacctatagcaaaaaaaatctatgcattttcgcaaggaaatatgcgttcacttcgaaatttagtctttcatgctgcggtaaatgcaattgaaaacgacaataactgcatcagtaaaaacgactgggaatttgcctctaatttgacatctggcaataaactctcaacatggaaaaacccttttgctcaaggcgtcaagatcacagatagaatgttgcaagaccaacctaatgacttaggttgggaagattatatgcgcaacaatagaggaaaaagaaaacctttcgatgagagtaagatctttagctaa

SEQ ID NO: 8 - protein, 392 aa, Photobacterium iliopiscarium
MFLQRPEPYSDESLESFFIRVANKNGYEDIHRFLIVTKKYLQDENHDRFKAFPTDIKTINPYSSKNNHGSRVAALHKIALMTFNEQPEILSLAINRTSLIYSPSTTALVRGSEVIPRSLLRKNIIPCCPRCLHEEGFGHYLWHFNGYNYCHKHNTPLIYKCACGADYDYRIAGLNGFCRECDTYLNPNQQHPDSAESKTSAWLSGKTIEQLPDLPQSYRWGLIHWWTHLTQKEFDGASFIDFWHRWPESFHHLIQQKIEFNLEHSIIHHDNLKLKDLLGDLFFNCIRLPERNLKYNIVLNELLRFIDDNLWGNNGRLANLKMNAVEVSIFLNCSCDQIVLMVEQRFLKPSRKAKPNKPLSFTDYLFHLGDVYCLWLAEFQTDEFNRTFIISR

SEQ ID NO: 9 - protein, 636 aa, Photobacterium iliopiscarium
MSTLAKIINSSSSDINVELKRAFRPLSPNIDITDNEDIALIILTNLAAKVDAKKNLTDRENCKKKLKDNNWWSRCINTMKFRQSHNVKFPEIKATGIIRAHPMGEIPKYLLSSSKLKQKTLAYANDSKDVSVANFLISEFILHGKICCLGVLLTDEQHPLWDRLTKLGCYQKTRKYVAKQLKKIPNSCIEVQLGANSVRQISLPDGNNSYLSLSPVTSQTMQSSCYQSLHKHYWLSKITRYSRASNMGVLPMTCGGTLRMLSHTISFEDVKHCQISPNSSWLTKDSIQAFYDYFDSSNWMMPHIQKQQKLKILKITITEMISRWLALQKNTIQHNQLELVHKLNFDLSNTKSLRQFAYDPKTTRFLFTLIENRSNTQQPNENFDNQYLLLPQISVCGASAMSSSITIGIPSLTAFYGFIHAFERNMQKVFSNLKVDSFAICIHTFHLQKRGMTRESIIKLTSNISSPATHDEWHCDLKFSLALKVNNLTHLNQSEILYALPKRLAGGYARINIDDIKTIQVSSSFKDLVKQIPTFQGQWLSLYKAKIDSFESIIQQIKASQNIIPTCVGYHYLEKPTERTFALRDYKHAFAEPIIGLIKPITIKEDTSLHDLFWDQINKPHYTTIETRNMNNEAPY

SEQ ID NO: 10 - protein, 349 aa, Photobacterium iliopiscarium
MKLPISLAYERSIHPSDVCFFTIWPDGNKAPLSFMSRTVLGQMETASLAYDTKGNPKQTATPEVLAQGNPHHMDFCSVPFGAEHIECVFSVSFSSELRKPYKCSEPEVKNTLIELVKLYESKIGWDELAGRFLTNICQGKWLWKNTKKTYQLNIEIKPWPWKNNPVIFEDIRTNYSTYNDCQSDTKWQELKQLIINAFSSKNGLAIFEIKAQLTLPTNAELYPSQAFIEKDKKEKKHQSSRIFQHTSINGQKSPIIGCYKAGAGIFMIDDWYPNAEEPIRIGRFGVHQQDVTCYRPPSTHQDLFSLLEQSEKYIALLKNRKKTPQKTINELHFLMANLIKGGLFQHKGE

SEQ ID NO: 11 - protein, 199 aa, Photobacterium iliopiscarium
MDWYYKTIRFIPTNCNNEFLAAKCINILHGFSYRYDTHSIGISFPNWCNETVGDRISFVSIEKMALDYLLTQNYFKEMQDLGYFDISKTLIVPIECNFACFKRYQKIDKSSAAGFHRKIKRLAKRAHNRGEVFDNKKYNIYKQCDIGHYHSLAEESKSKGYGFRLNIQRTNEISINNIPIFSSYGLANTAHALQSVPII

SEQ ID NO: 12 - protein, 228 aa, Photobacterium iliopiscarium
MQPSPATTYQTDSPLEFAFTQPARKLTKSRGKNIHRYVSIKMNTIISVESTLEFDACFHFDFNKNISRFCSQPIRYSYVIDGIIRTYVPDFLCEFYCGEMVLYEVKAPNAVNSKRFTKEFEAKRQYARKLFDVDLELIEEHDIRHIPLLKNLKRMHRYASHSELTYEQTTILKYLNKHGSTKLETIIDHFSNSKNIIPMIYDLLSKYLINTDLRVLLNNQTELKTTYV

SEQ ID NO: 13 - protein, 603 aa, Photobacterium iliopiscarium
MSRTSFSSFHGTPTSDSENASSKLTFIENDDLQDLTSFSKKAQNEAKLRLSILRNILNKSSEINLVLIERLRADLQQQGERSIPSAITIYRWWLTYKKSNFSILSLVPKTKQKGNRNTKVPKTISVYIDQAVEQVISANKINVSTAFRRVRRKVRQYNLTHKTKHTYPSYEAIRKRVKKITPYEKLAASKGDRIAKREFRRMGKKIQTEYLLERVEVDHTWLDLFAVHEEHRVPIGRPYLTQLVDCHSKSVIGFYLGFEPPSYMSVALALKNAIKRKDNLLQNYPSIQNEWLCYGIPDLLVTDNGKEFLSKDFTLACDSLLINIHQNRVETPDNKPHTERQYGTTNTELLNDLPGKTFSNYLQREGYNSSDEASLTLSEIKQIYLTWLVDIFHPRPNSRGTNCPNRVWKHAEKNWPPNEFLGTDEELDFYFTKLDKRMLRKEGIGLATELFYSSERLAEYRGKKGNHKVAIKYNRENMGFIWVLDEDTESYFKVPAIDYEYASTVTLWMHKKNLKIKQELNITEHNLDSEIDAELRIDEIVDQSIQKKKTTITNKRRAARYQENEQMAANARQPKEKKSANNQKTSTKNIEKEDSSGWDIDYV

SEQ ID NO: 14 - protein, 328 aa, Photobacterium iliopiscarium
MDDNITRITKAKKAFVSTPDVAAIINNIDRCRRLSKFGAEPSCMMVYGASGVGKTSIIRKYLKANDKDSTARQDIVPVVYIELPENAKPVDAARELLLYLEDPLALYESDLAILTKRIVDLVPTMKIELIIIDEFQHLVEKSSNRILSRVGDWLKMLINKTKCPVILFGMPYSKVVLAANSQLRSRFSIQFELKPFSYINNYGIYAGFLRRLDEALPFDQLSNLASEPIAKKIYAFSQGNMRSLRNLVFHAAVNAIENDNNCISKNDWEFASNLTSGNKLSTWKNPFAQGVKITDRMLQDQPNDLGWEDYMRNNRGKRKPFDESKIFS

SEQ ID NO: 15 - DNA, 28 bp, Photobacterium iliopiscarium
  source 1..28: mol_type = other DNA
ccattactactgaaaagtagctgataaa

SEQ ID NO: 16 - DNA, 28 bp, Photobacterium iliopiscarium
  source 1..28: mol_type = other DNA
atatactgctgaataagtagataattaa

SEQ ID NO: 17 - DNA, 28 bp, Photobacterium iliopiscarium
  source 1..28: mol_type = other DNA
gtgtactgccgagtaggcaactgataac

SEQ ID NO: 18 - DNA, 28 bp, Photobacterium iliopiscarium
  source 1..28: mol_type = other DNA
gtgtactgccgagtaggcagctgataac

SEQ ID NO: 19 - DNA, 28 bp, Photobacterium iliopiscarium
  source 1..28: mol_type = other DNA
gtgtactgccgagtaggtacatttatca

SEQ ID NO: 20 - DNA, 109 bp, Photobacterium iliopiscarium
  source 1..109: mol_type = other DNA
tgttgaaacaaccataagttgatatttacaaccataagctgatatttaatacaaccataacttgatattgcctctttttagaccatacttatgtaagtttactacatat

SEQ ID NO: 21 - DNA, 215 bp, Photobacterium iliopiscarium
  source 1..215: mol_type = other DNA
gatgccgtacaatattcaatactaatgccataaatatacttaaacagtaaaaaagatgtaagtaactttcttacaatcaatcttcataattagtaataaacctatgaaaatatcaacttatggttgttttgaataatatcaacatatggttgtatttttattaagaaattgatttatataaaaatattgaatatcaagttatggttgtatcaaca

SEQ ID NO: 22 - DNA, 1179 bp, Photobacterium pisicola
  source 1..1179: mol_type = genomic DNA
atgtttctacaaagaccagaaccacattcagatgaaagtttggaaagttttttcatcagagttgcaaacaataatggctatgaagatatccatcggttcctcattgtaacaaaacggtatttacaagatgaaaaccacgatagatttgaggctttccctactgatataaaaacaattaacccttattcgtccaaaaataatcatggttcaagagtagcagcactgcacaaaatagcccttatgacttttaatgagcaaactgagatactgtctttagcaataaatagaacatcgctaatatattcaccatcaacaaccgctctagtacgaggtagtgaggttattcctcgttcattattacgaaaaaatactattccatgttgcccaagatgtttatatgaggaaggttttggacattatttatggcatttcaacggttacaatcactgccacaaacataatgctctattaacatataagtgcgcatgtggcgcagattatgattatcgaatagctggtctaaatggactttgcagagaatgtgatacgtatcttaatccaaaccaacaacaaccagatagtgccgaaaacaaaacttctgcttggcttgcaggtaagactatcgaacaactgcctgatttgcctcagagctatcgttggggtctaatacattggtgggctcaccttactcaaaaagagtttgatggggcttccttcattgacttttggcatcgttggccagaatcctttcatcatttaatacaacaaaaaatcgaatttaatttagaacattcaattattcatcatgataatttgaagctaaaagatttactgggagatttgttctttagctgtattcgattacctgaaaggaatttaaagtacaatattgtacttaacgagttattgagatttatagataataatttatggcgtgacaatggactacttgcgaatctaaaaatgaatgctttagaagtctcgatatttcttaattgttcgcgcgagcaaattgtattaatggtcgaccaacgatttttaaaaaccagccggaaagctaaaccaaataaaccattaagtttcactgattacctgtttcacctaggggatgtttattgtctttggttagctgaatttcaaactgatgagtttaatcgaacatttatcatttcggggtag

SEQ ID NO: 23 - DNA, 1923 bp, Photobacterium pisicola
  source 1..1923: mol_type = genomic DNA
atgagtacactagctgaaattattaattcatcgtcatcagatataaatgttgaattcaaaagagcttttcgtccactgagtccaaatattgatatcacagacaacgaagatattgctctcatcattttaaccaacctaaccgcaaaagtagacgcaaagcaaattttgaccgatagagaaaactgtaggaaaaaattaaaagataacaactggtggtcacgttgtattaatacaatgaaatttcgccaaagccacaatgttaaatttcccgagataaaagcaactggaataattagagctgatccaataggtgaaatacctaagtacttcctctcatcttcaaaattaaaacaaaaaactttgccttacgcgaatgattcaaaggatgtcagtatcgctaattttttaaccagtgaatttatcttgaacggcaaaatttgttgcttgggcgtattacttgcagatgagcagcatcccctctgggatcgtttaacaaaactgggttgttatcaaaaaacacgcaagtacgtagcaaaacaacttgagaaaattccaaattcttgtattgaagtacacctaggtgctaattctgttcgacagatttcattaccagatgggaataactcttacttatctctatcccctgctacctctcaaacaatgcagagttcatgctatcagtctcttcataaacattattggttaagtaaaactacccgttatagccgagcttcaaatatgggtgtacttccgatgacctgcggtggagcattacgcatgttaagtcatactataagctttgaagatgtcaaacattgtcaaatatcacccgattcatattggttaactaaagatgcaatacaagctttctacgactattttgattattcaaactggctgatgcctcacactcaaaagcaacaaaagttaaaaacactaaaaatcacaatcacagaaatgatatctcggtggttatcattacaaaaaaaatcgataaaagataataacttagatcttgtgcataaactaaattttgatctatcaaatacaaaatccttaagacaatttgcctacgatccaaaaacaactagatttctatttaccttaattgaaaatagatcaaacacacagcaaccttatacttcacctaatgagaactttgataatcaatatcttcttttgcctcagatttctgtttgtggtgcatccgctttaaactcatctatcactattggaataccctctttaaccgcattttttggttttattcatgcatttgagagaaatatacaaaaggtatttccaaacttcaaagttaagtcatttgctatctgtattcatacgtttcacttacaaaaaagaggaatgactagggagagtgtagaaaaatcaaaagataatatatctcctccagcaactcatgatgaatggtattgtgatctaaaatttagtttagttctcaaaataaataatcttactaacctcaaccaaagcaagatattgtatgcattgcccaaacgacttgctggaggctgcgcaagaataagtatagatgacataaagacgatacaagtatcttctagctttaaggatttggttaaacaaatacccacttttcaaggacaatggctatccttatacaaagcggaaattgattcctttgaaaacattattcaacagataaaagcatcgcaaaatataaccccaacttgtgttgggtatcattacctagaaaagccgacagaacgccctttttccctcagagactacaaccatgtgtttgctgagcctatcattggactcatcaaaccaataactattaaagaagatacaaatttacatgatttgttttggaaccaaattaacaagccacactacactacaatagagacaaggaatatgaataatgaagatccctattag

SEQ ID NO: 24 - DNA, 1050 bp, Photobacterium pisicola
  source 1..1050: mol_type = genomic DNA
atgaagatccctattagtcttgcttatgaacgttcaatccatcccagcgatgtttgcttttttaccatttggccagatggaaacaaagcccctttgtcttttatgagccgaacagcacttggtcaaatggaaaccgcatcacgcgcttacgatactaaaggaaaccctaaaaaaacggctacacctgaagctcttgcacagggaaaccctcatcatatggatttctgtagtgttccctttggcgcagagcatattgaatgcgttttttcagtttcattctcttcagagcttcgcaaaccttacaagtgtagcgagcctgaagttaaaaacactttgattgagctggttaaattatatgagtcaaaaatcggttgggatgaactcgcaggtcggtttctaatcaatatttgtcaaggaaaatggttgtggaaaaacacgaaaaaagcgtaccaattaaatattgaaattaaaccttggccttggaaaaataatcccgtcattttcgaagatattagaacaaattacagtacttataatgactgtctatctgatcccaaatggcaagaattaaaacacctcatcatcaatgctttctcctcccaaaatggtctcactattttcgagataaaagcacaattaactctaccaacgaatgctgaactttacccaagccaaacatttattgaaaaagataagaaagaaaaaaaacatcaatctagccgtatatttcaacatacatcaatcaacggacaaaaaagccctattataggctgttataaagcaggagctggtatttttatgattgatgattggtaccctaatgctgaagaacctattcgtatagggagattcggtgtacaccaacaagatgtaacttgctatcgccacccctcgactcatcaagatctcttttctttattggagcagtctgaaaaatatatagctttattaaaaaatagaaaaaaaataccgcaaaaaacaataaatgaactgcactttttaatggctaaccttatcaaaggcgggttatgccagcataaaggagaatag

SEQ ID NO: 25 - DNA, 600 bp, Photobacterium pisicola
  source 1..600: mol_type = genomic DNA
atggattggtactataaaacgattcgatttgttcctactaattgcaataatgaatttctagcagctaagtgtatcaatatattacatggttttagctaccgttatgatactcgttctattggcgtttcttttccaaactggtgtaatgagacagtaggagatcgaatttcatttgtaagcatcgaaaaaatgacattagattacttactcactcaaaattactttaaagaaatgcaagatcttggttacttcaatatatcaaaaacattaattgttccaattgaatgtaactttgcctgttttaagcgttatcaaaagatcgacaaatcgtcagcggctggttttcatcgaaagataaagaggcttgctaaaagagcgcataatagaggtgaggtttttgataacaagaagtataacatttacaaacaatgcgatattggacactatcattcattagcagaagaaagtaaaagtaaagggtatggatttagattaaacattcaaagaactaatgaagtcagcatcaataatatacctatttttagtagctatggattagcaaacacagcacacacattacaatctgtccccataatttaa

SEQ ID NO: 26 - DNA, 687 bp, Photobacterium pisicola
  source 1..687: mol_type = genomic DNA
atgcaatcgattcctccagcgacatatcaaacagactctcctcttgagttagcatttattgatccagccagaaagctaaccaaatcccgaggaaaaaatatacatcgttatgctagcgttaaaatgaatactatcatttcagttgaatcgacattggaattcgatgcctgttttcattttgattttaataacaatatctctcgattttgctcacaacctattagatatagttatgtcattgatggaattattcgtacttatgttcctgactttttatgcgaatttgattgtggtgagatggttctatacgaagttaaggcaccaaatgctgttaattcaaaacgttttactaaagagtttgaggcaaaaagacagtatgcgagaaaactttttgatgtagatcttgaattgattgaagaacacgatattagacatattcctcttcttaaaaacttaaaacacatgcacagatatgcatctcattctgaattaacttatgagcaaacaactattcttaagtatttgaataaacatggctcaacaaagctagaaacaataattgatcattttagtaatagtaaaaatataattccgatgatttatgatttattgtctaagtacttgataaatactgatcttagagttcttctgaataaccaaactgaattaaagacaacttatgtctag

SEQ ID NO: 27 - DNA, 1812 bp, Photobacterium pisicola
  source 1..1812: mol_type = genomic DNA
atgtctagaacatcattttctagttttcatggaaccccaacatctgatagcgaaaatgcttccagtaagctcacatttatcgaaaatgatgatttgcaagatttaacatcatttagcaaaaaagctcaaaatgaagctaaattcagattaagtatacttcgaaatatattgaataaaagttcagaaataaacttagttttaattgaacgcttacgggctgatcttcaacaacaagaagagcgatcaataccaagtgcaattactatctatcgatggtggctaacttataaaaaatcaaattttagtattttatctttagtaccaaagactaagcaaaagggaaatagaaacactaaggtacctaaaacaatatcagtctatattgatcaagctgttgaacaagtaatttctgccaataaaatcaatgttagtacagcttttaggcgagtcagaagaaaagtaagacaatataatcttacccataaaacaaagcatacctacccttcatatgaagccataagaaagcgcgtaaaaaaaatcaccccctatgaaaaattagctgcaagcaaaggagatcgaatagcaaaacgagaattccgtcgtatggggaaaaaaatccaaactgagtacttactagaacgagtagaagttgatcatacatggttagatttatttgctgttcatgaagaacatcgagtacctataggtcgtccttatttaactcaactagtagattgtcatagtaaatcagtaattggtttctaccttggctttgaacctccaagttacatgtctgttgccttagcattaaaaaatgcaatcaaacgcaaagataatctattacaaaactatccatcaattcaaaatgaatggttatgttatggaatccctgacctacttgttacggataatggtaaagagtttttgtctaaagactttactcttgcttgtgacagtttacttataaacattcatcaaaacagagtagaaacacctgataataaaccgcatacagaacggcagtatggaaccactaacactgaattacttaatgatcttcctggtaaaacatttagtaattacttacaaagagagggctacaattccagcgacgaagcctcattaacattaagtgaaatcaaacaaatataccttacatggcttgttgatattttccatcctagaccaaattcaagagggacaaactgcccaaatagagtatggaaacacgctgaaaaaaactggcctccgaatgaatttcttggtaccgacgaagaacttgatttctattttactaagttagataaacgaatgttaagaaaagaagggattggactagcaacagagcttttttatagcagtgaacgattagcagaataccgaggtaagaaaggtaatcataaagttacgataaaatataaccgcgaaaatatgggcttcatatgggttcttgatgaagatacagaatcttatttcaaagtccctgctattgattatgaatatgcgagtacagtaactttatggatgcataaaaaaaacctaaaaataaaacaagaactcaatattaccgaacataacttagactctgaaattgatgctgaattaagaattgatgaaattgttgatcaatctattcaaaagaaaaaaacaacaataacaaataaacgtcgtgctgctcgatatcaagaaaacgaacaaatggctgctaatgctcgacaacctaaagaaaaaaaatctgctaataatcaaaagacatcaactaaaaatattgaaaaagaagatagtagtggatgggatattgattatgtatag

SEQ ID NO: 28 - DNA, 987 bp, Photobacterium pisicola
  source 1..987: mol_type = genomic DNA
atggatgacaacattacaagaattgccaaagcaaaaaaagcgtttatatctacgcctgatgtagctgctatcataaacaatatagatcgatgcagaaggctctctaaatttggggcagaaccatcgtgtatgatggtatatggcgcatctggcgttggcaaaacctcaattataagaaaatacctaaaagcgaatgataaagactcaacagcacgacaagatattgtccctgttgtctatatcgaattaccagagaatgcaaaacctgtagatgccgctcgtgaactcctgctctaccttgaagatcccttagctctttatgaatcagacttaactattttaacaaaacgaatagtagatttagtaccaacaatgaaaatagagcttattatcattgatgaatttcaacacttagtagaaaaatcttcaaatagaatactctcaagagttggtgactggctaaagatgttaataaataagacaaaatgccctgtcattctttttggtatgccctattcaaaagtagttttggcagcaaacactcagctgagaagtcgcttttcgatacagtttgaattaaaaccatttagttatatcaataattatggcatttatgctggttttctcagacgtttagatgaagccttaccttttgatcaattatcaaaccttgcatcagaacctatagcaaaaaaaatctatgcattttcgcaaggaaatatgcgttcacttcgaaatttagtctttcatgcggcggtaaatgcaattgaaaacgacaataactgcataagtaaaaacgactgggaatttgcatctaatttgacatctggtaataaactctcaacatggaaaaacccttttgttcaaggcgtcaagatcacagatagaatgttgcaagaccaacctaatgacttaggttgggaagattatatgcgcaacaatagaggaaaacgaaaacctttcgatgagagtaagatctttagctaa

SEQ ID NO: 29 - protein, 392 aa, Photobacterium pisicola
MFLQRPEPHSDESLESFFIRVANNNGYEDIHRFLIVTKRYLQDENHDRFEAFPTDIKTINPYSSKNNHGSRVAALHKIALMTFNEQTEILSLAINRTSLIYSPSTTALVRGSEVIPRSLLRKNTIPCCPRCLYEEGFGHYLWHFNGYNHCHKHNALLTYKCACGADYDYRIAGLNGLCRECDTYLNPNQQQPDSAENKTSAWLAGKTIEQLPDLPQSYRWGLIHWWAHLTQKEFDGASFIDFWHRWPESFHHLIQQKIEFNLEHSIIHHDNLKLKDLLGDLFFSCIRLPERNLKYNIVLNELLRFIDNNLWRDNGLLANLKMNALEVSIFLNCSREQIVLMVDQRFLKTSRKAKPNKPLSFTDYLFHLGDVYCLWLAEFQTDEFNRTFIISG

SEQ ID NO: 30 - protein, 640 aa, Photobacterium pisicola
MSTLAEIINSSSSDINVEFKRAFRPLSPNIDITDNEDIALIILTNLTAKVDAKQILTDRENCRKKLKDNNWWSRCINTMKFRQSHNVKFPEIKATGIIRADPIGEIPKYFLSSSKLKQKTLPYANDSKDVSIANFLTSEFILNGKICCLGVLLADEQHPLWDRLTKLGCYQKTRKYVAKQLEKIPNSCIEVHLGANSVRQISLPDGNNSYLSLSPATSQTMQSSCYQSLHKHYWLSKTTRYSRASNMGVLPMTCGGALRMLSHTISFEDVKHCQISPDSYWLTKDAIQAFYDYFDYSNWLMPHTQKQQKLKTLKITITEMISRWLSLQKKSIKDNNLDLVHKLNFDLSNTKSLRQFAYDPKTTRFLFTLIENRSNTQQPYTSPNENFDNQYLLLPQISVCGASALNSSITIGIPSLTAFFGFIHAFERNIQKVFPNFKVKSFAICIHTFHLQKRGMTRESVEKSKDNISPPATHDEWYCDLKFSLVLKINNLTNLNQSKILYALPKRLAGGCARISIDDIKTIQVSSSFKDLVKQIPTFQGQWLSLYKAEIDSFENIIQQIKASQNITPTCVGYHYLEKPTERPFSLRDYNHVFAEPIIGLIKPITIKEDTNLHDLFWNQINKPHYTTIETRNMNNEDPY

SEQ ID NO: 31 - protein, 349 aa, Photobacterium pisicola
MKIPISLAYERSIHPSDVCFFTIWPDGNKAPLSFMSRTALGQMETASRAYDTKGNPKKTATPEALAQGNPHHMDFCSVPFGAEHIECVFSVSFSSELRKPYKCSEPEVKNTLIELVKLYESKIGWDELAGRFLINICQGKWLWKNTKKAYQLNIEIKPWPWKNNPVIFEDIRTNYSTYNDCLSDPKWQELKHLIINAFSSQNGLTIFEIKAQLTLPTNAELYPSQTFIEKDKKEKKHQSSRIFQHTSINGQKSPIIGCYKAGAGIFMIDDWYPNAEEPIRIGRFGVHQQDVTCYRHPSTHQDLFSLLEQSEKYIALLKNRKKIPQKTINELHFLMANLIKGGLCQHKGE

SEQ ID NO: 32 - protein, 199 aa, Photobacterium pisicola
MDWYYKTIRFVPTNCNNEFLAAKCINILHGFSYRYDTRSIGVSFPNWCNETVGDRISFVSIEKMTLDYLLTQNYFKEMQDLGYFNISKTLIVPIECNFACFKRYQKIDKSSAAGFHRKIKRLAKRAHNRGEVFDNKKYNIYKQCDIGHYHSLAEESKSKGYGFRLNIQRTNEVSINNIPIFSSYGLANTAHTLQSVPII

SEQ ID NO: 33 - protein, 228 aa, Photobacterium pisicola
MQSIPPATYQTDSPLELAFIDPARKLTKSRGKNIHRYASVKMNTIISVESTLEFDACFHFDFNNNISRFCSQPIRYSYVIDGIIRTYVPDFLCEFDCGEMVLYEVKAPNAVNSKRFTKEFEAKRQYARKLFDVDLELIEEHDIRHIPLLKNLKHMHRYASHSELTYEQTTILKYLNKHGSTKLETIIDHFSNSKNIIPMIYDLLSKYLINTDLRVLLNNQTELKTTYV

SEQ ID NO: 34 - protein, 603 aa, Photobacterium pisicola
MSRTSFSSFHGTPTSDSENASSKLTFIENDDLQDLTSFSKKAQNEAKFRLSILRNILNKSSEINLVLIERLRADLQQQEERSIPSAITIYRWWLTYKKSNFSILSLVPKTKQKGNRNTKVPKTISVYIDQAVEQVISANKINVSTAFRRVRRKVRQYNLTHKTKHTYPSYEAIRKRVKKITPYEKLAASKGDRIAKREFRRMGKKIQTEYLLERVEVDHTWLDLFAVHEEHRVPIGRPYLTQLVDCHSKSVIGFYLGFEPPSYMSVALALKNAIKRKDNLLQNYPSIQNEWLCYGIPDLLVTDNGKEFLSKDFTLACDSLLINIHQNRVETPDNKPHTERQYGTTNTELLNDLPGKTFSNYLQREGYNSSDEASLTLSEIKQIYLTWLVDIFHPRPNSRGTNCPNRVWKHAEKNWPPNEFLGTDEELDFYFTKLDKRMLRKEGIGLATELFYSSERLAEYRGKKGNHKVTIKYNRENMGFIWVLDEDTESYFKVPAIDYEYASTVTLWMHKKNLKIKQELNITEHNLDSEIDAELRIDEIVDQSIQKKKTTITNKRRAARYQENEQMAANARQPKEKKSANNQKTSTKNIEKEDSSGWDIDYV

SEQ ID NO: 35 - protein, 328 aa, Photobacterium pisicola
MDDNITRIAKAKKAFISTPDVAAIINNIDRCRRLSKFGAEPSCMMVYGASGVGKTSIIRKYLKANDKDSTARQDIVPVVYIELPENAKPVDAARELLLYLEDPLALYESDLTILTKRIVDLVPTMKIELIIIDEFQHLVEKSSNRILSRVGDWLKMLINKTKCPVILFGMPYSKVVLAANTQLRSRFSIQFELKPFSYINNYGIYAGFLRRLDEALPFDQLSNLASEPIAKKIYAFSQGNMRSLRNLVFHAAVNAIENDNNCISKNDWEFASNLTSGNKLSTWKNPFVQGVKITDRMLQDQPNDLGWEDYMRNNRGKRKPFDESKIFS

SEQ ID NO: 36 - DNA, 28 bp, Photobacterium pisicola
  source 1..28: mol_type = other DNA
gtgtactgccgagtaggcagctgataac

SEQ ID NO: 37 - DNA, 28 bp, Photobacterium pisicola
  source 1..28: mol_type = other DNA
gtgtactgccgaataggtagatacattt

SEQ ID NO: 38 - DNA, 156 bp, Photobacterium pisicola
  source 1..156: mol_type = other DNA
tgttgaaacaaccataagttgatatttacaaccataagttgatatttaatacaaccataacttgatattgcctctttgtgtaccatacttacgtaagtttacgacatatggtgaattatagccatacagacccaacttgaagttgcagctagctaa

SEQ ID NO: 39 - DNA, 151 bp, Photobacterium pisicola
  source 1..151: mol_type = other DNA
cgcgacttacaaccaatcttcataattagtaataaacctatgaaaatatcaacttatggttgttttgaataatatcaacatatggttgtatttttattaaaaaattgatttatatgaaaatattgaatatcaacttatggttgtttcaaca

SEQ ID NO: 40 - DNA, 1191 bp, Aliiglaciecola sp.
  source 1..1191: mol_type = genomic DNA
atgctgctcatcaggacaaagccttttttagatgaaagccttgagagttacctgctaaggcttagtatccacaatggttacaataaatttcagtcgttttgggctggcgtgagatctcatcttaacgaatcaactcggggaattgattccgctttaccgagcgaattatcaaaaatcaatatatgccatgccaatgtatccagtgcaaaaagactggatgctttgcgccttgtgagtcagcttaccaatcacgaacctttgccacttctatcgttggccttgtttcgaggagggcaattgttttctagaaaacgcacatctgtattcaataacggagtcactattccatttcgtttcttaagaaccaaagggataccgatttgtccggcgtgcatcaaagagaacgtatacatcaggcagcattggcacttttcactatttgaagcttgcccggagcattctgtgttgcttcgtaatcactgtgactgtggcgaggaaattaactatttaagcagccacgagattgcacagtgtgcaaagtgcggcagtaatttggctgatttggaggcaactgtctcgtctgctcctcagcgggaaatcgcacattggctatcagggagactagtagaggggctgcctgctgtgattcaaagccattcatgggggatatgtttatggtggcaggaaactttcaatgatgggaaggacattgattccgaacaacttcacctgtttctcgcacaatggccagatagtttgcgtagctatttaaactgtaaactggcgcattcaaaggagtatgcgcttaaacccttcaatcagctgtcttttaaagatgtgtttgggcttttattgatacaagcttctcggctacccagtaccaatttgtcagaaaacatagtgttgaaggagattgtgcgttatctagaggaacatgttttcgaacctgaatgtctgttgagtgaccttaagctaaacagtatagaagcagcaattattttgggtacatcagtagagcaaatagctgttttagttgaccaaggtgaactacaaactaaatccagaatgaaggctaactcagttttaaacgcaaattggagagtgttgtccctgggagatgttttttgtctatggttagctaaatttcaaacggataactcccacagtaacgtgtttatttcacggtggtag

SEQ ID NO: 41 - DNA, 1935 bp, Aliiglaciecola sp.
  source 1..1935: mol_type = genomic DNA
atggcttcaaacgagataacgtcgttacttaatattgaaaatcacacagaccgaaacgtggcatggaaaaaggctctttctcctattactcctcctttagatgtcacaggaaatgaaaaactagcgtgtgtcgttttagcgaatttaacctggaagctgtcactaataaacaatgtctttgattcaaatgatgcaagagcaaagctaagggataagaattggattcaacgctgtataaaaacgtttagatatagacacactcacaatctgaagtatccagattatcgtgcaaaaggggccattcgcctgtctccaatcggtgttataccgaaaggctgctttagttcttcaaaactaattagtagtcgtttgggctggtctcagaattccgctgatatcaattatgcgacgtttctatgtgctgactttgtttggcaaggagagctgctaacgctaggtgaggctatcataggcgagaacatttcatttacgaaatctttgattgaatcggggatgtttaaaaaagatcttaagctgataagaaacgagttgtcccaaatcccaataaatcaaactgaatctgaatatcttagtcatcaattgacaaaccttcggtttccgaaacattcagatggatatgtttgtttgacgcccgtcccctctcatattgttcaggttgcaattcactcttggtctgtttccaattttcgccagtcggaaaccatgtattgtcctaggtcttccagtgttggctcactcccggcttgtgtcggaggaaaaatcaaagttctaaaaagtttgccaaaagggctcaatagtaagcacacaaaagatactcaaaaaagttcctggctgacggcagagaacttggctattttacactcattatcctcaagtagggattggcttttgcctgagaataaaaagaaaaagcggtataaagagttagtggctaaacttggagcaatgctggtacgttggatgtctttcaatcgaaagtctctagaacaattgttggaatccgagttcccgtcaaagcaaatcacacaacttttccatgcagatctgtctcgcttaaaaagtacagatgatatcgcttacaacccgaccttcatcaagatagtcgaacaggaattcaaaataatccttgaaaacgaaaaggaggattatccgttagtaataccgcagcaaaaacatacgcatcttgttttacctggccttagagtttctaatgccaatgcagagagctgtgcctatttggttggcttgccttctatgatcggcatttttggctttatacataatctccaaaggcaactcgatagtagatttggtttatctgctgggtttgagcaatttgctatttgcatgcatgaatatagtttccataaacggggcttaaccaaagagcaggttcaaatctccaaaaaacagcttagatcacctgcaataattgattccagacaatgtgacttcgccctgagtctcgtcattaaaactagtgcgatactgcaacgagaagaggtgttggcagcgttaccgcaaaaaatatgtggtggtgcggttcatattccactttctgaactagagggaataaacacccatcacagtttcgaatcggcggtaaacgcgatcccagtcaagaatgggaaatggataacgccttcttttaattccttgagtaccactaattttatcgatttcttggataaaacgtctgtttcgtacaacctcaatatcgcatgtgttggttatcactatctcgaaacacctttcaaaaagaatagtgcttcagatgatccagtccatgcatttgctgaacctattttggcaggcgtccaattgaattgtattgcttcatttggaaatatagagcgctttttttggcactacagtgaaacatcaactagcttatatttgggttctaaaatttga

SEQ ID NO: 42 - DNA, 1032 bp, Aliiglaciecola sp.
  source 1..1032: mol_type = genomic DNA
atgaggctaccaaacagactaagttatcaacgatcaatatcgcctggtattgcagtattctattcagtagatgaacagggcaatcaaaagccattagaaattaacacggtcaaaattctcgggcagaaaggtgggcctagcgaagcatttgctaatgatatgagtctgaaaaagggtgtggataataaaaagttagctgaaggaaaccctcacacaattgactattgttatgcacccgcagatgctaagcacaccttatgtaaattctctcttaacgtagatgcgagttcaattgagccgagagcctgtaatgatgacggtgttagaagtttactgactaatttcgctgctgagtatcgtaaattgggcggatacagatatctggcagagcgatatctgaacaatgtgctgagtggtaactggctgtggcgaaatcaaagaacgcttgatacaacaatcaaaatacagagttcaggaggattacagtgttcaataaaaggggtgaaccggaaacgattcgaaccaaattggattgatgagattactgaattcgatgggcttgtaaacgagtttgaaaatgctttagtagaccccaagaagtatctttttttagaggtaacggcagaattatcattaccattggctagcgaaatttaccccagtcaggcgtttgttgaacaagcgaacaagttagaacgcagcaggacatatcaaaacacgatagtggaaggaaaacgcacggcgattattggcgcttataagataggggctgcgatagcgagcattgacgattggtttgagggggcagatataccagtgagggtaggtagttttgcagttgatagagatagggcaacggtctatcggcatcctgaatccaagaaagatttttatacccttctaagtggcctcgaacaattgaacagcagattaaaatccaagaaaaagatgaaatcatcggaattgaacgatgcgcattttattgccgcgaatttggtaaagggtggattgttcagtttaggaagcaaatag

SEQ ID NO: 43 - DNA, 606 bp, Aliiglaciecola sp.
  source 1..606: mol_type = genomic DNA
atggcgtctcgctactatcgaaaaattacttttatcccggccgatagcaatcataactttctaattggcaagtgtctgaaagtgctccatggagtcaattgtcgacaccgattgaactcaattggtgtgacctttcctgattggtctgatgaatcacctggaaactctattgcctttgtgtcggtagatagtgcttgtatcgacttgttaattgaccaacattactatcaacaaatgcaagatttagagtatttcgaaatctctgcattaaaacctgttcctgagaatgggtcggaagaaataatgttttctcgaaatcaagctgttgatgaactaacgccagcaggtgtaagacgcaaactgagaagatgcgccagaagagctaagcagcgtggggagaactacaatgcagcttatctttctagtagtgagaaagtattcccacatttccacaaaataccaatgaacagcaagagtagtgatcgtaatttcagtctaaatatccaattagaaatggcgcaaaacgttacctatgggaattatacctcttatggtttgtcgaataaatcaagccggaaagcatcggtccctaaaaatctagattaa

SEQ ID NO: 44 - DNA, 627 bp, Aliiglaciecola sp.
  source 1..627: mol_type = genomic DNA
atgaaaaaacgtaaacttaccaaatctgcggtgaacaatattcatcgctttgccagttttaaaatggatgacttcattgaagttgaatccacgttagagtttgatgcctgttttcacttcgaatattccgctaaagttctcgaatttgaatctcaacccataggctttgaatacgagctagacggaaaaatccgcagttatacacctgattatcttgctcgtcttgaaacactcccatctactttttatgaagttaaactctataaaaaaacattgtcagaaatattcaaatctgaattcaaggccaagcaggtggctgcagaagccttgggaggaaggcttgaattaattacagaaaataacattagggtgtaccccttacttgataaccttaagattttgcatcgctatcattctgctgaaaatgatctcagcgaccagcagtatcaagctataactattctaggccgcgttgagcggcttagtattctggatttaattcataggatgggacagaactacagagaaattttcccggatatccttagtctggttgcactggatttactgaaattggacatgaacatgcctatcagtaccgattcgattatttggtgttcaaagtaa

SEQ ID NO: 45 - DNA, 1851 bp, Aliiglaciecola sp.
  source 1..1851: mol_type = genomic DNA
atggcttccgaagacacgttttcaggactcttcgatctagttgtcgaagaaaattgttcaatgccagatgggttgcaaccaactgagcctgcaacttttagggcgctttctgtttttactaccattcaacgtgatcaagcaatacatcgactaaatcttattaaatatcttttaaaagctggagtcagaagtttcacagaaaaaactatcacacccttattgcctgatctggtcacggaatttggcaatgatgtccccagttggcgaacgttggcaagatggtggtcactgtttaaagcttctgattttgatatcgtagctctcgtaccgcaaataacgaaaggaaactcgaacttcaaagcggatcctcttttagagccgctaatagcagaagcaatagggagaattatgtctgcagagagaccaaatcttgcagagggccatcgttttctggaaactttggtcttgagatacaacaaaggcaatgacacacagttgcaatgtatttcgtcagaagcgttacgcttgagagtgggaaaaattacgccttttgaggaaataaaagcgagaaaagggctaaccgctgccaataatgaatttcgagcaattggccaaaaaataaagacaacaagaattctggaaagggtcgaagttgaccacacaagactggacttgttcgttattgacgatatctatttcatacctatgggacgcccgtggttaacgatgttgatagattctttcagcttgtcggtagtgggtttttatttagggtttgaaccacccagttttgtttcagtaagccacgccctaaaaaatgcaatattgcctaaaagctatgtcaaagagaattatcctcaggtaaataatgaatggatatgttcgggactaatcgaattattagttacagataatgggcgagaatttgatgacaaagattttaaagtggcgtgtgcagagttgggtatgcatgttggcaaaaacccaactaagaagccctacctaaaagcttctgtggaacgtttctttggaactgtcaattctcgcttattagctagtccccctgggaaaactttccccaacattttcgagcgggacgactatgaccctgaaaaaaatgctgtcatttcgctctctaaaataaaccttctaatacataaatggatcattgacgactatcaacaggaccccaatgcgcgttggacaaatatgccgaatttgtcctggtcagttgcggcacaatctttccctccagccacgtacaatggttcgatagatgagttggattttaaattggggcgcaggtttgaacctaagctacgtaaagaaggaataaccaaagacaagcttcgatatcatagcgacagactggcatcttatcgtggaaggtatggagaccatagggttattgccaagcaagaccctaataacttgggtcgaattgttgtattggataatgacaaaaaagaatatttttttgtgcctgccgttgattttgattatgcgaacggactcactctttggcagcacaatcttcatcgtaaatatacaaaagaatttatcaaggcgaattacaaccaccaagatgtagtccaggcacgttcggaaattatcgatatagtggaaggttgcatggcagagatggcaactggaaaacgtaaaaagatcagtgtgacaaatcgtgttcgagcagggaggtatttagaagcagatcgcagacgggagctgccatcaccaaatacaagcgagaccgttgaacgtaatgaaaaaaaagaaatccctttctcagaagaaagttgggatgaagacgttgatatctccgaatggacatctagtcaggttaggaagtga

SEQ ID NO: 46 - DNA, 1008 bp, Aliiglaciecola sp.
  source 1..1008: mol_type = genomic DNA
atgtctgaatttggcgaaaaattaaagttggtcagagagctgtttattgctggcccttaccttgaatccttgatgtgcgaaatcgatgaatgcaaagaagattcaaagttgggtggagaagcgcagtgtatgtttattacgggaaacactggttcagggaaaacgacacttattcgaaaatatatggaaaactacccaagaaaggagttggctgacagaaccaaaatccccgttttttttacaagtctgcctgaaaacgccacaccagttcgtgcttctcagaaaatgttaactgatttgggagaccccttcagttgtgtaagcagtgatcttgaagaactcagaattaaactgatttgtttactggtgagttgtggtgttgagctaataattattgatgagtttcaacatttgattgagcgaaaaaacaacaaggtcttacatagagcagcagactggctaaaaaccattattattgattccaatattcctgttgtgctagtaggaatgccttacagttcagtaattcttgacgtgaactctcagttaaacgatcggatgctgtttaaaagaaggctaccaccatttagagttgaagaagaatcagaacgcaaagtgtatctgcaatttctgaaggtgtttgatttggcgttgccattccctgacagtagtagtctgcaaaccagggaggttgcactaagactttattctcattcaaagggtaatctgcgcaaacttcgtgagctacttaaccaagcatcacgggatgcactcctaatgtccgccaattgtattacgtcggaacactttaaaagtgccattgataagataaatggaaattactcggataccgttaacccttttaacgtgagtcatataaatgacgtcgctattgacgagccggacttggatataggttgggaagatttcaaaaacaaaccgggcgaaatccttgtcggtaaatcgagtcgtcaattcacagtaggagatattttcgctacccgttag

SEQ ID NO: 47 - protein, 396 aa, Aliiglaciecola sp.
MLLIRTKPFLDESLESYLLRLSIHNGYNKFQSFWAGVRSHLNESTRGIDSALPSELSKINICHANVSSAKRLDALRLVSQLTNHEPLPLLSLALFRGGQLFSRKRTSVFNNGVTIPFRFLRTKGIPICPACIKENVYIRQHWHFSLFEACPEHSVLLRNHCDCGEEINYLSSHEIAQCAKCGSNLADLEATVSSAPQREIAHWLSGRLVEGLPAVIQSHSWGICLWWQETFNDGKDIDSEQLHLFLAQWPDSLRSYLNCKLAHSKEYALKPFNQLSFKDVFGLLLIQASRLPSTNLSENIVLKEIVRYLEEHVFEPECLLSDLKLNSIEAAIILGTSVEQIAVLVDQGELQTKSRMKANSVLNANWRVLSLGDVFCLWLAKFQTDNSHSNVFISRW

SEQ ID NO: 48 - protein, 644 aa, Aliiglaciecola sp.
MASNEITSLLNIENHTDRNVAWKKALSPITPPLDVTGNEKLACVVLANLTWKLSLINNVFDSNDARAKLRDKNWIQRCIKTFRYRHTHNLKYPDYRAKGAIRLSPIGVIPKGCFSSSKLISSRLGWSQNSADINYATFLCADFVWQGELLTLGEAIIGENISFTKSLIESGMFKKDLKLIRNELSQIPINQTESEYLSHQLTNLRFPKHSDGYVCLTPVPSHIVQVAIHSWSVSNFRQSETMYCPRSSSVGSLPACVGGKIKVLKSLPKGLNSKHTKDTQKSSWLTAENLAILHSLSSSRDWLLPENKKKKRYKELVAKLGAMLVRWMSFNRKSLEQLLESEFPSKQITQLFHADLSRLKSTDDIAYNPTFIKIVEQEFKIILENEKEDYPLVIPQQKHTHLVLPGLRVSNANAESCAYLVGLPSMIGIFGFIHNLQRQLDSRFGLSAGFEQFAICMHEYSFHKRGLTKEQVQISKKQLRSPAIIDSRQCDFALSLVIKTSAILQREEVLAALPQKICGGAVHIPLSELEGINTHHSFESAVNAIPVKNGKWITPSFNSLSTTNFIDFLDKTSVSYNLNIACVGYHYLETPFKKNSASDDPVHAFAEPILAGVQLNCIASFGNIERFFWHYSETSTSLYLGSKI

SEQ ID NO: 49 - protein, 343 aa, Aliiglaciecola sp.
MRLPNRLSYQRSISPGIAVFYSVDEQGNQKPLEINTVKILGQKGGPSEAFANDMSLKKGVDNKKLAEGNPHTIDYCYAPADAKHTLCKFSLNVDASSIEPRACNDDGVRSLLTNFAAEYRKLGGYRYLAERYLNNVLSGNWLWRNQRTLDTTIKIQSSGGLQCSIKGVNRKRFEPNWIDEITEFDGLVNEFENALVDPKKYLFLEVTAELSLPLASEIYPSQAFVEQANKLERSRTYQNTIVEGKRTAIIGAYKIGAAIASIDDWFEGADIPVRVGSFAVDRDRATVYRHPESKKDFYTLLSGLEQLNSRLKSKKKMKSSELNDAHFIAANLVKGGLFSLGSK

SEQ ID NO: 50 - protein, 201 aa, Aliiglaciecola sp.
MASRYYRKITFIPADSNHNFLIGKCLKVLHGVNCRHRLNSIGVTFPDWSDESPGNSIAFVSVDSACIDLLIDQHYYQQMQDLEYFEISALKPVPENGSEEIMFSRNQAVDELTPAGVRRKLRRCARRAKQRGENYNAAYLSSSEKVFPHFHKIPMNSKSSDRNFSLNIQLEMAQNVTYGNYTSYGLSNKSSRKASVPKNLD

SEQ ID NO: 51 - protein, 208 aa, Aliiglaciecola sp.
MKKRKLTKSAVNNIHRFASFKMDDFIEVESTLEFDACFHFEYSAKVLEFESQPIGFEYELDGKIRSYTPDYLARLETLPSTFYEVKLYKKTLSEIFKSEFKAKQVAAEALGGRLELITENNIRVYPLLDNLKILHRYHSAENDLSDQQYQAITILGRVERLSILDLIHRMGQNYREIFPDILSLVALDLLKLDMNMPISTDSIIWCSK

SEQ ID NO: 52 - protein, 616 aa, Aliiglaciecola sp.
MASEDTFSGLFDLVVEENCSMPDGLQPTEPATFRALSVFTTIQRDQAIHRLNLIKYLLKAGVRSFTEKTITPLLPDLVTEFGNDVPSWRTLARWWSLFKASDFDIVALVPQITKGNSNFKADPLLEPLIAEAIGRIMSAERPNLAEGHRFLETLVLRYNKGNDTQLQCISSEALRLRVGKITPFEEIKARKGLTAANNEFRAIGQKIKTTRILERVEVDHTRLDLFVIDDIYFIPMGRPWLTMLIDSFSLSVVGFYLGFEPPSFVSVSHALKNAILPKSYVKENYPQVNNEWICSGLIELLVTDNGREFDDKDFKVACAELGMHVGKNPTKKPYLKASVERFFGTVNSRLLASPPGKTFPNIFERDDYDPEKNAVISLSKINLLIHKWIIDDYQQDPNARWTNMPNLSWSVAAQSFPPATYNGSIDELDFKLGRRFEPKLRKEGITKDKLRYHSDRLASYRGRYGDHRVIAKQDPNNLGRIVVLDNDKKEYFFVPAVDFDYANGLTLWQHNLHRKYTKEFIKANYNHQDVVQARSEIIDIVEGCMAEMATGKRKKISVTNRVRAGRYLEADRRRELPSPNTSETVERNEKKEIPFSEESWDEDVDISEWTSSQVRK

SEQ ID NO: 53 - protein, 335 aa, Aliiglaciecola sp.
MSEFGEKLKLVRELFIAGPYLESLMCEIDECKEDSKLGGEAQCMFITGNTGSGKTTLIRKYMENYPRKELADRTKIPVFFTSLPENATPVRASQKMLTDLGDPFSCVSSDLEELRIKLICLLVSCGVELIIIDEFQHLIERKNNKVLHRAADWLKTIIIDSNIPVVLVGMPYSSVILDVNSQLNDRMLFKRRLPPFRVEEESERKVYLQFLKVFDLALPFPDSSSLQTREVALRLYSHSKGNLRKLRELLNQASRDALLMSANCITSEHFKSAIDKINGNYSDTVNPFNVSHINDVAIDEPDLDIGWEDFKNKPGEILVGKSSRQFTVGDIFATR

SEQ ID NO: 54 - DNA, 28 bp, Aliiglaciecola sp.
  source 1..28: mol_type = other DNA
gtgttccgccgcataggcagctaaaaat

SEQ ID NO: 55 - DNA, 28 bp, Aliiglaciecola sp.
  source 1..28: mol_type = other DNA
agtttctgccgaaaaggcagttgatgaa

SEQ ID NO: 56 - DNA, 28 bp, Aliiglaciecola sp.
  source 1..28: mol_type = other DNA
tatttctgccggacaggtagctaaataa

SEQ ID NO: 57 - DNA, 28 bp, Aliiglaciecola sp.
  source 1..28: mol_type = other DNA
aatttccgccggataggcagctttcaat

SEQ ID NO: 58 - DNA, 28 bp, Aliiglaciecola sp.
  source 1..28: mol_type = other DNA
atgatccgccgcgtaggcagctaaaaat

SEQ ID NO: 59 - DNA, 132 bp, Aliiglaciecola sp.
  source 1..132: mol_type = other DNA
tgttgatccaagcataccttgacataatccaagcatacgttgacataagcaacaagcataccttggcatagttcaaaaaatagactacttttaagtaagtttacaacaataataaagcgctagacatgaaaa

SEQ ID NO: 60 - DNA, 192 bp, Aliiglaciecola sp.
  source 1..192: mol_type = other DNA
gtttctgccgaaaaggcagttgatgaaagaaagtttcttacattaattatctaagtaaaatttgcatgtccccgaatttgataaatgatgacaaggtatgcttgttattatttatgccaaggtatccttgtggaacaattaaatagttgatttatatcacttctatagtgtcaacgtatgcttggatcaaca

SEQ ID NO: 61 - DNA, 1239 bp, Halomonas titanicae
  source 1..1239: mol_type = genomic DNA
atgaaattgcttgtccgcccaagaccgttcattaacgagtcacttgagagctacatgctccgtttgtcgcaagagaatttcttcgaatactatcagcagcttagccgagcgataaaagactggctgcagttgcatgatcacgaagcagcaggggcgttcccagaagagttaagtcggctaaatgtctaccatgctgcgcagagtagtagccgacgcatcagagcgctcaagctagtggagtcgctaactgataatgaaaaacttccgctgctacacttgtcagtaatgcactccagcgagaaattttgttcacgctacgctagcgtgttctatgccggtagccacctacccagagcgttagtacgccaaaaggagatacctgtttgcccggattgccttactgaagctaactacattcgtcaggaatggcattggatgccatacgaagcttgtattaatcacggcaaacagatgctgcatgagtgtccaaaatgcgaagagaaattaaattacactcattcggaatgtttgcacacttgccgctgcggattcgatcttcgtaacgccgacacagagccagcagacgaatggcagctgatagcgagtcgcttggcagtaggtgagcattcgccttcaaggcatcctctattagacatccgacccatcagtttgcgacttgcgtgcttgctgtggtatcagctatacacatataaaaagctagacgccagcgatcaggttcccactcgtacgattgagcaagctatagagtatttcacgcattggcctgaagtgtttacacaggaactagagcagcaagctgcgctctcaggcgataagttgatatgtgattataataaaacctcatttcgcgatatttttggcaatattgtcggcatttcgcgccttcttttgcaggcttccccagagagtgatttcgtcttggcgcctttagaaagttttttagcccgattagtagagcagaatccgcaaacgcgtgtgccaaacgtcgcagactttcttatcagcatgtcagaggcagctattctgctaggcacatcgtatgagcaagtgtaccgactttatgaagaagggtatctaaaaattgcggtcagattgaagtctcacgaaaagctagtgaacggtaaaggggtgttttacttaagagaaatcatggagttacgtaaatcacgcatgccgattgagacaggcaactacaacaattatttaccagcgtggtaa

SEQ ID NO: 62 - DNA, 2058 bp, Halomonas titanicae
  source 1..2058: mol_type = genomic DNA
atgtcttttattaacttgttagaaatcgatgagccaagccgctcagaagcgattcgaaaagcctttgctccctacacgccgctgctagaggtttctgaaaatattagcgctgcaattttggcactgctcaacttatcgcataagcgtcagtttgcacccgatttattgaataaaaaaagagcagttgaaacactcaaagattgggagcatatagaaagctgcgcgcaagaaatgcaatggctgcactcacataacttaaagctccccgatacacgtgtcgcacatcagagattgttagtaagagctgagaagccctcggggaatgtcctttcttcatttaatagcgtgagtcgtttgggctggtcacacaactcagcagctgtaaataaagcaaagctttttggagctcaatttatatttaatggtgagacacactgtttggctactgtgattatggataatgaagaaagatggaaggaagaattcaagaagcttggtatatctgctggtcagtggatttaccttcagtcgcttttcgaggcatattttactgaaaatctattgccctcttatgtggataggtgttctgtacaggtgacatttatttaccaagggaaagatgtaagcattacaccggtgacttcacattcattgttggccgatattcaaattgctcgccgcaataaaactgggaatttttcaacaataaaacattggcacccctcaagtgttggagatttatcgtcatcacttggaggcaatatttcagctcttaattatccacctcgcttgctatctaatagctgttataaaaataagaatttttctgaaggatcttttttagattttcatcacagctctttgagaagtaagtcgttcatctttgcatgtaatgatattatcgaaagtaaaagttttttgacagcgaaggagaggcgagatgctagacgtgctggcatcaaattgctgaggcagtctttatctgaatggctatctccagttagctattggagaagtgtaggaagagaagtaatttcagagcggcataacaatacagcatgccttctgatttctgctcttgacgaggatgtacttggaattctacctgaggttaataaagaattgcacagtattttggtaagatatccccagacacaaagctttgcttaccgctcagatttattaattcctttcaagtctcagcttaaatttttactgacaaaattaaaaaaacaaaaaggtgaagacacagttgaaaataattttcattatcttcatttgaaaaacttgcatatctttgatgctcaagcgctatcttgtccctacatagtgggcctgccttcactgctagcagtatggggaaccgtttataactaccagctacgtttacgaaaaattcttaaacgaagcatcgtatttgaagaagttgcttggtttttgcggcaatatgagccttcttcgggtgccaagatacctgctccttaccaaccaccagtgaagccaggtggggctcctaaacgccccggcctgattgatatgcgcttttgcgatttacgcatggatttagttattcgctatcgtatagaggatagagacgataccccggtgggaaatgaagaactgccaatactgcagtctgctttccccggaaggttcgcgggggggactatgcagcccccgccacttcatgaagagttgcagtggtgccaagtatacgcagatgagaacagtttattggcagccatatcgcttctgccggacgaaggtcgttggattgttgactcagaaaaacaggtgcaaagcattgaaagcttggtggcatggttgtccaagcatcccaatcatttacctgctacgagtggctatcagttgcttgaagaaccttgctatcggtctggttcgcacagggagcaccatgcttatgctgagcctttggtcggtctaacagagacgttgtcaccggcaagcgtgaggctcaatggtaaagctgactttcttaaaaacgctttttggaggttgcaaagccagaatctgactatgctgatgaaaaaagcatag

SEQ ID NO: 63 - DNA, 1050 bp, Halomonas titanicae
  source 1..1050: mol_type = genomic DNA
atgatgaactctttcaggcatcttagctatgagcgctctctcaatccgggtaaggcggttttttattaccggacagattcgagcgaatttgagccgttgcaagctgaagtgacacggtttcgtggccccaaggcaacgtttagcgatggctatatggcatctgggactgcgcgcgcaaaagaaacaagtgatttaggattttctaatcttatcatgctcgaagcgtgttatgtaccaccgttagtcgatacggtgtattgtcgtttttctttacgcatcattgcaaacagtttagaaccaaatatctctgataatgcagaagcaacaaaagcattgagagaattttctgatacgtatagaagtttgggcggatactatgagttagcaacccgatatgctaaaaatattctttcagcagaatggctttggagaaataaagttgcaagaggagtagttgttgaagtttctacctctaatttaaaaaactactgtgttaaagatgtacagtacaaagaatggggttcttcttggggaggagatgatcttaaatcattagaaggactagcagccgagcttgaagaagcactttcttgcccacaaaaatttctttttgctgatgtaactgccaaaataaaaacagaattttgccaagaaatctttcccagtcaattatttgttgagaaagatgatagaggcaacgaaagtgcggctagacagtttgtgaagagcacaatgagtgatggtcgtcaagcagttagctttggcgcttataaagtcggagcagcaattcaaaaaattgatgactggtggttaactgaaggtgcagaatatcccttaagggtaagtgagtacggtacagatcgtagtcgagttttagctatgcgtgaacctactacaaaaagagatttttattcattgttagacgaaattataagtatgactgaggaaatgaataaatccaagcagcctagttacagtgctcactacttaatgtcagtgttagtaaagggtgggatgtttcaaaaaggaattaagaagggtgagaaatga

SEQ ID NO: 64 - DNA, 609 bp, Halomonas titanicae
  source 1..609: mol_type = genomic DNA
atgagatactttttccacattagatatctaatgccttcagccaatcatgcatttttagcaggtaggtgtattgcttgcttacatagctttatgactgggccaaagataacaaatcatggtgttggtgtttcttttccattatgggctacggatacagtcggaggttcaatagctttcgtgagtaaagataataatgccttgtctaatttatcatcagcgcgttattttaagaatatagctgacgaaggatttattgaagttagtggtgttaaaatggttccatccacattagaggaagtgaggtttattagaaaccagcatattgctaagtcttttccgggcgaaattaagagaagattggcccgtggcaaaaagcgggctgaaaaacgtggtgagacgttcatgcctattagcgttgtatcagatcgtttggttgatcactgccacgtaattcctattgatagccgctcgtcaggccaacgctttccgctgtatgtccagcttgaggcactcagcgaagaaagcaagcatgatagctacaatagctacggcttggctacacaacactcgcatactggctctgttccaaatcttaaacatattacctaa

SEQ ID NO: 65 - DNA, 624 bp, Halomonas titanicae
  source 1..624: mol_type = genomic DNA
atgtacaggcgtaagctccgtcattcccgtgtcaaaaacctctataaatttgctagcttcaaaactgcttctgctcacactgtagagtcgtccctagaatttgatgcctgctaccactttgaatactctcctcacgttaagtcttttattgcccagcctatgggttttacttactcaattcatgggaaaactaacccctacactcctgattttaagataataaataataatcaaaaaatggcctttattgaggtcaagccccatagtaaaaccctccatccagaattttttcaaaaattccaggcaaaaaaagaagcagcatgtcagttaggttttgagctgagcttagtaactgagcttcaaatcagaaagtatccagtcttgaataattttaagctgctacatcgttacgctggttttcagagccattgcgagttgtatgatagtgtttatggccttgtaaaaattcactcccctattttcttgcatgaaatttgtgctctatatgacattagcttccgtcctagagttattcgctcactggtttcgttaatagcctctgggaaattaaaggccaatatattggaaaagcaaattggtgacgatttgcttctttgggcatga

SEQ ID NO: 66 - DNA, 1896 bp, Halomonas titanicae
  source 1..1896: mol_type = genomic DNA
atgttggaagatcctttttttgatgagtttttagcaggcatagatttttcgcaagcagcgagtaaaaaaagccgtgatgaaattgaggatgtggcattcataacaatagatgatcttaatgaggattgcgccgataaagctttgttcaagtataaggtaataaagttagtcaataaaaggcttaatggaggctggacgaagaagaatgtagagccaattatctatgaattgtataatgagggtgttatagataaaaagcctgggtggcaaagcgtagctagatggaatgcaaagtatcgtgtcgataaaaatttgctttccttggttgatataagagctataaaaaataatttttgtgattttagttctttttcaaaagacactttcttttgggacgctgtcgaaaaaaaatatcttacacgagtgagggggtctgttgcgaccacttatcagttttataaggatttaatattagtccataataatgaaaatccagatgatcagtttgaagcggttgggcgaagtgccttttacgatagagttaaaaagcttccaccttatatttgtgacttgaagcgttatggcaagcgatatgcggataaaaaatatcgattgatcaatagtttcacaaaatctactagagtgatggaacgtgttgaaattgatcatactgctcttgacttgatcttgcttgatgataccttaaatacccctattggaaggccatttataactgtcttgattgatacatttagtaaatgtatcgtgggtttttatttaagctttagagggccgagctataattctgtaaggtgtgcagtgttaaatgcgtgcttggataaaaaagacttgttggaaaagtatccagacatcgaaaaagattggccatgtcaagggcgtattgaaacacttgttgttgataacggcgcagagttttggtcgaaagatcttgagagattttctacctcaataggaatgagcgttgagtacaatccagttggaaaaccgtggaagaaacctttagtcgagagaatatttaacacctacaatacagagtttattcatcagataccgggaaaaactttctcaagtgcaaaagatttagaaggttatgagcctgagaaagatgcgttattaccattttccaggtttttacatctgcttcatatatgggttgtagatatttataatcagcagccaaataaacgtagaaccaatatacctgctttatcttggcagattgggtgtcaagattttccaccggttgtttaccaaggggctgaaaaacaaagatttaaaatcgaaagctttcctaccgtctatcgagatttacgaccaattgggatagaggtggatcatatcagctatagcagcgaaatgttggtagagttcagaaaaaatcacccgccgccccccggtcagaaaaagcataagctttgtgttaagcgagacccgtctgatgtttcttttgtgtatgtattcttacctggattggagaagtatatcaaagttaatgctactagccaggagtttcatttagagggcgtctctattttccagtaccaagttatgagaaggttgttgaataatcatattgaaattaataccgatcatgaggggcttgcccttgcaaatatgaagctcagcgagagaatggatgatataaggggaaatgctttagtcaagaagagaagtagcctaaaaggaatgaaaagcgttgctgctttcataggcgtggatagtgagagtgatgcgacttttgaaagtgtgcaccgtgacatgaaaagtagagataacgctatgtgtgattcttcagaagacggcttaacagattcaaaaaactttgagcttgttgatgattggcacgatattgctgttgatgtggagccttattaa

SEQ ID NO: 67 - DNA, 1020 bp, Halomonas titanicae
  source 1..1020: mol_type = genomic DNA
atgagcaacatactgtctgaagctaaatgtagtcaattgatttattttagaaactgttttgtccaacatgcagccttaatacaggcgatgagttctttgaatcgattgaaaaataatcagctattaggcggagaacagcagtgtatgttgctgttaggagatacagggactggtaaaagtaaattaatcaatgagttttattcggcatttccaagctacagagagggcgatgttctagtaaagcctgtgctcgtttcgcgcataccaagtaagcctgatgttgaaaaaatgatgattcaattattgagcgatttggggtattttggttcagaagcaagaaaaggtagtcgtcgtgagacgggcctagcagaggcattagtcaaagtattaaaaaaatgtaaaacacaaataatcatcatcaatgaatttcaagagttaatagagtttaagtcggttgaagatcgtcaacgtgttgccaatagacttaagttgctcagcgagcaggcttgcatacccatcgttttagtaggtatgccctgggcttctgaaatatctaacgagcctcaatgggcctcgcgattaatgtgttcaatagaactgccgtattttaaaatttttaaacaaatcgatcgtgttgagtatgtaaggtttttaaaaggcttggcgttaaggatgggctttcatgaaccgccgccacttcatagcgatgagatactatttccacttttttctgcaacgcgaggtgaggtaagaaaaattaaacacattctcgatgaggttctttttttagccttgtcgcagggagataatactgttcataggcagcattttgttgatgttttggataacgttttttttgataaagataatccattcaagctgcctattgatgatgtcccactgagcgaagtatctcgatatgcatcttataatcgctactcatcggttgagtctgagacttttataggaactcagtttggtcggaaaatttcgactagagaattattttctaaaaattag

SEQ ID NO: 68 - protein, 412 aa, Halomonas titanicae
MKLLVRPRPFINESLESYMLRLSQENFFEYYQQLSRAIKDWLQLHDHEAAGAFPEELSRLNVYHAAQSSSRRIRALKLVESLTDNEKLPLLHLSVMHSSEKFCSRYASVFYAGSHLPRALVRQKEIPVCPDCLTEANYIRQEWHWMPYEACINHGKQMLHECPKCEEKLNYTHSECLHTCRCGFDLRNADTEPADEWQLIASRLAVGEHSPSRHPLLDIRPISLRLACLLWYQLYTYKKLDASDQVPTRTIEQAIEYFTHWPEVFTQELEQQAALSGDKLICDYNKTSFRDIFGNIVGISRLLLQASPESDFVLAPLESFLARLVEQNPQTRVPNVADFLISMSEAAILLGTSYEQVYRLYEEGYLKIAVRLKSHEKLVNGKGVFYLREIMELRKSRMPIETGNYNNYLPAW

SEQ ID NO: 69 - protein, 685 aa, Halomonas titanicae
MSFINLLEIDEPSRSEAIRKAFAPYTPLLEVSENISAAILALLNLSHKRQFAPDLLNKKRAVETLKDWEHIESCAQEMQWLHSHNLKLPDTRVAHQRLLVRAEKPSGNVLSSFNSVSRLGWSHNSAAVNKAKLFGAQFIFNGETHCLATVIMDNEERWKEEFKKLGISAGQWIYLQSLFEAYFTENLLPSYVDRCSVQVTFIYQGKDVSITPVTSHSLLADIQIARRNKTGNFSTIKHWHPSSVGDLSSSLGGNISALNYPPRLLSNSCYKNKNFSEGSFLDFHHSSLRSKSFIFACNDIIESKSFLTAKERRDARRAGIKLLRQSLSEWLSPVSYWRSVGREVISERHNNTACLLISALDEDVLGILPEVNKELHSILVRYPQTQSFAYRSDLLIPFKSQLKFLLTKLKKQKGEDTVENNFHYLHLKNLHIFDAQALSCPYIVGLPSLLAVWGTVYNYQLRLRKILKRSIVFEEVAWFLRQYEPSSGAKIPAPYQPPVKPGGAPKRPGLIDMRFCDLRMDLVIRYRIEDRDDTPVGNEELPILQSAFPGRFAGGTMQPPPLHEELQWCQVYADENSLLAAISLLPDEGRWIVDSEKQVQSIESLVAWLSKHPNHLPATSGYQLLEEPCYRSGSHREHHAYAEPLVGLTETLSPASVRLNGKADFLKNAFWRLQSQNLTMLMKKA

SEQ ID NO: 70 - protein, 349 aa, Halomonas titanicae
MMNSFRHLSYERSLNPGKAVFYYRTDSSEFEPLQAEVTRFRGPKATFSDGYMASGTARAKETSDLGFSNLIMLEACYVPPLVDTVYCRFSLRIIANSLEPNISDNAEATKALREFSDTYRSLGGYYELATRYAKNILSAEWLWRNKVARGVVVEVSTSNLKNYCVKDVQYKEWGSSWGGDDLKSLEGLAAELEEALSCPQKFLFADVTAKIKTEFCQEIFPSQLFVEKDDRGNESAARQFVKSTMSDGRQAVSFGAYKVGAAIQKIDDWWLTEGAEYPLRVSEYGTDRSRVLAMREPTTKRDFYSLLDEIISMTEEMNKSKQPSYSAHYLMSVLVKGGMFQKGIKKGEK

SEQ ID NO: 71 - protein, 202 aa, Halomonas titanicae
MRYFFHIRYLMPSANHAFLAGRCIACLHSFMTGPKITNHGVGVSFPLWATDTVGGSIAFVSKDNNALSNLSSARYFKNIADEGFIEVSGVKMVPSTLEEVRFIRNQHIAKSFPGEIKRRLARGKKRAEKRGETFMPISVVSDRLVDHCHVIPIDSRSSGQRFPLYVQLEALSEESKHDSYNSYGLATQHSHTGSVPNLKHIT

SEQ ID NO: 72 - protein, 207 aa, Halomonas titanicae
MYRRKLRHSRVKNLYKFASFKTASAHTVESSLEFDACYHFEYSPHVKSFIAQPMGFTYSIHGKTNPYTPDFKIINNNQKMAFIEVKPHSKTLHPEFFQKFQAKKEAACQLGFELSLVTELQIRKYPVLNNFKLLHRYAGFQSHCELYDSVYGLVKIHSPIFLHEICALYDISFRPRVIRSLVSLIASGKLKANILEKQIGDDLLLWA

SEQ ID NO: 73 - protein, 631 aa, Halomonas titanicae
MLEDPFFDEFLAGIDFSQAASKKSRDEIEDVAFITIDDLNEDCADKALFKYKVIKLVNKRLNGGWTKKNVEPIIYELYNEGVIDKKPGWQSVARWNAKYRVDKNLLSLVDIRAIKNNFCDFSSFSKDTFFWDAVEKKYLTRVRGSVATTYQFYKDLILVHNNENPDDQFEAVGRSAFYDRVKKLPPYICDLKRYGKRYADKKYRLINSFTKSTRVMERVEIDHTALDLILLDDTLNTPIGRPFITVLIDTFSKCIVGFYLSFRGPSYNSVRCAVLNACLDKKDLLEKYPDIEKDWPCQGRIETLVVDNGAEFWSKDLERFSTSIGMSVEYNPVGKPWKKPLVERIFNTYNTEFIHQIPGKTFSSAKDLEGYEPEKDALLPFSRFLHLLHIWVVDIYNQQPNKRRTNIPALSWQIGCQDFPPVVYQGAEKQRFKIESFPTVYRDLRPIGIEVDHISYSSEMLVEFRKNHPPPPGQKKHKLCVKRDPSDVSFVYVFLPGLEKYIKVNATSQEFHLEGVSIFQYQVMRRLLNNHIEINTDHEGLALANMKLSERMDDIRGNALVKKRSSLKGMKSVAAFIGVDSESDATFESVHRDMKSRDNAMCDSSEDGLTDSKNFELVDDWHDIAVDVEPY

SEQ ID NO: 74 - protein, 339 aa, Halomonas titanicae
MSNILSEAKCSQLIYFRNCFVQHAALIQAMSSLNRLKNNQLLGGEQQCMLLLGDTGTGKSKLINEFYSAFPSYREGDVLVKPVLVSRIPSKPDVEKMMIQLLSDLGYFGSEARKGSRRETGLAEALVKVLKKCKTQIIIINEFQELIEFKSVEDRQRVANRLKLLSEQACIPIVLVGMPWASEISNEPQWASRLMCSIELPYFKIFKQIDRVEYVRFLKGLALRMGFHEPPPLHSDEILFPLFSATRGEVRKIKHILDEVLFLALSQGDNTVHRQHFVDVLDNVFFDKDNPFKLPIDDVPLSEVSRYASYNRYSSVESETFIGTQFGRKISTRELFSKN

SEQ ID NO: 75 - DNA, 28 bp, Halomonas titanicae
  source 1..28: mol_type = other DNA
gtgaaccgccgcataggcagctgatatt

SEQ ID NO: 76 - DNA, 28 bp, Halomonas titanicae
  source 1..28: mol_type = other DNA
acacaccgccgcaatggcagtaattgtt

SEQ ID NO: 77 - DNA, 28 bp, Halomonas titanicae
  source 1..28: mol_type = other DNA
gtgaaccgtcgcataggcagctgaaaat

SEQ ID NO: 78 - DNA, 28 bp, Halomonas titanicae
  source 1..28: mol_type = other DNA
gtgaaccgccgcataggcagctgaaaat

SEQ ID NO: 79 - DNA, 121 bp, Halomonas titanicae
  source 1..121: mol_type = other DNA
tgtcgtttctggtctgttttgacataaaatccggttaaaatgacataatttgcggtttctcgtgacataacctatttttgtagtaaacctacgaaatggggtgctgtcatgtacaggcgta

SEQ ID NO: 80 - DNA, 190 bp, Halomonas titanicae
  source 1..190: mol_type = other DNA
cgctcagtttctgcggcaaaaaagcagataatcttaatgtatatttatgacgtgattttattacagaagcaaacgttcgttttattttatgtcatctaaaaatggaaaatttatgtcattttgcggtggaatttttacgtaagttactgatttttagatttgcctatgtcaagttgaaacggaaacgaca

SEQ ID NO: 81 - DNA, 621 bp, Neptunomonas qingdaonensis
  source 1..621: mol_type = genomic DNA
atgtatcgacgtaatcttaaacattcccgcgtaaaaaacctttttaagtttgccagtcttaaaattgatgacgtcattaccgttgaatccggcttagagttcgacgcctgctttcactttgagtattcacccgatgttatttctttcgacagtcaaccggaaggtttctactatagttttttgggtaaagagctgccttatacccctgacttcgttgtcgttgataaagtaaacggagcacgctttattgaagttaaacctgttgctaaagcagctaatactgattttagagcaaagtttatatgccgtcaggaacgggcggcggcactgggtattccggtgatattagtgactgagcgccagatccgcctaaatcctattttgaataaccttaaattattacatcgctatgcaggcctccaatcatttacggagctgcactttgctattttagagctagttaaaaagtcaggtatcgttagtgttcgcaacttgatatctagcactcaggctatagaaggtgatgtgcttgcttccacggtaacctggctctctttagggcggttgaaaacagatttatcctcatctgatttagggttagattctgtggtttggtgctag

SEQ ID NO: 82 - DNA, 1887 bp, Neptunomonas qingdaonensis
  source 1..1887: mol_type = genomic DNA
atggccacgccagatctgttatcaaagagttctggtttatttgaagatgaattctctccgaatgaacctgaaatccctttttttgaagaaccctctgcagaaatatttccgcgtgatttaaaaacttatccagctgagctgcgcgatgaagcccttaggcgcttagcttatattcagtgggttggaaaaaggcttgaaggtggctggactgagaaaaatctaacgccattgttacatgtggttggagataaattactagcgccgttgccttcatggcgcacacttgccggttggcgaaaactatatgtatcttcaggtcgttctgttgaagcgctaattcctaaacaccacaagaaaggtaaccgtcttgagcaaaatacaggtgatgacctttattactggcaggcggttaatcaaaagtatttacgtccagaacgtccctcaatcgcgtcagtgtatgaatactatagagaccttatcacgctggcgaataagggcgtagttcaagggaaagtaatgcacgttagtcgcagcggattttacaatcgaatcaataagctaccaccctatgatgtcgcacttaaacgttatggtaaacaatatgcagatcgaaaatatcgtcctgtaggcgctcatattaccctcacgcgagtgatggagagagttgaaatagaccacacgcccttggatcttatcttattagatgatgaattgaatattccactgggtagaccgtatttaaccctgctcatcgatagctacagccattgtgttgtgggcttctatctggggtatcgcgaacctagttatgattcagtttgtaaggcgctattaaatgcttgtttggacaaaagctatgtaaaaaaacagtatcctcatatccagcatgactgggaatgctcaggaaaaattgaaacgctggttgtcgataatggtgcagaattttggagtaaaaatttagatcagctatgccgagtagttgtgagtgatatccagtacaaccctgtagctcacccttggctcaaaccgttggtagagcggttttttggaactgttaacaaaaagcttctggtctcaataccgggaaaaacattctctaaaatagatgatttaaaggactacaaccctaaaaaagacgccgtcatgcgcttttcagccttcatagatgtgttttataagtggattattgatgtttatcatcgctctgctaatgcccgcgagacgcacattccttatttgtcgtggcaaatcggcgtttctgaattccctcccgctcaatatacgggagaaacagcagagcgactcattattgaattaggccaaacttttgagcttgttctacgaaaaggaggcttacatcttaaagggattcattatgaaaatgatgaactggtggaatacaggcgcagtacatcactaccgtcgggaaagaagagcctaaagctcctagtgaagctaaaccctatggatatgagttgcatctatgtgtatctgaaagatcaagatcgctatattaaagtccctagcgtagatccggataactacacacaaggattaacattacttcaacatcagaccaacaagaggttgattcgctatcaaacccgtcggcacgtcgatatggaaagcttagctgaagccagacagtatatagagcagcgggtacaggaagaggtcgataatattcaaagacggccaaagagaccaagcaaaagcattggcggtatgaaaagcttagccaagcatcaaaatgttggtagcgattcagtatcctcggttgttcccagcaaagtgccaatgaatgaaaaagtagaaaaccaaaagcctgaatctgacgatttactcgacaactgggacgatatggtctcggatctggatccttattaa

SEQ ID NO: 83 - DNA, 1008 bp, Neptunomonas qingdaonensis
  source 1..1008: mol_type = genomic DNA
atgatcactctcagcgaagagcagacaagccgcttactcgcgttcaacgactgctttgttgaacatccggccctaacaaccatcatcgaagattttgagcgcttacgtttcaaccgaactctggggggagagcagcagagtatgctcattacaggtgatacgggctcaggcaaaactcagcttatcaatcactacagtaagcgtttctttagtcagcaacggcgtaacgtgttaacaaagcctattctggtgagtcgtatcccaagcaagttaacgcttgagcatacaatcattcaactattgatggacttagggcagtttgggacagaatatcgtacaggacgtagtcatgagttagggctcacggaatcgctcgtgcgatgtttgcagctgtgtgagacagagctgattattattaacgaattccaagaattgattgaattcaagacagcccaataccggcagttaatcgctaatcgtctgaaatatatcactgaagaaacgaaaattcccattgtgttagtcggtatgccctgggctgaattgatcgcagaagagccgcagtgggcttctcgattgatttgcaggcgccaaataccttattttaaattatcggaagggccagaggacttcgtgcgattcttaaaaggtttagccatacgaatgccttttgatctacctccaaagttagaagaaaaacatacaacgctagcgctatttgcagcctgcagaggcgaggttcgtaagctaaagcacttattgaatgaggcggttaaaatggcactttttgacggtgctaaaactttgaccaaagagcatctagccaatgcctatcagcttttttatccaagcctgctaaatccctttctgcaaacgatcaatgacctaaaattcgcagaggttgctagttattcacgttacgacccgaatgcaatttctgttgatgaagccctgattgctacaagatttacggatcaaatgtcattgacccagttgttaaaaaagtag

SEQ ID NO: 84 - DNA, 1239 bp, Neptunomonas qingdaonensis
  source 1..1239: mol_type = genomic DNA
atgcagctacttgtcaggccagaaccttatgaagatgaatcgttagagagctacttgcttcggctctcatcggctaattttcttgaatcctacccgttactcagcggatctattcgggaatggttatgggaaaatgaccgcgatgctgccagcgcatttcctctcgctttatcccaaattaatatttaccacgccaaacaaagtagtagttttcgtatacgtgcgatccgcttagttgagtctctgactggtttagaacggttgccgttacttaaattagcgattcttaactcttctgataaatacggcaatggcttgagtgccgttgtccgtaatggcgtttttacacctcgctgttttttgcgcagtgacggtatacctgtttgtcctcattgcttagccgagtcctcttatatacgccagcattggcactttttacattattccgcctgcgttgaacacgaatgcgtattgctggagcgctgccctgcttgtaaagcgacgctgaattatttagaccatgagcaaattggaacatgtacctgcggtttcgagttgaagagtgccagcgttgaaagtgctgattgcgacgcactaacactgagccaaattgtagtaggtcacatctctaacacagaaagcgctttatctagtgaaccatcgctctcaatcaggtacggcgcgttgttgtggtattacttgagaagaaacaaaggattggagacggataaaaagattgaacctgcaggcctgaagggcgcgcttgagtatttcagtcgttggcctgagtcgctaaaaaatgagctacaaggctgtggtgatacagcagagcagcgtttaatagcaggcttcaacaaaacgtcatttcaacatgtttttggaaacatggtcttatctgccagtcattttccgagcgagcatgtgagtcagaactttatactggatgaggtattgtcattccttgaggctctagtcttagacaacccaaaaacaaagcgagctaatatagctgatgtattgctatctgtggcagaggttgccgttgttcttgcaacatctcatgagcaagtttatcggttataccaggagggttttctggagctttcgttgcggctgccgagtaagactaaacttggtagccatacgcctgcattttatctacgccaggtctttgagtttcgtgctgcgcatggtcagccatcaccacaagcccagaacgcctatctttcagggtggtaa

SEQ ID NO: 85 - DNA, 2082 bp, Neptunomonas qingdaonensis
  source 1..2082: mol_type = genomic DNA
atggactttctccagttacttaaccttaacgatcaagatgagcaatctctacagatacgcaaagccttcgcgccttacacgccgttgttagatgtaacgggaaatgaaattttagccctggtggttttgctgaatctgacgttaaagcgggatactgttaaatacctgttagatgaaaaacttgccaaagctacattgaaaaatcagaagcacctggatacttgtgtatcagaagtacgttggttgcatagccataatctaaaatatcccgataccagagtatctgggcagcgtttgatcgtagaagctcaaccaatgaccgaaggcgttatttcttcgtcactatttccactgatgcttggttggtctcacaatgcgagtgcggtgaataaagcgaagttgtttggatcatcttttttatggaagcaacgggtttgtaacctagcgaatttacttgttgaaaaagaacttgagtggcaaaaagcatttcaagcgttaggaatgacaaagcgtcaggtgtatgaactaagagagcaggtactgcaggcgataaacgaaaatcagataccgtcagaggtgagtgatttttcacctcagttacgtttcccctatgaaggtgattatatttccattacgccagtggtgtctcatgcggtacaagcatcacttcagcagcatagatctcaacacgtagggcattttgctgatgttagtcatgctcaccctgcgagcgtgggggatttgagttcatcgcttggagggcatatttctgtattgaattacccaccacctgtttggccttcaaatacaagcaacctgcatcattcaaggctggccagagtcttggcgggaaaagaagtttttgaaatcggagctatccgtaataaagcttttctacaggccttggacgcattggcgtcaagtaacaggcagcaaacacgacaactaagaaagaagagtcgtgttgttagcttacgatttattagaaagaaactggcagagtggattgccccacttattgagtggcgggaggtatgcctggaaaatgcttacagcattgatgattgcggaattggtgtattagaacgtcagttattagaagtcgactatgaccaatacccaacattgatctctttattaaaccagcgtttacaccagcaattacaaaggggtaacacaacgtcaaaatacgcttatcatccacagctactaacacccctgaaaagtcagctcttatggcttttgagaagctttggaaaggtagcaacaccacctgaagtcccctcatcttcgtcacagtatttgtatcttcaaggatcgcgggtgtttgatgccaactcattagcaaacccctatcttaccggcgttccttcactgaccgccttatggggctttgcgcataattttcaaagacgaatgaataagttgatgagccgtaaccttcgagtaaaaggcgtaggttggtttatacgtggttattcagcaaagccaacgtcaaagttacctgagttttctgttctgaaaagagatgatcaatccataaaacgccctggtgtgattacatcgagatactgcgatttagtcatggatgttgttcttgagcttgaacggaatgacgaagaggtactactcaccagtgccgatgtgcagtttttacaggcggcgctgccttctgctttcgccggtgggtgcttacaacccccatctttatatgagagtaaggactggtgtaaagtttactcagatcggcagttattattttctacattagtgcggttaccaagaaatggatgctgggtatgcccgtcaaaaacgaagctaaagggctttgacgatatatttgaaagctgcagtcgggatagttttgaaaaaccagcgatggtgggatacgcgttactggaagagcctggtgagcgttatggaagcatcgaacaacctcatgcttttgctgagccgcttattggtatggtcaattgtaaatcagccattgaggtacgtttgaaaggggagaaggcgtttttcaacgactcattttggaatatctatacagagaaaagatgtattcttataaaaaaagcataa

SEQ ID NO: 86 - DNA, 1035 bp, Neptunomonas qingdaonensis
  source 1..1035: mol_type = genomic DNA
atggagctctgcaaacatcttagttatcagcgatcgctgagccctagtaaagcagtctttttttataagactgatgtgagtgagtttgaacctctgcaggtcgaaattaatcatatccaaggtcagaaatgttcccactcagaagcttaccagtcgggaaaccagcctaaaaatatacaaccccaagatttggcttatagcaatcctgtcgcaatagaaacctgttatgtaccccccaccgtcaatgagctgtactgtcgcttttctctcagggtcgaagccaactcgcttcatccgttagtttgcagcgatgagtcagtacatcttcttctgagtgagttggcgcttaagtacaaagctgcaggaggatacctgcagcttgctgagcgctattgtaagaacatattgttgggtggctggttatggcgaaaccaacacgcaaaatctatgcagatagaaatactaacgtctgatgatcagtcttttgtgattgaagatgcgactcaattaacctgggacaattggccaaaaaaagaaagcggcgttcttaaagcgctagctgctggtatggagtctgctttaacagacccaagcgttttttggttcgcggatattaccgccaagataaagaccacgttttgccaagagatcatccccagccaatgctttaccgaccatttggccagaggtgaagcgtcgcgccaatatgcgaaggttgagttacctgatggaagaaacgccgcgagctttaatgcggagaaggtaggcgctgcaattcagagtattgatgattggtgggtagaaggcgctgaaaaaaagctacgagtccacgaatatggggctgatcgccgctttattatcgctcaacgtcgtccagacaaaggcaatgatttttactctcttattagacattgtgaagaatatataacgttattggcagactttaagaaagggagttccatacctgatgatatccattatgtgatgtcagttcttgtaaaagcaggcatgtttcagaaagggaaatctggatga

SEQ ID NO: 87 - DNA, 630 bp, Neptunomonas qingdaonensis
  source 1..630: mol_type = genomic DNA
atgaaacgctactttttttgtgtgaagtatcttttggatggatcagacccgcagttgttagctgggcggtgtatctctgttttgcatggctacatgatcaagcacttaatcagtagtattggtgttagttttcctcagtggagctacgaatcactagggttgtctattgcttttgttagcgatgatttagaacacttgcgcgcattacgagcccaatcgtattttaagatgatgtctgatgaaggtttttttgcaataacagaggttatgcaggtgcctgataatttgcaagaagtacggtttgttcgtaatcaatccatagctaagttatttgcgggagaaacaaggcgtcgattggcgagagcaaagcgacgagcagaagaacgcggcgatgtttttgagccggtagtatcaaatgataaacgtgaagttgggcactttcatcgcctcaaggctggtagcaaaagcaatggggaagattttatcctccatatccaaaaagagaacgttaatagaccgcttgaagatgaggttgatacggagtttaataattatggatttgcaacgaatattagtattagaggaatggttcctgagttaagtgaatatataacaaaatcaagaatttag

SEQ ID NO: 88 - protein, 206 aa, Neptunomonas qingdaonensis
MYRRNLKHSRVKNLFKFASLKIDDVITVESGLEFDACFHFEYSPDVISFDSQPEGFYYSFLGKELPYTPDFVVVDKVNGARFIEVKPVAKAANTDFRAKFICRQERAAALGIPVILVTERQIRLNPILNNLKLLHRYAGLQSFTELHFAILELVKKSGIVSVRNLISSTQAIEGDVLASTVTWLSLGRLKTDLSSSDLGLDSVVWC

SEQ ID NO: 89 - protein, 628 aa, Neptunomonas qingdaonensis
MATPDLLSKSSGLFEDEFSPNEPEIPFFEEPSAEIFPRDLKTYPAELRDEALRRLAYIQWVGKRLEGGWTEKNLTPLLHVVGDKLLAPLPSWRTLAGWRKLYVSSGRSVEALIPKHHKKGNRLEQNTGDDLYYWQAVNQKYLRPERPSIASVYEYYRDLITLANKGVVQGKVMHVSRSGFYNRINKLPPYDVALKRYGKQYADRKYRPVGAHITLTRVMERVEIDHTPLDLILLDDELNIPLGRPYLTLLIDSYSHCVVGFYLGYREPSYDSVCKALLNACLDKSYVKKQYPHIQHDWECSGKIETLVVDNGAEFWSKNLDQLCRVVVSDIQYNPVAHPWLKPLVERFFGTVNKKLLVSIPGKTFSKIDDLKDYNPKKDAVMRFSAFIDVFYKWIIDVYHRSANARETHIPYLSWQIGVSEFPPAQYTGETAERLIIELGQTFELVLRKGGLHLKGIHYENDELVEYRRSTSLPSGKKSLKLLVKLNPMDMSCIYVYLKDQDRYIKVPSVDPDNYTQGLTLLQHQTNKRLIRYQTRRHVDMESLAEARQYIEQRVQEEVDNIQRRPKRPSKSIGGMKSLAKHQNVGSDSVSSVVPSKVPMNEKVENQKPESDDLLDNWDDMVSDLDPY

SEQ ID NO: 90 - protein, 335 aa, Neptunomonas qingdaonensis
MITLSEEQTSRLLAFNDCFVEHPALTTIIEDFERLRFNRTLGGEQQSMLITGDTGSGKTQLINHYSKRFFSQQRRNVLTKPILVSRIPSKLTLEHTIIQLLMDLGQFGTEYRTGRSHELGLTESLVRCLQLCETELIIINEFQELIEFKTAQYRQLIANRLKYITEETKIPIVLVGMPWAELIAEEPQWASRLICRRQIPYFKLSEGPEDFVRFLKGLAIRMPFDLPPKLEEKHTTLALFAACRGEVRKLKHLLNEAVKMALFDGAKTLTKEHLANAYQLFYPSLLNPFLQTINDLKFAEVASYSRYDPNAISVDEALIATRFTDQMSLTQLLKK

SEQ ID NO: 91 - protein, 412 aa, Neptunomonas qingdaonensis
MQLLVRPEPYEDESLESYLLRLSSANFLESYPLLSGSIREWLWENDRDAASAFPLALSQINIYHAKQSSSFRIRAIRLVESLTGLERLPLLKLAILNSSDKYGNGLSAVVRNGVFTPRCFLRSDGIPVCPHCLAESSYIRQHWHFLHYSACVEHECVLLERCPACKATLNYLDHEQIGTCTCGFELKSASVESADCDALTLSQIVVGHISNTESALSSEPSLSIRYGALLWYYLRRNKGLETDKKIEPAGLKGALEYFSRWPESLKNELQGCGDTAEQRLIAGFNKTSFQHVFGNMVLSASHFPSEHVSQNFILDEVLSFLEALVLDNPKTKRANIADVLLSVAEVAVVLATSHEQVYRLYQEGFLELSLRLPSKTKLGSHTPAFYLRQVFEFRAAHGQPSPQAQNAYLSGW

SEQ ID NO: 92 - protein, 693 aa, Neptunomonas qingdaonensis
MDFLQLLNLNDQDEQSLQIRKAFAPYTPLLDVTGNEILALVVLLNLTLKRDTVKYLLDEKLAKATLKNQKHLDTCVSEVRWLHSHNLKYPDTRVSGQRLIVEAQPMTEGVISSSLFPLMLGWSHNASAVNKAKLFGSSFLWKQRVCNLANLLVEKELEWQKAFQALGMTKRQVYELREQVLQAINENQIPSEVSDFSPQLRFPYEGDYISITPVVSHAVQASLQQHRSQHVGHFADVSHAHPASVGDLSSSLGGHISVLNYPPPVWPSNTSNLHHSRLARVLAGKEVFEIGAIRNKAFLQALDALASSNRQQTRQLRKKSRVVSLRFIRKKLAEWIAPLIEWREVCLENAYSIDDCGIGVLERQLLEVDYDQYPTLISLLNQRLHQQLQRGNTTSKYAYHPQLLTPLKSQLLWLLRSFGKVATPPEVPSSSSQYLYLQGSRVFDANSLANPYLTGVPSLTALWGFAHNFQRRMNKLMSRNLRVKGVGWFIRGYSAKPTSKLPEFSVLKRDDQSIKRPGVITSRYCDLVMDVVLELERNDEEVLLTSADVQFLQAALPSAFAGGCLQPPSLYESKDWCKVYSDRQLLFSTLVRLPRNGCWVCPSKTKLKGFDDIFESCSRDSFEKPAMVGYALLEEPGERYGSIEQPHAFAEPLIGMVNCKSAIEVRLKGEKAFFNDSFWNIYTEKRCILIKKA

SEQ ID NO: 93 - protein, 344 aa, Neptunomonas qingdaonensis
MELCKHLSYQRSLSPSKAVFFYKTDVSEFEPLQVEINHIQGQKCSHSEAYQSGNQPKNIQPQDLAYSNPVAIETCYVPPTVNELYCRFSLRVEANSLHPLVCSDESVHLLLSELALKYKAAGGYLQLAERYCKNILLGGWLWRNQHAKSMQIEILTSDDQSFVIEDATQLTWDNWPKKESGVLKALAAGMESALTDPSVFWFADITAKIKTTFCQEIIPSQCFTDHLARGEASRQYAKVELPDGRNAASFNAEKVGAAIQSIDDWWVEGAEKKLRVHEYGADRRFIIAQRRPDKGNDFYSLIRHCEEYITLLADFKKGSSIPDDIHYVMSVLVKAGMFQKGKSG

SEQ ID NO: 94 - protein, 209 aa, Neptunomonas qingdaonensis
MKRYFFCVKYLLDGSDPQLLAGRCISVLHGYMIKHLISSIGVSFPQWSYESLGLSIAFVSDDLEHLRALRAQSYFKMMSDEGFFAITEVMQVPDNLQEVRFVRNQSIAKLFAGETRRRLARAKRRAEERGDVFEPVVSNDKREVGHFHRLKAGSKSNGEDFILHIQKENVNRPLEDEVDTEFNNYGFATNISIRGMVPELSEYITKSRI

SEQ ID NO: 95 - DNA, 28 bp, Neptunomonas qingdaonensis
  source 1..28: mol_type = other DNA
gtatcctgccgggtaggcagttgaaaat

SEQ ID NO: 96 - DNA, 28 bp, Neptunomonas qingdaonensis
  source 1..28: mol_type = other DNA
aaatcctgccgaaaggcagaatggtatt

SEQ ID NO: 97 - DNA, 28 bp, Neptunomonas qingdaonensis
  source 1..28: mol_type = other DNA
gtattttaccgagaaacaccttgagata

SEQ ID NO: 98 - DNA, 28 bp, Neptunomonas qingdaonensis
  source 1..28: mol_type = other DNA
aagtcctgctgtgaaatattaacgaatt

SEQ ID NO: 99 - DNA, 28 bp, Neptunomonas qingdaonensis
  source 1..28: mol_type = other DNA
tttccctgccgaataggcagctgaaaag

SEQ ID NO: 100 - DNA, 28 bp, Neptunomonas qingdaonensis
  source 1..28: mol_type = other DNA
atgccctgccgtgtaggcagctgaaaag

SEQ ID NO: 101 - DNA, 133 bp, Neptunomonas qingdaonensis
  source 1..133: mol_type = other DNA
tgtcgttttcagcataagtttgcataagatccacataagtctgcataaagtccatataagtttgcataatttgcacaaaaagcctgcataaggtatatttttgtagtaatactacgaagatggtggcaatgct

SEQ ID NO: 102 - DNA, 188 bp, Neptunomonas qingdaonensis
  source 1..188: mol_type = other DNA
ctaaggctgaatatcgcctcaggttatatttttcacatgtagtataattacaaaatgggttctaggttttttcgtgggtaactttatgcaatctttcactacatttttatgcaaacttatgctgcatattcccccgtaacttgttgttttacagttatcgtttatgcagactttcggtgcaaacgaca

SEQ ID NO: 103 - DNA, 2496 bp, Klebsiella oxytoca
  source 1..2496: mol_type = genomic DNA
atgtaccgtcggcatttacatcattctcgtgtaaaaaatctctttaaattcgctagtgttcggatggggatcgttttgactctcgagtcttcacttgaatttgacacttgctttcagctcgaatactctccggcagtcaaaacctatatctcgcaacctgaaggcttttattatgaatttgaagggaaatcatacccgtatactcctgattttttggtgaaagaccagaatgatcaggaatttttactagaggtgaagccgtcttcgcaaatagacgatatcgattttctccaacgttttccggcgaagcaaaaaaaagctaaggaactggcatcacctctaattcttattactgaaaaacaaattcggtccactcctcttctggacaacttgaagttagttcatcgttatgctggatttcacagcattatgccttcgtgcaatgaaatcatggagttgctaagagaacagaaggaagtggcaatttttaatctttgtgagtcaatagatattccgcaaggcgagatgtattcgtcaattttattattgctttcacgaggattaatttctgggaatttaatggaatctgaattcggtttggtcactttattgaaatacgctcaacagaatagtctttttatatgttcatttccatttgaagatgaatttactttatctcaagagaatgaagtgaagatgtctactgatgaaagtagtgatattattttacccgctactcttgattgctattctgaaatattaaaggaagagtctgtccggaggcttaactatatccagtgggtagagaagcgaataataggggggtggacagaaaaaaatattacgccattgattaatgaagttgcacaaacgctaagacctcctgctccacactggagacagctcgttcgatggcataaaaagtacttacagcatcgaaggcaaatcacggccttggtccctaaccataaaaataaaggtaataaaacgcaaagagtaagctccagggaggaaatttttattgagaatgctatactaaaatttcagagtaaagaaaggccatcaatatccagtatgtattgtttttattgcgatagtgttcggatttttaatctcagtaattctaccgaacgcattaagactgtatcattaaataccttttatcgcagaataaaaaaactttctgtataccaggttatgaatgcaagggatggccgtgttgctgcaaatatggaatttcaggctatagatagttttttaccaacgtcccgggttcttgagcgagttgaaattgatcacacgcctttagatttgatacttctggatgatgaattgctgctcccattaggaaggccaagcttaacactattgatagatgtttatagccattgtgcagtaggtttcaatctgtgtttcacacaacctggttatgagtctgtacgttgtgcattactacattcgctagttcgtaaagactatgtacaggaacaatatccctgtatcgagaacagttggataagttacggtaaaccagaaacacttgttgtggataatggcgctgaattctggagtagcagtttagagcatgcctgtctggaacttggtataaatacacaatataaccctgtaagaaaaccctggttgaagcctctgatagagagaatgtttggtacaataaatcgtaagtttttggagtcaattccagggaaaactttttcaaacattctggataaagctgattataacccgcaaaaggatgcggtgatgagattctcagtttttcttgaaattttccatcattggctactggacgtgtatcattatgagcctgatagcaggtataggtatgttccagcattagcgtggaagtatggttgtaaagtgtatcccccggcaactattgaaaaaaatgaattaaaaaaattagaaatcattctgagcattagtctcagacgtctccaccgacgtggggggatacatttacatcatttacggtatgacagtaaagaactgtctgccttacgaatgcagtattctctagaggaaaaagggaagaaaaaggttttagtaaaacttaatcccgcagatatgtcttacatatatgtctatattgataagatcaagagttacattcgagttccatgtgttgatccatgtaaatatactcagaatttaagcttacaacaacatcttattaatcttaggtttcatcgggattttattaatgaaaatatcaaccttgacagtttatcaaaagcaaggatttacatttcagagagaatccaaggtgaaattgataatgtaagacaatatgcgaagcgatcttctaaaaagggaatgaagaaaatagctagtcatcagggtgttacgagccagaataaaaaaacaattgcttctgataccatccattttccggcccaaaagggaaaaaatcgagatactcataccttgccggatgactgggatgatttcacttcagatctggagcctttttaa

SEQ ID NO: 104 - DNA, 1005 bp, Klebsiella oxytoca
  source 1..1005: mol_type = genomic DNA
atgaaactatccagcttgaaagaagaaaaattaatttcatttataaactgctttgttgagacccctttcctgaatgagatagaaaaggattttgaccgactacgctataatcggtttttgggaggggaaccacaatgtatgcttctgaccggggatacaggaaccgggaaaacatttttgcttcatcattacatgtctaagtatcctgcacagaatgggagtggttatcttcgtaagcctcttcttgtcagcaggatcccgagcaaaccatccctcgaatcgacaatggttgaattgctaaaagatctcgggcaatgggggagtaattatcgacgtaacagaagttcggcggagaatctgactgaatcactcatcaaatgtatgatgaggtgtgagacagagctaattttaattgatgagtttcaagagctcattgagaataaaacaagagaaagacgcaaccagattgccaacaggttaaaatatatcagcgagactgcaaggatccccattgttttagttggtatgccatgggcagctaaaatttctgaagaacctcagtggtcgtcacggctattgattcgtaaaacaattccctatttcaaattaactgatggactatcaatttttgttcgggtcatcaaggggttcgccgcaagaatgcctttcagaaagccccctgaaattgaaggaaaacatactattttaggcctatattcagctagccagggacggatgaggacgttaaaattcttactaaacgaagcggttaaacaagctttatccgaagattctgaaacactgacccatgaacatattggaaaagcatttcatatattttatccggagcatgaaaatccattttatataccccttgagaacattaaaatttatgaagtaagagagtattctggttatgaaattgatggtgcagggaaggaggatagattgatcccacaacagcttacggatcgtataccaataaatcagttgctgagaaaatga

SEQ ID NO: 105 - DNA, 1224 bp, Klebsiella oxytoca
  source 1..1224: mol_type = genomic DNA
atgcattttctcatacgcccggaacctgtgtgtgatgaatcactcgaaagctatttgttgaggctttctcaggataatggatttgaacattatcgaattctaagcggttcacttaaggagaggcttttgcagtccgattatgaggccgcgggagcatttccccttgaactggcgaaggttaacatcttccatgccagttattccagttatttgcgaatcagagcattatgcctgattgcggatctcacaggacagcctcataccaacctgctaaaggtcacattaatgcattctacagtgacttttggtcgcgggcacaaggcggtcagcagagataatacacacataccactttgttttattcgtacaaatagcattccatgttgtcctgaatgccttgctgaacatggatatgtccgtcaactctggcattacaaaccttatacggcctgtcacagacatcgaagaaaactgctcacaagatgcccggcatgtcatgaaagtctgaattatctttatagcgaattactgactcattgctcttgtggatatgacttacgtcaggcctttactccaccaactagtagtgatgatcttcaactttcctccatggtgagtgatgacaaatgtgaagctctaagcccagcctcagccagccaggacaagagcttacgatatggagcattgctctggttcattatgcgctatggagaatcctcaaacaatgaggagggcatgttaagcgctatgcattactttcgtgcatggccggacaacttcaccgctgaattgctggatatgatggcagcggccacaataaaacaaactaaatcattcaatcatatgagtctgacagatgtcttcggtaaaacattatctgattgtctgtatctgccagcacgtgatacccaccggaatttcattcttcacgcatttcttgattatttaaccaacctggtcatggaaaacccacggtcaaatattgctaatcctggtgatctgcttctctcaatacgggatgccgcatgtttgctctccacatctaatgcgcaggtatatcgcctgctagatgatggttttttgaaagtagccatcaggccaagggctggtatgaaggttaaaatcagtacccctgttttacatttacgtcaggttattgaatttcgtcttacacatatacctggcccacacgacaagggccacacttatctttctgcccggtga

SEQ ID NO: 106 - DNA, 2067 bp, Klebsiella oxytoca
  source 1..2067: mol_type = genomic DNA
atgcatatacgcgaattacttaagataaaggatcacagtgagagggatagagcattacgacatggtttttctcccatcagggaaaagattgatatggaggggtttgaatacgagacgcttgtggtattgcttaatatgacgctgaagcgtgaccttgtgcacaatctttttgatgtccggctcgccaggcagctgctgtttgataaaaaccatcttgcacattgcgtcaatgctgttcgctggttgcatacacataacctgaagtatccggacagcagagtccgagggcagcggcttatcatttgctcaccagcggttataccagggattgtctccagtgctgatctgccccaagagatggggtgggctaacaatggcgccgacataaattttgcaagactcttttgctcctttttcaggcataacggatctataacctgcctagctaaactcttgaccgaaggctgtagtggtatcgtcaaggcacttgagcgtctgggtacgtcaactgacgatatttgtcttttgcgtgtggcaattgctaataatatcagcgaatcggtaattccatcggatgtgagtatttattccaggcaattacggggtttcttgcaagggaaggatgttgccattacgccagtagtcagtcatgcactgatggcaagattgcagcaactaatatatcaacagaggaagccacacatcattatccgacatgaccaccctgccagtatgggaaaccttgtggcttcgacgggtggaaatattgctgtgatgtattatccgccgcttgtcagtgtacataaagagcgcagctttattcactccagagtagggcttctgcaggagcgggaacacctgtttgataataatgtcttgagagaaaaggagctttttaatgctctgcagaatcttgtgagtcacaatggaggtagtcaaagacaaatccgtcaacaacggttatcagcattacgctatctgagataccagttagttatctggttgaagcctgtgattgaatgtatagacgctctggaggaaaacagggaagacatattatctctgcctgaaagtattgaaaaaaaagtacttactcagtcggtgaatcggttggatgagttatcttctgaactcgccggacactttcatttaagtttacaacatcatccgctgtttagacgatttgcatttcattctgaactggttgtgtcagtagagtcccagttaaaatggatacttaagaacatatccagaagcgaccctgatactcctataactcagagttgcagggagttctatctgcacctttcggggctcaatatttacgatgccagtgccatgtccaatccatatctttgtggtattccatcgcttactgcacttgcgggattctgtcatgattatgagcgacgagtctctgctttaatggaacagaaggtgtgtttcactgaagttgcatggtatataggacattacaatctgatttccggcaggcagttaccggcagcaatgattcctgaaagaaaaaataccatctcatccctccgacgccctgggatcactgatgaaaagtgctgtgatatgggtatagagttggtcataaaattacagtttccggaggaatgtaaactgccagagtcaggtttactttacgccgcttccccatcccgttttgccggaggagttttacaccctccatcattcagcggagagaaatcatggtgtcaactttattctgaccaggacgcgttatattcagtgttatcccgattgccaggaagcggatgctggatatatccggtgagaacgactatcaccactcttgaagagatgttcactgaattatcttctgactatcgcttacggcctgtctcatctggatttattttgctggaagagatgcagtatcgagccgggagtctggcatctcaacatgtctacgcggagtcggctctgggactcgcacgatgccataatccgatcgaaataaggctggcaggtaagaaaaatttttataatcagggtttctggccactggattacgaggacagaactataattacataa

SEQ ID NO: 107 - DNA, 1038 bp, Klebsiella oxytoca
  source 1..1038: mol_type = genomic DNA
atggaactatgcactcatctcagctatatgcgctccatatctccggggaaggctgttttttactataaaagacccgaatgtgagtttgttcctttagaaatacaaacttcgaaaataagggggcaaaaatgcagttatagtgaagggtttagagaaaatcttcagccaagaaaactgcaacaacatgatctggcgtatgctaatccgttaaccattgagatctgttatgttccggcagacgtcaacgaaatatactgccgtttcacattacgaattgaggctaattccttacgaccttatgtctgtggtgatccccatgttctgaatactctgacagagcttgctctggagtataaaaaacacgacggatataaagaactggcaaaacgctacagcacgaacctgttaatggggtcgtggctgtggcgtaatcgttttacacagagtacgcagttagaaatcaaaacctcactgaattcgacatatcgaatcttagattctcgtgaactcaactggtccgaagcctggccagaatcagaacagcgtcaacgcgaactactggaaagggagattgaaaccgccctcagtgagcccggtgttttttggggggcagatgtcatcgccacattgcaaaccagtttttgtcaggagatttatccaagccagaaatttatcgaaaaaaccgtagattatagtattgccagccgccagctggcaacgacagagtgttcaaacggtaaacaagctgcatgtataaccgctcaaaaaatcggcgctgcattacaacggattgatgactggtggtctgctgatgctgattatccactacgcgttcatgagtatggggcagagcctgaaagactgactgcccgtcggcacccggtatcgggacatgatttttatcatttgttgacgaaagctgatatttttttaaacgactttaaatcaaagaaaatgaagaaaatttctggtgatatacatttcctgatgtccgtactggtaaaaggtggactcttccagaaaggaagaggggcatga

SEQ ID NO: 108 - DNA, 621 bp, Klebsiella oxytoca
  source 1..621: mol_type = genomic DNA
atgagaatgacgcgttattttttttctgtctactatcttcctgaagatgctgattaccctttgctggcgggtcgttgcatatcaacactgcatggatacacttctcatcatccggatacaagaattggcgtaagctttcctgactggacagataccacactcggcaggaccattgcgtttgtcagtgttaacaggagccatctggaacaattaaaggaacgtgcttattttaaaatcttaaaggaagaaaaaattttttcgatcagtcctgtacttaaggtaccggaatactgcccggatgtaatgttcattcgaaatcagactatagctaaatgttttgtcaaagaacgtaagagacgactggaacgtgcaaagcgtcgggcagaggccagaggcgaggtttttcaacctagagttaattctccgcttagaagtatagaggcttttcatggtatctttatgcagagcataagtaatggatgtagttttctgttacatatccagaaaaaagaagccagaatccaaagcaatcatatgtattgcagctatggtctggcatcaaatgaagtgtatacaggtcatgttccggatttgagtagtgtcgtaaaaaaactgttttaa

SEQ ID NO: 109 - protein, 831 aa, Klebsiella oxytoca
MYRRHLHHSRVKNLFKFASVRMGIVLTLESSLEFDTCFQLEYSPAVKTYISQPEGFYYEFEGKSYPYTPDFLVKDQNDQEFLLEVKPSSQIDDIDFLQRFPAKQKKAKELASPLILITEKQIRSTPLLDNLKLVHRYAGFHSIMPSCNEIMELLREQKEVAIFNLCESIDIPQGEMYSSILLLLSRGLISGNLMESEFGLVTLLKYAQQNSLFICSFPFEDEFTLSQENEVKMSTDESSDIILPATLDCYSEILKEESVRRLNYIQWVEKRIIGGWTEKNITPLINEVAQTLRPPAPHWRQLVRWHKKYLQHRRQITALVPNHKNKGNKTQRVSSREEIFIENAILKFQSKERPSISSMYCFYCDSVRIFNLSNSTERIKTVSLNTFYRRIKKLSVYQVMNARDGRVAANMEFQAIDSFLPTSRVLERVEIDHTPLDLILLDDELLLPLGRPSLTLLIDVYSHCAVGFNLCFTQPGYESVRCALLHSLVRKDYVQEQYPCIENSWISYGKPETLVVDNGAEFWSSSLEHACLELGINTQYNPVRKPWLKPLIERMFGTINRKFLESIPGKTFSNILDKADYNPQKDAVMRFSVFLEIFHHWLLDVYHYEPDSRYRYVPALAWKYGCKVYPPATIEKNELKKLEIILSISLRRLHRRGGIHLHHLRYDSKELSALRMQYSLEEKGKKKVLVKLNPADMSYIYVYIDKIKSYIRVPCVDPCKYTQNLSLQQHLINLRFHRDFINENINLDSLSKARIYISERIQGEIDNVRQYAKRSSKKGMKKIASHQGVTSQNKKTIASDTIHFPAQKGKNRDTHTLPDDWDDFTSDLEPF

SEQ ID NO: 110 - protein, 334 aa, Klebsiella oxytoca
MKLSSLKEEKLISFINCFVETPFLNEIEKDFDRLRYNRFLGGEPQCMLLTGDTGTGKTFLLHHYMSKYPAQNGSGYLRKPLLVSRIPSKPSLESTMVELLKDLGQWGSNYRRNRSSAENLTESLIKCMMRCETELILIDEFQELIENKTRERRNQIANRLKYISETARIPIVLVGMPWAAKISEEPQWSSRLLIRKTIPYFKLTDGLSIFVRVIKGFAARMPFRKPPEIEGKHTILGLYSASQGRMRTLKFLLNEAVKQALSEDSETLTHEHIGKAFHIFYPEHENPFYIPLENIKIYEVREYSGYEIDGAGKEDRLIPQQLTDRIPINQLLRK

SEQ ID NO: 111 - protein, 407 aa, Klebsiella oxytoca
MHFLIRPEPVCDESLESYLLRLSQDNGFEHYRILSGSLKERLLQSDYEAAGAFPLELAKVNIFHASYSSYLRIRALCLIADLTGQPHTNLLKVTLMHSTVTFGRGHKAVSRDNTHIPLCFIRTNSIPCCPECLAEHGYVRQLWHYKPYTACHRHRRKLLTRCPACHESLNYLYSELLTHCSCGYDLRQAFTPPTSSDDLQLSSMVSDDKCEALSPASASQDKSLRYGALLWFIMRYGESSNNEEGMLSAMHYFRAWPDNFTAELLDMMAAATIKQTKSFNHMSLTDVFGKTLSDCLYLPARDTHRNFILHAFLDYLTNLVMENPRSNIANPGDLLLSIRDAACLLSTSNAQVYRLLDDGFLKVAIRPRAGMKVKISTPVLHLRQVIEFRLTHIPGPHDKGHTYLSAR

SEQ ID NO: 112 - protein, 688 aa, Klebsiella oxytoca
MHIRELLKIKDHSERDRALRHGFSPIREKIDMEGFEYETLVVLLNMTLKRDLVHNLFDVRLARQLLFDKNHLAHCVNAVRWLHTHNLKYPDSRVRGQRLIICSPAVIPGIVSSADLPQEMGWANNGADINFARLFCSFFRHNGSITCLAKLLTEGCSGIVKALERLGTSTDDICLLRVAIANNISESVIPSDVSIYSRQLRGFLQGKDVAITPVVSHALMARLQQLIYQQRKPHIIIRHDHPASMGNLVASTGGNIAVMYYPPLVSVHKERSFIHSRVGLLQEREHLFDNNVLREKELFNALQNLVSHNGGSQRQIRQQRLSALRYLRYQLVIWLKPVIECIDALEENREDILSLPESIEKKVLTQSVNRLDELSSELAGHFHLSLQHHPLFRRFAFHSELVVSVESQLKWILKNISRSDPDTPITQSCREFYLHLSGLNIYDASAMSNPYLCGIPSLTALAGFCHDYERRVSALMEQKVCFTEVAWYIGHYNLISGRQLPAAMIPERKNTISSLRRPGITDEKCCDMGIELVIKLQFPEECKLPESGLLYAASPSRFAGGVLHPPSFSGEKSWCQLYSDQDALYSVLSRLPGSGCWIYPVRTTITTLEEMFTELSSDYRLRPVSSGFILLEEMQYRAGSLASQHVYAESALGLARCHNPIEIRLAGKKNFYNQGFWPLDYEDRTIIT

SEQ ID NO: 113 - protein, 345 aa, Klebsiella oxytoca
MELCTHLSYMRSISPGKAVFYYKRPECEFVPLEIQTSKIRGQKCSYSEGFRENLQPRKLQQHDLAYANPLTIEICYVPADVNEIYCRFTLRIEANSLRPYVCGDPHVLNTLTELALEYKKHDGYKELAKRYSTNLLMGSWLWRNRFTQSTQLEIKTSLNSTYRILDSRELNWSEAWPESEQRQRELLEREIETALSEPGVFWGADVIATLQTSFCQEIYPSQKFIEKTVDYSIASRQLATTECSNGKQAACITAQKIGAALQRIDDWWSADADYPLRVHEYGAEPERLTARRHPVSGHDFYHLLTKADIFLNDFKSKKMKKISGDIHFLMSVLVKGGLFQKGRGA

SEQ ID NO: 114 - protein, 206 aa, Klebsiella oxytoca
MRMTRYFFSVYYLPEDADYPLLAGRCISTLHGYTSHHPDTRIGVSFPDWTDTTLGRTIAFVSVNRSHLEQLKERAYFKILKEEKIFSISPVLKVPEYCPDVMFIRNQTIAKCFVKERKRRLERAKRRAEARGEVFQPRVNSPLRSIEAFHGIFMQSISNGCSFLLHIQKKEARIQSNHMYCSYGLASNEVYTGHVPDLSSVVKKLF

SEQ ID NO: 115 - DNA, 28 bp, Klebsiella oxytoca
  source 1..28: mol_type = other DNA
atgaactgccgtgcaggcagccaaaaat

SEQ ID NO: 116 - DNA, 28 bp, Klebsiella oxytoca
  source 1..28: mol_type = other DNA
atgaaatgccgaacaggcagacaaaaaa

SEQ ID NO: 117 - DNA, 28 bp, Klebsiella oxytoca
  source 1..28: mol_type = other DNA
gtgaattgccgtacaggcagccaaagac

SEQ ID NO: 118 - DNA, 125 bp, Klebsiella oxytoca
  source 1..125: mol_type = other DNA
tgtcgtttaatccataagttgacatatttaatgcataagctgacataactaggcaggataagctgacatagcctacatttgtagtaaagcaactacattaggagaggtctatttatcgtcggcac

SEQ ID NO: 119 - DNA, 155 bp, Klebsiella oxytoca
  source 1..155: mol_type = other DNA
acgtgtaattttactacataaaaaatctcgcgatgagtgatgaacaactttatgccatcttatgtgataaatttatgtcaatttatgcgtaaaattaaacgcttaacttattgatatttagttcatcgttatgtcaacttatgagttaagcgaca

SEQ ID NO: 120 - DNA, 1212 bp, Photobacterium aquae
  source 1..1212: mol_type = genomic DNA
gtgaatacggacattcagttttatcccgacgaatcgctcgaaagctttttgctgcggctatcgcatcatcagggttacgagcgatttgcgtattttgcagaagacatctggtatcagacccgtgatcagcatgaagcgatagcgggagctttcccgctagaactcaatcgtgtgaatgtatatcacgcgcacacaacaagccagatgcgcgtacgcgtccttatgcacttggagaatcagctaaatcttgatgatttcagagttctgcatattgtgctagcacactccaaatctcaattctctccagattttaaagccgtgcatcgttgcggggtggattaccctttcgcatttctacggaaacgctttatgccggtttgtcctttgtgtctcgctgagtctgcttatgttcggcaacattggcacttcattcctattcaagcctgtgagcaacatggttgcaagctcattcatcgttgcccagcttgtgatggcctacttgaataccaatcaacagagtgcatgactcattgtgagtgcggatttaacttacttagtacaccgacaacatcagcatctgcctctgagctattaatttctcgctggcttacgggaacgcagttagatgttgctggactcatgggtaaggcactttcgatctcagaacgttatggctttttactttggtatgtaaaccgttacggcgatcttgaagacatcagttttgacgttttcgttgaatattgcacgacatggccgaaatccttgcgaaaagatttggatgaatgcgtacaaaaagctgacgcgattagggttaaacgatggaagcaggtgtttttctctgaagccttcggcgcgttgttgaaagggtgtcgacagctgccaagtcggcagttgagtaaaaactgtgtacttgttgagatactgaattatttcaagcgattggttgcagataatcccaaatcatcgaaggggaatattgcggatgtactgctaagcccactcgaagcctcaacgttactttcctgcacaaccgatgaagtgtatcggctctatgagtttggtgaaatcaaggcggcgatgcgacccaaaatacatactaagatcgcaagccatgaatcagcttttaccttgagaagtgttgttgagactaggttgacacggatgtgttcagaaaatgatggccttagtgtgtatcttccagagtggtaa

SEQ ID NO: 121 - DNA, 2088 bp, Photobacterium aquae
  source 1..2088: mol_type = genomic DNA
atgaaaaaactgtgtgacgtattacaaattgaagacaacaccgaaaagcaggcaaccttaaaaaaggtgtttatgccttactctgcttgtatcgatattgatggctgtgaaaaagaagcactaacggtgttgctcaatctcagtacccatcgtaaaggttccccatgcggtgattggttagatattgaacgtgcgaaaagttacctaaaagatcaagctgacatagatgcatcactggcggagataaaatggtttcatacccataatctcaaatttccagactgtcgcgtgaaagaacagcgtctgattgctaaacctctttctacatcagagtcttttatttcaagtgtttcattggatcagggcttgggttgggcacataactcagcggtatatcgccatacgctgtggctgttgaattcctttaactggcaatcagagtctgtgaatatcttgtctctcgttcaggaagagaatcctgtttggttagagttacttcaagagtttgggttgaacattaagcagcaagatctgctgctaaagactattgaattacagataccggcttcaacttttcctgacagtgttagtccatacagtaagcagctgaggttcccttggaataatgactatctatcagtgacgccagtggtaagtcatgctattcagcgggaaatagaagtgaaagctcgagataaagcgagcaagcttagctttgtgacctcggcactccccaactctgcaagtattggtaatttatgcggtagtctcggtggctatatgaaagcgctgaattacccgctagatgtaaaatctgtggcggagcagaccctagcagcaagccgcaataagagcggtaagtatttcgatgacttccaggtaacgaattacaaaatctgtcaggtgctcaatcgcctcattggtgcagagccactaaaaaatcaaaagcaaagagagaaagcgcgtaaggttcaaagcaagatcttgagaaagcagatcgctttatggatgttgccattaatcgagctaagagacattgaagatgcagagccacacaatcagcaattggagcatgatgatccattagtcaaatcattcctatcattgcctgaatcggagtttccatctttggttcatgaattgaatcaacgtctacacttcgtttttcaggagaataaatttacggctaagtttgcctaccatcctaaattgattcaggtagtcaaggcgcagattgtgtgggtgcttgagcagctcagtaaaccaagtgatcatgaggatgcagcaagagagcagcaatacatctatctctcatccctacgagtgcaagatgcggtagcaatgagtagcccttatttgtgcggagctccttcactaacggctatctggggctttatgcatcactatcaaagagagtttaataagctggtaaatagtgattccccgtttgagttttctaggtttgccttttatgttcgaactgagaacattcaatcaacagctaagcttaccgagcctaactcattagccaaatcacgcaccctctctaatgctaaacggcccacgattcgaagcgaacgtttggcggatttggaaatagacctagttatcagagttgatagcgatagtaggatttcagatttcttgtcggaactaagggctgctctccctgctgcttttgccggcggggcgttatatcaaccgctgattttatcccaaattgattggttgcgaacgtttagcagtaagagtgaactttttcatgtgcttaaggggataccggcatatggaagttggttgtaccctagcgaaaagcagccaaccaactttaatgaactagagcatttgataacagaagacgcagataacctgccagtttcaattggctaccacttgctagagcacccaactgaacgtgaaaactcaattactgactgtcatgcttatgcagaaaacgctttaggcattgctaagcgtttgaatcccattgaagtacgttttagtggaagagatcacttttttgataatgctttttgggcattagagtctacgagtgcaactattcttataaaaaacgataggaattaa

SEQ ID NO: 122 - DNA, 1038 bp, Photobacterium aquae
  source 1..1038: mol_type = genomic DNA
atggaactgtgtaatcagcttaactatgtacgttcactctctgctggcaaagcatacttctaccatctatctaaaggtggtgagatgtgtcctcttgagatcgatagaacacgattacgcgctccaaaaggtggttatgccgaagcgtacaaggggagcaagttcgtacaaaagaatgttgcccctcaagatttagcttatgccaaccctcagttcattgaagagtgttatgtgaagcctggtgttgatgatatttactgtgctttttcattgcgtattaaggcaaactccctaggccctgatgtctgctgtgacgatgaagttcgcagcaagctttcttcgctcgctaagtcctataaggaactgagtggttattctgaacttgctcatcgttatgcgaaaaatatcctacttggtacctggttatggcgtaacagagagtgccgaaggttatctattgaggtaacaacgagcgattcagagaccttaattgttgaaaatgcgacgaaactgacttggtatgatcattgggataaagatgcagcggagtgtttggacaaacttacggcatatttgactcgggcattatccgacccaactgaatatttttatatggatgtcaaagccaaaatcgcagttgggtggggggatgaggtctatccaagccaagagtttttagataaccgtgaagacggtgttcctaccaagcaactagcaacggtagaacttgaaaatgggagagagacggtcgcttttcacggccaaaaagttggtgctgccttgcaatctatcgatgactggtggcatgaaaaggcggataagcctttgcgggttaatgagtatggcgcagatagagagtacgttattgcaaggcggcatgtgtcgctgaagaacgatttttaccagctgctacggaatactgaaaattggattgagagtatgaatacctctaatattatcccaaatgatgttcacttcattatgtcggtgttggttaaaggtggtctatttaactgttcaaagagcaaatctaagtag

SEQ ID NO: 123 - DNA, 606 bp, Photobacterium aquae
  source 1..606: mol_type = genomic DNA
atgacaacacggtattactttacgattcagtacattccaactcatgctgattttgggttactagctgggcggtgcatttatcaaatgcataaattcatggtcaacaacccgctagcgatgaatcaaatcggtgttagctttccaatgtgggaggatgggtctgttgggaatatcattgcttttatctctgaagataaggagttgatggtagggctattatttcaaccttacttttctttgatggtaaaagaaggtctttttgaaatatcgagtgtttgcgaagttcccacagatagtccagaagtgaggtttgtacgaaaccaaaccataggaaagagctttattgggtccaagaaacggcgaataaagcggagtatggcgagagctgagttgtctggtgctgagtattcattgccagttgcggtggaggaacgagttgtagaccatttccatcgagtaccaatttccagtggttcttctggacacgactacatcttacacattcaaaaagaggtcgctagtgaacgctctgtcgccaatttcaatagctacggattagccaccaaccaagaaaaaagagggactgtacctgacctatgcatatga

SEQ ID NO: 124 - DNA, 627 bp, Photobacterium aquae
  source 1..627: mol_type = genomic DNA
atgtacattcgtaatcttcgcaaaccctctccgaataaaaatatctataagtttgctagtagcaagaatagaaagacggttatgtgtgaaggcggccttgagaaagattgttgctaccactttgaatatgacccggaagttgtttgttacgagtcacagccggagggttattattatgagttttgtggtaaacagcttccctacacacctgactttttagtccactatattggtgggtatcaatgtttcgttgagagtaagccatatgggcaaacccttagcaaagagtttaagcaacagtttcaggctaggaagtcggcagctgagcgacttggttttgatcttatcttagtcactgaccggcaaatccgaaaagggtactatctagaaaactgtaaagttgttcaccgttatagtggttgtattaaaggggataacctacctgatgcgttgtatgatcaactgttggatactaaacccataaaaattatagacttagcgttaaaggttgaattgtctgttggtgtggtatttgctgctgtcttacgcttagttactttgggtaaggctttgattgaccttgattctgccaagcttaatgaaaccacgttagttatggtgaaatag

SEQ ID NO: 125 - DNA, 1926 bp, Photobacterium aquae
  source 1..1926: mol_type = genomic DNA
atggcagggcgatttaaggatgaattcgatgccaattactctgaggatgatgaaaaagagttcttagaatcacctgagtcaaaacgaaaccggcttcaatatgggtctttagattcagccaaaatcattgagcgagacctcgattctttccccgaagaacaaaaaacaaaagccttggaacgctataagttacttagcttagtcagcaaagagctggttggtggatggacacctaaaaatctcaacccactaatagacaaatattttgaaaaaacaacccttacccaaaagccaagctataaaactttaatacgttggcataattcatttaaccaagccaaaggtagttttactggtttggtagataagcatcatcaaaaaggtaatcgaacagctagagtagtgggcgatgagtcctattatgaaaaagccttagaaaggtttttagatgcagtcaggccatctattagagccgcctataatgtttattgtgataatattacggtcgcaaacgaaaacattgtttctggcaaaatccctcaagtttcctatcaaaccttcaaaaatcgtatccagaaagaacagccgtatagcgttgctttagcaaggcatggtaaatactacgcagacaagctctataactattaccaatcggttgatatgcctactcggatacttgagcgggtagaaatggatcataccccactcgacttaatcctacttcatgatgacctactggttcccttgggaagggcccacctaactcttttagttgatatcttcagcggctgcatcattggctttcatctgggatttaaacaccctagctatgtttctgcatctaaagctattatccatgcgactaagaataaagactacatttcaggtttacctattgagtttgaaaataagtggttatgtgaagggaaaattgaaaatctagtcgttgataatggccctgaattttggtcaaaaagccttgaagattcatgtttagaagcaggtattaatgttgtatttaacaaagttagaaaaccttggttgaagccgtttgtagaaagaaagtttggagagataattcagggtattgttggttgggttcctgggaagacgttctcgaacgttcttgaaaaagaagattataaccctgaaaaagacgcagttatgcgctttagtgtctttgttgaagaattgcaccgttggatagtcgatgtccacaatgctagcgcagacagccgcaaggctcggatacctaatctgtattggcggaaaagttatgaggtcatgcctccgttaaagctattacctgaaaatgagcatacctttaccattgcaatgggaagtctccatcatcgaaaactaacgagtaagggtatcaagtttaagcatatagattatgattcaactgctctggcgcaataccgaaaggaataccctcaaacaaaagcatctgctattaagaaaataaaagtagatcctgatgatatatcgacaatttatatctatttggaagagttaaatgggtatgttgaggttccaagtaaggactcgaaaggttacacccgcaagttaagtttgtgcgagcatgaaaagttagttaaagctcaccgagactatatcgatggtgagattgatgtgttgtcattagcaaaagctcgattagcgttgcatgaacgaattcaatcagagcaagaaaatctacagcatatgtctttgtctgaacggaaacgcaaggctaaagcaactaagaaaattgctgaactatcaagtgtaaatagtgatacgccaaaagcacaactgaccgataaacttcccccaaacccttcaatgagtggctgttccggttcaccggctgaaaaagaaattaatccaattgagaactttcggtctaaatggaataagcgaagaaaagagaggaatggatga

SEQ ID NO: 126 - DNA, 984 bp, Photobacterium aquae
  source 1..984: mol_type = genomic DNA
atgcctgattctaacctcgagctgtccatcgataccactctagcgacataccatgcgtcatttacgatctaccctgaggtagagaaggtgttttcgggactcgattggttagttaagcgtcgttgttttggttcgtttgtgccatcaatgttgttaactggagggacggggtctggtaagtcagcgttaatcaaacactatatatcgaaatgcctatcagaaaatgaggtattgctcactcgtgtgcgacctactttaaaggaaacgttactttggatagtgaatgaaatagataaatacaaaaaataccgagctaagggatccgtccttggattaatagattatgtcattaggtgcgttaagcggacagaactaaagttattagtgattgaagagtgtcaggagctttttgaatgtaccagtcataaagaacgacaagaaattcgagataaactgaaaatgatcagtgatgaatgtcgactaccaattgtttttgttggcattcctagtgcgaagttgatacttgaagactcccaatgggatagacggatcatggtgaagcgggaattaccctactttaaaattacggatgaagcgtcaattgatagatacttagacctacttgaggccatggaaagggcagtaccgctcccttttgacgttgacttaatggatgttgaaatagcgatgaggctattagccgctagccatggcatgcttggaatgttaaaagaactgatagctgttgggctagaatctgcgttgatttcaaataaggcggctattcagcttgaagacttcattttaggctacgaaatgatttttggtttagatgagataaatccattctctgttgatatcaatgagttggtgattaagcaaattgagtcttatgaggaatacgtaccggatgcagagactggagagctcaagtttgttggacagatattcaatgcattaacaatcaaacagctattaggttaa

SEQ ID NO: 127 - protein, 403 aa, Photobacterium aquae
MNTDIQFYPDESLESFLLRLSHHQGYERFAYFAEDIWYQTRDQHEAIAGAFPLELNRVNVYHAHTTSQMRVRVLMHLENQLNLDDFRVLHIVLAHSKSQFSPDFKAVHRCGVDYPFAFLRKRFMPVCPLCLAESAYVRQHWHFIPIQACEQHGCKLIHRCPACDGLLEYQSTECMTHCECGFNLLSTPTTSASASELLISRWLTGTQLDVAGLMGKALSISERYGFLLWYVNRYGDLEDISFDVFVEYCTTWPKSLRKDLDECVQKADAIRVKRWKQVFFSEAFGALLKGCRQLPSRQLSKNCVLVEILNYFKRLVADNPKSSKGNIADVLLSPLEASTLLSCTTDEVYRLYEFGEIKAAMRPKIHTKIASHESAFTLRSVVETRLTRMCSENDGLSVYLPEW

SEQ ID NO: 128 - protein, 695 aa, Photobacterium aquae
MKKLCDVLQIEDNTEKQATLKKVFMPYSACIDIDGCEKEALTVLLNLSTHRKGSPCGDWLDIERAKSYLKDQADIDASLAEIKWFHTHNLKFPDCRVKEQRLIAKPLSTSESFISSVSLDQGLGWAHNSAVYRHTLWLLNSFNWQSESVNILSLVQEENPVWLELLQEFGLNIKQQDLLLKTIELQIPASTFPDSVSPYSKQLRFPWNNDYLSVTPVVSHAIQREIEVKARDKASKLSFVTSALPNSASIGNLCGSLGGYMKALNYPLDVKSVAEQTLAASRNKSGKYFDDFQVTNYKICQVLNRLIGAEPLKNQKQREKARKVQSKILRKQIALWMLPLIELRDIEDAEPHNQQLEHDDPLVKSFLSLPESEFPSLVHELNQRLHFVFQENKFTAKFAYHPKLIQVVKAQIVWVLEQLSKPSDHEDAAREQQYIYLSSLRVQDAVAMSSPYLCGAPSLTAIWGFMHHYQREFNKLVNSDSPFEFSRFAFYVRTENIQSTAKLTEPNSLAKSRTLSNAKRPTIRSERLADLEIDLVIRVDSDSRISDFLSELRAALPAAFAGGALYQPLILSQIDWLRTFSSKSELFHVLKGIPAYGSWLYPSEKQPTNFNELEHLITEDADNLPVSIGYHLLEHPTERENSITDCHAYAENALGIAKRLNPIEVRFSGRDHFFDNAFWALESTSATILIKNDRN

SEQ ID NO: 129 - protein, 345 aa, Photobacterium aquae
MELCNQLNYVRSLSAGKAYFYHLSKGGEMCPLEIDRTRLRAPKGGYAEAYKGSKFVQKNVAPQDLAYANPQFIEECYVKPGVDDIYCAFSLRIKANSLGPDVCCDDEVRSKLSSLAKSYKELSGYSELAHRYAKNILLGTWLWRNRECRRLSIEVTTSDSETLIVENATKLTWYDHWDKDAAECLDKLTAYLTRALSDPTEYFYMDVKAKIAVGWGDEVYPSQEFLDNREDGVPTKQLATVELENGRETVAFHGQKVGAALQSIDDWWHEKADKPLRVNEYGADREYVIARRHVSLKNDFYQLLRNTENWIESMNTSNIIPNDVHFIMSVLVKGGLFNCSKSKSK

SEQ ID NO: 130 - protein, 201 aa, Photobacterium aquae
MTTRYYFTIQYIPTHADFGLLAGRCIYQMHKFMVNNPLAMNQIGVSFPMWEDGSVGNIIAFISEDKELMVGLLFQPYFSLMVKEGLFEISSVCEVPTDSPEVRFVRNQTIGKSFIGSKKRRIKRSMARAELSGAEYSLPVAVEERVVDHFHRVPISSGSSGHDYILHIQKEVASERSVANFNSYGLATNQEKRGTVPDLCI

SEQ ID NO: 131 - protein, 208 aa, Photobacterium aquae
MYIRNLRKPSPNKNIYKFASSKNRKTVMCEGGLEKDCCYHFEYDPEVVCYESQPEGYYYEFCGKQLPYTPDFLVHYIGGYQCFVESKPYGQTLSKEFKQQFQARKSAAERLGFDLILVTDRQIRKGYYLENCKVVHRYSGCIKGDNLPDALYDQLLDTKPIKIIDLALKVELSVGVVFAAVLRLVTLGKALIDLDSAKLNETTLVMVK

SEQ ID NO: 132 - protein, 641 aa, Photobacterium aquae
MAGRFKDEFDANYSEDDEKEFLESPESKRNRLQYGSLDSAKIIERDLDSFPEEQKTKALERYKLLSLVSKELVGGWTPKNLNPLIDKYFEKTTLTQKPSYKTLIRWHNSFNQAKGSFTGLVDKHHQKGNRTARVVGDESYYEKALERFLDAVRPSIRAAYNVYCDNITVANENIVSGKIPQVSYQTFKNRIQKEQPYSVALARHGKYYADKLYNYYQSVDMPTRILERVEMDHTPLDLILLHDDLLVPLGRAHLTLLVDIFSGCIIGFHLGFKHPSYVSASKAIIHATKNKDYISGLPIEFENKWLCEGKIENLVVDNGPEFWSKSLEDSCLEAGINVVFNKVRKPWLKPFVERKFGEIIQGIVGWVPGKTFSNVLEKEDYNPEKDAVMRFSVFVEELHRWIVDVHNASADSRKARIPNLYWRKSYEVMPPLKLLPENEHTFTIAMGSLHHRKLTSKGIKFKHIDYDSTALAQYRKEYPQTKASAIKKIKVDPDDISTIYIYLEELNGYVEVPSKDSKGYTRKLSLCEHEKLVKAHRDYIDGEIDVLSLAKARLALHERIQSEQENLQHMSLSERKRKAKATKKIAELSSVNSDTPKAQLTDKLPPNPSMSGCSGSPAEKEINPIENFRSKWNKRRKERNG

SEQ ID NO: 133 - protein, 327 aa, Photobacterium aquae
MPDSNLELSIDTTLATYHASFTIYPEVEKVFSGLDWLVKRRCFGSFVPSMLLTGGTGSGKSALIKHYISKCLSENEVLLTRVRPTLKETLLWIVNEIDKYKKYRAKGSVLGLIDYVIRCVKRTELKLLVIEECQELFECTSHKERQEIRDKLKMISDECRLPIVFVGIPSAKLILEDSQWDRRIMVKRELPYFKITDEASIDRYLDLLEAMERAVPLPFDVDLMDVEIAMRLLAASHGMLGMLKELIAVGLESALISNKAAIQLEDFILGYEMIFGLDEINPFSVDINELVIKQIESYEEYVPDAETGELKFVGQIFNALTIKQLLG

SEQ ID NO: 134 - DNA, 28 bp, Photobacterium aquae
  source 1..28: mol_type = other DNA
actacctactgaataagtagctgagaat

SEQ ID NO: 135 - DNA, 28 bp, Photobacterium aquae
  source 1..28: mol_type = other DNA
ccaacctgctgaaaaagcagtatttaac

SEQ ID NO: 136 - DNA, 28 bp, Photobacterium aquae
  source 1..28: mol_type = other DNA
gtgccctgccgagtaggcagccagaaaa

SEQ ID NO: 137 - DNA, 28 bp, Photobacterium aquae
  source 1..28: mol_type = other DNA
gtgccctgccgtataggcagttaaaaat

SEQ ID NO: 138 - DNA, 110 bp, Photobacterium aquae
  source 1..110: mol_type = other DNA
tgtcgcaaaaaccatatccagtcccaaaaaatccataagttgtccttttaaaagtcaaaaagtgtcctaatcttaatttggttgttttcttacaacattaagggacgcac

SEQ ID NO: 139 - DNA, 110 bp, Photobacterium aquae
  source 1..110: mol_type = other DNA
tacgaaaaggatatcttatggtttaatatttggacatgatatggttttcataatggcctaaattattgaaatatagctggttcattggacgctttatgatttcagcgaca

SEQ ID NO: 140 - DNA, 2100 bp, Vibrio sp.
  source 1..2100: mol_type = genomic DNA
atgaagctaagtgatgtattgcgaattgaagatgaagtgctcaagcagacgacgttcaaaaaagtattcatgccttatagtgaagatatcgaaattgatggctgtgaaaaagaagctttaatcatactgcttaacttaagttactaccccaaaggaacgaagcatattaactggctggatgatgaacgagcgctagattatctcacagagcaagacaacctaacagcctcgctggcagaggtacaatggttccacacccataacctaaaatacccagattgccgagtaagtaagcagaagattattggtgaacctcttcctgcagatgacgttttcatttcaagtgctacgttgaagccaatcttaggctgggcgcataattcggctgcttatcgctacacgatttggttgctgaattcttttatttggcagtctcaacccaccaatattcttaccttaatagagcaacaaaacccaatttggctagacttgctgagggcctttggtttgcgcgaaaagtcgcttgaactccttagaaccgaaattgagttgcagctttcctcacaatcgtttcctcgctatgttgattcgtactctaagcagctgcgtttcccttggaacggagattatttatcggttactcccgttgtgagccatgcgatgcaacgggaattggaacatcgttatagaaacgcagagagtcatctcaagtttgtgactttgtcattccctaactcagcaagtatcgggaatttatgtggtagtgtgggtggtaacatgcaagtactgaattacccattagatgtgccgtcatcgacaaataggtcaacattaagaaaaacactagccgacagcaggttagcgagtggtcggtattttgatgatttccagctaacgaatgaaagaatatgtaaagttctaagtcgtttgactggaacagaaacatctacaacacacaaacgaagaattaaatcgcgaaaagatcaaagtagaattttgagaaagcaggttgccttgtggatgctaccgttaattgaactgcgcgatcgtttcgatagcgatgaacgagagggcgttattgaggagcatgaaagcttggttcaagactttctaacgctatctgaatcagatctacccgtgctggtaagccagttcaatcagcgcttacattatgtatttcaagagaataaatttaccagaaaatttgcttatcatcctaaactgctacaagttgttaaatctcagatagtttgggttcttaacaaactgagcaagcctcaggaagatgaagttagtggccagggtgaacagtatatctacctttcttcactgcgggtgcaagactccctggcgatgagttgtccatacttatgtggtgttccatcactaacggctatttggggctttgttcatcactatcaaagagagttcaatagatcgattaacagtgatgtgttttatgagtttgccggcttttcgatatatgtgcgaagtcaaagcattactgtgggcgcgaagttgacggagcccaattcagtagaaaaagtccgaaccctttcaaatgcaaagcgtccaacgattcgaactgataggtttgctgacttagaaatagatttagttatatgcgtaaaaagcaatggcagattatcggattaccgcgcagcacttaagtctgtactcccgctttctttggccggaggctctttgttccaacctctcatctcatcaaaaatcgactggctgagaacgttcgatagtcaatctagtttgtttcatgcattaaagggcttaccagcttatggacggtggttatacccctgtgagctgcaacctgatagttttgatgaattggaatcgactttggatcagaatagtggctgtctaccagtctcaaatggataccactttttagagattcctatacatcgaaataatgccctcactgcgttacacacttatgccgaaaatacattgactgtagctaaacaggttattcctatagagatgcgttttgccggaagcaaacagttttttcaggaggctttttggtcgctagaatgcagttcaacaactattcttgtaaaaaaatacaaggaatag

SEQ ID NO: 141 - DNA, 1056 bp, Vibrio sp.
  source 1..1056: mol_type = genomic DNA
atggaactgtgtcgtcaactcaattatttgcggtctatctctccagggaaagcgtatttttactacttggcttcgaatggagataggtgccctttagcgattgataaaactcacatacgggcaccaaaaggcgggtatgcggaagcttatcaaggtagttcatttgtcaaaaaaaatgttgcacctcaggacttatcttattccaatcctcaatttattgaagaatgctatgttccaccattgaccaatgaaatcatctgtgagttttcacttcgaatcagagctaactcgttacatcctgatgtatgtagtgatgaaaaagtgcgcgagcaattaatgtcgcttgccaaagtttataaagagctgaacggttatcaagaacttgcttatcgttatgcgaaaaatatactgctcgggagttggttatggagaaataaagattgccgtggagttacaatccaagtgatgacgagtgatggtgaatccattgaagtctatgatgcaacaaagctttcttggtatggtcactgggatgagcaatcaacacaaagcctagaacaattaacgagctatttgtcgagagcattgtctgacagatctcaatgcttctatatggacgtgaaagctgtaatgtcagtcggacgaggggatgaggtctatccaagccaagagtttatagatgttaaacaagagggaattcccacaaggcagttagctaaggtgcctttgaactatgaacaagagacagctgcatttcatgctcaaaaaattggtgcagcattacagtcaatcgatgattggtggcatgaaaatgcagataagccgttaagagtgaatgagtatggggctgacagggaatatgtaattgcaagacgtcattcactattaggtaacgatttttatcagttgattcgtcgtactgaaaagtggatagaagagatggataaatctaaatcgatacctaatgatgttcattttattttgtcggtgcttattaaaggtggtttattcaattgctcgaaaaccaaatctaaatccaaatctaaatctaaatga

SEQ ID NO: 142 - DNA, 603 bp, Vibrio sp.
  source 1..603: mol_type = genomic DNA
atggaaagaagatattacttttcgattcgctatgttccatcatatgcggattttggactcttggctggtcgctgtatataccagatgcacttgttttcagttaacaatcctgaggtgaaaaataaagtgggagtttgttttcctcgctggaattcgaaggatgttggtgacatgatcgcctttgttatggaagataaagaagcgctattggggctagctttccaaccttacttttccaggatgacaaaagaaggcgtatttgaactttcgaaagttgatgaggttccaaaatcgtcttctgaagtgaggtttgtaagaaatcaagcgattggcaagagttttattgcttctaaaaaacgaaggataaaacgaagcatgacgagagctgaattattggattttgagcataccccggttgctgttgaggaacgtgttgtggaacattatcaccgcattccaatttctagtggctcatctggccaggattatatattacatattcaaaaagaacgcgtcgaatcaagagggcagcaagattttagtagctatggcttagcgacaaagcaagaaaaaagagggacggtccctgcgctgtatatttga

SEQ ID NO: 143 - DNA, 1212 bp, Vibrio sp.
  source 1..1212: mol_type = genomic DNA
gtgattacaaatattcaactataccctgatgaatcgctcgaaagctttctgttgcgactatcgcaagagcaagggtacgagcgattttcccattttgcagaagacatctggtatcaaacacttaatgaaaatgaggcgatgtcaggtgctttcccactagagcttaattgcatcaacatttatcacgggcatacaaccagcgaaatgagagcgcgtgtcctcattgacctagaacgccgaattaaactcaacgactttggtgttttacgtttggccttaatgcactcaaaagccaatttttcaccaaaattcaaagctgtgcatcgtttcggtatggattatcccttttcctttctgagaaagagatttacaccaatttgtcctatgtgtttgggtgatgcgccgtatatacgacagaattggcagtttatacctgttcaatcatgtgcggagcacggctgcaagttgctccatcaatgcccagaatgcggttgccgacttgagtatcagaattcagagcgcattcaatattgtgaatgtggttctaaccttgccgaagctgaggcaaaagtatcgtttgaaagcgagttgatggtagctcgctggctagccggaaagtcacctatggaagagggcgtcatgagcaaagatatgacaacctcagagcgctatggttttttgctttggtacgtgaaccgctatggcgatctagaagatatcagctttcatgcctttgtaaaatattgtgcagagtggccaaaaccattacaccacgaactggataagctggtagataaagctgatgtaattcgagtgaagcaatggagaaaggtatttttcagagaagtgtttggggaattgttgaaagagtgccgcgaactcccaagtcgccagttaagtaagaatattgtgttagttgagattttacattatctcactcgtttagttgccgacagttcctcgtcgcctaaaggaaatatcgcagatgttttgttgagcccgtttgaagcgtctacgttgctttcatgtagtacagatgaagtatatcgtttgtataatttcggtgagattcaagcagcatttcgaccgaaaatacacactaaactagctcgccacgaaccagtatttactttgagaggaatgatagagacaaagttggttcgtatgtgttccgagagtgatggccttagtgtctatctctcgaactggtaa

SEQ ID NO: 144 - DNA, 636 bp, Vibrio sp.
  source 1..636: mol_type = genomic DNA
atgtacccacataccatagataagccacacgctaagaaaaatatcttcaagttcattagtgtgaaaaataaagccattatcatgtgcgaatcatcacttgaatttgatgcttgttttcaccttgaataccatcctgacgttgcatcttttgagagccagccgttttacttggaataccagttagaagatggttcacatagctacactcctgactttcttgtaacgctgaatgatggtaagaaatacctacaagaagtcaaaaattccaagttatgtcttacgcctgaatatcttcatgttttcgaagcaatgcaaagaggtagtgaagatattggatttcccctttatctcgttacagaaagacaaattcgcaaagccttcatcttagataaccttaaattgatccaccgttatgcaggttcaaagcacatttgttctttcaaacaatcacttttagaagtcattcaaaaccaagggttgtcgtcatctgaaaaactcgccggtatatttggtaagtccataggctttatgaacagagaattgttagagctaatgagccttggccttgtttctgcccattttgaaacatccatgttcgatgagagaactgcaatttgggtgagtgaacaagtatga

SEQ ID NO: 145 - DNA, 1917 bp, Vibrio sp.
  source 1..1917: mol_type = genomic DNA
atgagtggtccatttgttgacgaatctaaaggtgaaccgcctaataatggtgaaggtggtctagtacaagatgtaaatattggtgatgttacaacagactcttctgatctttgttaccgccctctaaatactttaacggtttacgaacgagacttagacagttttcctgaagagcttaaaaatgaagctttagagcgatttaagcttctgtcacttattggcaaagagttcgatggtccttggccatttaaacagatacaaaagttaattgaaaagtacaaaaatgacgtttctattccaactcctagcccaagaacggttcagcgttggagggaacgttatgagaagagtaacggcgacttaaaatctcttattgttagaaattatgctaaaggtaatcgtaaaccaaagatcataggtgatgaatattactttgatttagccgtgcaaagttggctcgaggctgaaagaccgaacatcacccgtgcgtatgaacgttactgtgactcaattgaagtagctaacgaatcaattgttgtgggtaaaatacctagcgctagctacaaatcattctcaaggcgacttaaacaactccctccctatgctgttgctctacaaagacatggaaaatattttgctgatctttggttcagacacaacgctaagcacaagccgcctacacgtattctggagcgagttgaaattgatcatacacaactggatttgatgttactccatgatgagtatcttgttcctattggacgcccctgcttgacgatgcttatcgatgtatttagcggttgtatcattggctttcatcttggttttcatgcaccgggttatgcaacggtagcaaaggcattgttaaatgcgatgaagcctaaagattacgtaaaagatttacctatagagttaaataatgaatggatttgtgaaggaaaaattgaaaaattggtcatggacaatggggctgagttctggtcgaaaagtattgacgatgcctgtaaagaactcaacattgctgttcaatacaatccagtcaaaaagccttggttaaaaccctttattgaaaggtcattcggtattctaaacaaaacgttactctccactattccgggaaagacgttttcaaatgttcttgaaaagggggactatgatgcagccaataaagctgtaatgaagttctctacgtttgttgaagaacttcatcgttggatcatcgatgtacataatgccaaacctgactcgagaaacaatcgcttgcctaacttatattggagccaaggcgtcaaaacacttcctccagctagactcccaattaaagacagcgaacaactgtctattattatgggtatattggtgaagcgaaaactcacggagaaaggtattcaatacgaagacttattttatcgtagtcaagcccttgccgattaccgagcaaggttcccacagacgaaagaatcagcgataaaaaccatcaaagtagaccccgatgatctgtcgagaatttttatctttttggaggaacttaatggttatatcaaagttccctgtgatgaccccgaaggatacacaaaacatctttcacttcatgaacacattattatcaaacgagcccataaacagtatatcaaagggcacgttgataccttgtcgcttgcgaaagcacgtctagctttggcagcgagaatggaagaagaaacagaagagttgcgttctttcaagagaaagcgtaagccgccgaaaaacattaaaaagatggctgagtattcgggactaagcagtgcagctattgaatcgaagtcaccagctttggataaaatgtcatcgagaaaaagcaacgccgaagaaccaaaggatattgccaattttttggatgattgggaggcgatactcggagatttaagtgatgactag

SEQ ID NO: 146 - DNA, 1023 bp, Vibrio sp.
  source 1..1023: mol_type = genomic DNA
gtgatgactagcaactctgagaacgttcaacgtcttgtttccaactttaatcagtcttttgctctgtttcctccattcgatgcaattcttagtgatctggaaaaactacgaaacaaaagtgcaagagaagggtttaaaccatcgatgttgatctatggagatacaggtgcaggtaagtctgcattacttgagcatttcactaaggagagcaaatccaaaacaggccgtaaggtactgagaacaagagtgaggccttcactgcaagagactcttagctggacacttcatgttctaaatcctctccggagaaataatcgatttgtaaaaaatgcatctgaaattggcttaaccgacatgcttatccgcgaacttaaacaagcaaacataggcattatgattattgatgaatgccaagagtttgtagaaattcgttcgaatgatgacaaaaaagaaataagtattcgattgaaaatgatcagcgaagaagcttctgtctcaatgatatttgttggtatgccttggtcaaaagaaatcaccagagatagtcagtgggagtccagaatacgtttagttcgagaaatcccttacttcaaagtgatcaatgagaatggtagtaataacaaaaaagagatgaagcgatttgctctgagtttaatggagatatcaaagttgatgccattagataaacaaccccaattagagcttcctgagttcagtttcccacttctggcttactcgaggggagaaatgcgagcactgaaagatattttatccgatgctctcgaaatagctcttaacgaaggcgcaaaggaactaacgagatatcatttacaagaggctgccaagttttctgtcgaaggcgaaaatccattcgatgaaaaagtcaatatgattcaaattcagacgattcagcagtacacacgttttgagctagatgataaaactggcagaagagagaggtttgatcgtgcttttacagcattgcagcaaattcctatcaataagttactcagtaaacgctag

SEQ ID NO: 147 - protein, 699 aa, Vibrio sp.
MKLSDVLRIEDEVLKQTTFKKVFMPYSEDIEIDGCEKEALIILLNLSYYPKGTKHINWLDDERALDYLTEQDNLTASLAEVQWFHTHNLKYPDCRVSKQKIIGEPLPADDVFISSATLKPILGWAHNSAAYRYTIWLLNSFIWQSQPTNILTLIEQQNPIWLDLLRAFGLREKSLELLRTEIELQLSSQSFPRYVDSYSKQLRFPWNGDYLSVTPVVSHAMQRELEHRYRNAESHLKFVTLSFPNSASIGNLCGSVGGNMQVLNYPLDVPSSTNRSTLRKTLADSRLASGRYFDDFQLTNERICKVLSRLTGTETSTTHKRRIKSRKDQSRILRKQVALWMLPLIELRDRFDSDEREGVIEEHESLVQDFLTLSESDLPVLVSQFNQRLHYVFQENKFTRKFAYHPKLLQVVKSQIVWVLNKLSKPQEDEVSGQGEQYIYLSSLRVQDSLAMSCPYLCGVPSLTAIWGFVHHYQREFNRSINSDVFYEFAGFSIYVRSQSITVGAKLTEPNSVEKVRTLSNAKRPTIRTDRFADLEIDLVICVKSNGRLSDYRAALKSVLPLSLAGGSLFQPLISSKIDWLRTFDSQSSLFHALKGLPAYGRWLYPCELQPDSFDELESTLDQNSGCLPVSNGYHFLEIPIHRNNALTALHTYAENTLTVAKQVIPIEMRFAGSKQFFQEAFWSLECSSTTILVKKYKE

SEQ ID NO: 148 - protein, 351 aa, Vibrio sp.
MELCRQLNYLRSISPGKAYFYYLASNGDRCPLAIDKTHIRAPKGGYAEAYQGSSFVKKNVAPQDLSYSNPQFIEECYVPPLTNEIICEFSLRIRANSLHPDVCSDEKVREQLMSLAKVYKELNGYQELAYRYAKNILLGSWLWRNKDCRGVTIQVMTSDGESIEVYDATKLSWYGHWDEQSTQSLEQLTSYLSRALSDRSQCFYMDVKAVMSVGRGDEVYPSQEFIDVKQEGIPTRQLAKVPLNYEQETAAFHAQKIGAALQSIDDWWHENADKPLRVNEYGADREYVIARRHSLLGNDFYQLIRRTEKWIEEMDKSKSIPNDVHFILSVLIKGGLFNCSKTKSKSKSKSK

SEQ ID NO: 149 - protein, 200 aa, Vibrio sp.
MERRYYFSIRYVPSYADFGLLAGRCIYQMHLFSVNNPEVKNKVGVCFPRWNSKDVGDMIAFVMEDKEALLGLAFQPYFSRMTKEGVFELSKVDEVPKSSSEVRFVRNQAIGKSFIASKKRRIKRSMTRAELLDFEHTPVAVEERVVEHYHRIPISSGSSGQDYILHIQKERVESRGQQDFSSYGLATKQEKRGTVPALYI

SEQ ID NO: 150 - protein, 403 aa, Vibrio sp.
MITNIQLYPDESLESFLLRLSQEQGYERFSHFAEDIWYQTLNENEAMSGAFPLELNCINIYHGHTTSEMRARVLIDLERRIKLNDFGVLRLALMHSKANFSPKFKAVHRFGMDYPFSFLRKRFTPICPMCLGDAPYIRQNWQFIPVQSCAEHGCKLLHQCPECGCRLEYQNSERIQYCECGSNLAEAEAKVSFESELMVARWLAGKSPMEEGVMSKDMTTSERYGFLLWYVNRYGDLEDISFHAFVKYCAEWPKPLHHELDKLVDKADVIRVKQWRKVFFREVFGELLKECRELPSRQLSKNIVLVEILHYLTRLVADSSSSPKGNIADVLLSPFEASTLLSCSTDEVYRLYNFGEIQAAFRPKIHTKLARHEPVFTLRGMIETKLVRMCSESDGLSVYLSNW

SEQ ID NO: 151 - protein, 211 aa, Vibrio sp.
MYPHTIDKPHAKKNIFKFISVKNKAIIMCESSLEFDACFHLEYHPDVASFESQPFYLEYQLEDGSHSYTPDFLVTLNDGKKYLQEVKNSKLCLTPEYLHVFEAMQRGSEDIGFPLYLVTERQIRKAFILDNLKLIHRYAGSKHICSFKQSLLEVIQNQGLSSSEKLAGIFGKSIGFMNRELLELMSLGLVSAHFETSMFDERTAIWVSEQV

SEQ ID NO: 152 - protein, 638 aa, Vibrio sp.
MSGPFVDESKGEPPNNGEGGLVQDVNIGDVTTDSSDLCYRPLNTLTVYERDLDSFPEELKNEALERFKLLSLIGKEFDGPWPFKQIQKLIEKYKNDVSIPTPSPRTVQRWRERYEKSNGDLKSLIVRNYAKGNRKPKIIGDEYYFDLAVQSWLEAERPNITRAYERYCDSIEVANESIVVGKIPSASYKSFSRRLKQLPPYAVALQRHGKYFADLWFRHNAKHKPPTRILERVEIDHTQLDLMLLHDEYLVPIGRPCLTMLIDVFSGCIIGFHLGFHAPGYATVAKALLNAMKPKDYVKDLPIELNNEWICEGKIEKLVMDNGAEFWSKSIDDACKELNIAVQYNPVKKPWLKPFIERSFGILNKTLLSTIPGKTFSNVLEKGDYDAANKAVMKFSTFVEELHRWIIDVHNAKPDSRNNRLPNLYWSQGVKTLPPARLPIKDSEQLSIIMGILVKRKLTEKGIQYEDLFYRSQALADYRARFPQTKESAIKTIKVDPDDLSRIFIFLEELNGYIKVPCDDPEGYTKHLSLHEHIIIKRAHKQYIKGHVDTLSLAKARLALAARMEEETEELRSFKRKRKPPKNIKKMAEYSGLSSAAIESKSPALDKMSSRKSNAEEPKDIANFLDDWEAILGDLSDD

SEQ ID NO: 153 - protein, 340 aa, Vibrio sp.
MMTSNSENVQRLVSNFNQSFALFPPFDAILSDLEKLRNKSAREGFKPSMLIYGDTGAGKSALLEHFTKESKSKTGRKVLRTRVRPSLQETLSWTLHVLNPLRRNNRFVKNASEIGLTDMLIRELKQANIGIMIIDECQEFVEIRSNDDKKEISIRLKMISEEASVSMIFVGMPWSKEITRDSQWESRIRLVREIPYFKVINENGSNNKKEMKRFALSLMEISKLMPLDKQPQLELPEFSFPLLAYSRGEMRALKDILSDALEIALNEGAKELTRYHLQEAAKFSVEGENPFDEKVNMIQIQTIQQYTRFELDDKTGRRERFDRAFTALQQIPINKLLSKR

SEQ ID NO: 154 - DNA, 28 bp, Vibrio sp.
  source 1..28: mol_type = other DNA
gtgacctgccgagtaggcagctaagaaa

SEQ ID NO: 155 - DNA, 28 bp, Vibrio sp.
  source 1..28: mol_type = other DNA
aaaccctgccgaataggtagtttgaatt

SEQ ID NO: 156 - DNA, 112 bp, Vibrio sp.
  source 1..112: mol_type = other DNA
tgtggctggaaccataagatgacattttagacacaatagttgtcattttggacagataagttgtcatggtctacttttaggttgtaaatttactacttagggctaggccatg

SEQ ID NO: 157 - DNA, 243 bp, Vibrio sp.
  source 1..243: mol_type = other DNA
aaactgctgcacttttaacccagcagcagtttagtattgtcatgattatctgatcacttattttgaagcatgatagacatgcaaagctgtaagaatgtttcttacattcaaattgctaaggcgttgccgatttttagtaaatgacactttatggtaaccataaatgtcactatatggttcttataatcatgtaaggctctgacttttaaaaggtaagggtgtcaccttatggtcctagccaca

SEQ ID NO: 158 - DNA, 2100 bp, Psychromonas sp.
  source 1..2100: mol_type = genomic DNA
atggtgaatagtatgcatttaaaagagttactagaaattcaagatattactgagcgaaataaactattgaggcgtgcttttagcgcttatactgaaaccattgatataaccggctgtgaagagtttgcgcttattattctattaaatttaacttatcgtagaaaccaagtggaggacttacttgataaggtattagcaaaaaaaacgctaaataatgaggattacattggtaaatgtattaatgaagtcgaatggtttcatactcataatttgaaatatcccgatattagagtaagtaaacaaacgttagcagtaaatccacctccattacatcctcatgttttgagtagtgctaattatgataaaatctttggttggtcacatgattcagccaaagtaaatgtggttaagttatttcttagctattttaaatggcaagatgatagctattgtcttgcacaaattctagtatctctgcctgatgattggaaagctgcattcacttcattgggaatgaaagttaaggtgcttttaaatttatgtggcagagtttcaagtgttttgccggaagaggttattccaagttttgtagatcgttattgtcgtcaaattagaatgccttatcacgatggttacactgccattacacctgtcattagtcattctatacaatctaaaatacaacaagctgcaattcaaaagaaaggttcatttactaaagtcgaatttacgcgtacagctgcggttagtgagctagtagcttctatcggcgggtttgttaatgcattgagttatcctcctgacgtaggtataagccatcatggtttaagtcagtctcgattgtttcaaatacaaaatggaaagcaaatatttaatggaaacgtgttattaaagccgcagtttatcggagcactagaagggataatctttaaccatagtgcactagcactaaagcaacgtcggcagttaagagttaaaagtattaaagaattaagaaatacactttcagaatggttcgcacctattctagagtggcgtttagatatcattgaaaacagagttaacctaattgactttgaaagtattaatgatcaattagaatataaattagtatcaacttctgacgataaattaccgtcattggttattcccttatttggttcgctaaatagtatgcttgctaatataccgatgatgcaaaaatatgcttttcaccctaagttaatggagcctttgaagtttgctttaaaatggctgctaagtaacatcgggaatgaaagtgatgttgccataaaagacgatgacttgccttttaggtatttacacttaagtggtgttcgggtctttgatgcgcaagctctatcgaatccttattgctcgggtataccatcgttaaccgctgtttgggggatgttgcatcattatcagagggagcttaatcaggcccttggtcttggtgttcgctttacttctttttcatggtttattagggattactctttgattccgggaaaaaaacttcctgaaataaatttacatggcactaaaccgaacgatgtaaagcgacctggaattatagacaatcaatattgtgatttaacatttgatttagtggttcatattgatggttatcaggatgaattattgaaattggatgatgagcctgaattgttgaaagctcactttccatcaaactttgcgggtggtgtgatgcatcaacctgaattagattccaatattgattggtgtcgtttatatcataaggaaaaatttctatttgaaaaaattcgacgattaccattatctggttgctgggtgatacctactgaatacaaggtacatgacttcgaatcattatttgcaattttaaataatgattcaaaattgagtccttccatgatgggttatatgttattaaataagccagagcctagacctggatcactagaaaaaatgcattgctatgcagaacctgttattggggtcgttgaatactcaccggcaattaatattaggttaaaaggtaaaagaaattacttccataaggctttttggatgttagatgcacaagaaaagtttatgctaatgaaaaaaatttaa

SEQ ID NO: 159 - DNA, 1053 bp, Psychromonas sp.
  source 1..1053: mol_type = genomic DNA
atggaactatgtaatatattaaaatatgatcgctccctttatcctagtaaggctgtgtttttttataaaacagatgattccgactttgttcctcttgaagcagatatatgccgtattcgagggcctaagtctggtttcactgaggcatttacaccgcaatttttacccaaaaatctatccactcaagatttaacacataataatatattaacattggaagaatgttacgtgcctccgaatgttgatcatctatattgtcgtttttctttacgagtacaagctaattcactagctccttctggttgtagtgatcctgaagtattctccttacttaaagagtttgcagaggtattcaaagagcgtggaggttataaagagttggccgttcggtattgccgcaatattttattgggcacatggttatggcgtaatcaaaatacaggtaatacggaaattaaaattaaaaccagctctggcaatgactatttaatcaataatactcgtaaagttgcatgggaaagtaaatggtctgatgaagagttaaaaacgttagaagatttaagtaatgaaattcaaaatgcgctgatcgaccctaatgtattttggagcgctgatataactgcaaaaatagaaagtgtattttgtcaggaggtttaccctagccaaatacttaccgataaagtttcacaaagtgaagcttcaaagcaatttgtaaaatccaaatttagtgatgggcgttatgccgttagctttaattcagtaaaaataggtgctgccttacaatctattgatgattggtgggatgatggggcaagtaaacgactacgagtacatgagtttggtgtagataaagaaataggaacgactcgtcgccctcctcattctgaacataatttttatcatatattaaaaagcgctgaaaggtatttgagcgaactgaataaatttaatgctaataacggtgaggtcgttaatccaaatatctattatcttttttctgtattaattaaaggtggaatgttccaaaaaaaagcagaaagtaaaaaaggataa

SEQ ID NO: 160 - DNA, 612 bp, Psychromonas sp.
  source 1..612: mol_type = genomic DNA
atgaaaagatattattttatgattcattttttgcctaaagaagcaaatctagctttattgacaggtcgttgtatttctatcatgcatggttttattttaaagcgagatattcaaggtgtaggtgtaacattgcctgcttggtctgatttatcaattggtaatgtcattgcttttgtccattccgacaagaaagttttgcaaatgctgaaagagcagtcttattttgaagatatgcagaactgtggtttttttgaactaacttctgttactgcagtgcctgataattgtgaagaagtttattttaaacgtaaccaagcaattgcaaaaatatttacaggtgaaagtagaagacgtttaaaacgtttagagaaaagggcattatctagaggcgaaacctttaatccacaaaaatgtgtagtttcaagggagtttgatgtgtttcatcgcattgccataagtagcaatagtaatcaaaatgactacatacttcatatccaaaagcagaagggggggactaaagttgggtctacatttagtaactatggtttttcttctaatgaaaagtttgatgggagtgtccccgatttatctcatttagtaatgaaattataa

SEQ ID NO: 161 - DNA, 1230 bp, Psychromonas sp.
  source 1..1230: mol_type = genomic DNA
atggcttttttattttctccaaaggcaactccattttctgatgaatcattagagagttacttattacgtattgtttctgaaaatttctttgattcttatgaagagcttagcctcgcaattagagaggagttacatgaacttgattttgaagctcatggggcttttcctatcgacttaaaaagattgaatgtttatcacgcgaaacataatagtcatttcagaataagggcgctaggtttactcgaagccctgttagatcttcctcgctttgaactgcaaaaaattgctttactaaaatcagatgtaacctttaattcttcggctgctttatacagaaatggggtagatattcctcagaaatttattcgttatcaaggtaataggaaagcctattctattcctatttgtccgcattgcttacgagaggaagcttatataaagcaaagttggcacatcgaatgggtaaatatctgtgttaaacatcaatgtactctcattcataattgtcctgaatgtaatttaccaattaattatatcgaaaatgagtctattactcattgttcatgcggctttgaattagcctctgcagatagtctgccggtaaaagaaaaaagtattaaatatttacatgcattgttggattcaaatatatgctctggtagtaatcctttgtttaattttacgacttcaagtgaccgttttgcagctcttctctggtttcataaaaggtattcccataaaaatgtgttttgcttagatgaagccgtagaatattttagtgagtggccagcaagtttttataaagagctaaatggcttaactaataatgcagaaatgaaactcattgatttgttcaataagactgagtttaggtttatttttgaagatctgatcttatcctctcctaacactcaaatattagagaagcctcattttattcttattaccttattagattatttagcggcattaatagaagaaaaccctaaatcaaagaaagcgaatatcggcgatttattggtgagtgtttcagaaactgctttgctgcttggtacatccatagatcaagtttatagattatatcaagatggtatttttcaaactgcattcagacttaagctgaatcagcgaattaatcctaataaaggcgtattctttttaaggcaagtcatagagtacaaagcaagttttggaaatgataaaaatagaatgtacgtatcggcatggtga

SEQ ID NO: 162 - DNA, 627 bp, Psychromonas sp.
  source 1..627: mol_type = genomic DNA
atgtatatacgcaatttaagaaagccttctcctaataaaaatgtattcaaattcgctagcacaaaaatgggtgaagttattttgtgtgaaagtacattagaatttgacgcttgttttcatcatgaatataacgacgaaattgaaagctacggtagtcaaccacaaggttttaaatatgaatttaatgggaaggttttaccttatactcctgatgcattaattttatataaaaatggtgttcataaataccatgaatataaaccttacagtaaaatttcatctacattatttagagataaatttgaagcaaagagacagacttcattaacgatggggattgatcttattttagtgactgataggcaaattcgagttaatcccattttaaacaatttaaagttacttcatcgatattctggtgtgtatggggtgagtaaagttcaagttgaattactgaaaattattcggtattcagaaacaataaaattaaatgatgtgagttctcaatgtaatctttcgataggtgaaactcgatcttttatttatggcttaatacataaaggttttttgaaagctgatctaggaaaagaagacttaagcaataatcctactttacgggcagtataa

SEQ ID NO: 163 - DNA, 1842 bp, Psychromonas sp.
  source 1..1842: mol_type = genomic DNA
atgagtgactttattaatgaatttgataactcaattgtgccgatcaaaccagaaactcccgatcaatacgttaaattggaagatgctaccttaattaaacgtgatcttgatactttccctgattttttaaaagataaggcatttgataagtacaaactgattagcataattgaaaaggaaatttcaggtggctggactcaaaaaaatatcgatccgattcttgaaaacctatttgaagaaaataatgcaaaaaaaccaaattggcgtactgttgttcgttggcgaaaagcatatatagacagtaatggagatctttcttctcttgttgttaagcagcataaaatgggtaatagaactaagcgaatagacggcgatgaatttttttttgataaagctttagaacgctttttagatgctaaaagacctaaagtcaccactgcataccagtattataaagatcttattatgattgcgaatgaaaatgttgttgaaggtgaaattcctgtaatatcatattcggcatttaataagagaattaaatcgttacccccttatccaacagcaatagcaaggcatggtaaatttaaagctgatcagtggtttgcttattgcggttctcatatcccacctactagaatcctagaacgtgtagagattgatcatactccacttgatttaattttactggatgatgagttacttattcccataggcagaccttatttaacattacttgtagatgtctttagcggttgtattcttggttttcacctaagttataaatctccttcttatgtatctgctgctaaagcgattagtcatgctatcaaacccaaatcacttgagcatcttagtattgcattacagaatgattggccttgttatgggaaaattgagaatttagtggttgataatggagcagagttttggtctaaaagcttagagcatgcatgtcacgcgactggaataaatattcagtataatccagtgcgtaaaccgtggctgaaaccatttgtagaacgtttttttggtatgataaatcaatgttttttatcagaaatcccagggaaaactttttcaaatatactagaaaaagaagaatataaacctgaaaaagatgcaattatgcgattctctacttttgttgaagagtttcatcgttggatcgttgatgtatatcatcaagatagtaactctcgcgagactcgtgtacctattaaaagatggcaacaaggttttgatgtatatccacctttaaaaatgaatgatgaagaggatgctcgattttctatattaatgcgagtatcagatacaagaacgttaaccagaaatggttttaaatttgaagagttaatgtatgactcaactgctttagctgattatcgtaaacattatccgcaaacaaaagaaagcgtaaaaaaaattattaaagttgatcctgatgatatttcaaaaatccatgtctatttagaagaattggagagttacctcgaagttccatgtacagaaactactggttacagtgatggattgagtctttatgagcacaaaacaattaaaaaaattaaccgagaaataatacgtgaatcaaaagatagcttagggttagctaaagcgagaatggcgattcatgaacgaataaagcaagagcaagaagtatttattaagtcaaaaactaaagcaaagttaacagcagttaaaaaacaagctcagattgcagatgtaagtaatactggcaaaggaagtatcaaagttccagaaattgaatccatacaaccgaaaagaagtaatattagcgacgtattagatgattgggaagatgaggtagaggcctttgaatga

SEQ ID NO: 164 - DNA, 1014 bp, Psychromonas sp.
  source 1..1014: mol_type = genomic DNA
ttgaatgacttaactgagctacagattcaacagttacgagattttagtgactgtattgttgtacaccctcaaataaaaataatttttgatgacttcgatgagttaagatttaatcgtaagtttcaatcagaccaacagtgcatgcttttaattggggatactggagtagggaaaagtcaccttattaatcattataaaaaaagagtattggctactcaaaattatagtagaggtaccataccagtattagtcagtcgcatatcaagaggtaagggactcgaagcaactttaattcaaatgctggctgatcttgagttatttggtagcagccaaataaagaaaagagggtacaagatagacttaaccaaaaaattagtggaaaacttaataaaagctcaggtagagttgctcattattaatgaatttcaagagttaatagagtttaaaagtgtacaagaacgccaactaattgctaatgggttaaagtttattagtgaagaggctaaagtgcctattgtgttggtgggaatgccatgggcagaaagaatagctgaagagccacagtgggcttctaggttaataagaaaaagacagcttgaatattttagtctaaaaaataacagtaaatactttcgtcaatatttaatggggttagctaagaaaatgccgtttgacgaacctccaaaacttaaagtaaaacatactacattagctattttttctgcttgtaaaggagaaaatagagcgctaaagcatctactgactgaagctttaaaacaagcgctaagttgtaatgaacatcttgaaaataaacactttgtttttgcttttgataaactttacctctctgatacgagttcattcagtcaacaaaaattaacgattaaaaaccctttcaagcaagatattaaagatattaaaatctatgaagttagtaaaaactcatcatataaccctaatgcaattgatccagaagacgcattaacaggcagagaattttcattaatagcatga

SEQ ID NO: 165 - protein, 699 aa, Psychromonas sp.
MVNSMHLKELLEIQDITERNKLLRRAFSAYTETIDITGCEEFALIILLNLTYRRNQVEDLLDKVLAKKTLNNEDYIGKCINEVEWFHTHNLKYPDIRVSKQTLAVNPPPLHPHVLSSANYDKIFGWSHDSAKVNVVKLFLSYFKWQDDSYCLAQILVSLPDDWKAAFTSLGMKVKVLLNLCGRVSSVLPEEVIPSFVDRYCRQIRMPYHDGYTAITPVISHSIQSKIQQAAIQKKGSFTKVEFTRTAAVSELVASIGGFVNALSYPPDVGISHHGLSQSRLFQIQNGKQIFNGNVLLKPQFIGALEGIIFNHSALALKQRRQLRVKSIKELRNTLSEWFAPILEWRLDIIENRVNLIDFESINDQLEYKLVSTSDDKLPSLVIPLFGSLNSMLANIPMMQKYAFHPKLMEPLKFALKWLLSNIGNESDVAIKDDDLPFRYLHLSGVRVFDAQALSNPYCSGIPSLTAVWGMLHHYQRELNQALGLGVRFTSFSWFIRDYSLIPGKKLPEINLHGTKPNDVKRPGIIDNQYCDLTFDLVVHIDGYQDELLKLDDEPELLKAHFPSNFAGGVMHQPELDSNIDWCRLYHKEKFLFEKIRRLPLSGCWVIPTEYKVHDFESLFAILNNDSKLSPSMMGYMLLNKPEPRPGSLEKMHCYAEPVIGVVEYSPAINIRLKGKRNYFHKAFWMLDAQEKFMLMKKI

SEQ ID NO: 166 - protein, 350 aa, Psychromonas sp.
MELCNILKYDRSLYPSKAVFFYKTDDSDFVPLEADICRIRGPKSGFTEAFTPQFLPKNLSTQDLTHNNILTLEECYVPPNVDHLYCRFSLRVQANSLAPSGCSDPEVFSLLKEFAEVFKERGGYKELAVRYCRNILLGTWLWRNQNTGNTEIKIKTSSGNDYLINNTRKVAWESKWSDEELKTLEDLSNEIQNALIDPNVFWSADITAKIESVFCQEVYPSQILTDKVSQSEASKQFVKSKFSDGRYAVSFNSVKIGAALQSIDDWWDDGASKRLRVHEFGVDKEIGTTRRPPHSEHNFYHILKSAERYLSELNKFNANNGEVVNPNIYYLFSVLIKGGMFQKKAESKKG

SEQ ID NO: 167 - protein, 203 aa, Psychromonas sp.
MKRYYFMIHFLPKEANLALLTGRCISIMHGFILKRDIQGVGVTLPAWSDLSIGNVIAFVHSDKKVLQMLKEQSYFEDMQNCGFFELTSVTAVPDNCEEVYFKRNQAIAKIFTGESRRRLKRLEKRALSRGETFNPQKCVVSREFDVFHRIAISSNSNQNDYILHIQKQKGGTKVGSTFSNYGFSSNEKFDGSVPDLSHLVMKL

SEQ ID NO: 168 - protein, 409 aa, Psychromonas sp.
MAFLFSPKATPFSDESLESYLLRIVSENFFDSYEELSLAIREELHELDFEAHGAFPIDLKRLNVYHAKHNSHFRIRALGLLEALLDLPRFELQKIALLKSDVTFNSSAALYRNGVDIPQKFIRYQGNRKAYSIPICPHCLREEAYIKQSWHIEWVNICVKHQCTLIHNCPECNLPINYIENESITHCSCGFELASADSLPVKEKSIKYLHALLDSNICSGSNPLFNFTTSSDRFAALLWFHKRYSHKNVFCLDEAVEYFSEWPASFYKELNGLTNNAEMKLIDLFNKTEFRFIFEDLILSSPNTQILEKPHFILITLLDYLAALIEENPKSKKANIGDLLVSVSETALLLGTSIDQVYRLYQDGIFQTAFRLKLNQRINPNKGVFFLRQVIEYKASFGNDKNRMYVSAW

SEQ ID NO: 169 - protein, 208 aa, Psychromonas sp.
MYIRNLRKPSPNKNVFKFASTKMGEVILCESTLEFDACFHHEYNDEIESYGSQPQGFKYEFNGKVLPYTPDALILYKNGVHKYHEYKPYSKISSTLFRDKFEAKRQTSLTMGIDLILVTDRQIRVNPILNNLKLLHRYSGVYGVSKVQVELLKIIRYSETIKLNDVSSQCNLSIGETRSFIYGLIHKGFLKADLGKEDLSNNPTLRAV

SEQ ID NO: 170 - protein, 613 aa, Psychromonas sp.
MSDFINEFDNSIVPIKPETPDQYVKLEDATLIKRDLDTFPDFLKDKAFDKYKLISIIEKEISGGWTQKNIDPILENLFEENNAKKPNWRTVVRWRKAYIDSNGDLSSLVVKQHKMGNRTKRIDGDEFFFDKALERFLDAKRPKVTTAYQYYKDLIMIANENVVEGEIPVISYSAFNKRIKSLPPYPTAIARHGKFKADQWFAYCGSHIPPTRILERVEIDHTPLDLILLDDELLIPIGRPYLTLLVDVFSGCILGFHLSYKSPSYVSAAKAISHAIKPKSLEHLSIALQNDWPCYGKIENLVVDNGAEFWSKSLEHACHATGINIQYNPVRKPWLKPFVERFFGMINQCFLSEIPGKTFSNILEKEEYKPEKDAIMRFSTFVEEFHRWIVDVYHQDSNSRETRVPIKRWQQGFDVYPPLKMNDEEDARFSILMRVSDTRTLTRNGFKFEELMYDSTALADYRKHYPQTKESVKKIIKVDPDDISKIHVYLEELESYLEVPCTETTGYSDGLSLYEHKTIKKINREIIRESKDSLGLAKARMAIHERIKQEQEVFIKSKTKAKLTAVKKQAQIADVSNTGKGSIKVPEIESIQPKRSNISDVLDDWEDEVEAFE

SEQ ID NO: 171 - protein, 337 aa, Psychromonas sp.
MNDLTELQIQQLRDFSDCIVVHPQIKIIFDDFDELRFNRKFQSDQQCMLLIGDTGVGKSHLINHYKKRVLATQNYSRGTIPVLVSRISRGKGLEATLIQMLADLELFGSSQIKKRGYKIDLTKKLVENLIKAQVELLIINEFQELIEFKSVQERQLIANGLKFISEEAKVPIVLVGMPWAERIAEEPQWASRLIRKRQLEYFSLKNNSKYFRQYLMGLAKKMPFDEPPKLKVKHTTLAIFSACKGENRALKHLLTEALKQALSCNEHLENKHFVFAFDKLYLSDTSSFSQQKLTIKNPFKQDIKDIKIYEVSKNSSYNPNAIDPEDALTGREFSLIA

SEQ ID NO: 172 - DNA, 28 bp, Psychromonas sp.
  source 1..28: mol_type = other DNA
gtgtcctgccgaataggcagctgataat

SEQ ID NO: 173 - DNA, 28 bp, Psychromonas sp.
  source 1..28: mol_type = other DNA
aatttctgccgaacaggcagttttatat

SEQ ID NO: 174 - DNA, 28 bp, Psychromonas sp.
  source 1..28: mol_type = other DNA
gtgtcctgccgaacaggcagctgagaat

SEQ ID NO: 175 - DNA, 211 bp, Psychromonas sp.
  source 1..211: mol_type = other DNA
tgtcgctgaaaccatacattgacataatataaccatactttgacaaaattaaagcatactctgacataaaatataaaactgtaattttattacacttttatttttctttttaatcagttgcttatgattttattagcgcatactctgacaaattgtgtaatttagttgtttagtttatactcagtagagataaaattaactggattactct

SEQ ID NO: 176 - DNA, 199 bp, Psychromonas sp.
  source 1..199: mol_type = other DNA
aaagagagtatagggtctttatatacctaaatagttgaataattatttgtaatttaattacatattgaaaatggcttgaatattaatttttttaatttgtcagtgtatggttgtattttatgtcaaagtttacttttatatatgatgtaagcaaatgatttactgtaccatgttatgtcagagtatgatttcagcgaca

SEQ ID NO: 177 - DNA, 1245 bp, Colwellia polaris
  source 1..1245: mol_type = genomic DNA
gtggcttttttattttctcctaaagttcgtgcgttttctgatgaaacattagaaagctatttactgcgtgttgtttcagaaaatttttttgactcatacgaacagttaagcttagctattcgggaagcgcttcatgaactcgactttgatgcccatggtgcttttccaatcgatttaaaaaggttgaacgtttaccacgctaaacacaatagccactttagaatgcgagcaatcggtcttctggaaacattgttagacctccctcgttttgaattgcaaaaacttgctttattaaaatcagataaaaagtttaactcgtccgttgcagttcatcgtgatggtatcgatatcccattaaagtttattcgttctaatggcgcggatggagaatgctctctacctatatgccctcaatgtttagctgaagaaccctatataaaacaaagttggcacataagatggataaacatttgcactaaacataaatgtacattaatccatcagtgtcccgactgtaacttacctataaattatattgaaaatgaatcaattgcccattgtttatgtggctttgatttggcctcagctaccaacagtaatacaacctctattactgtaaagcgtaaagatgctgagttaatacataacctgctcaataacgaaactttaattgataatccattgtttcgtgagactacaatatcccaacgttttgcggcattactttggtatcaagaaagatatagctgcatagataacttttgtttaaatgatgcagctaccttctttagtaagtggcctgaaaatttatataacgaattagaccatttaagtaaaaacgctaatatgaagttgattgagatgtttaataaaactatatttcgatttattttcggtgaggttattttatcagtaccacatagtattcaaagagaaggtcagcgtcatttcattcgaatagcgctaattgattatttaattcgtttagttgcaaacaatcctaaatcgaaaaagcctaatgttgctgatatgttagtgagcgtgactgaggtagcgataattttaggtacgtcacatgagcaagtttataggttataccaagatggtattttgcaaaatgcttttcgtcaaaaaatgaatagcagaatagatccccatactggtgtctttttcttaaggcaagtaatagagtataaatctagctttggtgacgataaacctaaaatgcacttgtcggcttggtaa

SEQ ID NO: 178 - DNA, 2097 bp, Colwellia polaris
  source 1..2097: mol_type = genomic DNA
atgcatttacaagaactgttagctattgaaagtgttccagatcgcgatcaatctttaaggcgtgcttttagcgcttatacagaagaaatagacattacaggttatgaatcaatcgcattaactattctgttaaacctcacttaccgtaaggtgcaaattgataacctacttgataaaaaactagccaagaaagcgctaaataatgaaagcctacttaataaatgtattgatgaacttcagtggtttcatactcataacttgaaatatcctgatattagagtcagtaaacaaaacttagtagttgattcacccatgttacatcctttggttttgagtagtgcaaattatgatagagcctacggatggacccataattcagctcaagtaaatttagttaagttatttgttagttatttttattggcagggaaataaatgttgtcttgcagaaattttagctgcacagccaatcgaagtagaatggaaagccgcatttcaatcacttggtatgcaagttaaattatttataaatatctgcggtagagttaaaggctttttaccacaagtaatgatcccagatactgtcgatagatactcaccccaagttagaatgccataccatgatggttatgtagccgttacaccagttgttagccatgtgctgcaatctaagatacaacaagctgctataaacaagaatggaaaattcagccaaattgaattcactaggtcagccgccgtaagtcaattagtcgcgtcccttggaggcgttgttaaggcattaagttatccgttgtatttcaataataaacaccatggtttgcatgattctcgtcgattaaaagtgcaaaatggacaaagtgtatttaaccttatcgctttaacaacaacacactttattaatgctttaaatggcctaatatataatggtaatgcattagcacttaaacaacgcagacagcaaaaagtgatttgtataaaggctgtcagaaatgctttgtctgagtggttgtctcctatttttgagtggcgttttagtatcattgaaaataaaagccaattagaacaattggacaaaatttctgatacgttagagtaccagctgttaacaacattagatgatgatttgcccgagttagttaatcctttatttggtgtgttaaatgcaatgttttcacacgatacatcaactcaaaagtatgcctttcaccctaaattaatagggtctataaaagcgagtttaaagtggctgttaagtaatgcagccattatcgataactcaccagcttttcacgataatgaagaacaatatcgatacttacatttacgggatataagggtgtttgatgctcaagcattatcaaacccgtattgtgctggtacgccgtcattaacagctgcttggggaatgatgcatcattaccaacgaaaactaaataatgcactagatacaaatgtaagatttacttctttttcttggtttatcaaagattattctaatgtgccaggtaaaaagttacctgaaatgcggttacaaggagcaaagcaaaatgagcttaagcgcccaggtattatagataataagcattgcgatttagtgtttgatttagtaattcatatagatggcaatgaagaagatttattattaatcgatgaacaacctgaaatgcttaaagcgcactttccaacaacttttgctggaggtgtgatgcatccacccgagctagatttagctattaattggtgtagattatatgacgatgaaaataaactgtttgaaaaactaaaacgtcttcctttatctggtcgatgggttatgccaacaaaacataaaatctgtgacttagatgagttgcttttgttgctcaacaatgatcttaagttaagccctattatgtttggatatttactactagataaacctaaaaaaaggtgtgggtctttagaagaattacattgctatgctgagccagctataggattagttgaatatatcacagccattaatataagattaaaagggaaaaacttttactttaatcatggtttttggatgttagaagctaaagaacagtttatgctaatgaagggagtttaa

SEQ ID NO: 179 - DNA, 1053 bp, Colwellia polaris
  source 1..1053: mol_type = genomic DNA
atggatatatgcaatatattaaaatacgatcgctcgctttaccctggtaaagctgttttcttttataagacaaaagataatgattttattcctcttgaggctgacattaataagattcgcggaccaaaagcaggttttactgaagcctttactccacaattttcacccaagaaattagctcctcaagatttaactcataataatattttaacgcttgaagagtgttatgttcctcctaatgtagagcacgtttattgtcgtttttcattaagagtgcaagccaattcattaaagccctctggctgtagtgatcctaaagtattttctttactaaaagaactagcaacagtatttacagatagtggtggctacaaagaattagcaactcgttattgtagaaatattttacttggtacttggttatggcgtaatcaaaatactggcaacaccgaaattgagattaaaaccagctcaggaaactcctataaaataaggaatacccgtaaagttgcatgggaaagttcatggccttctgaagatcaaaacgtattaaatgaattaagtgatgaaatgcaaaacgcattaatcgatccgaatatattttggagtgcagatataaccgcgaagattgagactgctttttgtcaggagatttacccaagccaaattctcagcgacaagccagcacaaggcgatgcttctaaacaatttgtaaaaaccaaatgtatagacggacgttatgctgtgagtttcaattcggtaaaagtaggcgcggcactccaatcaattgacgattggtgggatgaagatgcagataaaagattacgagttcatgaatttggtgctgataaggaacttagtgtaactcgtcgccctcctgattcaggcagaaatttttatcacttattgaaaaatactgaacaatatattgacgagctaaacgtaaaaaacacaacgacaaagtacactattaactcaaacatatattacatgttttcagtgttaataaagggcggactgtttcagaaaaaacttgaatcaaagaaggggtaa

SEQ ID NO: 180 - DNA, 612 bp, Colwellia polaris
  source 1..612: mol_type = genomic DNA
atgttacggcattattttatggtggagttttgtccaaaagaagccaatttccctttactttccggtcgctgcatttcgattatgcatggttttatttgcaaaaataatattcaagggttgggagtatcgcttcctgcttggtctgataacacaattggaaataaaattgctttcatccactatgatgatgtgactttaaatagtttaaaacaacaaccctattttcaagatatgaaagactgtggcttttttaaattgagcgacgtttgtgttgtaccaaatgaatgtaaagaggttaggtttaagcgaaatcaatcagtagctaaaatttttgtaggtgaaagccggagaagactcaagcggcttgaaaaaagggcactggcaagaggcgaagtttttagccctaaaaagtcgtcttacacaagggaattagatacttttcaccggattccaatgagtagtagctgcaatcttgaagattttattctccatatacaacgagaatacgtagattttaaaggtgaacaaagttttggtagttatggtttcgcaacgaataagaagtttcaaggaacggttcctgacttatctactttaactaataatatttaa

SEQ ID NO: 181 - DNA, 639 bp, Colwellia polaris
  source 1..639: mol_type = genomic DNA
atgtatattcgaaatttaagaaagccttcacctaataaaaatgttttcaaatttgctagcgcaaaaattaataatgttattatgtgtgagagtacattagagtttaatgcttgtttccatcatgaatacaatgatgaaattgaaagttatggcagccaaccaaaaggattcaaatatgagttcaatgggaaaaatttaccatacacccctgatacgctaatcatctataaaaatggtaatgagaaatatcacgaatataagccatttagcaaaatatcatctcccttatttagggaaaaattcaaggctaaaaaacaagcatctttaatattaggccgtgaattaatccttataacagacaaacaaatttgtgttaacccgattttaaacaacctaaaattacttcatagatattcagggatttatggtgttaatgcagttcaaaaggaattgcttaatcttattaaaaaatcagatgttatcaaatttaatgatgttagctctcagcttgggttatcaattggtgaagcccgctctttcttatatgcattgatacataaaggttttgtaaaagcggacttaagtcatgatgatttaacaaataatccaaccttatgggctgcctatgacgggttttaa

SEQ ID NO: 182 - DNA, 1842 bp, Colwellia polaris
  source 1..1842: mol_type = genomic DNA
atgacgggttttaatgatgagtttgatgaatcgatagtacctaataagcctaagacccccatccaatatataagattagaagacgcgaacttaattaagcgtgatttggacactttaccagaggctttaaaaaacaatacattagataagtataaaattattagttatatagcaaaggaactaactgaaggttggactcaaaagaatattgacccaattcttgataaactctttgataatgatgataaaaaacgtcctaattggcgaacaatagtccgttggcgaaaaaagtataatgagagtaatggagaccttacctcactcatttctgaacaccataaaatgggtaatagaaaaaaacgcatatcaggagatgaagtgttttttgataaagcattagaacgattcttaaatgctaaacggccaacagtatctaccgcttatcagtattataaagatcttatcattattgaaaatcaacatctttttgaaaataaaataccaacaatatcttatgtcgcttttaataaacgcattaaatcattacccccatataaagttgctgttgcaaggcacggtatatttaaagccgctcaatggtttgcttattgcggcgcacataacccacccacacggatattagagcgagtagaaatagatcatacaccacttgatttaatccttctcgatgatgaattactgatccctatcggcagaccttatttaacactacttattgatgtatttagtggctgtattatggggttccatttaagttataaatgcccttcctatgtatcagcagcaaaagcaatttctcatgcaatcaagcctaagtcattagattcaattggtatagagttacagaacaattggccctgttatggaaagattgagaacttagttgttgataacggtgctgaattttggtctaaaagcttggagcatgcttgccaatcagtaggaataaatattcaatataatcctgtacgtaaaccatggttaaaaccttttgttgaacgattttttggagtaatcaatcaatactttatatcagagttaccagggaaaactttttccaatatattagaaaaagaagaatacaaacctgagaaagatgcgattatgcgtttttcaacttttgtagaagagtttcatcgttggattgttgatatatatcaccaagatagtgattcgcgtgacactcgtatcccaataaaaaaatggcaaactggttttgaggtttatccgccacttgaaatgaatcaggaagaagaagaaaaattttctattttaatgaatatttctgacgaaagaacattaactagaaatggctttaaatttgaagagttaatgtatgattcaacagccctatctgaatatagaaagcattatccgcaaactaaagaaaccattaaaaaaattattaaagtggatcctgatgatatttcaaaaatctatgtctacttagaagagctgaatagttatattgaagtgccttgtacagaccctacaggttatacaaaaggtctaagtattcatgagcataaaacaattaaaaaaattaatcgggaaatgatacgagggtctaaagacgtattaggattagctaccgctagaatggctatacatgaacgagtaaaacaagaacaagaactttttattacgtctaaaaataaaagaaagatatcatccgttaaaaaacaagctcaattagcagatgtgagcaataccggcacaggaactattaaagtttccgaacaaaaatcaactacaaaacaagaaaatataagtaatgatattttagaaaattgggatgatgatttagaggctttcgagtga

SEQ ID NO: 183 - DNA, 1032 bp, Colwellia polaris
  source 1..1032: mol_type = genomic DNA
gtgaacaccttaactgaaacacaaattgagcaattacgccaattcaatgactgtattgttgaacaccctcaaataaaatcaatatttaatgattttgatgaattaaggcaaaaccgtctatttcaactggatcaacaatgtatgcttttgagtggggatgcaggtgttgggaaaagccacattatcaatcactataggaagagggtgttagctaatcaaaattatagtcggagtacgatacctatattgattagtcgcatctctaagggaaaaggtttagaagcgactcttattcaagtattagctgatttggatttattcgggagtgaacaaagaaagaaacgtggctataaaactgatctaactaaaaaattaatagaaagtttgataaaagcccaagttgagctactcattattaatgaatttcaagaattaattgaattcaaaagtcgacaagagcggcaacatatagctaatgaattaaaattaattagtgaagaggctaaagtgccaatcgtattagttggaatgccatgggcggagctaatagctgaggaaccacagtggtcttctcgattagtaagaaaaagaaagctaagttactttagtttaaaaaatgataaaaattattttattcgttacataatgggcttagctaataaaatgccttttgaggaacctcccactttgggagataaacagacagcgacggctatattttctgcttgtcgaggagaaaatagatcgctgaaacatctactatcagaatctttaaaaattgctcttttatctaatgaaaacttagaaataaaacacttggctttagcttttgataagctggaactattaggaaaagaagcacaaagacagaacaaaaagaaagaaggcaaggagaaaagcgaagacattgttatagataatcctttcaatcaaagtttaaaagatattgatatttctgaagtaattaaacattctcaatatgccccgaatgccttagatcctgaggatattttaactggaaggtttttttaa

SEQ ID NO: 184 - protein, 414 aa, Colwellia polaris
MAFLFSPKVRAFSDETLESYLLRVVSENFFDSYEQLSLAIREALHELDFDAHGAFPIDLKRLNVYHAKHNSHFRMRAIGLLETLLDLPRFELQKLALLKSDKKFNSSVAVHRDGIDIPLKFIRSNGADGECSLPICPQCLAEEPYIKQSWHIRWINICTKHKCTLIHQCPDCNLPINYIENESIAHCLCGFDLASATNSNTTSITVKRKDAELIHNLLNNETLIDNPLFRETTISQRFAALLWYQERYSCIDNFCLNDAATFFSKWPENLYNELDHLSKNANMKLIEMFNKTIFRFIFGEVILSVPHSIQREGQRHFIRIALIDYLIRLVANNPKSKKPNVADMLVSVTEVAIILGTSHEQVYRLYQDGILQNAFRQKMNSRIDPHTGVFFLRQVIEYKSSFGDDKPKMHLSAW

SEQ ID NO: 185 - protein, 698 aa, Colwellia polaris
MHLQELLAIESVPDRDQSLRRAFSAYTEEIDITGYESIALTILLNLTYRKVQIDNLLDKKLAKKALNNESLLNKCIDELQWFHTHNLKYPDIRVSKQNLVVDSPMLHPLVLSSANYDRAYGWTHNSAQVNLVKLFVSYFYWQGNKCCLAEILAAQPIEVEWKAAFQSLGMQVKLFINICGRVKGFLPQVMIPDTVDRYSPQVRMPYHDGYVAVTPVVSHVLQSKIQQAAINKNGKFSQIEFTRSAAVSQLVASLGGVVKALSYPLYFNNKHHGLHDSRRLKVQNGQSVFNLIALTTTHFINALNGLIYNGNALALKQRRQQKVICIKAVRNALSEWLSPIFEWRFSIIENKSQLEQLDKISDTLEYQLLTTLDDDLPELVNPLFGVLNAMFSHDTSTQKYAFHPKLIGSIKASLKWLLSNAAIIDNSPAFHDNEEQYRYLHLRDIRVFDAQALSNPYCAGTPSLTAAWGMMHHYQRKLNNALDTNVRFTSFSWFIKDYSNVPGKKLPEMRLQGAKQNELKRPGIIDNKHCDLVFDLVIHIDGNEEDLLLIDEQPEMLKAHFPTTFAGGVMHPPELDLAINWCRLYDDENKLFEKLKRLPLSGRWVMPTKHKICDLDELLLLLNNDLKLSPIMFGYLLLDKPKKRCGSLEELHCYAEPAIGLVEYITAINIRLKGKNFYFNHGFWMLEAKEQFMLMKGV

SEQ ID NO: 186 - protein, 350 aa, Colwellia polaris
MDICNILKYDRSLYPGKAVFFYKTKDNDFIPLEADINKIRGPKAGFTEAFTPQFSPKKLAPQDLTHNNILTLEECYVPPNVEHVYCRFSLRVQANSLKPSGCSDPKVFSLLKELATVFTDSGGYKELATRYCRNILLGTWLWRNQNTGNTEIEIKTSSGNSYKIRNTRKVAWESSWPSEDQNVLNELSDEMQNALIDPNIFWSADITAKIETAFCQEIYPSQILSDKPAQGDASKQFVKTKCIDGRYAVSFNSVKVGAALQSIDDWWDEDADKRLRVHEFGADKELSVTRRPPDSGRNFYHLLKNTEQYIDELNVKNTTTKYTINSNIYYMFSVLIKGGLFQKKLESKKG

SEQ ID NO: 187 - protein, 203 aa, Colwellia polaris
MLRHYFMVEFCPKEANFPLLSGRCISIMHGFICKNNIQGLGVSLPAWSDNTIGNKIAFIHYDDVTLNSLKQQPYFQDMKDCGFFKLSDVCVVPNECKEVRFKRNQSVAKIFVGESRRRLKRLEKRALARGEVFSPKKSSYTRELDTFHRIPMSSSCNLEDFILHIQREYVDFKGEQSFGSYGFATNKKFQGTVPDLSTLTNNI

SEQ ID NO: 188 - protein, 212 aa, Colwellia polaris
MYIRNLRKPSPNKNVFKFASAKINNVIMCESTLEFNACFHHEYNDEIESYGSQPKGFKYEFNGKNLPYTPDTLIIYKNGNEKYHEYKPFSKISSPLFREKFKAKKQASLILGRELILITDKQICVNPILNNLKLLHRYSGIYGVNAVQKELLNLIKKSDVIKFNDVSSQLGLSIGEARSFLYALIHKGFVKADLSHDDLTNNPTLWAAYDGF

SEQ ID NO: 189 - protein, 613 aa, Colwellia polaris
MTGFNDEFDESIVPNKPKTPIQYIRLEDANLIKRDLDTLPEALKNNTLDKYKIISYIAKELTEGWTQKNIDPILDKLFDNDDKKRPNWRTIVRWRKKYNESNGDLTSLISEHHKMGNRKKRISGDEVFFDKALERFLNAKRPTVSTAYQYYKDLIIIENQHLFENKIPTISYVAFNKRIKSLPPYKVAVARHGIFKAAQWFAYCGAHNPPTRILERVEIDHTPLDLILLDDELLIPIGRPYLTLLIDVFSGCIMGFHLSYKCPSYVSAAKAISHAIKPKSLDSIGIELQNNWPCYGKIENLVVDNGAEFWSKSLEHACQSVGINIQYNPVRKPWLKPFVERFFGVINQYFISELPGKTFSNILEKEEYKPEKDAIMRFSTFVEEFHRWIVDIYHQDSDSRDTRIPIKKWQTGFEVYPPLEMNQEEEEKFSILMNISDERTLTRNGFKFEELMYDSTALSEYRKHYPQTKETIKKIIKVDPDDISKIYVYLEELNSYIEVPCTDPTGYTKGLSIHEHKTIKKINREMIRGSKDVLGLATARMAIHERVKQEQELFITSKNKRKISSVKKQAQLADVSNTGTGTIKVSEQKSTTKQENISNDILENWDDDLEAFE

SEQ ID NO: 190 - protein, 343 aa, Colwellia polaris
MNTLTETQIEQLRQFNDCIVEHPQIKSIFNDFDELRQNRLFQLDQQCMLLSGDAGVGKSHIINHYRKRVLANQNYSRSTIPILISRISKGKGLEATLIQVLADLDLFGSEQRKKRGYKTDLTKKLIESLIKAQVELLIINEFQELIEFKSRQERQHIANELKLISEEAKVPIVLVGMPWAELIAEEPQWSSRLVRKRKLSYFSLKNDKNYFIRYIMGLANKMPFEEPPTLGDKQTATAIFSACRGENRSLKHLLSESLKIALLSNENLEIKHLALAFDKLELLGKEAQRQNKKKEGKEKSEDIVIDNPFNQSLKDIDISEVIKHSQYAPNALDPEDILTGRFF

SEQ ID NO: 191 - DNA, 28 bp, Colwellia polaris
  source 1..28: mol_type = other DNA
gtgacctgccgcataggcagctgaaaat

SEQ ID NO: 192 - DNA, 28 bp, Colwellia polaris
  source 1..28: mol_type = other DNA
gacatttgccgcacaggcagagatcaga

SEQ ID NO: 193 - DNA, 156 bp, Colwellia polaris
  source 1..156: mol_type = other DNA
tgtcgctgaaaccatactttgacataatgcaaccatagaatgacaaatttaaaccatactttgacataaaacttaattttgtaatttaattacaaaattaaattcctttaaaataagcaacttaaaatataaataaagcatagaatgacaaattgt

SEQ ID NO: 194 - DNA, 166 bp, Colwellia polaris
  source 1..166: mol_type = other DNA
ctctcgaatatttatgtaattttattacaagattaaataaaatatttttatttggttatttatttgtcatagtatggttatattttatgtcactgtatggttttacagtgttgctaagtccttgttttttagttgtggcttatgtcatgctttgggttcagcgaca

SEQ ID NO: 195 - DNA, 27 bp, Photobacterium iliopiscarium
  source 1..27: mol_type = other DNA
dwwacaaccatawsytgatattnhhhh

SEQ ID NO: 196 - DNA, 27 bp, Photobacterium pisicola
  source 1..27: mol_type = other DNA
dwwacaaccatawsttgatattnhhhh

SEQ ID NO: 197 - DNA, 27 bp, Aliiglaciecola sp.
  source 1..27: mol_type = other DNA
dhhmcaagsatacsttgdcayhrdhma

SEQ ID NO: 198 - DNA, 27 bp, Halomonas titanicae
  source 1..27: mol_type = other DNA
dwwwbyvbnbynwhdykacrwardhdd

SEQ ID NO: 199 - DNA, 27 bp, Neptunomonas qingdaonensis
  source 1..27: mol_type = other DNA
rwddkymvhrwaaghytgcataadddh

SEQ ID NO: 200 - DNA, 27 bp, Klebsiella oxytoca
  source 1..27: mol_type = other DNA
vywkdhhnsataarhtgrcatadhbwd

SEQ ID NO: 201 - DNA, 27 bp, Photobacterium aquae
  source 1..27: mol_type = other DNA
dhddaadycawawvmhrtcchwwdmdh

SEQ ID NO: 202 - DNA, 27 bp, Psychromonas sp.
  source 1..27: mol_type = other DNA
dmkdhrmbyawwydytkacawmwhdwr

SEQ ID NO: 203 - DNA, 27 bp, Colwellia polaris
  source 1..27: mol_type = other DNA
dhwdhamscawashdtgacawawddhw

SEQ ID NO: 204 - DNA, 27 bp, Vibrio sp.
  source 1..27: mol_type = other DNA
dydrkdmmsawwwrdtgwcaybbwydv

SEQ ID NO: 205 - DNA, 150 bp, Psychromonas sp.
  source 1..150: mol_type = other DNA
taatttaattacatattgaaaatggcttgaatattaatttttttaatttgtcagtgtatggttgtattttatgtcaaagtttacttttatatatgatgtaagcaaatgatttactgtaccatgttatgtcagagtatgatttcagcgaca

SEQ ID NO: 206 - DNA, 109 bp, Psychromonas sp.
  source 1..109: mol_type = other DNA
tttaatttgtcagtgtatggttgtattttatgtcaaagtttacttttatatatgatgtaagcaaatgatttactgtaccatgttatgtcagagtatgatttcagcgaca

SEQ ID NO: 207 - DNA, 86 bp, Psychromonas sp.
  source 1..86: mol_type = other DNA
tattttatgtcaaagtttacttttatatatgatgtaagcaaatgatttactgtaccatgttatgtcagagtatgatttcagcgaca

SEQ ID NO: 208 - DNA, 59 bp, Psychromonas sp.
  source 1..59: mol_type = other DNA
tatgatgtaagcaaatgatttactgtaccatgttatgtcagagtatgatttcagcgaca

SEQ ID NO: 209 - DNA, 156 bp, Psychromonas sp.
  source 1..156: mol_type = other DNA
tgtcgctgaaaccatacattgacataatataaccatactttgacaaaattaaagcatactctgacataaaatataaaactgtaattttattacacttttatttttctttttaatcagttgcttatgattttattagcgcatactctgacaaattgt

SEQ ID NO: 210 - DNA, 129 bp, Psychromonas sp.
  source 1..129: mol_type = other DNA
tgtcgctgaaaccatacattgacataatataaccatactttgacaaaattaaagcatactctgacataaaatataaaactgtaattttattacacttttatttttctttttaatcagttgcttatgatt

SEQ ID NO: 211 - DNA, 72 bp, Psychromonas sp.
  source 1..72: mol_type = other DNA
tgtcgctgaaaccatacattgacataatataaccatactttgacaaaattaaagcatactctgacataaaat

SEQ ID NO: 212 - DNA, 50 bp, Psychromonas sp.
  source 1..50: mol_type = other DNA
tgtcgctgaaaccatacattgacataatataaccatactttgacaaaatt

SEQ ID NO: 213 - DNA, 609 bp, synthetic construct
  source 1..609: mol_type = other DNA
atgaaaaggtactacttcatgatccacttcctccccaaggaggccaacttggcattattgactgggcgctgcatttccatcatgcatggttttatcctcaagagggacatacaaggagttggtgtgacgctcccagcgtggagcgacctctcaataggaaatgtgattgcatttgttcattctgacaagaaggtgctgcagatgctgaaagaacaaagctattttgaggacatgcagaactgcggcttcttcgagctgactagtgttactgctgtacctgataattgcgaggaggtgtacttcaagagaaatcaggccatcgccaagattttcaccggtgaaagtcgccgacggttgaaaagacttgagaagcgcgcgctatcgcggggcgagacattcaacccgcagaaatgtgtggtgtcccgggaatttgatgttttccacaggattgctatatctagcaactccaaccagaatgattatattctgcacatccagaaacaaaagggcggcaccaaggtcgggtcaacattttcaaactacgggttctccagcaatgaaaaatttgacggctcggtgccagatctttctcatctcgtcatgaagctt

SEQ ID NO: 214 - DNA, 1050 bp, synthetic construct
  source 1..1050: mol_type = other DNA
atggagctctgcaacattttgaagtatgaccgatccctttatcctagcaaagccgtcttcttctacaagactgatgattcagattttgttccgctggaggccgatatctgccggatccgaggtcccaaaagcggcttcactgaagcatttacaccacagttcctacccaagaatctctcaacgcaggacttaacgcacaacaatattttgacccttgaggagtgctacgtgccgccaaatgttgaccacctctactgtaggttttcactgcgggtacaggccaactcgttggctccatctggatgctccgacccggaggtgttttcgcttttgaaggaatttgccgaggtattcaaggagcggggcgggtacaaagaacttgctgttagatattgtcgcaacatcttgctgggcacctggctgtggcgcaaccaaaatactggtaataccgagatcaagattaagacaagctctggcaacgactacctcataaacaacacgaggaaggtcgcatgggagtctaaatggagtgatgaagaactgaaaacactggaagatctgagtaatgaaattcagaacgcacttatagatcccaacgtgttctggagcgcggacattactgcaaagattgagagcgtgttctgccaggaggtttacccttcacaaatactgacggacaaggtatcccaaagcgaagcgtcaaagcaatttgtgaaaagtaagtttagtgatggtcgttatgctgtttctttcaattcggtcaagatcggggcggcgctccagagcatagacgactggtgggacgatggcgcctcgaaacggcttcgcgtccatgagttcggggtggataaagaaatcggaaccacacgtcgaccgcctcactccgagcataatttctatcatatcttaaagtcggccgagaggtatctctctgagctgaataagttcaatgccaataatggggaggtcgtgaacccaaacatctactacctcttctcggtgctaattaaaggtggcatgttccagaagaaagctgaatcaaagaagggc

SEQ ID NO: 215 - DNA, 2097 bp, synthetic construct
  source 1..2097: mol_type = other DNA
atggtgaacagcatgcacctaaaagaactactggaaatccaggacatcacagaacggaacaaactgctcaggcgcgccttcagcgcctatactgaaaccatagatatcactggctgtgaggaatttgcactgataatccttcttaacttgacctaccgccgtaatcaggtggaggacctactcgataaagtgttagccaagaaaacactcaacaatgaagattacattggcaaatgtataaacgaagtggagtggttccacacacataatttaaaatatcctgatattcgagtttccaagcagacgttggctgtcaaccccccgcccctgcacccacatgtgctcagttcagccaactacgacaagatttttggatggtcccatgattcggcgaaggtgaatgtcgtcaaattgtttttgagttatttcaagtggcaggatgattcttattgtctcgcgcagatactagtttctctccctgatgattggaaggcggcgtttacgagtttggggatgaaggtcaaagtattgctcaatctctgcggccgtgttagctcagttctgccagaggaggtcataccatcctttgtggaccgctactgtcggcagatacgcatgccatatcatgacggttatactgcgataacaccagtcatctcacattccatccagtccaagatccaacaagccgccatccaaaagaagggtagttttacgaaagtcgagttcacccggacagcggcagtatcggagctcgtcgcatccattggaggctttgttaatgcactatcttatccgcctgatgttggaatttcacatcacgggctctcgcaaagtaggcttttccagattcagaatggaaagcaaatattcaatggaaatgtacttctgaagcctcagttcattggtgctctggagggaattatttttaaccattctgcgctcgctctcaagcagaggagacaactaagagtgaagtccatcaaggaactgagaaataccttgtcagaatggttcgcgccaattctggagtggagactagacatcatagagaatcgcgtaaacctgattgattttgagtcgataaatgatcaactcgagtacaagcttgtttccacttccgacgataagcttccatcgctggtgattccgctgttcggcagcttaaactcaatgctcgccaacatccctatgatgcagaagtacgccttccacccaaagctgatggagccgctcaagttcgccttgaaatggctccttagcaacattggcaatgagtccgatgtggccatcaaggacgacgacctccccttccggtatctgcatctctcaggggtcagagtttttgatgcacaggcacttagtaacccttactgctcgggcatcccctccctcacagccgtctgggggatgcttcaccactaccagagggagctgaatcaagctctcggccttggtgtgcgatttacttcgttcagttggtttattcgggactactctctcatcccgggtaagaagcttcccgagatcaatttgcatgggaccaaacctaatgatgtgaagcgtcctggaatcatcgacaaccagtactgcgacctgacattcgacctggtggtccacattgacggctatcaagatgagcttctcaagttggatgacgagccggagctgctgaaggctcatttcccgtcaaatttcgctggcggtgttatgcaccaacctgaattagattcaaacatcgactggtgcaggctgtaccacaaagaaaagttcctgttcgagaagatccgcaggctccccctgagcgggtgctgggtgataccgacggaatataaagttcacgacttcgagtctctgtttgctatattaaacaacgacagcaagctatctccctccatgatgggttacatgcttttaaacaagccagaaccacgccccggatccttagaaaagatgcactgctacgcagagcctgtaatcggggtggttgaatactcgccggcgatcaacattagacttaaaggcaagaggaactattttcataaagctttctggatgttggacgcgcaggagaaattcatgcttatgaaaaaaatt

SEQ ID NO: 216 - DNA, 1227 bp, synthetic construct
  source 1..1227: mol_type = other DNA
atggctttccttttctccccaaaagcgacacccttttcagatgaaagcctcgaatcgtacctgctgaggatagtttcagaaaacttctttgatagctatgaagaactgtcgttggccattcgggaggagctgcatgagctcgattttgaggcccacggcgcttttccaattgatctgaaacggctgaacgtctaccatgccaagcataattcccacttcaggattagagcgttggggcttttggaggcgctgctggaccttccgcgctttgagttgcaaaaaatcgcattacttaaatctgatgttactttcaatagcagcgcggcgctctataggaatggtgttgatatcccgcagaagtttatacgctaccagggtaatagaaaggcatatagcattcctatatgcccgcactgtctccgtgaggaggcttacatcaaacagagctggcacatcgaatgggtcaacatctgtgtgaagcaccagtgcaccctcatccacaactgcccagagtgcaacttgcccataaactacattgaaaatgaaagcatcacccactgtagttgtgggtttgaactagcctcggctgattctttgcccgtcaaggagaagtctatcaagtacctccatgcacttcttgatagtaatatttgcagtggctcaaatccgctgtttaattttacgacatcttctgacagattcgccgcccttctgtggttccacaagaggtactcccataaaaatgtgttctgcttggacgaggcagtggagtacttctcggagtggcctgcttccttctacaaggaattgaatgggctcaccaacaatgctgagatgaagctgatagacctcttcaacaaaacagagtttcgcttcattttcgaggacctcatccttagcagcccgaatactcagatattagagaaacctcatttcatactgatcacgctcctggactaccttgcagcactcattgaggagaaccccaagtcaaagaaggctaacattggagatctattagtgtccgtttcagaaacggcactgttgctcggtactagtattgaccaagtatatcgactgtatcaagacggcatcttccaaacagccttccgcctcaagctcaaccagcggataaatcctaacaagggcgtgttcttcttaaggcaagtgatcgagtataaagcgtcatttggcaacgacaagaacagaatgtacgtgagcgcctgg

SEQ ID NO: 217 - DNA, 624 bp, synthetic construct
  source 1..624: mol_type = other DNA
atgtacatcaggaacttaagaaaaccatcgcccaacaagaacgtgttcaaatttgcttccaccaagatgggggaggtcatactttgtgaatcaacgttggaatttgatgcgtgcttccaccacgagtacaacgacgagattgaaagttatggcagccagccgcagggcttcaagtatgagttcaatgggaaagttcttccatatactcctgatgctctaatactctacaagaatggtgtccacaagtaccatgaatataaaccgtacagcaaaatttctagtacactcttccgcgacaagtttgaggccaagaggcagacatcactcacaatgggcattgatcttatactcgtcactgacagacaaatacgtgtcaaccccatccttaacaacttaaagctccttcatcgatattctggagtgtacggcgtgtccaaggtgcaagttgagctgctgaagatcatccggtactcagaaacaatcaagctgaatgatgttagcagccagtgcaacctgtccattggtgagacgcgttccttcatctacgggttgattcataaaggatttttgaaggccgacctcggtaaggaggacctgagcaataatcctacgctgcgcgcagta

SEQ ID NO: 218 - DNA, 1839 bp, synthetic construct
  source 1..1839: mol_type = other DNA
atgtcggattttattaatgaattcgacaacagcatcgtccccattaagcctgaaacaccagatcaatatgtgaagctggaggatgcgacgcttatcaaacgcgacctcgacacctttcctgacttcctcaaagacaaggcgttcgacaagtacaagttgatcagcattatcgagaaagagatttccggcggctggacccaaaagaacatagatccaattctcgagaacctttttgaggaaaacaatgctaagaagccaaactggcgtacggttgtgaggtggcggaaggcctacatcgattctaacggcgacttgtcgtcactcgtggttaaacaacataagatgggcaacaggactaaacgtattgacggtgatgagttcttcttcgataaggcactagaacgcttcctcgacgccaaacgacccaaggtaacaactgcgtaccagtattacaaagatctgataatgattgccaatgagaatgttgtcgaaggggagatcccagtgatcagctatagcgcgttcaacaagaggattaagagtctgcctccctaccctactgctatagcaagacacggcaaatttaaggcagatcagtggttcgcctattgtggatcccacataccgcccactaggatacttgagagggtagagatcgaccacacaccgcttgacctaattctgctggacgacgaattactcatccctattggcaggccgtatttaaccctgctggtagatgtgttttctggctgcatccttgggttccatctttcatataagtcgcccagctatgtctcggctgcgaaagcaatctcccacgctattaaaccaaagtctcttgaacatctgtccattgcactgcagaacgactggccgtgctacggcaagatagaaaatctggtggtggacaatggagcggagttctggtcgaagtccttagagcatgcttgtcatgccactggtatcaacatccagtacaaccccgtgaggaagccgtggttgaaaccatttgtggaacgcttctttggaatgataaatcagtgcttcttgtcagagataccggggaaaaccttctctaacatcctcgagaaggaggagtacaagccagagaaggatgctataatgagattttcaacattcgtcgaagagttccacagatggattgtcgatgtataccaccaagattccaatagccgtgagactcgtgttcctatcaagcgatggcagcagggttttgacgtttatccgccgctgaagatgaatgatgaagaggatgcacggttttctatattgatgcgggtgtctgatactcgcacgttgacacgcaatgggttcaagttcgaggagctgatgtacgactccacggcgctcgccgactaccgcaaacactacccgcagaccaaggaatcagtcaagaagatcattaaagttgatcctgatgacatctcaaagattcatgtctacctcgaggagctggagtcctatttggaggttccctgcacggagacaaccggttacagcgacggcctcagtctatatgagcacaagaccatcaagaaaatcaacagggaaatcatccgtgaatcgaaggatagtcttggacttgctaaagccagaatggccatccatgaaagaataaaacaagagcaggaggtctttatcaagagcaaaacgaaggccaaacttacagcggtgaaaaagcaggcacagatcgccgatgtttcaaatacagggaagggaagtattaaggtaccagaaattgaaagtattcaaccgaagcggtctaatatttctgacgtcctcgatgattgggaagacgaagtcgaggcgtttgaa

SEQ ID NO: 219 - DNA, 1011 bp, synthetic construct
  source 1..1011: mol_type = other DNA
atgaatgatctgaccgagctgcagattcaacaactgagggacttctctgactgcattgtcgtccatccacagatcaagatcattttcgatgattttgacgagttgcgcttcaaccgcaagttccagagcgaccaacagtgcatgctgctgatcggcgacacgggcgtaggaaaaagtcacctaattaatcactacaagaaacgcgtattagctacgcagaactactcccggggcaccattccagttttggtttcaagaatctcaaggggtaaaggtctagaagctacattaatccagatgctcgcggacttggaactctttgggagcagtcaaataaaaaagagaggttataaaattgatctcacgaagaaactggttgaaaatctcatcaaggcgcaggtggagctccttatcatcaacgagttccaagaactcatcgagtttaagtccgtccaggagaggcaattaatagccaatgggcttaaattcatctcggaagaagcgaaagttccgatagtgctcgtcggcatgccgtgggccgagcgtatcgcagaggagccgcagtgggcgtccagactgatacggaagcgccagcttgagtacttcagcttgaagaacaacagcaagtactttcggcagtacctgatggggctcgccaagaagatgccttttgatgagccgcccaagctaaaggtgaagcataccacactcgctatattttcagcctgcaagggcgaaaaccgcgctctgaagcatctccttactgaagccttgaaacaggcgttgtcctgtaatgagcacctggagaacaagcacttcgtgttcgccttcgacaagctttatttgagtgatacttcgtcgttttctcaacagaagcttacaattaaaaaccccttcaaacaagatattaaggacatcaaaatttatgaggtgtccaaaaattcttcatataatcctaatgcaatagatcctgaggacgcacttaccggtagggaattttctctgattgca

SEQ ID NO: 220 - DNA, 6354 bp, synthetic construct
  source 1..6354: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaaaaggtactacttcatgatccacttcctccccaaggaggccaacttggcattattgactgggcgctgcatttccatcatgcatggttttatcctcaagagggacatacaaggagttggtgtgacgctcccagcgtggagcgacctctcaataggaaatgtgattgcatttgttcattctgacaagaaggtgctgcagatgctgaaagaacaaagctattttgaggacatgcagaactgcggcttcttcgagctgactagtgttactgctgtacctgataattgcgaggaggtgtacttcaagagaaatcaggccatcgccaagattttcaccggtgaaagtcgccgacggttgaaaagacttgagaagcgcgcgctatcgcggggcgagacattcaacccgcagaaatgtgtggtgtcccgggaatttgatgttttccacaggattgctatatctagcaactccaaccagaatgattatattctgcacatccagaaacaaaagggcggcaccaaggtcgggtcaacattttcaaactacgggttctccagcaatgaaaaatttgacggctcggtgccagatctttctcatctcgtcatgaagcttgggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 221 - DNA, 6795 bp, synthetic construct
  source 1..6795: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggagctctgcaacattttgaagtatgaccgatccctttatcctagcaaagccgtcttcttctacaagactgatgattcagattttgttccgctggaggccgatatctgccggatccgaggtcccaaaagcggcttcactgaagcatttacaccacagttcctacccaagaatctctcaacgcaggacttaacgcacaacaatattttgacccttgaggagtgctacgtgccgccaaatgttgaccacctctactgtaggttttcactgcgggtacaggccaactcgttggctccatctggatgctccgacccggaggtgttttcgcttttgaaggaatttgccgaggtattcaaggagcggggcgggtacaaagaacttgctgttagatattgtcgcaacatcttgctgggcacctggctgtggcgcaaccaaaatactggtaataccgagatcaagattaagacaagctctggcaacgactacctcataaacaacacgaggaaggtcgcatgggagtctaaatggagtgatgaagaactgaaaacactggaagatctgagtaatgaaattcagaacgcacttatagatcccaacgtgttctggagcgcggacattactgcaaagattgagagcgtgttctgccaggaggtttacccttcacaaatactgacggacaaggtatcccaaagcgaagcgtcaaagcaatttgtgaaaagtaagtttagtgatggtcgttatgctgtttctttcaattcggtcaagatcggggcggcgctccagagcatagacgactggtgggacgatggcgcctcgaaacggcttcgcgtccatgagttcggggtggataaagaaatcggaaccacacgtcgaccgcctcactccgagcataatttctatcatatcttaaagtcggccgagaggtatctctctgagctgaataagttcaatgccaataatggggaggtcgtgaacccaaacatctactacctcttctcggtgctaattaaaggtggcatgttccagaagaaagctgaatcaaagaagggcgggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 222 - DNA, 7842 bp, synthetic construct
  source 1..7842: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggtgaacagcatgcacctaaaagaactactggaaatccaggacatcacagaacggaacaaactgctcaggcgcgccttcagcgcctatactgaaaccatagatatcactggctgtgaggaatttgcactgataatccttcttaacttgacctaccgccgtaatcaggtggaggacctactcgataaagtgttagccaagaaaacactcaacaatgaagattacattggcaaatgtataaacgaagtggagtggttccacacacataatttaaaatatcctgatattcgagtttccaagcagacgttggctgtcaaccccccgcccctgcacccacatgtgctcagttcagccaactacgacaagatttttggatggtcccatgattcggcgaaggtgaatgtcgtcaaattgtttttgagttatttcaagtggcaggatgattcttattgtctcgcgcagatactagtttctctccctgatgattggaaggcggcgtttacgagtttggggatgaaggtcaaagtattgctcaatctctgcggccgtgttagctcagttctgccagaggaggtcataccatcctttgtggaccgctactgtcggcagatacgcatgccatatcatgacggttatactgcgataacaccagtcatctcacattccatccagtccaagatccaacaagccgccatccaaaagaagggtagttttacgaaagtcgagttcacccggacagcggcagtatcggagctcgtcgcatccattggaggctttgttaatgcactatcttatccgcctgatgttggaatttcacatcacgggctctcgcaaagtaggcttttccagattcagaatggaaagcaaatattcaatggaaatgtacttctgaagcctcagttcattggtgctctggagggaattatttttaaccattctgcgctcgctctcaagcagaggagacaactaagagtgaagtccatcaaggaactgagaaataccttgtcagaatggttcgcgccaattctggagtggagactagacatcatagagaatcgcgtaaacctgattgattttgagtcgataaatgatcaactcgagtacaagcttgtttccacttccgacgataagcttccatcgctggtgattccgctgttcggcagcttaaactcaatgctcgccaacatccctatgatgcagaagtacgccttccacccaaagctgatggagccgctcaagttcgccttgaaatggctccttagcaacattggcaatgagtccgatgtggccatcaaggacgacgacctccccttccggtatctgcatctctcaggggtcagagtttttgatgcacaggcacttagtaacccttactgctcgggcatcccctccctcacagccgtctgggggatgcttcaccactaccagagggagctgaatcaagctctcggccttggtgtgcgatttacttcgttcagttggtttattcgggactactctctcatcccgggtaagaagcttcccgagatcaatttgcatgggaccaaacctaatgatgtgaagcgtcctggaatcatcgacaaccagtactgcgacctgacattcgacctggtggtccacattgacggctatcaagatgagcttctcaagttggatgacgagccggagctgctgaaggctcatttcccgtcaaatttcgctggcggtgttatgcaccaacctgaattagattcaaacatcgactggtgcaggctgtaccacaaagaaaagttcctgttcgagaagatccgcaggctccccctgagcgggtgctgggtgataccgacggaatataaagttcacgacttcgagtctctgtttgctatattaaacaacgacagcaagctatctccctccatgatgggttacatgcttttaaacaagccagaaccacgccccggatccttagaaaagatgcactgctacgcagagcctgtaatcggggtggttgaatactcgccggcgatcaacattagacttaaaggcaagaggaactattttcataaagctttctggatgttggacgcgcaggagaaattcatgcttatgaaaaaaattgggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 223 - DNA, 7836 bp, synthetic construct
  source 1..7836: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgcccgggggcgggggctcaggcgggggcgggagcggcggcgggggctctgggggcggcggcagcggcgggggcggcagcgggggcggcgggtcgatggtgaacagcatgcacctaaaagaactactggaaatccaggacatcacagaacggaacaaactgctcaggcgcgccttcagcgcctatactgaaaccatagatatcactggctgtgaggaatttgcactgataatccttcttaacttgacctaccgccgtaatcaggtggaggacctactcgataaagtgttagccaagaaaacactcaacaatgaagattacattggcaaatgtataaacgaagtggagtggttccacacacataatttaaaatatcctgatattcgagtttccaagcagacgttggctgtcaaccccccgcccctgcacccacatgtgctcagttcagccaactacgacaagatttttggatggtcccatgattcggcgaaggtgaatgtcgtcaaattgtttttgagttatttcaagtggcaggatgattcttattgtctcgcgcagatactagtttctctccctgatgattggaaggcggcgtttacgagtttggggatgaaggtcaaagtattgctcaatctctgcggccgtgttagctcagttctgccagaggaggtcataccatcctttgtggaccgctactgtcggcagatacgcatgccatatcatgacggttatactgcgataacaccagtcatctcacattccatccagtccaagatccaacaagccgccatccaaaagaagggtagttttacgaaagtcgagttcacccggacagcggcagtatcggagctcgtcgcatccattggaggctttgttaatgcactatcttatccgcctgatgttggaatttcacatcacgggctctcgcaaagtaggcttttccagattcagaatggaaagcaaatattcaatggaaatgtacttctgaagcctcagttcattggtgctctggagggaattatttttaaccattctgcgctcgctctcaagcagaggagacaactaagagtgaagtccatcaaggaactgagaaataccttgtcagaatggttcgcgccaattctggagtggagactagacatcatagagaatcgcgtaaacctgattgattttgagtcgataaatgatcaactcgagtacaagcttgtttccacttccgacgataagcttccatcgctggtgattccgctgttcggcagcttaaactcaatgctcgccaacatccctatgatgcagaagtacgccttccacccaaagctgatggagccgctcaagttcgccttgaaatggctccttagcaacattggcaatgagtccgatgtggccatcaaggacgacgacctccccttccggtatctgcatctctcaggggtcagagtttttgatgcacaggcacttagtaacccttactgctcgggcatcccctccctcacagccgtctgggggatgcttcaccactaccagagggagctgaatcaagctctcggccttggtgtgcgatttacttcgttcagttggtttattcgggactactctctcatcccgggtaagaagcttcccgagatcaatttgcatgggaccaaacctaatgatgtgaagcgtcctggaatcatcgacaaccagtactgcgacctgacattcgacctggtggtccacattgacggctatcaagatgagcttctcaagttggatgacgagccggagctgctgaaggctcatttcccgtcaaatttcgctggcggtgttatgcaccaacctgaattagattcaaacatcgactggtgcaggctgtaccacaaagaaaagttcctgttcgagaagatccgcaggctccccctgagcgggtgctgggtgataccgacggaatataaagttcacgacttcgagtctctgtttgctatattaaacaacgacagcaagctatctccctccatgatgggttacatgcttttaaacaagccagaaccacgccccggatccttagaaaagatgcactgctacgcagagcctgtaatcggggtggttgaatactcgccggcgatcaacattagacttaaaggcaagaggaactattttcataaagctttctggatgttggacgcgcaggagaaattcatgcttatgaaaaaaatttccggcggcagctccggcggcagcccgaagaagaagcgcaaggtgtgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 224 - DNA, 6972 bp, synthetic construct
  source 1..6972: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggctttccttttctccccaaaagcgacacccttttcagatgaaagcctcgaatcgtacctgctgaggatagtttcagaaaacttctttgatagctatgaagaactgtcgttggccattcgggaggagctgcatgagctcgattttgaggcccacggcgcttttccaattgatctgaaacggctgaacgtctaccatgccaagcataattcccacttcaggattagagcgttggggcttttggaggcgctgctggaccttccgcgctttgagttgcaaaaaatcgcattacttaaatctgatgttactttcaatagcagcgcggcgctctataggaatggtgttgatatcccgcagaagtttatacgctaccagggtaatagaaaggcatatagcattcctatatgcccgcactgtctccgtgaggaggcttacatcaaacagagctggcacatcgaatgggtcaacatctgtgtgaagcaccagtgcaccctcatccacaactgcccagagtgcaacttgcccataaactacattgaaaatgaaagcatcacccactgtagttgtgggtttgaactagcctcggctgattctttgcccgtcaaggagaagtctatcaagtacctccatgcacttcttgatagtaatatttgcagtggctcaaatccgctgtttaattttacgacatcttctgacagattcgccgcccttctgtggttccacaagaggtactcccataaaaatgtgttctgcttggacgaggcagtggagtacttctcggagtggcctgcttccttctacaaggaattgaatgggctcaccaacaatgctgagatgaagctgatagacctcttcaacaaaacagagtttcgcttcattttcgaggacctcatccttagcagcccgaatactcagatattagagaaacctcatttcatactgatcacgctcctggactaccttgcagcactcattgaggagaaccccaagtcaaagaaggctaacattggagatctattagtgtccgtttcagaaacggcactgttgctcggtactagtattgaccaagtatatcgactgtatcaagacggcatcttccaaacagccttccgcctcaagctcaaccagcggataaatcctaacaagggcgtgttcttcttaaggcaagtgatcgagtataaagcgtcatttggcaacgacaagaacagaatgtacgtgagcgcctgggggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 225 - DNA, 6369 bp, synthetic construct
  source 1..6369: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgtacatcaggaacttaagaaaaccatcgcccaacaagaacgtgttcaaatttgcttccaccaagatgggggaggtcatactttgtgaatcaacgttggaatttgatgcgtgcttccaccacgagtacaacgacgagattgaaagttatggcagccagccgcagggcttcaagtatgagttcaatgggaaagttcttccatatactcctgatgctctaatactctacaagaatggtgtccacaagtaccatgaatataaaccgtacagcaaaatttctagtacactcttccgcgacaagtttgaggccaagaggcagacatcactcacaatgggcattgatcttatactcgtcactgacagacaaatacgtgtcaaccccatccttaacaacttaaagctccttcatcgatattctggagtgtacggcgtgtccaaggtgcaagttgagctgctgaagatcatccggtactcagaaacaatcaagctgaatgatgttagcagccagtgcaacctgtccattggtgagacgcgttccttcatctacgggttgattcataaaggatttttgaaggccgacctcggtaaggaggacctgagcaataatcctacgctgcgcgcagtagggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 226 - DNA, 7584 bp, synthetic construct
  source 1..7584: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgtcggattttattaatgaattcgacaacagcatcgtccccattaagcctgaaacaccagatcaatatgtgaagctggaggatgcgacgcttatcaaacgcgacctcgacacctttcctgacttcctcaaagacaaggcgttcgacaagtacaagttgatcagcattatcgagaaagagatttccggcggctggacccaaaagaacatagatccaattctcgagaacctttttgaggaaaacaatgctaagaagccaaactggcgtacggttgtgaggtggcggaaggcctacatcgattctaacggcgacttgtcgtcactcgtggttaaacaacataagatgggcaacaggactaaacgtattgacggtgatgagttcttcttcgataaggcactagaacgcttcctcgacgccaaacgacccaaggtaacaactgcgtaccagtattacaaagatctgataatgattgccaatgagaatgttgtcgaaggggagatcccagtgatcagctatagcgcgttcaacaagaggattaagagtctgcctccctaccctactgctatagcaagacacggcaaatttaaggcagatcagtggttcgcctattgtggatcccacataccgcccactaggatacttgagagggtagagatcgaccacacaccgcttgacctaattctgctggacgacgaattactcatccctattggcaggccgtatttaaccctgctggtagatgtgttttctggctgcatccttgggttccatctttcatataagtcgcccagctatgtctcggctgcgaaagcaatctcccacgctattaaaccaaagtctcttgaacatctgtccattgcactgcagaacgactggccgtgctacggcaagatagaaaatctggtggtggacaatggagcggagttctggtcgaagtccttagagcatgcttgtcatgccactggtatcaacatccagtacaaccccgtgaggaagccgtggttgaaaccatttgtggaacgcttctttggaatgataaatcagtgcttcttgtcagagataccggggaaaaccttctctaacatcctcgagaaggaggagtacaagccagagaaggatgctataatgagattttcaacattcgtcgaagagttccacagatggattgtcgatgtataccaccaagattccaatagccgtgagactcgtgttcctatcaagcgatggcagcagggttttgacgtttatccgccgctgaagatgaatgatgaagaggatgcacggttttctatattgatgcgggtgtctgatactcgcacgttgacacgcaatgggttcaagttcgaggagctgatgtacgactccacggcgctcgccgactaccgcaaacactacccgcagaccaaggaatcagtcaagaagatcattaaagttgatcctgatgacatctcaaagattcatgtctacctcgaggagctggagtcctatttggaggttccctgcacggagacaaccggttacagcgacggcctcagtctatatgagcacaagaccatcaagaaaatcaacagggaaatcatccgtgaatcgaaggatagtcttggacttgctaaagccagaatggccatccatgaaagaataaaacaagagcaggaggtctttatcaagagcaaaacgaaggccaaacttacagcggtgaaaaagcaggcacagatcgccgatgtttcaaatacagggaagggaagtattaaggtaccagaaattgaaagtattcaaccgaagcggtctaatatttctgacgtcctcgatgattgggaagacgaagtcgaggcgtttgaagggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 227 - DNA, 6756 bp, synthetic construct
  source 1..6756: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaatgatctgaccgagctgcagattcaacaactgagggacttctctgactgcattgtcgtccatccacagatcaagatcattttcgatgattttgacgagttgcgcttcaaccgcaagttccagagcgaccaacagtgcatgctgctgatcggcgacacgggcgtaggaaaaagtcacctaattaatcactacaagaaacgcgtattagctacgcagaactactcccggggcaccattccagttttggtttcaagaatctcaaggggtaaaggtctagaagctacattaatccagatgctcgcggacttggaactctttgggagcagtcaaataaaaaagagaggttataaaattgatctcacgaagaaactggttgaaaatctcatcaaggcgcaggtggagctccttatcatcaacgagttccaagaactcatcgagtttaagtccgtccaggagaggcaattaatagccaatgggcttaaattcatctcggaagaagcgaaagttccgatagtgctcgtcggcatgccgtgggccgagcgtatcgcagaggagccgcagtgggcgtccagactgatacggaagcgccagcttgagtacttcagcttgaagaacaacagcaagtactttcggcagtacctgatggggctcgccaagaagatgccttttgatgagccgcccaagctaaaggtgaagcataccacactcgctatattttcagcctgcaagggcgaaaaccgcgctctgaagcatctccttactgaagccttgaaacaggcgttgtcctgtaatgagcacctggagaacaagcacttcgtgttcgccttcgacaagctttatttgagtgatacttcgtcgttttctcaacagaagcttacaattaaaaaccccttcaaacaagatattaaggacatcaaaatttatgaggtgtccaaaaattcttcatataatcctaatgcaatagatcctgaggacgcacttaccggtagggaattttctctgattgcagggggcgggggctcaggcgggggcgggagcggcggcgggggctctgggggcggcggcagcggcgggggcggcagcgggggcggcgggtcgatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggaaggtggccccttgcccttcgccgaggacatcttgtccgccgccttcatgtacggcaaccgcgtgttcaccgagtacccccaggacatcgtcgactacttcaagaactcctgccctgcaggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacttgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgcgatcgcctccggctccgccttgccctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 228 - DNA, 7059 bp, synthetic construct
  source 1..7059: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggtgaacagcatgcacctaaaagaactactggaaatccaggacatcacagaacggaacaaactgctcaggcgcgccttcagcgcctatactgaaaccatagatatcactggctgtgaggaatttgcactgataatccttcttaacttgacctaccgccgtaatcaggtggaggacctactcgataaagtgttagccaagaaaacactcaacaatgaagattacattggcaaatgtataaacgaagtggagtggttccacacacataatttaaaatatcctgatattcgagtttccaagcagacgttggctgtcaaccccccgcccctgcacccacatgtgctcagttcagccaactacgacaagatttttggatggtcccatgattcggcgaaggtgaatgtcgtcaaattgtttttgagttatttcaagtggcaggatgattcttattgtctcgcgcagatactagtttctctccctgatgattggaaggcggcgtttacgagtttggggatgaaggtcaaagtattgctcaatctctgcggccgtgttagctcagttctgccagaggaggtcataccatcctttgtggaccgctactgtcggcagatacgcatgccatatcatgacggttatactgcgataacaccagtcatctcacattccatccagtccaagatccaacaagccgccatccaaaagaagggtagttttacgaaagtcgagttcacccggacagcggcagtatcggagctcgtcgcatccattggaggctttgttaatgcactatcttatccgcctgatgttggaatttcacatcacgggctctcgcaaagtaggcttttccagattcagaatggaaagcaaatattcaatggaaatgtacttctgaagcctcagttcattggtgctctggagggaattatttttaaccattctgcgctcgctctcaagcagaggagacaactaagagtgaagtccatcaaggaactgagaaataccttgtcagaatggttcgcgccaattctggagtggagactagacatcatagagaatcgcgtaaacctgattgattttgagtcgataaatgatcaactcgagtacaagcttgtttccacttccgacgataagcttccatcgctggtgattccgctgttcggcagcttaaactcaatgctcgccaacatccctatgatgcagaagtacgccttccacccaaagctgatggagccgctcaagttcgccttgaaatggctccttagcaacattggcaatgagtccgatgtggccatcaaggacgacgacctccccttccggtatctgcatctctcaggggtcagagtttttgatgcacaggcacttagtaacccttactgctcgggcatcccctccctcacagccgtctgggggatgcttcaccactaccagagggagctgaatcaagctctcggccttggtgtgcgatttacttcgttcagttggtttattcgggactactctctcatcccgggtaagaagcttcccgagatcaatttgcatgggaccaaacctaatgatgtgaagcgtcctggaatcatcgacaaccagtactgcgacctgacattcgacctggtggtccacattgacggctatcaagatgagcttctcaagttggatgacgagccggagctgctgaaggctcatttcccgtcaaatttcgctggcggtgttatgcaccaacctgaattagattcaaacatcgactggtgcaggctgtaccacaaagaaaagttcctgttcgagaagatccgcaggctccccctgagcgggtgctgggtgataccgacggaatataaagttcacgacttcgagtctctgtttgctatattaaacaacgacagcaagctatctccctccatgatgggttacatgcttttaaacaagccagaaccacgccccggatccttagaaaagatgcactgctacgcagagcctgtaatcggggtggttgaatactcgccggcgatcaacattagacttaaaggcaagaggaactattttcataaagctttctggatgttggacgcgcaggagaaattcatgcttatgaaaaaaatttgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 229 - DNA, 7057 bp, synthetic construct
  source 1..7057: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggatggtgaacagcatgcacctaaaagaactactggaaatccaggacatcacagaacggaacaaactgctcaggcgcgccttcagcgcctatactgaaaccatagatatcactggctgtgaggaatttgcactgataatccttcttaacttgacctaccgccgtaatcaggtggaggacctactcgataaagtgttagccaagaaaacactcaacaatgaagattacattggcaaatgtataaacgaagtggagtggttccacacacataatttaaaatatcctgatattcgagtttccaagcagacgttggctgtcaaccccccgcccctgcacccacatgtgctcagttcagccaactacgacaagatttttggatggtcccatgattcggcgaaggtgaatgtcgtcaaattgtttttgagttatttcaagtggcaggatgattcttattgtctcgcgcagatactagtttctctccctgatgattggaaggcggcgtttacgagtttggggatgaaggtcaaagtattgctcaatctctgcggccgtgttagctcagttctgccagaggaggtcataccatcctttgtggaccgctactgtcggcagatacgcatgccatatcatgacggttatactgcgataacaccagtcatctcacattccatccagtccaagatccaacaagccgccatccaaaagaagggtagttttacgaaagtcgagttcacccggacagcggcagtatcggagctcgtcgcatccattggaggctttgttaatgcactatcttatccgcctgatgttggaatttcacatcacgggctctcgcaaagtaggcttttccagattcagaatggaaagcaaatattcaatggaaatgtacttctgaagcctcagttcattggtgctctggagggaattatttttaaccattctgcgctcgctctcaagcagaggagacaactaagagtgaagtccatcaaggaactgagaaataccttgtcagaatggttcgcgccaattctggagtggagactagacatcatagagaatcgcgtaaacctgattgattttgagtcgataaatgatcaactcgagtacaagcttgtttccacttccgacgataagcttccatcgctggtgattccgctgttcggcagcttaaactcaatgctcgccaacatccctatgatgcagaagtacgccttccacccaaagctgatggagccgctcaagttcgccttgaaatggctccttagcaacattggcaatgagtccgatgtggccatcaaggacgacgacctccccttccggtatctgcatctctcaggggtcagagtttttgatgcacaggcacttagtaacccttactgctcgggcatcccctccctcacagccgtctgggggatgcttcaccactaccagagggagctgaatcaagctctcggccttggtgtgcgatttacttcgttcagttggtttattcgggactactctctcatcccgggtaagaagcttcccgagatcaatttgcatgggaccaaacctaatgatgtgaagcgtcctggaatcatcgacaaccagtactgcgacctgacattcgacctggtggtccacattgacggctatcaagatgagcttctcaagttggatgacgagccggagctgctgaaggctcatttcccgtcaaatttcgctggcggtgttatgcaccaacctgaattagattcaaacatcgactggtgcaggctgtaccacaaagaaaagttcctgttcgagaagatccgcaggctccccctgagcgggtgctgggtgataccgacggaatataaagttcacgacttcgagtctctgtttgctatattaaacaacgacagcaagctatctccctccatgatgggttacatgcttttaaacaagccagaaccacgccccggatccttagaaaagatgcactgctacgcagagcctgtaatcggggtggttgaatactcgccggcgatcaacattagacttaaaggcaagaggaactattttcataaagctttctggatgttggacgcgcaggagaaattcatgcttatgaaaaaaatttccggcggcagctccggcggcagcccgaagaagaagcgcaaggtgtgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 230 - DNA, 1050 bp, synthetic construct
  source 1..1050: mol_type = other DNA
atggagttgtgcaacatcctgaagtacgaccgcagcctctaccccagcaaggcagtattcttctacaagaccgacgattcggacttcgtccccctggaggccgacatctgccgcatcaggggcccgaagagcggcttcaccgaggccttcactccacagttcctgcccaagaacctcagcacccaggacctcacgcataacaacatcctgaccctcgaagagtgctacgtcccgccgaacgtcgatcacctctactgccgcttctccttgcgcgtccaggccaactcgctcgcgccatccggctgcagcgatcctgaggtcttctctctgctcaaggagttcgccgaggtcttcaaggagaggggtgggtacaaggagctggccgtccgctactgccgcaacatccttctgggcacctggctgtggcgcaaccagaacacgggcaacacagagatcaagatcaagacgtcctccggcaacgactacctcatcaacaacacccgcaaggtggcctgggagtccaagtggtccgacgaggagcttaagaccctggaggacctcagcaacgagatccagaacgccctgatcgaccccaacgtcttctggtccgccgacatcaccgcgaagatcgagtctgtgttctgccaggaggtgtacccaagccagatcttgaccgacaaggtcagccagtccgaggcttccaagcagttcgtgaagtccaagttctccgacggcaggtacgcggtcagctttaactcggtcaagatcggtgccgccctacagtccatcgacgattggtgggacgacggcgccagcaagaggctgcgcgtgcacgagttcggcgtcgataaggagatcggtaccacccgccgccctccccactcggagcacaacttctaccacatcctgaagtcggcggagcgctacctctccgaactcaacaagttcaacgcgaacaacggtgaggtggtcaacccgaacatctactacctgttctccgtgctcatcaagggtggcatgttccagaagaaggccgagagcaagaagggc

SEQ ID NO: 231 - DNA, 1050 bp, synthetic construct
  source 1..1050: mol_type = other DNA
atggagctttgcaacatcctgaagtacgatcgcagcctctaccccagcaaggcggtattcttctacaagacggacgactccgacttcgtgccgctcgaggctgacatctgccgcatccgcgggcctaagtccggtttcaccgaggccttcacgccgcagttcctgccgaagaacctctcgacgcaggacctcacccacaacaatatcctgaccctcgaagagtgctacgtcccgcccaacgtcgatcacctctattgccgcttctcgctgagggtccaggccaactccctggccccgtccggctgcagcgaccccgaggtcttcagcctcctcaaggagttcgccgaggtcttcaaggagcgcggcggctacaaggagctggccgtccgctactgccgcaacatcctccttggcacctggctctggaggaaccagaacaccggtaacacggagatcaagatcaagacgtcctcaggcaacgactacctgatcaacaacacgcggaaggtcgcctgggagtccaagtggtcggacgaggagctaaagaccctcgaggacctctccaacgagatccagaacgccctcatcgacccgaacgtcttctggtcagcggacatcaccgccaaaattgagtccgttttctgccaggaagtgtacccgtcccagatcctcacggacaaggtcagccagtccgaggcctccaagcagttcgtcaagtccaagttctccgacggcaggtacgccgtgagcttcaactccgtcaagatcggcgcggccctccagtcgatcgacgactggtgggacgacggggcctccaagcgcctgcgcgtccacgagttcggcgtcgataaggagatcggcaccacgcgacgccctccacactcggagcacaacttctaccacatcctgaagtctgctgagaggtacctctccgagctgaacaagttcaacgctaacaacggcgaggtggtgaacccgaacatctactacctgttctcggtcctcatcaagggcggcatgttccagaagaaggccgagtctaagaagggg

SEQ ID NO: 232 - DNA, 1050 bp, synthetic construct
  source 1..1050: mol_type = other DNA
atggagctgtgcaacatcctcaagtacgaccgctccctctacccgtccaaggccgtcttcttttacaagaccgacgacagcgactttgtccctctcgaggccgatatctgccgcatccgcgggccgaagtccggcttcacggaagcgttcacgccgcagttcctcccaaagaacctcagcacccaggacctgacgcacaacaacatcctcacgctggaggagtgctacgtcccgccgaacgtcgatcacctctactgccgcttcagcttgcgcgtgcaggccaactccctcgccccgtccggctgctccgaccccgaggtgttctcgctcctcaaggagttcgccgaagtcttcaaggagcgcggcggctacaaggagcttgccgtacggtactgccgcaacatcctcctggggacctggctgtggcgcaaccagaacaccggtaacacggagatcaagatcaagacatccagcggcaacgactacctcatcaacaacacccgcaaggtggcctgggaatccaagtggtcagacgaggagcttaagaccctcgaggacctgtccaacgagatccagaacgcgctgatcgacccaaacgtcttctggtctgcggatatcacggccaagatcgagtccgtcttctgccaggaggtctatcccagccagatccttaccgacaaggtgtcccagtccgaggcctccaagcagttcgtgaagtccaagttctccgacgggcgctacgcggtgtccttcaactcggtgaagatcggcgcggccctgcagtcgatcgacgattggtgggacgacggcgcgtccaagaggctgcgcgtccacgagttcggtgtggataaggagatcggcaccacccgccgcccgccacactcggagcacaacttctaccacatcctgaagtctgccgagcgctacctctctgagctgaacaagttcaacgccaacaacggcgaagtcgtgaacccaaacatctactatctcttcagcgtgctcatcaagggcgggatgttccagaagaaggccgagtccaagaagggc

SEQ ID NO: 233 - DNA, 1050 bp, synthetic construct
  source 1..1050: mol_type = other DNA
atggagctttgcaacatcctcaagtacgaccgctccctctaccctagcaaggccgttttcttctacaagaccgacgactccgacttcgtccctctcgaggcggacatctgccgcatccgcggacctaagtctggcttcaccgaggcgttcacccctcagttcctcccgaagaacctgagcacgcaggacctcacgcacaacaacatcctcaccctcgaggagtgctacgtgccacccaacgtcgatcatctctactgcaggttctctctccgcgtgcaggccaactccctggccccgtcgggctgctccgaccccgaggtgttctcgctgctgaaggagttcgccgaggtgttcaaggagcgcggcggttacaaggagctggccgtgcggtactgcaggaacatcctgctcggcacctggctctggcgcaaccagaacacgggtaacaccgagatcaagatcaagacgtcttccggcaacgactacctgatcaataacacccgcaaggtcgcctgggaatcgaagtggagcgacgaggagctaaagaccctggaggacctgtccaacgagatccagaacgcgctcatcgaccccaacgtcttctggtccgccgacatcaccgccaagatcgagagcgtcttctgccaggaggtttacccgtcgcagatcctgaccgacaaggtcagccagtcggaggccagcaagcagttcgtcaagtccaagttctcggacggtcgctacgccgtcagcttcaactccgtcaagatcggtgccgccctgcagtcaatcgacgattggtgggacgacggcgcctcgaagaggctgcgcgtgcacgagttcggcgtcgataaggagatcggcaccacccgcaggcctccccactccgagcacaacttctaccatatcctcaagagtgccgaacgatacctctccgagctgaacaaattcaacgcaaacaacggcgaggtcgtgaacccgaacatctactacttattctccgtgctcatcaagggtggcatgttccagaagaaggcggagtccaagaagggg

SEQ ID NO: 234 - DNA, 1011 bp, synthetic construct
  source 1..1011: mol_type = other DNA
atgaacgacctcaccgagctccagattcagcagctgcgcgatttcagcgactgcatcgtcgtgcacccccagatcaagatcatcttcgacgacttcgacgagctccgcttcaaccgcaagttccagtcggatcagcagtgcatgctcctcatcggcgacacgggcgtcggtaaatcccacctcatcaaccactacaagaagcgcgtcctcgccacccagaactactcccggggtaccatcccggtcctcgtcagccgcatctccaggggtaaaggcctcgaggccaccctcatccagatgcttgccgacctggagctattcgggagctcgcagatcaagaagcgcggctacaagatcgacctcaccaagaagctggtcgagaaccttatcaaggcccaggtcgaactgctaatcatcaacgagttccaagagctcatcgagttcaagtccgtgcaggagcggcagctgatcgccaacgggctgaagttcatctcagaggaggccaaggtccccatcgtcctcgtgggcatgccctgggccgagcgcatcgccgaggaacctcagtgggcgtcgaggctcatccgcaagcgccagctggagtacttcagcctcaagaacaactccaagtacttcaggcagtacctcatgggcctcgcgaagaagatgccattcgacgagccccctaagctgaaggtcaagcacaccaccctcgccatcttcagcgcctgcaagggcgagaaccgtgccctcaagcacctgctcaccgaggcgctcaagcaggccctctcctgcaacgagcacctggagaacaagcacttcgtcttcgccttcgacaagctctacctatccgacacgtcgtccttctcccagcagaagctcaccatcaagaacccattcaagcaggatatcaaggacatcaagatctacgaggtgagcaagaactcctcgtacaacccgaacgccatcgaccccgaggacgctctgaccggcagggagttctccctgatcgcc

SEQ ID NO: 235 - DNA, 1011 bp, synthetic construct
  source 1..1011: mol_type = other DNA
atgaacgacctcacggagctccagatccagcagctccgggacttctcggactgcatcgtggtccacccccagatcaagatcatcttcgacgacttcgacgaactccgcttcaaccgcaagttccagtccgaccagcagtgcatgctcctgatcggcgacacgggcgtcggcaagtcacatctcatcaaccactacaagaagcgcgttctggcgacccagaactactcgcggggtaccatccctgtgctcgtcagccgcatctcgcgcggtaaaggcctcgaggccaccctcatccagatgcttgctgaccttgagctcttcggctcgagccagatcaagaagcgcggctacaagatcgacctcacgaagaagctggtcgagaaccttatcaaggcgcaggtcgagctcctgatcatcaacgagttccaggagctcatcgagtttaagtccgtacaggagaggcagcttatcgccaacggcctgaagttcatcagcgaggaggccaaggtgccgatcgtcctcgtgggcatgccctgggccgagcgcatcgccgaggagccccagtgggccagccgcctcattcggaagcgccagctcgagtacttcagcctgaagaacaactccaagtacttccgccagtacctcatgggcctcgcgaagaagatgcctttcgacgagccgccgaagctcaaggtcaagcacaccacgctggccatcttctcggcctgcaagggcgaaaaccgtgccctcaagcacctgctcacagaagccctcaagcaggccctgtcctgcaacgagcacctcgagaacaagcacttcgtgttcgccttcgacaagctctacttgtccgacaccagctccttctcccagcagaagctcaccatcaagaacccttttaagcaagacatcaaggatatcaagatctacgaggtgtccaagaactcctcctacaaccccaacgccatcgaccccgaagacgccctcaccggccgcgagtttagcctgatcgcc

SEQ ID NO: 236 - DNA, 1011 bp, synthetic construct
  source 1..1011: mol_type = other DNA
atgaacgatctcaccgaactccagatccagcagctccgcgacttctccgattgcatcgtcgtccacccccagatcaagatcatcttcgacgacttcgacgagctgaggttcaaccgcaagttccagtcggaccagcagtgcatgctcctcatcggcgacaccggcgtcggcaagagccacctcatcaaccactacaagaagcgcgtcctggccacccagaactactcgaggggtacgatccccgtgctggtcagccgcatcagccgcggcaagggcctggaagccaccctcatccagatgctggctgacctggagctgttcggctcctcccagatcaagaagcgcggctataagatcgacctcaccaagaagctcgtggagaacctcatcaaggcccaggtcgagctcctcatcatcaacgagttccaggagctcatcgaattcaagagcgtccaggagcgccagctcatcgccaacggcctgaagttcatttcggaggaggccaaggtgcctatcgtcctggtcggcatgccctgggccgagaggatcgccgaggagccccagtgggcctcccgcctcatccgcaagcgccagctggaatacttcagcctgaagaacaactcgaagtacttccgccagtacctgatgggcctcgccaagaagatgcccttcgacgagccgccgaagctgaaggtcaagcacaccacgctcgcgattttctccgcctgcaagggcgagaaccgcgccctcaagcacctcctcacggaagccctgaagcaggccctctcctgcaacgagcacctggagaacaagcacttcgtgttcgcgttcgacaagctgtacctctccgataccagcagcttctcccagcagaagctcacgatcaagaacccgttcaagcaggacatcaaggacatcaagatctacgaggtcagcaagaactcctcctacaacccaaacgccatcgaccccgaggacgcgctgaccgggcgcgagttctccctgatcgcg

SEQ ID NO: 237 - DNA, 1011 bp, synthetic construct
  source 1..1011: mol_type = other DNA
atgaacgacctcaccgagctgcagatacagcagctgcgcgacttctccgactgcatcgtggtgcaccctcagatcaagatcatcttcgacgacttcgacgagctccgcttcaacaggaagttccagtccgatcagcagtgcatgctcctcatcggcgacacgggtgtgggcaaaagccatctcatcaaccactacaagaagcgcgtcctcgccacgcagaactactcccgcggcaccatcccggtcctcgtcagcaggatctccaggggcaagggcctcgaggccaccctcatccagatgctcgccgacctcgagctcttcggctcctcgcagatcaagaagcgcggctacaagatcgacctgaccaagaagctcgtcgagaacctgatcaaggcccaagtggagctgctcatcatcaacgagttccaggagctcatcgagttcaagagcgtccaggagcgccagctcatcgccaacggcctgaagttcatctccgaggaagccaaggtccctatcgtgctcgtgggcatgccctgggccgagcgcatcgctgaagagccgcagtgggcctcccgcctcattcgcaagcgtcagctcgagtacttctccctcaagaacaattccaagtacttccgacagtacctgatgggcctggcgaagaagatgcccttcgacgagcccccgaagctcaaggtgaagcacaccacgctcgccatcttctcggcctgcaagggcgagaaccgcgccctcaagcacctcctcaccgaggccctcaagcaggctctcagctgcaacgagcacctcgagaacaagcactttgtcttcgcgttcgataagctctacctttccgacaccagctcgttctcccagcagaagctcacgatcaagaaccccttcaagcaggacatcaaggacatcaagatctacgaggtgtccaagaactcgtcctacaacccgaacgccatcgacccagaggacgcgctcaccggccgagagttctccctgatcgcc

SEQ ID NO: 238 - DNA, 6012 bp, synthetic construct
  source 1..6012: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggagttgtgcaacatcctgaagtacgaccgcagcctctaccccagcaaggcagtattcttctacaagaccgacgattcggacttcgtccccctggaggccgacatctgccgcatcaggggcccgaagagcggcttcaccgaggccttcactccacagttcctgcccaagaacctcagcacccaggacctcacgcataacaacatcctgaccctcgaagagtgctacgtcccgccgaacgtcgatcacctctactgccgcttctccttgcgcgtccaggccaactcgctcgcgccatccggctgcagcgatcctgaggtcttctctctgctcaaggagttcgccgaggtcttcaaggagaggggtgggtacaaggagctggccgtccgctactgccgcaacatccttctgggcacctggctgtggcgcaaccagaacacgggcaacacagagatcaagatcaagacgtcctccggcaacgactacctcatcaacaacacccgcaaggtggcctgggagtccaagtggtccgacgaggagcttaagaccctggaggacctcagcaacgagatccagaacgccctgatcgaccccaacgtcttctggtccgccgacatcaccgcgaagatcgagtctgtgttctgccaggaggtgtacccaagccagatcttgaccgacaaggtcagccagtccgaggcttccaagcagttcgtgaagtccaagttctccgacggcaggtacgcggtcagctttaactcggtcaagatcggtgccgccctacagtccatcgacgattggtgggacgacggcgccagcaagaggctgcgcgtgcacgagttcggcgtcgataaggagatcggtaccacccgccgccctccccactcggagcacaacttctaccacatcctgaagtcggcggagcgctacctctccgaactcaacaagttcaacgcgaacaacggtgaggtggtcaacccgaacatctactacctgttctccgtgctcatcaagggtggcatgttccagaagaaggccgagagcaagaagggctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 239 - DNA, 6012 bp, synthetic construct
  source 1..6012: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggagctttgcaacatcctgaagtacgatcgcagcctctaccccagcaaggcggtattcttctacaagacggacgactccgacttcgtgccgctcgaggctgacatctgccgcatccgcgggcctaagtccggtttcaccgaggccttcacgccgcagttcctgccgaagaacctctcgacgcaggacctcacccacaacaatatcctgaccctcgaagagtgctacgtcccgcccaacgtcgatcacctctattgccgcttctcgctgagggtccaggccaactccctggccccgtccggctgcagcgaccccgaggtcttcagcctcctcaaggagttcgccgaggtcttcaaggagcgcggcggctacaaggagctggccgtccgctactgccgcaacatcctccttggcacctggctctggaggaaccagaacaccggtaacacggagatcaagatcaagacgtcctcaggcaacgactacctgatcaacaacacgcggaaggtcgcctgggagtccaagtggtcggacgaggagctaaagaccctcgaggacctctccaacgagatccagaacgccctcatcgacccgaacgtcttctggtcagcggacatcaccgccaaaattgagtccgttttctgccaggaagtgtacccgtcccagatcctcacggacaaggtcagccagtccgaggcctccaagcagttcgtcaagtccaagttctccgacggcaggtacgccgtgagcttcaactccgtcaagatcggcgcggccctccagtcgatcgacgactggtgggacgacggggcctccaagcgcctgcgcgtccacgagttcggcgtcgataaggagatcggcaccacgcgacgccctccacactcggagcacaacttctaccacatcctgaagtctgctgagaggtacctctccgagctgaacaagttcaacgctaacaacggcgaggtggtgaacccgaacatctactacctgttctcggtcctcatcaagggcggcatgttccagaagaaggccgagtctaagaaggggtgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 240 - DNA, 6012 bp, synthetic construct
  source 1..6012: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggagctgtgcaacatcctcaagtacgaccgctccctctacccgtccaaggccgtcttcttttacaagaccgacgacagcgactttgtccctctcgaggccgatatctgccgcatccgcgggccgaagtccggcttcacggaagcgttcacgccgcagttcctcccaaagaacctcagcacccaggacctgacgcacaacaacatcctcacgctggaggagtgctacgtcccgccgaacgtcgatcacctctactgccgcttcagcttgcgcgtgcaggccaactccctcgccccgtccggctgctccgaccccgaggtgttctcgctcctcaaggagttcgccgaagtcttcaaggagcgcggcggctacaaggagcttgccgtacggtactgccgcaacatcctcctggggacctggctgtggcgcaaccagaacaccggtaacacggagatcaagatcaagacatccagcggcaacgactacctcatcaacaacacccgcaaggtggcctgggaatccaagtggtcagacgaggagcttaagaccctcgaggacctgtccaacgagatccagaacgcgctgatcgacccaaacgtcttctggtctgcggatatcacggccaagatcgagtccgtcttctgccaggaggtctatcccagccagatccttaccgacaaggtgtcccagtccgaggcctccaagcagttcgtgaagtccaagttctccgacgggcgctacgcggtgtccttcaactcggtgaagatcggcgcggccctgcagtcgatcgacgattggtgggacgacggcgcgtccaagaggctgcgcgtccacgagttcggtgtggataaggagatcggcaccacccgccgcccgccacactcggagcacaacttctaccacatcctgaagtctgccgagcgctacctctctgagctgaacaagttcaacgccaacaacggcgaagtcgtgaacccaaacatctactatctcttcagcgtgctcatcaagggcgggatgttccagaagaaggccgagtccaagaagggctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 241 - DNA, 6012 bp, synthetic construct
  source 1..6012: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatggagctttgcaacatcctcaagtacgaccgctccctctaccctagcaaggccgttttcttctacaagaccgacgactccgacttcgtccctctcgaggcggacatctgccgcatccgcggacctaagtctggcttcaccgaggcgttcacccctcagttcctcccgaagaacctgagcacgcaggacctcacgcacaacaacatcctcaccctcgaggagtgctacgtgccacccaacgtcgatcatctctactgcaggttctctctccgcgtgcaggccaactccctggccccgtcgggctgctccgaccccgaggtgttctcgctgctgaaggagttcgccgaggtgttcaaggagcgcggcggttacaaggagctggccgtgcggtactgcaggaacatcctgctcggcacctggctctggcgcaaccagaacacgggtaacaccgagatcaagatcaagacgtcttccggcaacgactacctgatcaataacacccgcaaggtcgcctgggaatcgaagtggagcgacgaggagctaaagaccctggaggacctgtccaacgagatccagaacgcgctcatcgaccccaacgtcttctggtccgccgacatcaccgccaagatcgagagcgtcttctgccaggaggtttacccgtcgcagatcctgaccgacaaggtcagccagtcggaggccagcaagcagttcgtcaagtccaagttctcggacggtcgctacgccgtcagcttcaactccgtcaagatcggtgccgccctgcagtcaatcgacgattggtgggacgacggcgcctcgaagaggctgcgcgtgcacgagttcggcgtcgataaggagatcggcaccacccgcaggcctccccactccgagcacaacttctaccatatcctcaagagtgccgaacgatacctctccgagctgaacaaattcaacgcaaacaacggcgaggtcgtgaacccgaacatctactacttattctccgtgctcatcaagggtggcatgttccagaagaaggcggagtccaagaaggggtgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 242 - DNA, 5973 bp, synthetic construct
  source 1..5973: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaacgacctcaccgagctccagattcagcagctgcgcgatttcagcgactgcatcgtcgtgcacccccagatcaagatcatcttcgacgacttcgacgagctccgcttcaaccgcaagttccagtcggatcagcagtgcatgctcctcatcggcgacacgggcgtcggtaaatcccacctcatcaaccactacaagaagcgcgtcctcgccacccagaactactcccggggtaccatcccggtcctcgtcagccgcatctccaggggtaaaggcctcgaggccaccctcatccagatgcttgccgacctggagctattcgggagctcgcagatcaagaagcgcggctacaagatcgacctcaccaagaagctggtcgagaaccttatcaaggcccaggtcgaactgctaatcatcaacgagttccaagagctcatcgagttcaagtccgtgcaggagcggcagctgatcgccaacgggctgaagttcatctcagaggaggccaaggtccccatcgtcctcgtgggcatgccctgggccgagcgcatcgccgaggaacctcagtgggcgtcgaggctcatccgcaagcgccagctggagtacttcagcctcaagaacaactccaagtacttcaggcagtacctcatgggcctcgcgaagaagatgccattcgacgagccccctaagctgaaggtcaagcacaccaccctcgccatcttcagcgcctgcaagggcgagaaccgtgccctcaagcacctgctcaccgaggcgctcaagcaggccctctcctgcaacgagcacctggagaacaagcacttcgtcttcgccttcgacaagctctacctatccgacacgtcgtccttctcccagcagaagctcaccatcaagaacccattcaagcaggatatcaaggacatcaagatctacgaggtgagcaagaactcctcgtacaacccgaacgccatcgaccccgaggacgctctgaccggcagggagttctccctgatcgcctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 243 - DNA, 5973 bp, synthetic construct
  source 1..5973: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaacgacctcacggagctccagatccagcagctccgggacttctcggactgcatcgtggtccacccccagatcaagatcatcttcgacgacttcgacgaactccgcttcaaccgcaagttccagtccgaccagcagtgcatgctcctgatcggcgacacgggcgtcggcaagtcacatctcatcaaccactacaagaagcgcgttctggcgacccagaactactcgcggggtaccatccctgtgctcgtcagccgcatctcgcgcggtaaaggcctcgaggccaccctcatccagatgcttgctgaccttgagctcttcggctcgagccagatcaagaagcgcggctacaagatcgacctcacgaagaagctggtcgagaaccttatcaaggcgcaggtcgagctcctgatcatcaacgagttccaggagctcatcgagtttaagtccgtacaggagaggcagcttatcgccaacggcctgaagttcatcagcgaggaggccaaggtgccgatcgtcctcgtgggcatgccctgggccgagcgcatcgccgaggagccccagtgggccagccgcctcattcggaagcgccagctcgagtacttcagcctgaagaacaactccaagtacttccgccagtacctcatgggcctcgcgaagaagatgcctttcgacgagccgccgaagctcaaggtcaagcacaccacgctggccatcttctcggcctgcaagggcgaaaaccgtgccctcaagcacctgctcacagaagccctcaagcaggccctgtcctgcaacgagcacctcgagaacaagcacttcgtgttcgccttcgacaagctctacttgtccgacaccagctccttctcccagcagaagctcaccatcaagaacccttttaagcaagacatcaaggatatcaagatctacgaggtgtccaagaactcctcctacaaccccaacgccatcgaccccgaagacgccctcaccggccgcgagtttagcctgatcgcctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 244 - DNA, 5973 bp, synthetic construct
  source 1..5973: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaacgatctcaccgaactccagatccagcagctccgcgacttctccgattgcatcgtcgtccacccccagatcaagatcatcttcgacgacttcgacgagctgaggttcaaccgcaagttccagtcggaccagcagtgcatgctcctcatcggcgacaccggcgtcggcaagagccacctcatcaaccactacaagaagcgcgtcctggccacccagaactactcgaggggtacgatccccgtgctggtcagccgcatcagccgcggcaagggcctggaagccaccctcatccagatgctggctgacctggagctgttcggctcctcccagatcaagaagcgcggctataagatcgacctcaccaagaagctcgtggagaacctcatcaaggcccaggtcgagctcctcatcatcaacgagttccaggagctcatcgaattcaagagcgtccaggagcgccagctcatcgccaacggcctgaagttcatttcggaggaggccaaggtgcctatcgtcctggtcggcatgccctgggccgagaggatcgccgaggagccccagtgggcctcccgcctcatccgcaagcgccagctggaatacttcagcctgaagaacaactcgaagtacttccgccagtacctgatgggcctcgccaagaagatgcccttcgacgagccgccgaagctgaaggtcaagcacaccacgctcgcgattttctccgcctgcaagggcgagaaccgcgccctcaagcacctcctcacggaagccctgaagcaggccctctcctgcaacgagcacctggagaacaagcacttcgtgttcgcgttcgacaagctgtacctctccgataccagcagcttctcccagcagaagctcacgatcaagaacccgttcaagcaggacatcaaggacatcaagatctacgaggtcagcaagaactcctcctacaacccaaacgccatcgaccccgaggacgcgctgaccgggcgcgagttctccctgatcgcgtgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

SEQ ID NO: 245 - DNA, 5973 bp, synthetic construct
  source 1..5973: mol_type = other DNA
ctaattagctaactgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcaggatcctctaagtagcagccatggctccgaagaagaagcgcaaggtgtccggcggcagctccggcggcagcatgaacgacctcaccgagctgcagatacagcagctgcgcgacttctccgactgcatcgtggtgcaccctcagatcaagatcatcttcgacgacttcgacgagctccgcttcaacaggaagttccagtccgatcagcagtgcatgctcctcatcggcgacacgggtgtgggcaaaagccatctcatcaaccactacaagaagcgcgtcctcgccacgcagaactactcccgcggcaccatcccggtcctcgtcagcaggatctccaggggcaagggcctcgaggccaccctcatccagatgctcgccgacctcgagctcttcggctcctcgcagatcaagaagcgcggctacaagatcgacctgaccaagaagctcgtcgagaacctgatcaaggcccaagtggagctgctcatcatcaacgagttccaggagctcatcgagttcaagagcgtccaggagcgccagctcatcgccaacggcctgaagttcatctccgaggaagccaaggtccctatcgtgctcgtgggcatgccctgggccgagcgcatcgctgaagagccgcagtgggcctcccgcctcattcgcaagcgtcagctcgagtacttctccctcaagaacaattccaagtacttccgacagtacctgatgggcctggcgaagaagatgcccttcgacgagcccccgaagctcaaggtgaagcacaccacgctcgccatcttctcggcctgcaagggcgagaaccgcgccctcaagcacctcctcaccgaggccctcaagcaggctctcagctgcaacgagcacctcgagaacaagcactttgtcttcgcgttcgataagctctacctttccgacaccagctcgttctcccagcagaagctcacgatcaagaaccccttcaagcaggacatcaaggacatcaagatctacgaggtgtccaagaactcgtcctacaacccgaacgccatcgacccagaggacgcgctcaccggccgagagttctccctgatcgcctgatccggactgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccagctaattagctaacggaccgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgggaccc

(19) United States
(12) Patent Application Publication
(10) Pub. No.: US 2026/0110001 A1
(43) Pub. Date: Apr. 23, 2026

(54) RECOMBINANT TYPE I-F3 TRANSPOSON-ASSOCIATED CRISPR-CAS SYSTEMS AND METHODS OF USE

(71) Applicant: NORTH CAROLINA STATE UNIVERSITY, Raleigh, NC (US)

(72) Inventors: Rodolphe BARRANGOU, Raleigh, NC (US); Avery ROBERTS, Raleigh, NC (US)

(21) Appl. No.: 18/872,321

(22) PCT Filed: Jun. 7, 2023
(86) PCT No.: PCT/US2023/068018

§ 371 Date: Dec. 6, 2024

Related U.S. Application Data
(60) Provisional application No. 63/350,173, filed on Jun. 8, 2022.
(60) Provisional application No. 63/373,946, filed on Aug. 30, 2022.

(51) Int. Cl. C12N 15/90; C12N 9/22; C12N 15/11; C12N 15/74

(52) U.S. Cl. CPC C12N 15/902; C12N 9/224; C12N 15/11; C12N 15/74; C12N 2310/20

(57) ABSTRACT

This invention relates to recombinant nucleic acid constructs encoding Clustered Regularly Interspaced Short Palindromic Repeats (CRISPR)-Cas systems and transposon 7-like (Tn7-like) transposon systems for DNA integration, as well as methods of using the same.

### STATEMENT OF PRIORITY

This application claims the benefit, under 35 U.S.C. § 119 (e), of U.S. Provisional Application No. 63/373,946 filed on Aug. 30, 2022 and U.S. Provisional Application No. 63/350,173 filed on Jun. 8, 2022, the entire contents of each of which are incorporated by reference herein.

### STATEMENT REGARDING ELECTRONIC FILING OF A SEQUENCE LISTING

A Sequence Listing in XML format, entitled 5051-1005WO_ST26.xml, 465, 121 bytes in size, generated on May 30, 2023 and filed herewith, is hereby incorporated by reference into the specification for its disclosures.

### FIELD OF THE INVENTION

This invention relates to recombinant nucleic acid constructs encoding Clustered Regularly Interspaced Short Palindromic Repeats (CRISPR)-Cas systems and transposon 7-like (Tn7-like) transposon systems for DNA integration, as well as methods of using the same.

### BACKGROUND OF THE INVENTION

Over a decade ago, the biological function of CRISPR-Cas systems was elucidated in the dairy starter culture Streptococcus thermophilus (Barrangou et al. 2007. Science. 315:1709-1712), and this arguably gave rise to the subsequent interest in CRISPR loci and their accompanying Cas nucleases (Garneau et al. 2010. Nature. 468:67-71), ultimately leading to the genome editing revolution (Barrangou and Horvath 2017. Nat. Microbiol. doi: 10.1038/nmicrobiol.2017.92). Indeed, endogenous CRISPR (clustered regularly interspaced short palindromic repeats) arrays and Cas (CRISPR-associated) proteins comprise CRISPR-Cas systems, which function as adaptive immune systems to defend against invading nucleic acid material such as phages that prey upon dairy starter cultures (Barrangou et al. 2007. Science. 315:1709-1712).

CRISPR-Cas systems are present within approximately 85% of archaea and 40% of bacteria, and notably found in approximately 63% of Lactobacillus, 77% of Bifidobacterium, and 100% of S. thermophilus strains (Horvath et al. 2009. Int. J. Food Microbiol. 131(1):62-70; Sun et al. 2015. Nat. Commun. 6:8322; Crawley et al. 2018. CRISPR J. 1(2):171-181; Makarova et al. 2019. FEMS Microbiol. Lett. 366(7):fnz079). The CRISPR component of the CRISPR-Cas system is comprised of an array of conserved repeats that separate unique spacers that act as the genetic records of foreign invaders, such as phage and plasmids (Barrangou et al. 2007. Science. 315:1709-1712). Typically, repeats are 28-37 bp in length while spacers are 29-38 bp in length, but in some cases these lengths vary considerably (Barrangou and Marraffini 2014. Mol. Cell. 54(2):234-244). CRISPR-Cas systems operate in three distinct phases: adaptation (or acquisition), expression (or crRNA biogenesis), and interference (Makarova et al. 2015. Nat. Rev. Microbiol. 13(11):722-736; Hille and Charpentier 2016. Philos. Trans. R. Soc. Lond. B Biol. Sci. 371(1707):20150496). During the adaptation phase, Cas1 and Cas2 proteins form an adaptation complex and act as spacer-acquisition machinery to add new spacers to the CRISPR array (Yosef et al. 2012. Nucleic Acids Res. 40(12) 5569-5576; Nuñez et al. 2014. Nat. Struct. Mol. Biol. 21(6):58-534). The repeat-spacer array is immediately downstream of a leader sequence, an AT-rich sequence involved with spacer acquisition events and promoting transcription of the CRISPR array, and is typically flanked by cas genes (Kieper et al. 2019. FEMS Microbiol. Lett. 366(11):fnz129).

During the expression phase, the CRISPR array is generally transcribed as a single transcript known as a pre-crRNA (pre-CRISPR RNA) before being processed into individual, mature crRNAs, which each possess a single spacer present within the CRISPR array (Deltcheva et al. 2011. Nature 471:602-607). Mature crRNAs are used by a Cas effector complex, made up of a single or multiple Cas proteins, to guide the complex to the target (protospacer) sequence that matches the crRNA's spacer sequence from the CRISPR array (Brouns et al. 2008. Science 321(5891):960-964). This crRNA-guided targeting defines the interference phase of CRISPR-Cas immunity and results in cleavage of the target sequence by the effector complex (Garneau et al. 2010. Nature. 468:67-71; Gasiunas et al. 2012. Proc. Natl. Acad. Sci. 109: E2579-E2586; Jinek et al. 2012. Science 337(6096):816-821). Generally, CRISPR-Cas systems are defined as DNA-encoded (Barrangou et al. 2007. Science. 315:1709-1712), RNA-mediated (Brouns et al. 2008. Science 321(5891):960-964), DNA-targeting (Marraffini and Sontheimer 2008. Science. 322:1843-1845) bacterial immune systems, but Cas-derived molecular machines have been repurposed for a diversity of applications encompassing genome editing.

CRISPR-Cas systems are classified first into classes, then types, subtypes, and finally variants based on the presence of signature cas genes, loci organization, and phylogenetic analysis (Makarova et al. 2015. Nat. Rev. Microbiol. 13(11):722-736; FIG. 1). In late 2019, an updated classification scheme was presented, which added an additional type (type VI) and more than doubled the number of subtypes for a new total of 6 types and 33 subtypes (Makarova et al. 2019. FEMS Microbiol. Lett. 366(7):fnz079). Class 1 systems encompass type I, III, and IV systems and their respective subtypes, while class 2 systems are comprised of type II, V, and VI systems and their respective subtypes (Makarova et al. 2015. Nat. Rev. Microbiol. 13(11):722-736). Class 1 systems possess a multi-protein effector complex, such as the type I Cascade (CRISPR-associated complex for antiviral defense) complex, while class 2 systems utilize a single-protein effector complex, such as the prominent type II Cas9 (Makarova et al. 2015. Nat. Rev. Microbiol. 13(11):722-736; Shmakov et al. 2015. Mol. Cell 60(3):385-397). While the adaptation phase is conserved with few exceptions across both class 1 and class 2 systems, expression and interference phases vary greatly between the classes and within their respective types (Makarova et al. 2015. Nat. Rev. Microbiol. 13(11):722-736).

### SUMMARY OF THE INVENTION

One aspect of the invention provides a system for RNA-guided DNA integration, the system comprising: (A) one or more vectors heterologous to Aliiglaciecola sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Aliiglaciecola sp.; (B) one or more vectors heterologous to Vibrio sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Vibrio sp.; (C) one or more vectors heterologous to Halomonas titanicae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from H. titanicae; (D) one or more vectors heterologous to Photobacterium aquae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. aquae; (E) one or more vectors heterologous to Photobacterium iliopiscarium encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. iliopiscarium; (F) one or more vectors heterologous to Photobacterium piscicola encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. piscicola; (G) one or more vectors heterologous to Psychromonas sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Psychromonas sp.; (H) one or more vectors heterologous to Klebsiella oxytoca encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein AB (TnsAB), (b) Transposon 7 protein C (TnsC), and (c) transposition of integron protein Q (TniQ), wherein the engineered Tn7-like transposon system is derived from K. oxytoca; (I) one or more vectors heterologous to Colwellia polaris encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from C. polaris; or (J) one or more vectors heterologous to Neptunomonas qingdaonensis encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from N. qingdaonensis.

A second aspect provides a recombinant cell comprising the system of the invention.

A third aspect provides a method for RNA-guided DNA integration, the method comprising introducing the system of the invention into a cell, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid.

A fourth aspect provides a method of modifying the genome of a target organism, the method comprising introducing the system of the invention into a cell, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the genome of the target organism.

A fifth aspect provides a method of modifying (e.g., editing) a nucleic acid (e.g., target region; target DNA) in the genome of a cell, the method comprising introducing into a cell the system of the invention, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the nucleic acid in the genome of the cell.

A sixth aspect provides a method of killing a cell, the method comprising introducing the system of the invention into a cell, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into an essential gene in the nucleic acid, thereby killing the cell.

A seventh aspect provides a method of screening for non-essential genes in a cell, the method comprising introducing into a cell the system of the invention, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into a gene in the nucleic acid, wherein when the cell survives, the gene is non-essential, thereby screening for non-essential genes in the cell.

An eighth aspect provides a method of altering the expression of a gene in a cell, the method comprising introducing the system of the invention into a cell, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into a regulator region of a gene in the nucleic acid, thereby altering the expression of a gene in a cell.

These and other aspects of the invention are set forth in more detail in the description of the invention below.

### BRIEF DESCRIPTION OF THE DRAWINGS

FIG. 1. Impact of incubation temperature on integration efficiency at a single target site. Integration efficiency is shown, as measured by qPCR, at two different incubation temperatures, 30° C. and 25° C., for a single target site with a 5′-CCTCC-3′ PAM. Each system integrated a transposon approximately 1 kb in size. Data is shown for 3 biological replicates (n=3).

FIG. 2. Impact of incubation temperature on integration orientation bias at a single target site. Related to FIG. 1. Orientation bias is shown, as measured by qPCR, at two different incubation temperatures, 30° C. and 25° C., for a single target site with a 5′-CCTCC-3′ PAM. Data is shown for three biological replicates (n=3).

FIG. 3. Protospacer adjacent motif (PAM) profiles of CasTn systems. WebLogos depicting PAM preferences were generated using the top 10% of enriched PAM sequences within sequencing data generated from a plasmid library assay. The nucleotides of the PAM are presented as positions −5 to −1, with position −1 occurring immediately before the first nucleotide of the target sequence.

FIG. 4. Impact of PAM sequence on integration efficiency. Each CasTn was directed to integrate a transposon of approximately 1 kb in size at six target sites with distinct PAM sequences (shown in legend). Integration efficiency, as measured by qPCR, is shown for three biological replicates (n=3).

FIG. 5. Impact of PAM sequence on integration orientation bias. Orientation bias, as measured by qPCR, is shown for six target sites (shown in legend) for two CasTn systems. Data is shown for three biological replicates (n=3).

FIG. 6. Shows orthogonality testing (by integration efficiency) of three selected systems, including the V. cholerae system, the P. aquae system and the Psychromonas sp. system.

FIG. 7. Impact of transposon size on integration efficiency. Integration efficiency, as measured by qPCR, is shown for transposons approximately 1 kb and 10 kb in size integrated at a single target site with a 5′-CCTCC-3′ PAM. Data is shown for three biological replicates (n=3).

FIG. 8. Panel A shows integration distance patterns at a target site with a 5′-CCTCC-3′ PAM and Panel B shows integration distance patterns for V. cholerae and Psychromonas sp. at multiple target sites with distinct PAM sequences. Data is shown as the percentage of reads corresponding to distinct integration distances from the target site.

FIG. 9. Characterization of self-targeting spacers. The N. qingdaonensis system was tested at 30° C. for RNA-guided DNA transposition at the 5′-CCTCC-3′ PAM lacZ target site using different native CRISPR repeat pairs. Integration efficiency with each CRISPR repeat pair is relative to the mean of the typical CRISPR repeats data. Typical=Typical=SEQ ID NO:99+SEQ ID NO:100; Atypical Set 1=SEQ ID NO:95+SEQ ID NO:96; Atypical Set 2=SEQ ID NO: 97+SEQ ID NO:98.

FIG. 10. Impact of incubation temperature on RNA-guided DNA transposition using the Psychromonas sp. system. Integration efficiency is shown, as measured by qPCR, at two different incubation temperatures, 30° C. and 25° C., for a single target site with a 5′-CCTCC-3′ PAM with different combinations of native CRISPR repeats. The shown data is normalized to the mean of the wild-type (WT) data for the respective temperature. WT=SEQ ID NO: 172+SEQ ID NO: 173; Repeat #1-2=SEQ ID NO: 174+SEQ ID NO:172; Repeat #1-1=SEQ ID NO: 174+SEQ ID NO: 174; Repeat #2-2=SEQ ID NO: 172+SEQ ID NO: 172; Repeat #3-3=SEQ ID NO: 173+SEQ ID NO:173.

FIG. 11. Impact of the length of transposon ends on integration efficiency. The Psychromonas sp. system was tested at 25° C. for RNA-guided DNA transposition at the 5′-CCTCC-3′ PAM lacZ target site with different lengths of the associated left and right transposon ends. The original transposon end lengths are noted with an asterisk (*) and correspond to SEQ ID NO: 176 (199 bp) and SEQ ID NO: 175 (211 bp). For these experiments, the transposon end that was not truncated remained at its original length (e.g., when the left transposon end was truncated, the right end remained at 211 bp). The shown data is normalized to the mean of the original transposon end length (noted with an asterisk) data for the respective transposon end. 150-bp transposon left end including 3 TnsB binding sites, SEQ ID NO:205; 109-bp transposon left end including 3 TnsB binding sites, SEQ ID NO:206; 86-bp transposon left end including 2 TnsB binding sites, SEQ ID NO:207; 59-bp transposon left end including 1 TnsB binding site, SEQ ID NO:208; 156-bp transposon right end including 4 TnsB binding sites, SEQ ID NO: 209; 129-bp transposon right end including 3 TnsB binding sites, SEQ ID NO:210; 72-bp transposon right end including 3 TnsB binding sites, SEQ ID NO:211; 50-bp transposon right end including 2 TnsB binding sites, SEQ ID NO:212.

FIG. 12. Impact of the length of transposon ends on RL orientation frequency. The Psychromonas sp. system was tested at 25° C. for RNA-guided DNA transposition at the 5′-CCTCC-3′ PAM lacZ target site with different lengths of the associated left and right transposon ends. The original transposon end lengths are noted with an asterisk (*) and correspond to SEQ ID NO: 176 (199 bp) and SEQ ID NO:175 (211 bp). Integrated transposon orientation data is shown as RL orientation frequency, or the frequency with which the transposon was integrated in the right-to-left orientation. The frequency is based on the captured RL integration activity within the total integration activity which is calculated by combining the quantified RL and LR integration activities. 150-bp transposon left end, SEQ ID NO:205; 109-bp transposon left end, SEQ ID NO:206; 86-bp transposon left end, SEQ ID NO: 207; 59-bp transposon left end, SEQ ID NO: 208; 156-bp transposon right end, SEQ ID NO: 209; 129-bp transposon right end, SEQ ID NO:210; 72-bp transposon right end, SEQ ID NO:211; 50-bp transposon right end, SEQ ID NO:212.

FIG. 13. Expression analysis of Psychromonas sp. system in maize cells.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancement,” “improve” and “improvement” (and the like and grammatical variations thereof) describe an elevation of at least about 5%, 10%, 15%, 20%, 25%, 50%, 75%, 100%, 150%, 200%, 300%, 400%, 500%, 750%, or 1000%, or more as compared to a control.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

As used herein, the phrase “substantially complementary,” or “substantial complementarity” in the context of two nucleic acid molecules, nucleotide sequences or protein sequences, refers to two or more sequences or subsequences that are at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue complementary, when compared and aligned for maximum correspondence, as measured using one of the following sequence comparison algorithms or by visual inspection. In some embodiments, substantial complementarity can refer to two or more sequences or subsequences that have at least about 80%, at least about 85%, at least about 90%, at least about 95, 96, 96, 97, 98, or 99% complementarity (e.g., about 80% to about 90%, about 80% to about 95%, about 80% to about 96%, about 80% to about 97%, about 80% to about 98%, about 80% to about 99% or more, about 85% to about 90%, about 85% to about 95%, about 85% to about 96%, about 85% to about 97%, about 85% to about 98%, about 85% to about 99% or more, about 90% to about 95%, about 90% to about 96%, about 90% to about 97%, about 90% to about 98%, about 90% to about 99% or more, about 95% to about 97%, about 95% to about 98%, about 95% to about 99% or more). Two nucleotide sequences can be considered to be substantially complementary when the two sequences hybridize to each other under stringent conditions. In some representative embodiments, two nucleotide sequences considered to be substantially complementary hybridize to each other under highly stringent conditions.

A “control” as used herein is the same organism, e.g., a prokaryotic cell (e.g., a bacterium, an archaeon, and the like), a eukaryotic cell (e.g., a plant, an animal, a mammal, a primate and the like) that is typically the same as the organism that has been contacted by one or more of the systems of this invention, but the control has not been similarly contacted and therefore is devoid of the modification resulting from contact with the system.

As used herein, “contact,” contacting,” “contacted,” and grammatical variations thereof, refers to placing the components of a desired reaction together under conditions suitable for carrying out the desired reaction (e.g., integration, transformation, site-specific cleavage (nicking, cleaving), amplifying, site specific targeting of a polypeptide of interest and the like). The methods and conditions for carrying out such reactions are well known in the art (See, e.g., Gasiunas et al. (2012) Proc. Natl. Acad. Sci. 109: E2579-E2586; M. R. Green and J. Sambrook (2012) Molecular Cloning: A Laboratory Manual. 4th Ed., Cold Spring Harbor Laboratory Press, Cold Spring Harbor, NY).

A “fragment” or “portion” of a nucleic acid will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising a nucleotide sequence of contiguous nucleotides that are identical or almost identical (e.g., 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. In some embodiments, a fragment of a polynucleotide can be a fragment that encodes a polypeptide that retains its function (e.g., encodes a fragment of a polypeptide of a Type 1-F CRISPR-Cas system (e.g., a Type 1-F3 CRISPR-Cas system) as described herein that is reduce in length as compared to the wild type polypeptide but which retains at least one function of a Type 1-F CRISPR-Cas system protein (e.g., binds DNA, forms a complex and/or transposes DNA). In some embodiments, a fragment of a polynucleotide can be a fragment of a native repeat sequence (e.g., a native repeat sequence from a Type 1-F CRISPR-Cas system as described herein, optionally wherein the repeat fragment is shortened by about 1 nucleotide to about 8 nucleotides from the 3′ end as compared to the native repeat sequence).

As used herein, “chimeric” refers to a nucleic acid molecule or a polypeptide in which at least two components are derived from different sources (e.g., different organisms, different coding regions).

A “heterologous” or a “recombinant” nucleic acid is a nucleic acid not naturally associated with a host cell into which it is introduced, including non-naturally occurring multiple copies of a naturally occurring nucleic acid.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to the nucleotide sequence of the invention.

As used herein, hybridization, hybridize, hybridizing, and grammatical variations thereof, refer to the binding of two complementary nucleotide sequences or substantially complementary sequences in which some mismatched base pairs are present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. 2012. Proc. Natl. Acad. Sci. 109: E2579-E2586; M. R. Green and J. Sambrook 2012. Molecular Cloning: A Laboratory Manual. 4th Ed., Cold Spring Harbor Laboratory Press, Cold Spring Harbor, NY).

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is a mRNA that is naturally occurring in or endogenous to the organism. A “homologous” nucleic acid is a nucleic acid naturally associated with a host cell into which it is introduced.

As used herein, the terms “nucleic acid,” “nucleic acid molecule,” “nucleic acid construct,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA. Thus, although the nucleic acid constructs of this invention may be described and used in the form of DNA, depending on the intended use, they may also be described and used in the form of RNA.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “synthetic” nucleic acid or nucleotide sequence, as used herein, refers to a nucleic acid or nucleotide sequence that is not found in nature but is constructed by a human and as a consequence is not a product of nature.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide,” and “polynucleotide” are also used interchangeably herein to refer to a heteropolymer of nucleotides. Except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.26. A “5′ region” as used herein can mean the region of a polynucleotide that is nearest the 5′ end. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end. Thus, for example, an element in the 3′ region of a polynucleotide can be located anywhere from the first nucleotide located at the 3′ end of the polynucleotide to the nucleotide located halfway through the polynucleotide. An element that is described as being “at the 5′ end” or “at the 3′ end” of a polynucleotide (5′ to 3′) refers to an element located immediately adjacent to (upstream of) the first nucleotide at the 5′ end of the polynucleotide, or immediately adjacent to (downstream of) the last nucleotide located at the 3′ end of the polynucleotide, respectively.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

The terms “complementary” or “complementarity,” as used herein, refer to the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complementary” as used herein can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

As used herein, a “hairpin sequence” is a nucleotide sequence comprising hairpins. A hairpin (e.g., stem-loop, fold-back) refers to a nucleic acid molecule having a secondary structure that includes a region of nucleotides that form a single strand that are further flanked on either side by a double stranded-region. Such structures are well known in the art. As known in the art, the double stranded region can comprise some mismatches in base pairing or can be perfectly complementary. In some embodiments, a repeat sequence may comprise, consist essentially of, consist of a hairpin sequence that is located within the repeat nucleotide sequence (i.e., at least one nucleotide (e.g., one, two, three, four, five, six, seven, eight, nine, ten, or more) of the repeat nucleotide sequence is present on either side of the hairpin that is within the repeat nucleotide sequence).

A “CRISPR array” as used herein means a nucleic acid molecule that comprises at least two CRISPR repeat nucleotide sequences, or a portion(s) thereof, and at least one spacer sequence, wherein one of the two repeat nucleotide sequences, or a portion thereof, is linked to the 5′ end of the spacer sequence and the other of the two repeat nucleotide sequences, or portion thereof, is linked to the 3′ end of the spacer sequence (e.g., a first repeat sequence, or a portion thereof, may be linked to the 5′ end of the spacer sequence and a second repeat sequence, or portion thereof, may be is linked to the 3′ end of the spacer sequence). In a recombinant CRISPR array of the invention, the combination of repeat nucleotide sequences and spacer sequences is synthetic and not found in nature. The CRISPR array may be introduced into a cell or cell free system as RNA or as DNA in an expression cassette or vector (e.g., plasmid, retrovirus, bacteriophage). In some embodiments, a “CRISPR array” can be referred to a guide nucleic acid, a guide RNA, gRNA; and/or, when processed, a CRISPR array may be referred to as a crRNA.

As used herein, the term “spacer sequence” refers to a nucleotide sequence that is complementary (e.g., at least about 65% complementary) to a targeted portion (i.e., “protospacer”) of a nucleic acid or a genome. The term “genome,” as used herein, refers to both chromosomal and non-chromosomal elements (i.e., extrachromosomal (e.g., mitochondrial, plasmid, plastid, chloroplast, and/or extrachromosomal circular DNA (eccDNA)) of a target organism. The spacer sequence guides the CRISPR machinery to the targeted portion of the genome, wherein the targeted portion of the genome may be, for example, modified (e.g., a deletion, an insertion, a single base pair addition, a single base pair substitution, a single base pair removal, a stop codon insertion, and/or a conversion of one base pair to another base pair (base editing)). The systems of this invention (e.g., CRISPR-Cas systems and transposon 7-like (Tn7-like) transposon systems, e.g., type I-F3 CRISPR-Cas systems, type I-F3 transposon-associated CRISPR-Cas systems) can tolerate multiple mismatches between the spacer and target sequence, that is they can edit, including integration of DNA (e.g., cargo DNA), using spacers that are not 100% complementary with the target site. Thus, in some embodiments, the percent complementarity of a spacer to a target site can be less than 100%, e.g., about 65% to about 99.9% (e.g., about 65, 66, 67, 68, 69, 70, 71, 72, 73, 74, 75, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% complementarity).

A “target sequence” or “protospacer” refers to a targeted portion of a genome or of a cell free nucleic acid that is complementary to the spacer sequence of a recombinant CRISPR array. In some embodiments, a target sequence or protospacer useful with this invention is located immediately adjacent to the 3′ end of a PAM (protospacer adjacent motif) (e.g., 5′-PAM-Protospacer-3′). In some embodiments, a PAM may comprise, consist essentially of, or consist of a sequence of 5′-NNNNN-3′ that is immediately adjacent to and 5′ of the target site (protospacer). Thus, in some embodiments, a PAM is not required, see, e.g., Psychromonas sp. type I-F3 system as described herein. In some embodiments, the PAM comprises a nucleotide sequence of 5′-NNNCN-3′ or 5′-NNNCC-3′ that is immediately adjacent to and 5′ of the target site. Thus, the PAM (protospacer adjacent motif) requirements for the type I-F3 systems as described herein are slim. Often there is only preference for a single nucleotide within a PAM that is useful with the type I-F3 systems of this invention (e.g., 5′-NNNCN-3′ or 5′-NNNCC-3′). Notably, successful DNA transposition can occur with PAMs that are not preferred. Further, the type I-F3 systems of this invention can tolerate multiple mismatches between the spacer and target sequence. The PAM and spacer requirements for the systems of this invention contrast with the strict targeting requirements of, for example, Streptococcus pyogenes Cas9, which typically requires a 5′-NGG-3′ PAM and near-complete spacer-target sequence complementary. Despite the flexibility of the type I-F3 systems as described herein, they have been shown to be capable of, for example, highly specific and efficient RNA-guided DNA transposition.

As used herein, the terms “target genome” or “targeted genome” refer to a genome of an organism of interest.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” in the context of two nucleic acid molecules, nucleotide sequences or protein sequences, refers to two or more sequences or subsequences that have at least about 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In particular embodiments, substantial identity can refer to two or more sequences or subsequences that have at least about 80%, at least about 85%, at least about 90%, at least about 95, 96, 96, 97, 98, or 99% identity.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff 1989. Proc. Natl. Acad. Sci. USA 89:10915).

In addition to calculating percent sequence identity, the BLAST algorithm also performs a statistical analysis of the similarity between two sequences (see, e.g., Karlin & Altschul 1993. Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen 1993. Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tm) for the specific sequence at a defined ionic strength and pH.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC, 0.1% SDS at 50° C. In another embodiment, the reference nucleotide sequence hybridizes to the “test” nucleotide sequence in 7% SDS, 0.5 M NaPO₄, 1 mM EDTA at 50° C. with washing in 1×SSC, 0.1% SDS at 50° C. or in 7% SDS, 0.5 M NaPO₄, 1 mM EDTA at 50° C. with washing in 0.5×SSC, 0.1% SDS at 50° C. In still further embodiments, the reference nucleotide sequence hybridizes to the “test” nucleotide sequence in 7% SDS, 0.5 M NaPO₄, 1 mM EDTA at 50° C. with washing in 0.1×SSC, 0.1% SDS at 50° C., or in 7% SDS, 0.5 M NaPO₄, 1 mM EDTA at 50° C. with washing in 0.1×SSC, 0.1% SDS at 65° C.

Any polynucleotide and/or nucleic acid construct useful with this invention may be optimized for expression (e.g., enhanced or optimal expression) in any species of interest. For example, the presence/absence, location and/or number of introns; the presence/absence, location and/or number of nuclear localization signals; GC content; codon usage; and/or mRNA structure (e.g., opening intra-molecularly formed stems or removal of reverse complement repeats) can be optimized to facilitate heterologous expression of a polynucleotide and/or nucleic acid construct of this invention. In addition, several DNA- and/or mRNA-based sequence motifs may play a role in modulating gene expression. For example, internal ribosomal entry sites may lead to truncated products during heterologous expression and UpA-dinucleotides, preferred targets of endoribonuclease cleavage, may impact mRNA stability. In addition, AU-rich (ARE)-elements in the 3′ untranslated region of mRNAs may be determinants of mRNA instability. Sequence optimization for enhanced or optimal expression is well known in the art. By way of illustration, codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. As is understood in the art, optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function (and in some embodiments, the same structure) as that encoded by the original nucleotide sequence. Thus, in some embodiments of the invention, polynucleotides and/or nucleic acid constructs useful with the invention may be optimized for expression in the particular organism/species of interest.

In some embodiments, the polynucleotides and polypeptides of the invention are “isolated.” An “isolated” polynucleotide sequence or an “isolated” polypeptide is a polynucleotide or polypeptide that, by human intervention, exists apart from its native environment and is therefore not a product of nature. An isolated polynucleotide or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated polynucleotide and/or the isolated polypeptide may be at least about 1%, 5%, 10%, 20%, 30%, 40%, 50%, 60%, 70%, 80%, 90%, 95%, or more pure.

In other embodiments, an isolated polynucleotide or polypeptide may exist in a non-natural environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the polynucleotides and their encoded polypeptides are “isolated” in that, through human intervention, they exist apart from their native environment and therefore are not products of nature, however, in some embodiments, they can be introduced into and exist in a recombinant host cell.

In some embodiments of the invention, a system for RNA-guided DNA integration of the invention (e.g., a type I-F3 system as described herein) may be operatively associated with a variety of promoters, terminators and other regulatory elements for expression in various organisms or cells. Thus, in some embodiments, at least one promoter and/or at least one terminator may be operably linked to a recombinant nucleic acid of the invention comprising/encoding (i) an engineered Clustered Regularly Interspaced Short Palindromic Repeats (CRISPR)-CRISPR associated (Cas) (CRISPR-Cas) system, wherein the engineered CRISPR-Cas system comprises Cas6, Cas7 and Cas8, and (ii) an engineered transposon 7-like (Tn7-like) transposon system, wherein the engineered Tn7-like transposon system comprises (a) Transposon 7 protein A (TnsA), (b) Transposon 7 protein B (TnsB) (optionally, TnsAB, TnsC, and TniQ, (c) Transposon 7 protein C (TnsC), and (d) transposition of integron protein Q (TniQ). In some embodiments, the engineered CRISPR-Cas system and the engineered transposon 7-like (Tn7-like) transposon system may be encoded on the same recombinant nucleic acid (e.g., the same polynucleotide, the same expression cassette, the same vector) or they may be encoded on two or more separate recombinant nucleic acids (e.g., two or more separate polynucleotides, expression cassettes, and/or vectors), each operably linked to a separate promoter (independent promoters) and/or terminator, which can be the same or different promoters and/or terminators. As used herein, the term “engineered” refers to the aspect of having been manipulated by the hand of a man In some embodiments, Cas6, Cas7 and Cas8 of the CRISPR-Cas system may be encoded on same recombinant nucleic acid or they may be encoded on two or more separate (independent) recombinant nucleic acids, in any combination, each recombinant nucleic acid operably linked to a separate (independent) promoter and/or terminator, which separate promoters and/or separate terminators can be the same or different. In some embodiments, TnsA, TnsB, TnsC, and TniQ, optionally TnsAB, TnsC, and TniQ, of the engineered transposon system may be encoded on same recombinant nucleic acid or they may be encoded on two or more separate recombinant nucleic acids, in any combination, each recombinant nucleic acid operably linked to a separate promoter and/or terminator, which separate promoters and/or separate terminators can be the same or different. Further, when comprised in the same nucleic acid construct (e.g., polynucleotide, expression cassette, vector), the engineered CRISPR-Cas system and the engineered transposon 7-like (Tn7-like) transposon system and/or components thereof (e.g., Cas6, Cas7, Cas8, TnsA, TnsB, TnsC, and TniQ, optionally Cas6, Cas7, Cas8, TnsAB, TnsC, and TniQ), may be operably linked to separate (independent) promoters that may be the same promoter or a different promoter. In some embodiments, when comprised in the same nucleic acid construct (e.g., polynucleotide, expression cassette, vector), a recombinant nucleic acid comprising an engineered CRISPR-Cas system and the engineered transposon 7-like (Tn7-like) transposon system may be operably linked to a single promoter and/or a single terminator.

Any promoter that initiates transcription of a recombinant nucleic acid construct of the invention in an organism/cell of interest may be used. A promoter useful with this invention can include, but is not limited to, a constitutive, inducible, developmentally regulated, tissue-specific/preferred-promoter, and the like, as described herein. A regulatory element as used herein can be endogenous or heterologous. In some embodiments, an endogenous regulatory element derived from the subject organism can be inserted into a genetic context in which it does not naturally occur (e.g., a different position in the genome than as found in nature (e.g., a different position in a chromosome or in a plasmid), thereby producing a recombinant or non-native nucleic acid. In some embodiments, promoters useful with the constructs of the invention may be any combination of heterologous and/or endogenous promoters.

By “operably linked,” “operably associated” or “linked” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of the nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” These various types of promoters are known in the art. Thus, expression can be made constitutive, inducible, temporally regulated, developmentally regulated, chemically regulated, tissue-preferred and/or tissue-specific promoters using the recombinant nucleic acid constructs of the invention operatively linked to the appropriate promoter functional in an organism of interest. Expression may also be made reversible using the recombinant nucleic acid constructs of the invention operatively linked to, for example, an inducible promoter functional in an organism of interest.

The choice of promoter will vary depending on the quantitative, temporal and spatial requirements for expression, and also depending on the host cell of interest. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

Exemplary promoters include, but are not limited to, promoters functional in eukaryotes and prokaryotes including but not limited to, plants, viruses, bacteria, fungi, animals, and mammals. For example, promoters useful with archaea include, but are not limited to, Haloferax volcanii tRNA (Lys) promoter (Palmer et al. 1995. J. Bacteriol. 177(7):1844-1849), Pyrococcus furiosus gdh promoter (Waege et al. 2010. Appl. Environ. Microbiol. 76:3308-3313), Sulfolobus sulfataricus 16S/23S rRNA gene core promoter (DeYoung et al. 2011. FEMS Microbiol. Lett. 321:92-99).

Exemplary promoters useful with yeast can include a promoter from phosphoglycerate kinase (PGK), glyceraldehyde-3-phosphate dehydrogenase (GAP), triose phosphate isomerase (TPI), galactose-regulon (GAL1, GAL10), alcohol dehydrogenase (ADH1, ADH2), phosphatase (PHO5), copper-activated metallothionine (CUP1), MFα1, PGK/α2 operator, TPI/α2 operator, GAP/GAL, PGK/GAL, GAP/ADH2, GAP/PHO5, iso-1-cytochrome c/glucocorticoid response element (CYC/GRE), phosphoglycerate kinase/androgen response element (PGK/ARE), transcription elongation factor EF-1α (TEF1), triose phosphate dehydrogenase (TDH3), phosphoglycerate kinase 1 (PGK1), pyruvate kinase 1 (PYK1), and/or hexose transporter (HXT7) (See, Romanos et al. 1992. Yeast 8:423-488; Partow et al. 2010. Yeast 27:955-964).

Exemplary promoters useful with bacteria can include, but are not limited to, L-arabinose inducible (araBAD, P_{BAD}) promoter, any lac promoter, L-rhamnose inducible (rhaP_{BAD}) promoter, T7 RNA polymerase promoter, trc promoter, tac promoter, lambda phage promoter (p_{L}, p_{L}-9G-50), anhydrotetracycline-inducible (tetA) promoter, trp, Ipp, phoA, recA, proU, cst-1, cadA, nar, Ipp-lac, cspA, T7-lac operator, T3-lac operator, T4 gene 32, T5-lac operator, nprM-lac operator, Vhb, Protein A, corynebacterial-Escherichia coli like promoters, thr, hom, diphtheria toxin promoter, sig A, sig B, nusG, SoxS, katb, α-amylase (Pamy), Ptms, P43 (comprised of two overlapping RNA polymerase σ factor recognition sites, σA, σB), Ptms, P43, rpIK-rplA, ferredoxin promoter, and/or xylose promoter. (See, Terpe 2006. Appl. Microbiol, Biotechnol. 72:211-222; Hannig et al. 1998. Trends Biotechnol. 16:54-60; Srivastava 2005. Protein Expr. Purif. 40:221-229).

Translation elongation factor promoters may be used with the invention. Translation elongation factor promoters may include but are not limited to elongation factor Tu promoter (Tuf) (e.g., Ventura et al. 2003. Appl. Environ. Microbiol. 69:6908-6922), elongation factor P (Pefp) (e.g., Tauer et al. 2014. Microb. Cell Fact.s 13:150), rRNA promoters including but not limited to a P3, a P6 a P15 promoter (e.g., Djordjevic et al. 1997. Can. J. Microbiol. 43:61-69; Russell & Klaenhammer 2001. Appl. Environ. Microbiol. 67:1253-1261) and/or a P11 promoter. In some embodiments, a promoter may be a synthetic promoter derived from a natural promoter (e.g., Rud et al. 2006. Microbiology 152:1011-1019). In some embodiments, a sakacin promoter may be used with the recombinant nucleic acid constructs of the invention (e.g., Mathiesen et al. 2004. J. Appl. Microbiol. 96:819-827).

A promoter useful with the recombinant nucleic acid constructs of the invention may be a promoter from any bacterial species. In some embodiments, a promoter from Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis may be operably linked to a recombinant nucleic acid construct of the invention.

Non-limiting examples of a promoter functional in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (see, Walker et al. 2005. Plant Cell Rep. 23:727-735; Li et al. 2007. Gene 403:132-142; Li et al. 2010. Mol Biol. Rep. 37:1143-1154). PrbcS1 and Pactin are constitutive promoters and Pnr and Pdca1 are inducible promoters. Pnr is induced by nitrate and repressed by ammonium (Li et al. 2007. Gene 403:132-142) and Pdca1 is induced by salt (Li et al. 2010. Mol Biol. Rep. 37:1143-1154).

Examples of constitutive promoters useful for plants include, but are not limited to, cestrum virus promoter (cmp) (U.S. Pat. No. 7,166,770), the rice actin 1 promoter (Wang et al. 1992. Mol. Cell. Biol. 12:3399-3406; as well as U.S. Pat. No. 5,641,876), CaMV 35S promoter (Odell et al. 1985. Nature 313:810-812), CaMV 19S promoter (Lawton et al. 1987. Plant Mol. Biol. 9:315-324), nos promoter (Ebert et al. 1987. Proc. Natl. Acad. Sci USA 84:5745-5749), Adh promoter (Walker et al. 1987. Proc. Natl. Acad. Sci. USA 84:6624-6629), sucrose synthase promoter (Yang & Russell 1990. Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al. 1991. Plant Science 79:87-94), maize (Christensen et al. 1989. Plant Mol. Biol. 12:619-632), and arabidopsis (Norris et al. 1993. Plant Mol. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (1991. Mol. Gen. Genet. 231:150-160) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. 1991. Seed Sci. Res. 1:209-219; as well as EP U.S. Pat. No. 255,378). Additional examples of plant tissue-specific/tissue preferred promoters include, but are not limited to, the root hair-specific cis-elements (RHEs) (Kim et al. 2006. Plant Cell 18:2958-2970), the root-specific promoters RCc3 (Jeong et al. 2010. Plant Physiol. 153:185-197) and RB7 (U.S. Pat. No. 5,459,252), the lectin promoter (Lindstrom et al. 1990. Der. Genet. 11:160-167; and Vodkin 1983. Prog. Clin. Biol. Res. 138:87-98), corn alcohol dehydrogenase 1 promoter (Dennis et al. 1984. Nucleic Acids Res. 12:3983-4000), and/or S-adenosyl-L-methionine synthetase (SAMS) (Vander Mijnsbrugge et al. 1996. Plant Cell Physiol. 37(8):1108-1115).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the RNAs and/or the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression. In some aspects, a promoter can also include a light-inducible promoter, where application of specific wavelengths of light induces gene expression (Levskaya et al. 2005. Nature 438:441-442). In other aspects, a promoter can include a light-repressible promoter, where application of specific wavelengths of light repress gene expression (Ye et al. 2011. Science 332:1565-1568).

Chemically inducible promoters useful with plants are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid-responsive promoters (see, e.g., the glucocorticoid-inducible promoter in Schena et al. 1991. Proc. Natl. Acad. Sci. USA 88:10421-10425; McNellis et al. 1998. Plant J. 14:247-257; and Aoyama et al. 1997. Plant J. 11:605-612), tetracycline-inducible and tetracycline-repressible promoters (see, e.g., Gatz et al. 1991. Mol. Gen. Genet. 227:229-237, and U.S. Pat. Nos. 5,814,618 and 5,789,156), Lac repressor system promoters, copper-inducible system promoters, salicylate-inducible system promoters (e.g., the PR1a system), and ecdysone-inducible system promoters.

In some embodiments, promoters useful with algae include, but are not limited to, the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (see, Walker et al. 2005. Plant Cell Rep. 23:727-735; Li et al. 2007. Gene 403:132-142; Li et al. 2010. Mol Biol. Rep. 37:1143-1154), the promoter of the g70-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (see, e.g., De Cosa et al. 2001. Nat. Biotechnol. 19:71-74; Daniell et al. 2009. BMC Biotechnol. 9:33; Muto et al. 2009. BMC Biotechnol. 9:26; Surzycki et al. 2009. Biologicals 37:133-138).

In some embodiments, a promoter useful with this invention can include, but is not limited to, pol III promoters such as the human U6 small nuclear promoter (U6) and the human H1 promoter (H1) (Mäkinen et al. 2006. J. Gene Med. 8(4):433-41), and pol II promoters such as the CMV (Cytomegalovirus) promoter (Barrow et al. 2006. Meth. Mol. Biol. 329:283-294), the SV40 (Simian Virus 40)-derived initial promoter, the EF-1α (Elongation Factor-1a) promoter, the Ubc (Human Ubiquitin C) promoter, the PGK (Murine Phosphoglycerate Kinase-1) promoter and/or constitutive protein gene promoters such as the β-actin gene promoter, the tRNA promoter and the like.

Moreover, tissue-specific regulated nucleic acids and/or promoters as well as tumor-specific regulated nucleic acids and/or promoters have been reported. Thus, in some embodiments, tissue-specific or tumor-specific promoters can be used. Some reported tissue-specific nucleic acids include, without limitation, B29 (B cells), CD14 (monocytic cells), CD43 (leukocytes and platelets), CD45 (hematopoietic cells), CD68 (macrophages), desmin (muscle), elastase-1 (pancreatic acinar cells), endoglin (endothelial cells), fibronectin (differentiating cells and healing tissues), FLT-1 (endothelial cells), GFAP (astrocytes), GPIIb (megakaryocytes), ICAM-2 (endothelial cells), INF-β (hematopoietic cells), Mb (muscle), NPHSI (podocytes), OG-2 (osteoblasts, SP-B (lungs), SYN1 (neurons), and WASP (hematopoietic cells). Some reported tumor-specific nucleic acids and promoters include, without limitation, AFP (hepatocellular carcinoma), CCKAR (pancreatic cancer), CEA (epithelial cancer), c-erbB2 (breast and pancreatic cancer), COX-2, CXCR4, E2F-1, HE4, LP, MUC1 (carcinoma), PRC1 (breast cancer), PSA (prostate cancer), RRM2 (breast cancer), survivin, TRP1 (melanoma), and TYR (melanoma).

In some embodiments, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters, and ecdysone-inducible system promoters.

In some embodiments of this invention, one or more terminators may be operably linked to a polynucleotide encoding an engineered CRISPR-Cas system and the engineered transposon 7-like (Tn7-like) transposon system of the invention. In some embodiments, a terminator sequence may be operably linked to the 3′ end of a terminal repeat in a guide nucleic acid (e.g., a CRISPR array, crRNA, gRNA (guide RNA)).

In some embodiments, when comprised in the same nucleic acid construct (e.g., polynucleotide, expression cassette, vector), each of the elements of an engineered CRISPR-Cas system and/or an engineered Tn7-like transposon system of the invention (e.g., Cas6, Cas7, Cas8, and/or TnsA, TnsB, TnsC, and TniQ, optionally, Cas6, Cas7, Cas8, and/or TnsAB, TnsC, and TniQ) may be operably linked to separate (independent) terminators (that may be the same terminator or a different terminator) or to a single terminator. In some embodiments, only the CRISPR array may be operably linked to a terminator. Thus, in some embodiments, a terminator sequence may be operably linked to the 3′ end of a CRISPR array (e.g., linked to the 3′ end of the repeat sequence located at the 3′ end of the CRISPR array).

Any terminator that is useful for defining the end of a transcriptional unit (such as the end of a guide nucleic acid, a Cas6, Cas7, Cas8, TnsA, TnsB, TnsC, and/or TniQ (optionally, Cas6, Cas7, Cas8, TnsAB, TnsC, and TniQ), or any combination thereof) and initiating the process of releasing the newly synthesized RNA from the transcription machinery may be used with this invention (e.g., an terminator that is functional with a polynucleotide comprising a guide nucleic acid, a polynucleotide (one or more) encoding an engineered CRISPR-Cas system as described herein and/or a polynucleotide (one or more) encoding an engineered Tn7-like transposon system as described herein may be utilized (e.g., that can define the end of a transcriptional unit (such as the end of a guide nucleic acid, an engineered CRISPR-Cas system and/or an engineered Tn7-like transposon system and/or elements thereof) and initiate the process of releasing the newly synthesized RNA from the transcription machinery).

In some embodiments, a recombinant nucleic acid construct of the invention may be an “expression cassette” or may be comprised within an expression cassette. As used herein, “expression cassette” means a recombinant nucleic acid construct comprising a polynucleotide of interest (e.g., an engineered CRISPR-Cas system of the invention and/or elements thereof, e.g., Cas6, Cas7, Cas8 and/or an engineered Tn7-like transposon system of the invention and/or elements thereof, e.g., TnsA, TnsB, TnsC, and/or TniQ (optionally TnsAB, TnsC, and TniQ), and/or guide nucleic acids), wherein the polynucleotide of interest is operably associated with at least one control sequence (e.g., a promoter). Thus, some aspects of the invention provide expression cassettes designed to express the polynucleotides of the invention.

An expression cassette or vector comprising a nucleotide sequence of interest may be chimeric, meaning that at least one of its components is heterologous with respect to at least one of its other components. Thus, for example, an expression cassette or vector useful with this invention may be heterologous to the organism from which the engineered CRISPR-Cas system and/or the engineered Tn7-like transposon system of the invention is derived. In some embodiments, an expression cassette or vector may be heterologous to one or more of the bacteria of Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis. In some embodiments, an expression cassette or vector useful with the invention is heterologous to Aliiglaciecola sp. strain M165, Vibrio sp. strain EJY3, H. titanicae strain BH1, P. aquae strain CGMCC 1.12159, P. iliopiscarium strain ATCC 51760, P. piscicola sp. strain NCCB 100098, Psychromonas sp. strain RZ5, K. oxytoca strain 67, C. polaris strain MCCC 1C00015; and/or N. qingdaonensis strain CGMCC 1.10971. An expression cassette may also be one that is naturally occurring but has been obtained in a recombinant form useful for heterologous expression.

An expression cassette may also optionally include a transcriptional and/or translational termination region (i.e., termination region) that is functional in the selected host cell. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked polynucleotide of interest, may be native to the host cell, or may be derived from another source (i.e., foreign or heterologous to the promoter, to the polynucleotide of interest, to the host, or any combination thereof).

An expression cassette (e.g., recombinant nucleic acid constructs and the like) may also include a nucleotide sequence for a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein. In some embodiments, a selectable marker useful with this invention includes polynucleotide encoding a polypeptide conferring resistance to an antibiotic. Non-limiting examples of antibiotics useful with this invention include tetracycline, chloramphenicol, and/or erythromycin. Thus, in some embodiments, a polynucleotide encoding a gene for resistance to an antibiotic may be introduced into the organism, thereby conferring resistance to the antibiotic to that organism.

In addition to expression cassettes, the nucleic acid construct and nucleotide sequences described herein may be used in connection with vectors. The term “vector” refers to a composition for transferring, delivering or introducing a nucleic acid (or nucleic acids) into a cell. A vector comprises a nucleic acid construct comprising the nucleotide sequence(s) to be transferred, delivered or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self-transmissible or mobilizable. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). A nucleic acid construct within a vector may be under the control of, and operably linked to, an appropriate promoter or other regulatory elements for transcription in a host cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Accordingly, the recombinant nucleic acid constructs of this invention and/or expression cassettes comprising the recombinant nucleic acid constructs of this invention may be comprised in vectors as described herein and as known in the art. In some embodiments, the constructs of the invention may be delivered in combination with polypeptides (e.g., Cas6, Cas7, Cas8, TnsA, TnsB (or TnsAB), TnsC, and TniQ) as ribonucleoprotein particles (RNPs). Thus, for example, a polypeptide of the invention may be introduced as a DNA expression plasmid, e.g., in vitro transcripts, or as a recombinant protein bound to the RNA portion in a ribonucleoprotein particle (RNP), whereas a guide nucleic acid, e.g., gRNA, can be delivered either expressed as a DNA plasmid or as an in vitro transcript.

In some embodiments, the invention provides a recombinant nucleic acid construct comprising a Clustered Regularly Interspaced Short Palindromic Repeats (CRISPR) array (e.g., guide nucleic acid) comprising two or more repeat sequences and one or more spacer sequence(s), wherein each spacer sequence and each repeat sequence have a 5′ end and a 3′ end and each spacer sequence is linked at its 5′ end and at its 3′ end to a repeat sequence, and the spacer sequence is complementary to a target sequence (protospacer) in a target DNA of a target organism, optionally wherein the target sequence is located immediately adjacent (3′) to a protospacer adjacent motif (PAM). A guide nucleic acid of the present invention comprises a minimum of two repeats, flanking a spacer, to be expressed as a premature CRISPR RNA (pre-crRNA) that will be processed internally in the cell to constitute the final mature CRISPR RNA (crRNA).

In some embodiments, a repeat sequence (i.e., CRISPR repeat sequence) as used herein may comprise any known repeat sequence of a wild type Aliiglaciecola sp., optionally, Aliiglaciecola sp. strain M165, a wild type Vibrio sp., optionally, Vibrio sp. strain EJY3, a wild-type Halomonas. titanicae, optionally, H. titanicae strain BH1, a wild type Photobacterium aquae, optionally, P. aquae strain CGMCC 1.12159, a wild type Photobacterium iliopiscarium, optionally, P. iliopiscarium strain ATCC 51760, a wild type Photobacterium piscicol, optionally, P. piscicola sp. strain NCCB 100098, a wild type Psychromonas sp., optionally, Psychromonas sp. strain RZ5, a wild type Klebsiella oxytoca, optionally, K. oxytoca strain 67, a wild type Colwellia polaris, optionally, C. polaris strain MCCC 1C00015, or a wild type Neptunomonas qingdaonensis, optionally, N. qingdaonensis strain CGMCC 1.10971. In some embodiments, a repeat sequence useful with the invention may include a synthetic repeat sequence having a different nucleotide sequence than those known in the art for Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis but sharing similar structure to that of the wild-type Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis repeat sequences of a hairpin structure with a loop region. Thus, in some embodiments, a repeat sequence may be identical to (i.e., having 100% identity) or substantially identical (e.g., having 80% to 99% identity (e.g., 80, 81, 82, 83, 84, 85, 86, 87, 88, 89, 90, 91, 92, 93, 94, 95, 96, 97, 98, 99% identity)) to a repeat sequence from a wild type Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis.

In some embodiments, a repeat useful for a system as described herein may be a repeat from Aliiglaciecola sp. strain M165 (e.g., SEQ ID NOs: 54-58, in any combination, optionally, SEQ ID NO:54 and/or SEQ ID NO:55), from Vibrio sp. strain EJY3 (e.g., SEQ ID NO: 154 and/or SEQ ID NO: 155), from H. titanicae strain BH1 (e.g., SEQ ID NOs: 75-78, in any combination, optionally, SEQ ID NO:75 and/or SEQ ID NO:76), from P. aquae strain CGMCC 1.12159 (e.g., SEQ ID NOs: 134-137, in any combination, optionally, SEQ ID NO: 134 and/or SEQ ID NO: 135), from P. iliopiscarium strain ATCC 51760 (e.g., SEQ ID NOs: 15-19, in any combination, optionally, SEQ ID NO: 15 and/or SEQ ID NO:16), from P. piscicola sp. strain NCCB 100098 (e.g., SEQ ID NOs: 15, 16, 36 or 37, in any combination, optionally, SEQ ID NO: 15 and/or SEQ ID NO: 16), from Psychromonas sp. strain RZ5 (e.g., SEQ ID NOs: 172-174, in any combination, optionally, SEQ ID NO:172 and/or SEQ ID NO: 173), from K. oxytoca strain 67 (e.g., SEQ ID NOs: 115-117, in any combination, optionally, SEQ ID NO:115 and/or SEQ ID NO: 116), from C. polaris strain MCCC 1C00015 e.g., SEQ ID NO: 191 and/or SEQ ID NO: 192), or from N. qingdaonensis strain CGMCC 1.10971 (e.g., SEQ ID NOs: 95-100, in any combination, optionally, SEQ ID NO: 95, SEQ ID NO: 96, SEQ ID NO:97 and/or SEQ ID NO:98).

The length of a CRISPR repeat sequence useful with this invention may be the full length of a repeat (e.g., a length of about 28 nucleotides) from any one of Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis.

In some embodiments, the two or more repeat sequences in a guide nucleic acid may comprise the same repeat sequence, may comprise different repeat sequences, or any combination thereof. In some embodiments, each of the two or more repeat sequences in a guide nucleic acid may comprise, consist essentially of, or consist of the same repeat sequence. In some embodiments, each of the two or more repeat sequences in a guide nucleic acid may comprise, consist essentially of, or consist of different repeat sequences.

Notably, Type I-F transposon-associated CRISPR-Cas systems (CasTns) possess self-targeting spacers for directing the transposon to a target genomic locus. These self-targeting spacers are usually flanked by at least one atypical CRISPR repeat, deemed “atypical” due to the significant sequence divergence from typical type I-F CRISPR repeats and, importantly, from the other associated CRISPR repeats within the same CRISPR array. In some cases, the self-targeting spacer can be flanked by two atypical repeats and may reside within an independent, miniature CRISPR array that is distinct from the main CRISPR array for a given system, making the process of identifying and classifying the structure as a standard CRISPR repeat (i.e., a canonical repeat sequence) difficult. These independent CRISPR arrays possessing significantly diverged repeat sequences were initially overlooked in the literature and, even now, published in silico approaches may still fail to capture atypical repeats for a given system.

Atypical repeats useful with this invention have now been identified for the type I-F3 CRISPR-Cas systems described herein and have been found to be more efficient than the canonical repeat sequences for these systems. For example, for Aliiglaciecola sp., the atypical repeat sequences useful for a guide nucleic acid may be SEQ ID NO:54 and/or SEQ ID NO:55; for Vibrio sp., the atypical repeat sequences useful for a guide nucleic acid may be SEQ ID NO: 154 and/or SEQ ID NO: 155; for Halomonas titanicae, the atypical repeat sequences useful for a guide nucleic acid may be SEQ ID NO:75 and/or SEQ ID NO:76; for Photobacterium aquae, the atypical repeat sequences useful for a guide nucleic acid may be SEQ ID NO:134 and/or SEQ ID NO:135; for Photobacterium iliopiscarium, the atypical repeat sequences useful for a guide nucleic acid may be SEQ ID NO: 15 and/or SEQ ID NO:16; for Photobacterium piscicola, the atypical repeat sequences useful for a guide nucleic acid may be SEQ ID NO:15 and/or SEQ ID NO: 16), for Psychromonas sp. the atypical repeat sequences useful for a guide nucleic acid may be SEQ ID NO: 172 and/or SEQ ID NO: 173; for Klebsiella oxytoca, the atypical repeat sequences useful for a guide nucleic acid may be SEQ ID NO: 115 and/or SEQ ID NO: 116; for Colwellia polaris, the atypical repeat sequences useful for a guide nucleic acid may be SEQ ID NO: 191 and/or SEQ ID NO: 192) and for Neptunomonas qingdaonensis, the atypical repeat sequences useful for a guide nucleic acid may be SEQ ID NO:95, SEQ ID NO: 96, SEQ ID NO:97 and/or SEQ ID NO: 98, optionally SEQ ID NO:95 and/or SEQ ID NO:96, or optionally SEQ ID NO:97 and/or SEQ ID NO:98.

A guide nucleic acid of the invention may comprise one spacer sequence or more than one spacer sequence, wherein each spacer sequence is flanked by a repeat sequence. When more than one spacer sequence is present in a guide nucleic acid of the invention, each spacer sequence is separated from the next spacer sequence by a repeat sequence (or portion thereof (e.g., a handle). Thus, each spacer sequence is linked at the 3′ end and at the 5′ end to a repeat sequence. The repeat sequence that is linked to each end of the one or more spacers may be the same repeat sequence or it may be a different repeat sequence or any combination thereof.

In some embodiments, the one or more spacer sequences of the present invention may be about 20 nucleotides to about 45 nucleotides in length (e.g., about 20, 21, 22, 23, 24, 25, 26, 27, 28, 29, 30, 31, 32, 33, 34, 35, 36, 37, 38, 39, 40, 41, 42, 43, 44, or 45 nucleotides in length, and any value or range therein). In some embodiments, a spacer sequence may be a length of about 25 to about 35 nucleotides (e.g., about 25, 26, 27, 28, 29, 30, 31, 32, 33, 34, 35 nucleotides in length, and any value or range therein) or about 30 to about 35 nucleotides (e.g., about 30, 31, 32, 33, 34, 35 nucleotides in length, and any value or range therein). In some embodiments, a spacer sequence may comprise, consist essentially of, or consist of a length of about 33 nucleotides.

In some embodiments, a spacer sequence may be fully complementary to a target sequence (e.g., 100% complementary to a target sequence across its full length). In some embodiments, a spacer sequence may be substantially complementary (e.g., at least about 65% complementary (e.g., about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more complementary)) to a target sequence from a target genome. Thus, in some embodiments, a spacer sequence may have one, two, three, four, five or more mismatches that may be contiguous or noncontiguous as compared to a target sequence from a target genome. In some embodiments, a spacer sequence may be about 70% to 100% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 98.5%, 99%, 99.5%, or more) or about 80% to 100% (e.g., about 80%, 81%, 82%, 83%, 84%, 85%, 86%, 87%, 88%, 89%, 90%, 91%, 92%, 93%, 94%, 95%, 96%, 97%, 98%, 99%, or 100)) complementary to a target sequence from a target genome. In some embodiments, a spacer sequence may be about 85% to 100% (e.g., about 85%, 86%, 87%, 88%, 89%, 90%, 91%, 92%, 93%, 94%, 95%, 96%, 97%, 98%, 99%, or 100%)) complementary to a target sequence from a target genome. In some embodiments, a spacer sequence may be about 90% to 100% (e.g., about 90%, 91%, 92%, 93%, 94%, 95%, 96%, 97%, 98%, 99%, or 100%)) complementary to a target sequence from a target genome. In some embodiments, a spacer sequence may be about 95% to 100% (e.g., about 95%, 96%, 97%, 98%, 98.5%, 99%, 99.5% or 100%) complementary to a target sequence from a target genome.

In some embodiments, the 5′ region of a spacer sequence may be fully complementary to a target sequence while the 3′ region of the spacer sequence may be substantially complementary to the target sequence. Accordingly, in some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) may be about 100% complementary to a target sequence, while the remainder of the spacer sequence may be about 80% or more complementary to the target sequence.

In some embodiments, at least the first eight contiguous nucleotides at the 5′ end of a spacer sequence of the invention are fully complementary to the portion of the target sequence adjacent to the PAM (termed a “seed sequence”). Thus, in some embodiments, the seed sequence may comprise the first 8 nucleotides of the 5′ end of each of one or more spacer sequence(s), which first 8 nucleotides are fully complementary (100%) to the target sequence, and the remaining portion of the one or more spacer sequence(s) (3′ to the seed sequence) may be at least about 80% complementarity (e.g., about 80, 81, 82, 83, 84, 85, 86, 87, 88, 89, 90, 91, 92, 93, 94, 95, 96, 97, 98, 99, or 100% complementarity) to the target sequence. Thus, for example, a spacer sequence having a length of 28 nucleotides may comprise a seed sequence of eight contiguous nucleotides located at the 5′ end of the spacer sequence, which is 100% complementary to the target sequence, while the remaining 20 nucleotides may be about 80% to about 100% complimentary to the target sequence (e.g., 0 to 4 non-complementary nucleotides out of the remaining 20 in the spacer sequence). As another example, a spacer sequence having a length of 33 nucleotides may comprise a seed sequence of eight nucleotides from the 5′ end, which is 100% complementary to the target sequence, while the remaining 25 nucleotides may be at least about 80% (e.g., 0 to 5 non-complementary nucleotides out of the remaining 25 nucleotides in the spacer sequence).

A guide nucleic acid of the invention comprising more than one spacer sequence may be designed to target one or more than one target sequence (protospacer). Thus, in some embodiments, when a recombinant nucleic acid construct of the invention comprises a guide nucleic acid that comprises at least two spacer sequences, the at least two spacer sequences may be complementary to two or more different target sequences. In some embodiments, when a recombinant nucleic acid construct of the invention comprises a guide nucleic acid that comprises at least two spacer sequences, the at least two spacer sequences may be complementary to the same target sequence. In some embodiments, a guide nucleic acid comprising at least two spacer sequences, the at least two spacer sequences may be complementary different portions of one gene.

In some embodiments, a target sequence is a sequence that is located immediately adjacent to a PAM sequence (e.g., 5′-NNNNN-3′, optionally 5′-NNNCN-3′). In some embodiments, a PAM is not required for targeting the genome of an organism for integration of DNA using the systems of this invention. In some embodiments, when the CasTn system (e.g., an engineered CRISPR-Cas system, and an engineered Tn7-like transposon system as described herein) is derived from (a) Aliiglaciecola sp., optionally from Aliiglaciecola sp. strain M165, the PAM is 5′-NNNCN-3′, (b) Vibrio sp., optionally from Vibrio sp. strain EJY3, the PAM is 5′-NNNCC-3′, (c) H. titanicae, optionally from H. titanicae strain BH1, the PAM is 5′-NNNCN-3′, (d) P. aquae, optionally from P. aquae strain CGMCC 1.12159, the PAM is 5′-NNNCN-3′, (e) P. iliopiscarium, optionally from P. iliopiscarium strain ATCC 51760, the PAM is 5′-NNNCN-3′, (f) P. piscicola, optionally from P. piscicola sp. strain NCCB 100098, the PAM is 5′-NNNCN-3′, (g) Psychromonas sp., optionally from is Psychromonas sp. strain RZ5, the PAM is 5′-NNNNN-3′ (e.g., no PAM requirement), (h) K. oxytoca, optionally from K. oxytoca strain 67, the PAM is 5′-NNNCC-3′, (i) C. polaris, optionally from C. polaris strain MCCC 1C00015, the PAM is 5′-NNNCC-3′, and/or (j) N. qingdaonensis, optionally from N. qingdaonensis strain CGMCC 1.10971, the PAM is 5′-NNNCN-3′. The PAM requirements for the CasTn system of this invention differ from that of V. cholerae HE-45, which has a PAM requirement of NNNBN, and which functions very poorly (very inefficiently) with an “A” at position-2). Though the type I-F3 transposon associated CRISPR-Cas systems as described herein as having remarkably flexible PAM requirements overall, the extent of flexibility varies considerably between systems. This flexibility affects the range of target sites for which a given system can efficiently integrate DNA. It was not previously known that these systems would overall gravitate toward the same general PAM (“NNNCN”), that some would experience more severe decreases in integration efficiency than others when testing similar PAMs (e.g., NNNCC to NNNCG), and that some would be, in effect, PAM-less (e.g., Psychromonas).

In some embodiments, a system of the invention (e.g., one or more vectors encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), optionally wherein TnsA and TnsB are fused, e.g., a Transposon 7 protein AB (TnsAB), (c) Transposon 7 protein C (TnsC), and (d) transposition of integron protein Q (TniQ), optionally where TnsA and TnsB may be fused, e.g., TnsAB) may target, for example, coding regions, non-coding regions, intragenic regions, and intergenic regions for genome modification and other uses. In some embodiments, a target sequence may be located in a target DNA of a target organism. In some embodiments, a target sequence may be located on a chromosome. In some embodiments, a target sequence may be located on an extrachromosomal nucleic acid.

In some embodiments, a target sequence may be located in a gene, which can be in the upper (sense, coding) strand or in the bottom (antisense, non-coding) strand. In some embodiments, a target sequence may be located in an intragenic region of a gene (e.g., an intron), optionally located in the upper (sense, coding) strand or in the bottom (antisense, non-coding) strand. In some embodiments, a gene that is targeted by constructs of this invention may encode a transcription factor or a promoter. In some embodiments, a gene that is targeted may encode non-coding RNA, including, but not limited to, miRNA, siRNA, piRNA (piwi-interacting RNA) and lncRNA (long non-coding RNA). In some embodiments, a target sequence may be located in an intergenic region, optionally in the upper (plus) strand or in the bottom (minus) strand. In some embodiments, a target sequence may be located in an intergenic region wherein the DNA is cleaved and a gene inserted that may be expressed under the control of the promoter of the previous open reading frame. In some embodiments, a target sequence may be an extrachromosomal nucleic acid.

As used herein, “extrachromosomal nucleic acid” refers to nucleic acid from a mitochondrion, a plasmid, a plastid (e.g., chloroplast, amyloplast, leucoplast, proplastid, chromoplast, etioplast, elaiosplast, proteinoplast, tannosome), and/or an extrachromosomal circular DNA (eccDNA)). In some embodiments, an extrachromosomal nucleic acid may be referred to as “extranuclear DNA” or “cytoplasmic DNA.” In some embodiments, a plasmid may be targeted (e.g., the target sequence is located on a plasmid), for example, for plasmid curing to eliminate undesired DNA like antibiotic resistance genes or virulence factors.

In some embodiments, a target sequence may be located on a mobile element (e.g., a transposon, a plasmid, a bacteriophage element (e.g., Mu), a group I and group II intron). Thus, for example, mobile elements located in the chromosome or transposons may be targeted to force the mobile elements to jump out of the chromosome.

A system of the invention for RNA-guided DNA integration may be comprised in a vector (e.g., a plasmid, a bacteriophage, and/or a retrovirus). Thus, in some embodiments, the invention further provides vectors, plasmids, bacteriophage, viruses and/or retroviruses comprising the recombinant nucleic acid constructs of the invention.

Plasmids useful with the invention may be dependent on the target organism, that is, dependent on where the plasmid is to replicate. Non-limiting examples of plasmids that express in Lactobacillus include pNZ and derivatives, pGK12 and derivatives, pTRK687 and derivatives, pTRKH2 and derivatives, pIL252, and/or pIL253. Additional, non-limiting plasmids of interest include pORI-based plasmids or other derivatives and homologs.

The present invention is directed to compositions and methods for RNA-guided DNA integration. Specifically, the compositions comprise engineered Clustered Regularly Interspaced Short Palindromic Repeats (CRISPR)-CRISPR associated (Cas) (CRISPR-Cas) systems, the engineered CRISPR-Cas systems comprising: Cas6, Cas7 and Cas8; and engineered transposon 7-like (Tn7-like) transposon systems, the engineered Tn7-like transposon systems comprising: (a) Transposon 7 protein A (TnsA), (b) Transposon 7 protein B (TnsB) (optionally, Transposon 7 protein AB (TnsAB)), (c) Transposon 7 protein C (TnsC), and (d) transposition of integron protein Q (TniQ) from type I-F3 CRISPR-Cas systems.

Type I-F3 CRISPR-Cas systems are Tn7-like transposon-associated systems with non-canonical function. Unlike typical CRISPR-Cas systems that play a direct role in bacterial immunity via RNA-guided nucleic acid targeting and degradation, type I-F3 CRISPR-Cas systems coevolved with transposition machinery for RNA-guided DNA transposition. Rather than degrading DNA at a target site specified by a guide RNA, type I-F3 systems integrate DNA downstream of a target site.

Type I-F3 CRISPR-Cas systems possess multiple genes encoding proteins that are required for functional CRISPR-based DNA targeting and transposition. Other, non-essential “cargo” genes are also found within these transposons but are dispensable for transposition. The essential genes include cas8 (which encodes a natural Cas8-5 fusion), cas7, and cas6, and transposition proteins tniQ, tnsA, tnsB, and tnsC, optionally tniQ, tnsAB and tnsC. Together, Cas8, Cas7, and Cas6 form the Cascade complex, which binds to a crRNA and targets DNA specified by the spacer sequence of the crRNA. In this context, Cascade is not present with Cas3, the helicase-nuclease protein that is commonly found alongside Cascade in typical type I CRISPR-Cas systems. In other words, Cascade binds to a target DNA sequence but lacks DNA cleavage capabilities. Instead, the transposition protein TniQ binds to Cascade and interacts with the rest of the transposition machinery. TnsA and TnsB (optionally, tnsAB) form a heteromeric transposase, and TnsB facilities transposition due to its recognition and binding of specific motifs present on the terminal ends of the transposon (and the presence of these motifs is essential for transposition, and these terminal ends are present within the integrated product). TnsC acts as a regulator protein to regulate transposition via intermediary interactions with the TniQ-Cascade and TnsAB complexes. Notably, the transposon may integrate in a right-to-left (RL) or left-to-right (LR) orientation, where ends of the native transposon are classified as the “Right” or “Left” end.

The CRISPR array or guide nucleic acid for type I-F3 CRISPR-Cas systems are typically short, only comprised of a few spacers, and canonically possess at least one “atypical” repeat with significant sequence deviation from the other CRISPR repeats present within the same system. Interestingly, these atypical repeats may be found downstream of the main CRISPR array in the form of a single repeat-spacer-repeat array. These atypical repeats are adjacent to or flank a self-targeting spacer that, unlike typical CRISPR-Cas systems that target phages or plasmids, guides the Cascade complex to integrate the transposon about 50 bp downstream of a chromosomal target site. These self-targeting spacers are often truncated, occasionally being almost half as short as the expected spacer length. To facilitate transposition in a variety of hosts, type I-F3 CRISPR-Cas systems are typically guided to one of four conserved target chromosomal integration sites (Petassi et al. 2020 Cell 183(7):1757-1771). DNA-targeting of the type I-F3 Cascade is unusually flexible: Cascade can tolerate multiple mismatches between the spacer and target sequence (e.g., as low as 65% complementarity), and PAM (protospacer adjacent motif) requirements for Cascade are remarkably slim or even absent. Often there is only preference for a single nucleotide within the type I-F3 PAM (e.g., 5′-NNNCN-3′), but successful DNA transposition can occur with PAMs that are not preferred, albeit at lesser efficiencies.

Accordingly, the present invention is directed to a system for RNA-guided DNA integration, the system comprising: (A) one or more vectors heterologous to Aliiglaciecola sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Aliiglaciecola sp.; (B) one or more vectors heterologous to Vibrio sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (Tns B), (c) Transposon 7 protein C (TnsC), and (d) transposition of integron protein Q (TniQ), wherein the engineered Tn7-like transposon system is derived from Vibrio sp.; (C) one or more vectors heterologous to Halomonas titanicae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from H. titanicae; (D) one or more vectors heterologous to Photobacterium aquae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. aquae; (E) one or more vectors heterologous to Photobacterium iliopiscarium encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. iliopiscarium; (F) one or more vectors heterologous to Photobacterium piscicola encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. piscicola; (G) one or more vectors heterologous to Psychromonas sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Psychromonas sp.; (H) one or more vectors heterologous to Klebsiella oxytoca encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein AB (TnsAB), (b) Transposon 7 protein C (TnsC), and (c) transposition of integron protein Q (TniQ), wherein the engineered Tn7-like transposon system is derived from K. oxytoca; (I) one or more vectors heterologous to Colwellia polaris encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from C. polaris; and/or (J) one or more vectors heterologous to Neptunomonas qingdaonensis encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from N. qingdaonensis.

CRISPR-associate transposons may also be referred to as CasTn, CAST, and CRISPR-Tn. The systems of the invention comprising engineered CRISPR-Cas systems and an engineered Tn7-like transposon systems are nuclease-deficient (e.g., are devoid of a nuclease).

In some embodiments, the engineered CRISPR-Cas system and the engineered Tn7-like transposon system is derived from (A) Aliiglaciecola sp., optionally from Aliiglaciecola sp. strain M165; (B) Vibrio sp., optionally from Vibrio sp. strain EJY3; (C) H. titanicae, optionally from H. titanicae strain BH1; (D) P. aquae, optionally from P. aquae strain CGMCC 1.12159; (E) P. iliopiscarium, optionally from P. iliopiscarium strain ATCC 51760; (F) P. piscicola, optionally from P. piscicola sp. strain NCCB 100098; (G) Psychromonas sp., optionally from Psychromonas sp. strain RZ5; (H) K. oxytoca, optionally from K. oxytoca strain 67; (I) C. polaris, optionally from C. polaris strain MCCC 1C00015; and/or (J) N. qingdaonensis, optionally from N. qingdaonensis strain CGMCC 1.10971.

In some embodiments, when the engineered CRISPR-Cas system and the engineered Tn7-like transposon system (e.g., the type I-F3 transposon associated CRISPR-Cas system, e.g., the CasTns system) is derived from Aliiglaciecola sp., Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:43 and/or comprises an amino acid sequence having at least 90% sequence identity to SEQ ID NO:50, Cas7 is encoded by a nucleotide sequence having at least 90% sequence identity to SEQ ID NO:42 and/or comprises an amino acid sequence having at least 90% sequence identity to SEQ ID NO:49, Cas8 is encoded by a nucleotide sequence having at least 90% sequence identity to SEQ ID NO:41 and/or comprises an amino acid sequence having at least 90% sequence identity to SEQ ID NO:48, TnsA is encoded by a nucleotide sequence having at least 90% sequence identity to SEQ ID NO:44 and/or comprises an amino acid sequence having at least 90% sequence identity to SEQ ID NO: 51, TnsB is encoded by a nucleotide sequence having at least 90% sequence identity to SEQ ID NO:45 and/or comprises an amino acid sequence having at least 90% sequence identity to SEQ ID NO:52, TnsC is encoded by a nucleotide sequence having at least 90% sequence identity to SEQ ID NO:46 and/or comprises an amino acid sequence having at least 90% sequence identity to SEQ ID NO:53, and/or TniQ is encoded by a nucleotide sequence having at least 90% sequence identity to SEQ ID NO:40 and/or comprises an amino acid sequence having at least 90% sequence identity to SEQ ID NO:47.

In some embodiments, when the engineered CRISPR-Cas system and the engineered Tn7-like transposon system is derived from Vibrio sp., Cas6 encoded by a nucleotide sequence having at least 90% (e.g., 90%, 91%, 92%, 93%, 94%, 95%, 96%, 97%, 98%, 99%, and/or 100%) sequence identity to SEQ ID NO:143 and/or comprises an amino acid sequence having at least 90% sequence identity to SEQ ID NO: 150, Cas7 is encoded by a nucleotide sequence having at least 90% sequence identity to SEQ ID NO: 142 and/or comprises an amino acid sequence having at least 90% sequence identity to SEQ ID NO:149, Cas8 is encoded by a nucleotide sequence having at least 90% sequence identity to SEQ ID NO: 141 and/or comprises an amino acid sequence having at least 90% sequence identity to SEQ ID NO:148, TnsA is encoded by a nucleotide sequence having at least 90% sequence identity to SEQ ID NO: 144 and/or comprises an amino acid sequence having at least 90% sequence identity to SEQ ID NO: 151, TnsB is encoded by a nucleotide sequence having at least 90% sequence identity to SEQ ID NO: 145 and/or comprises an amino acid sequence having at least 90% sequence identity to SEQ ID NO:152, TnsC is encoded by a nucleotide sequence having at least 90% sequence identity to SEQ ID NO:146 and/or comprises an amino acid sequence having at least 90% sequence identity to SEQ ID NO: 153, and/or TniQ is encoded by a nucleotide sequence having at least 90% sequence identity to SEQ ID NO: 140 and/or comprises an amino acid sequence having at least 90% sequence identity to SEQ ID NO:147.

In some embodiments, when the engineered CRISPR-Cas system and the engineered Tn7-like transposon system is derived from H. titanicae, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:64 and/or comprises an amino acid sequence having at least 90% sequence identity to SEQ ID NO:71, Cas7 is encoded by a nucleotide sequence having at least 90% sequence identity to SEQ ID NO:63 and/or comprises an amino acid sequence having at least 90% sequence identity to SEQ ID NO:70, Cas8 is encoded by a nucleotide sequence having at least 90% sequence identity to SEQ ID NO:62 and/or comprises an amino acid sequence having at least 90% sequence identity to SEQ ID NO:69, TnsA is encoded by a nucleotide sequence having at least 90% sequence identity to SEQ ID NO:65 and/or comprises an amino acid sequence having at least 90% sequence identity to SEQ ID NO: 72, TnsB is encoded by a nucleotide sequence having at least 90% sequence identity to SEQ ID NO:66 and/or comprises an amino acid sequence having at least 90% sequence identity to SEQ ID NO:73, TnsC is encoded by a nucleotide sequence having at least 90% sequence identity to SEQ ID NO:67 and/or comprises an amino acid sequence having at least 90% sequence identity to SEQ ID NO:74, and/or TniQ is encoded by a nucleotide sequence having at least 90% sequence identity to SEQ ID NO:61 and/or comprises an amino acid sequence having at least 90% sequence identity to SEQ ID NO:68.

In some embodiments, when the engineered CRISPR-Cas system and the engineered Tn7-like transposon system is derived from P. aquae, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO: 123 and/or comprises an amino acid sequence having at least 90% sequence identity to SEQ ID NO:130, Cas7 is encoded by a nucleotide sequence having at least 90% sequence identity to SEQ ID NO:122 and/or comprises an amino acid sequence having at least 90% sequence identity to SEQ ID NO: 129, Cas8 is encoded by a nucleotide sequence having at least 90% sequence identity to SEQ ID NO: 121 and/or comprises an amino acid sequence having at least 90% sequence identity to SEQ ID NO:128, TnsA is encoded by a nucleotide sequence having at least 90% sequence identity to SEQ ID NO: 124 and/or comprises an amino acid sequence having at least 90% sequence identity to SEQ ID NO: 131, TnsB is encoded by a nucleotide sequence having at least 90% sequence identity to SEQ ID NO:125 and/or comprises an amino acid sequence having at least 90% sequence identity to SEQ ID NO:132, TnsC is encoded by a nucleotide sequence having at least 90% sequence identity to SEQ ID NO: 126 and/or comprises an amino acid sequence having at least 90% sequence identity to SEQ ID NO:133, and/or TniQ is encoded by a nucleotide sequence having at least 90% sequence identity to SEQ ID NO:120 and/or comprises an amino acid sequence having at least 90% sequence identity to SEQ ID NO:127.

In some embodiments, when the engineered CRISPR-Cas system and the engineered Tn7-like transposon system is derived from P. iliopiscarium, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:4 and/or comprises an amino acid sequence having at least 90% sequence identity to SEQ ID NO:11, Cas7 is encoded by a nucleotide sequence having at least 90% sequence identity to SEQ ID NO:3 and/or comprises an amino acid sequence having at least 90% sequence identity to SEQ ID NO: 10, Cas8 is encoded by a nucleotide sequence having at least 90% sequence identity to SEQ ID NO:2 and/or comprises an amino acid sequence having at least 90% sequence identity to SEQ ID NO:9, TnsA is encoded by a nucleotide sequence having at least 90% sequence identity to SEQ ID NO:5 and/or comprises an amino acid sequence having at least 90% sequence identity to SEQ ID NO: 12, TnsB is encoded by a nucleotide sequence having at least 90% sequence identity to SEQ ID NO:6 and/or comprises an amino acid sequence having at least 90% sequence identity to SEQ ID NO:13, TnsC encoded by a nucleotide sequence having at least 90% sequence identity to SEQ ID NO:7 and/or comprises an amino acid sequence having at least 90% sequence identity to SEQ ID NO:14, and/or TniQ is encoded by a nucleotide sequence having at least 90% sequence identity to SEQ ID NO:1 and/or comprises an amino acid sequence having at least 90% sequence identity to SEQ ID NO:8.

In some embodiments, when the engineered CRISPR-Cas system and the engineered Tn7-like transposon system is derived from P. piscicola, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:25 and/or comprises an amino acid sequence having at least 90% sequence identity to SEQ ID NO:32, Cas7 is encoded by a nucleotide sequence having at least 90% sequence identity to SEQ ID NO:24 and/or comprises an amino acid sequence having at least 90% sequence identity to SEQ ID NO:31, Cas8 is encoded by a nucleotide sequence having at least 90% sequence identity to SEQ ID NO:23 and/or comprises an amino acid sequence having at least 90% sequence identity to SEQ ID NO:30, TnsA is encoded by a nucleotide sequence having at least 90% sequence identity to SEQ ID NO:26 and/or comprises an amino acid sequence having at least 90% sequence identity to SEQ ID NO: 33, TnsB is encoded by a nucleotide sequence having at least 90% sequence identity to SEQ ID NO:27 and/or comprises an amino acid sequence having at least 90% sequence identity to SEQ ID NO:34, TnsC is encoded by a nucleotide sequence having at least 90% sequence identity to SEQ ID NO:28 and/or comprises an amino acid sequence having at least 90% sequence identity to SEQ ID NO:35, and/or TniQ is encoded by a nucleotide sequence having at least 90% sequence identity to SEQ ID NO:22 and/or comprises an amino acid sequence having at least 90% sequence identity to SEQ ID NO:29.

In some embodiments, when the engineered CRISPR-Cas system and the engineered Tn7-like transposon system is derived from Psychromonas sp., Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO: 161 and/or comprises an amino acid sequence having at least 90% sequence identity to SEQ ID NO: 168, Cas7 is encoded by a nucleotide sequence having at least 90% sequence identity to SEQ ID NO: 160 and/or comprises an amino acid sequence having at least 90% sequence identity to SEQ ID NO: 167, Cas8 is encoded by a nucleotide sequence having at least 90% sequence identity to SEQ ID NO: 159 and/or comprises an amino acid sequence having at least 90% sequence identity to SEQ ID NO: 166, TnsA is encoded by a nucleotide sequence having at least 90% sequence identity to SEQ ID NO:162 and/or comprises an amino acid sequence having at least 90% sequence identity to SEQ ID NO: 169, TnsB is encoded by a nucleotide sequence having at least 90% sequence identity to SEQ ID NO: 163 and/or comprises an amino acid sequence having at least 90% sequence identity to SEQ ID NO:170, TnsC is encoded by a nucleotide sequence having at least 90% sequence identity to SEQ ID NO:164 and/or comprises an amino acid sequence having at least 90% sequence identity to SEQ ID NO: 171, and/or TniQ is encoded by a nucleotide sequence having at least 90% sequence identity to SEQ ID NO: 158 and/or comprises an amino acid sequence having at least 90% sequence identity to SEQ ID NO: 165. In some embodiments, when the engineered CRISPR-Cas system and the engineered Tn7-like transposon system is derived from Psychromonas sp., the nucleic acid sequences may be optimized for expression in a plant, wherein the Cas6 is encoded by a nucleotide sequence having at least 90% sequence identity to SEQ ID NO:213, the Cas7 encoded by a nucleotide sequence having at least 90% sequence identity to any one of SEQ ID NO: 214 or SEQ ID NO:230-233, the Cas8 is encoded by a nucleotide sequence having at least 90% sequence identity to SEQ ID NO:215, the TnsA is encoded by a nucleotide sequence having at least 90% sequence identity to SEQ ID NO:217, the TnsB is encoded by a nucleotide sequence having at least 90% sequence identity to SEQ ID NO:218, the TnsC encoded by a nucleotide sequence having at least 90% sequence identity to any one of SEQ ID NO:219 or SEQ ID NO:234-237 and/or the TniQ is encoded by a nucleotide sequence having at least 90% sequence identity to SEQ ID NO:216.

In some embodiments, when the engineered CRISPR-Cas system and the engineered Tn7-like transposon system is derived from K. oxytoca, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:106 and/or comprises an amino acid sequence having at least 90% sequence identity to SEQ ID NO:112, Cas7 is encoded by a nucleotide sequence having at least 90% sequence identity to SEQ ID NO:105 and/or comprises an amino acid sequence having at least 90% sequence identity to SEQ ID NO:111, Cas8 encoded by a nucleotide sequence having at least 90% sequence identity to SEQ ID NO:104 and/or comprises an amino acid sequence having at least 90% sequence identity to SEQ ID NO:110, TnsAB is encoded by a nucleotide sequence having at least 90% sequence identity to SEQ ID NO: 107 and/or comprises an amino acid sequence having at least 90% sequence identity to SEQ ID NO: 113, TnsC is encoded by a nucleotide sequence having at least 90% sequence identity to SEQ ID NO: 108 and/or comprises an amino acid sequence having at least 90% sequence identity to SEQ ID NO:114, and/or TniQ is encoded by a nucleotide sequence having at least 90% sequence identity to SEQ ID NO:103 and/or comprises an amino acid sequence having at least 90% sequence identity to SEQ ID NO:109.

In some embodiments, when the engineered CRISPR-Cas system and the engineered Tn7-like transposon system is derived from C. polaris, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:180 and/or comprises an amino acid sequence having at least 90% sequence identity to SEQ ID NO: 187, Cas7 is encoded by a nucleotide sequence having at least 90% sequence identity to SEQ ID NO: 179 and/or comprises an amino acid sequence having at least 90% sequence identity to SEQ ID NO: 186, Cas8 is encoded by a nucleotide sequence having at least 90% sequence identity to SEQ ID NO:178 and/or comprises an amino acid sequence having at least 90% sequence identity to SEQ ID NO:185, TnsA is encoded by a nucleotide sequence having at least 90% sequence identity to SEQ ID NO: 181 and/or comprises an amino acid sequence having at least 90% sequence identity to SEQ ID NO: 188, TnsB is encoded by a nucleotide sequence having at least 90% sequence identity to SEQ ID NO:182 and/or comprises an amino acid sequence having at least 90% sequence identity to SEQ ID NO:189, TnsC is encoded by a nucleotide sequence having at least 90% sequence identity to SEQ ID NO:183 and/or comprises an amino acid sequence having at least 90% sequence identity to SEQ ID NO: 190, and/or TniQ is encoded by a nucleotide sequence having at least 90% sequence identity to SEQ ID NO:177 and/or comprises an amino acid sequence having at least 90% sequence identity to SEQ ID NO:184.

In some embodiments, when the engineered CRISPR-Cas system and the engineered Tn7-like transposon system is derived from N. qingdaonensis, Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:84 and/or comprises an amino acid sequence having at least 90% sequence identity to SEQ ID NO:91, Cas7 is encoded by a nucleotide sequence having at least 90% sequence identity to SEQ ID NO:83 and/or comprises an amino acid sequence having at least 90% sequence identity to SEQ ID NO:90, Cas8 is encoded by a nucleotide sequence having at least 90% sequence identity to SEQ ID NO:82 and/or comprises an amino acid sequence having at least 90% sequence identity to SEQ ID NO:89, TnsA is encoded by a nucleotide sequence having at least 90% sequence identity to SEQ ID NO:85 and/or comprises an amino acid sequence having at least 90% sequence identity to SEQ ID NO: 92, TnsB is encoded by a nucleotide sequence having at least 90% sequence identity to SEQ ID NO:86 and/or comprises an amino acid sequence having at least 90% sequence identity to SEQ ID NO:93, TnsC is encoded by a nucleotide sequence having at least 90% sequence identity to SEQ ID NO:87 and/or comprises an amino acid sequence having at least 90% sequence identity to SEQ ID NO:94, and/or TniQ is encoded by a nucleotide sequence having at least 90% sequence identity to SEQ ID NO:81 and/or comprises an amino acid sequence having at least 90% sequence identity to SEQ ID NO:88.

A system of the invention comprising an engineered Clustered Regularly Interspaced Short Palindromic Repeats (CRISPR)-CRISPR associated (Cas) (CRISPR-Cas) system and an engineered transposon 7-like (Tn7-like) transposon system, further comprises a guide nucleic acid (e.g., guide RNA, gRNA; e.g., CRISPR array (e.g., crRNA when processed)), wherein the guide nucleic acid comprises one or more (e.g., 1, 2, 3, 4, 5, 6 or more) spacer sequences having complementarity to a target site. In some embodiments, the spacer sequence of a guide nucleic acid useful with this invention is oriented 5′-3′. In some embodiments, the spacer sequence of a guide nucleic acid useful with this invention is oriented 3′-5′. In some embodiments, a target site may be located immediately adjacent (3′) to a protospacer adjacent motif (PAM), optionally, wherein the PAM comprises a nucleotide sequence of 5′-NNNNN-3′ that is immediately adjacent to and 5′ of the target site (protospacer). In some embodiments, a PAM may comprise a nucleotide sequence of 5′-NNNCN-3′ or 5′-NNNCC-3′ that is immediately adjacent to and 5′ of the target site. In some embodiments, a PAM is not required for the system to function (e.g., function to integrate DNA, e.g., cargo DNA) (e.g., a system derived from Psychromonas sp. PAM (protospacer adjacent motif) requirements for the type I-F3 systems are not stringent as compared to other CRISPR-Cas systems. As noted above, in some systems, the preference is for only a single nucleotide within a PAM (e.g., 5′-NNNCN-3′ or 5′-NNNCC-3′), but successful DNA transposition can occur with PAMs that are not preferred, albeit at lesser efficiencies.

In some embodiments, each of the one or more spacer sequences of a guide nucleic acid may be linked at its 5′-end and at its 3′-end to a repeat sequence (e.g., repeat-spacer-repeat, repeat-spacer-repeat-spacer-repeat, repeat-spacer-repeat-spacer-repeat-spacer-repeat, and the like).

In some embodiments, a repeat sequence for a guide nucleic acid (e.g., CRISPR array) of the invention may be derived from (A) Aliiglaciecola sp., optionally from Aliiglaciecola sp. strain M165; (B) Vibrio sp., optionally from Vibrio sp. strain EJY3; (C) Halomonas titanicae, optionally from H. titanicae strain BH1; (D) Photobacterium aquae, optionally from P. aquae strain CGMCC 1.12159; (E) Photobacterium iliopiscarium, optionally from P. iliopiscarium strain ATCC 51760; (F) Photobacterium piscicola, optionally from P. piscicola sp. strain NCCB 100098; (G) Psychromonas sp., optionally from Psychromonas sp. strain RZ5; (H) Klebsiella oxytoca, optionally from K. oxytoca strain 67; (I) Colwellia polaris, optionally from C. polaris strain MCCC 1C00015; and/or (J) Neptunomonas qingdaonensis, optionally from N. qingdaonensis strain CGMCC 1.10971.

In some embodiments, when the system (e.g., the engineered CRISPR-Cas system and the engineered Tn7-like transposon system) is derived from Aliiglaciecola sp., the repeat sequence(s) may be any one of the nucleotide sequences of SEQ ID NOs: 54-58, in any combination, optionally, the repeat sequences may be SEQ ID NO:54 and/or SEQ ID NO:55. In some embodiments, when the system is derived from Vibrio sp., the repeat sequence(s) may be any one of the nucleotide sequences of SEQ ID NO: 154 and/or SEQ ID NO: 155. In some embodiments, when the system is derived from Halomonas titanicae, the repeat sequence(s) may be any one of the nucleotide sequences of SEQ ID NOs: 75-78, in any combination, optionally, the repeat sequences may be SEQ ID NO:75 and/or SEQ ID NO:76. In some embodiments, when the system is derived from Photobacterium aquae, the repeat sequence(s) may be any one of the nucleotide sequences of SEQ ID NOs: 134-137, in any combination, optionally, the repeat sequences may be SEQ ID NO:134 and/or SEQ ID NO: 135. In some embodiments, when the system is derived from Photobacterium iliopiscarium, the repeat sequence(s) may be any one of the nucleotide sequences of SEQ ID NOs: 15-19, in any combination, optionally, the repeat sequences may be SEQ ID NO: 15 and/or SEQ ID NO: 16. In some embodiments, when the system is derived from Photobacterium piscicola, the repeat sequence(s) may be any one of the nucleotide sequences of SEQ ID NOs: 15, 16, 36 or 37, in any combination, optionally, the repeat sequences may be SEQ ID NO:15 and/or SEQ ID NO: 16. In some embodiments, when the system is derived from Psychromonas sp., the repeat sequence(s) may be any one of the nucleotide sequences of SEQ ID NOs: 172-174, in any combination, optionally, the repeat sequences may be SEQ ID NO: 172 and/or SEQ ID NO: 173. In some embodiments, when the system is derived from Klebsiella oxytoca, the repeat sequence(s) may be any one of the nucleotide sequences of SEQ ID NOs: 115-117, in any combination, optionally, the repeat sequences may be SEQ ID NO: 115 and/or SEQ ID NO:116. In some embodiments, when the system is derived from Colwellia polaris, the repeat sequence(s) may be any one of the nucleotide sequences of SEQ ID NO: 191 and/or SEQ ID NO: 192. In some embodiments, when the system is derived from Neptunomonas qingdaonensis, the repeat sequence(s) may be any one of the nucleotide sequences of SEQ ID NOs: 95-100, in any combination, optionally, the repeat sequences may be SEQ ID NO:95, SEQ ID NO:96, SEQ ID NO:97 and/or SEQ ID NO:98, in any combination.

A system of the invention comprising an engineered Clustered Regularly Interspaced Short Palindromic Repeats (CRISPR)-CRISPR associated (Cas) (CRISPR-Cas) system and an engineered transposon 7-like (Tn7-like) transposon system, further comprises a donor DNA to be integrated, wherein the donor DNA comprises a cargo nucleic acid sequence and a first transposon end sequence and a second transposon end sequence, wherein the cargo nucleic acid sequence is flanked by the first transposon end sequence and the second transposon end sequence, and wherein each of the first transposon end sequence and the second transposon end sequence comprises at least one TnsB binding site.

The term “flanked” refers to a nucleic acid sequence that is immediately adjacent to a reference nucleic acid sequence or that is in between an upstream reference nucleic acid sequence and/or a downstream reference nucleic acid sequence, i.e., S′ and/or 3′, relative to the reference nucleic acid sequence.

A “donor DNA” is a synthetic transposon comprised of “cargo” nucleic acid sequence (e.g., a cargo DNA) flanked by the predicted terminal ends of a given transposon. In some embodiments, a donor DNA may be linear or it may be comprised on a vector such as a plasmid.

In some embodiments, a cargo nucleic acid sequence may comprise a length of about 100 consecutive nucleotides to about 100,000 consecutive nucleotides in length (e.g., about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 100, 1500, 200, 2500, 3000, 3500, 4000, 4500, 5000, 6000, 7000, 8000, 9000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 nucleotides in length).

The first transposon end sequence and the second transposon end sequence comprised in the donor DNA and flanking the cargo DNA are Tn7-like transposon end sequences. Transposon end sequences are important for deploying the systems described herein in targeted integration of the cargo DNA into a genome. Determining the structure of the transposon end sequences with high confidence can be difficult. Fragmented, unclosed genomes can act as a barrier for predicting the terminal transposon ends, and other mobile genetic elements with similar repetitive elements can blur predictions. Mutated terminal inverted repeats and target site duplication events and significantly diverged TnsB binding sites within a single system can also cause difficulties when attempting to confidently predict both ends of a transposon sequence. The present inventors have identified the structures of the transposon end sequences for each of the CasTns systems described herein, thereby allowing the opportunity to now convert the transposon from its native context into a genome engineering tool.

In some embodiments, a first transposon end sequence and a second transposon end sequence may each comprise at least two (e.g., 2, 3, or 4 or more) TnsB binding sites. In some embodiments, the at least two TnsB binding sites may be immediately adjacent to each other (e.g., no intervening nucleotides (e.g., no variable region)). In some embodiments, a variable region may be located between the at least two TnsB binding sites. In some embodiments, when three or more TnsB binding sites are present, the TnsB binding sites may be immediately adjacent to each other and may comprise a variable region between the TnsB binding sites, in any combination. In some embodiments, a variable region may have a length of about 1 nucleotide to about 80 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 nucleotides to about 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 70, 75, or 80 nucleotides), optionally a length of about 1 nucleotide to about 10 nucleotides (e.g., 1, 2, or 3 nucleotides to about 4, 5, 6, 7, 8, 9, or 10 nucleotides), optionally, a length of about 1 nucleotide to about 3 nucleotides (e.g., 1, 2, or 3 nucleotides). In some embodiments, a system of the invention may comprise a first transposon end sequence and/or second transposon end sequence that may each comprise three or four TnsB binding sites, optionally, wherein at least two of the three or four TnsB binding sites are immediately adjacent to each other (e.g., no intervening nucleotides (no variable region) between the at least two of the three or four TnsB binding sites), and/or wherein a variable region of 1 nucleotide to about 80 nucleotides is located between at least two of the three to four TnsB binding sites.

TnsB binding sites useful with the invention include but are not limited to those provided in Table 1.

TABLE 1
TnsB binding sites

| | | SEQ |
|---|---|---|
| Source | Consensus Sequence | ID NO: |
| Photobacterium | (A/G/T)(A/T)(T/A)ACAACCATA(A/T)(G/C) | 195 |
| iliopiscarium | (T/C)TGATATT(T/A/C/G)(A/T/C)(C/A/T) | |
| | (T/A/C)(A/C/T) | |
| | DWWACAACCATAWSYTGATATTNHHHH | |
| Photobacterium | (A/G/T)(A/T)(A/T)ACAACCATA(A/T)(G/C) | 196 |
| pisicola | TTGATATT(T/A/C/G)(A/T/C)(C/A/T)(T/A/C) | |
| | (A/C/T) | |
| | DWWACAACCATAWSTTGATATTNHHHH | |
| Aliiglaciecola | (G/A/T)(A/C/T)(T/A/C)(A/C)CAAG(C/G)ATAC | 197 |
| sp. | (C/G)TTG(A/G/T)CA(T/C)(A/C/T)(A/G)(T/A/G) | |
| | (T/C/A)(A/C)A | |
| | DHHMCAAGSATACSTTGDCAYHRDHMA | |
| Halomonas | (A/G/T)(T/A)(T/A)(T/A)(C/T/G)(C/T)(G/A/C) | 198 |
| titanicae | (G/T/C)(T/A/C/G)(T/G/C)(T/C)(A/C/G/T)(A/T) | |
| | (A/C/T)(A/T/G)(T/C)(G/T)AC(A/G)(T/A)A(A/G) | |
| | (A/T/G)(A/C/T)(T/A/G)(A/G/T) | |
| | DWWWBYVBNBYNWHDYKACRWARDHDD | |
| Neptunomonas | (A/G)(T/A)(A/T/G)(T/A/G)(G/T)(C/T)(A/C) | 199 |
| qingdaonensis | (G/A/C)(C/T/A)(A/G)(T/A)AAG(T/A/C)(T/C) | |
| | TGCATAA(A/G/T)(G/A/T)(T/A/G)(A/C/T) | |
| | RWDDKYMVHRWAAGHYTGCATAADDDH | |
| Klebsiella | (A/G/C)(T/C)(T/A)(T/G)(A/G/T)(A/C/T)(C/T/A) | 200 |
| oxytoca | (G/A/C/T)(C/G)ATAA(G/A)(T/C/A)TG(A/G)CA | |
| | TA(A/G/T)(C/A/T)(T/G/C)(A/T)(T/A/G) | |
| | VYWKDHHNSATAARHTGRCATADHBWD | |
| Photobacterium | (A/G/T)(A/C/T)(A/T/G)(A/G/T)AA(A/G/T)(C/T) | 201 |
| aquae | CA(T/A)A(A/T)(A/C/G)(A/C)(T/A/C)(G/A)TCC | |
| | (T/A/C)(A/T)(A/T)(T/A/G)(A/C)(A/T/G)(T/A/C) | |
| | DHDDAADYCAWAWVMHRTCCHWWDMDH | |
| Psychromonas | (A/G/T)(A/C)(T/G)(A/T/G)(A/C/T)(A/G)(A/C) | 202 |
| sp. | (C/G/T)(C/T)A(T/A)(A/T)(C/T)(T/A/G)(C/T)T | |
| | (G/T)ACA(T/A)(A/C)(A/T)(T/A/C)(A/G/T)(T/A) | |
| | (A/G) | |
| | DMKDHRMBYAWWYDYTKACAWMWHDWR | |
| Colwellia | (A/G/T)(A/C/T)(T/A)(G/A/T)(A/C/T)A(A/C)(C/G) | 203 |
| polaris | CA(T/A)A(C/G)(A/T/C)(A/T/G)TGACA(T/A)A | |
| | (A/T)(A/T/G)(A/G/T)(C/A/T)(A/T) | |
| | DHWDHAMSCAWASHDTGACAWAWDDHW | |
| Vibrio sp. | (A/G/T)(T/C)(T/A/G)(A/G)(G/T)(A/G/T)(A/C)(C/ | 204 |
| | A) | |
| | (C/G)A(T/A)(A/T)(A/T)(G/A)(G/T/A)TG(A/T)CA | |
| | (T/C)(T/C/G)(T/C/G)(T/A)(C/T)(G/T/A)(A/C/G) | |
| | DYDRKDMMSAWWWRDTGWCAYBBWYDV | |

W = A/T, S = C/G, M = A/C, K = G/T, R = A/G, Y = C/T, B = C/G/T, D = A/G/T, H = A/C/T, V = A/C/G, N = A/C/G/T.

Thus, in some embodiments, a Tns binding site for first transposon end sequence and a second transposon end sequence may be derived from (A) Aliiglaciecola sp., optionally from Aliiglaciecola sp. strain M165, and may have the nucleotide sequence of SEQ ID NO: 197; (B) Vibrio sp., optionally from Vibrio sp. strain EJY3, and may have the nucleotide sequence of SEQ ID NO:204; (C) Halomonas titanicae, optionally from H. titanicae strain BH1, and may have the nucleotide sequence of SEQ ID NO: 198; (D) Photobacterium aquae, optionally from P. aquae strain CGMCC 1.12159, and may have the nucleotide sequence of SEQ ID NO:201; (E) Photobacterium iliopiscarium, optionally from P. iliopiscarium strain ATCC 51760, and may have the nucleotide sequence of SEQ ID NO: 195; (F) Photobacterium piscicola, optionally from P. piscicola sp. strain NCCB 100098, and may have the nucleotide sequence of SEQ ID NO: 196; (G) Psychromonas sp., optionally from Psychromonas sp. strain RZ5, and may have the nucleotide sequence of SEQ ID NO:202; (H) Klebsiella oxytoca, optionally from K. oxytoca strain 67, and may have the nucleotide sequence of SEQ ID NO:200; (I) Colwellia polaris, optionally from C. polaris strain MCCC 1C00015, and may have the nucleotide sequence of SEQ ID NO:203; and/or (J) Neptunomonas qingdaonensis, optionally from N. qingdaonensis strain CGMCC 1.10971, and may have the nucleotide sequence of SEQ ID NO: 199.

In some embodiments, the 5′ end of a first transposon end sequence and the 3′ end of a second transposon end sequence useful with the invention comprise a terminal inverted repeat (TIR), optionally wherein the TIR for the second transposon end sequence may be the reverse complement of the TIR of the first transposon end sequence, optionally wherein the reverse complement of the second transposon end sequence may be a perfect reverse complement or wherein the reverse complement may have one or more mismatches as compared to the TIR of the first transposon end sequence. In some embodiments, a TIR of a first transposon end sequence and a TIR of a second transposon end sequence may each comprise a length of about 8 base pairs. In some embodiments, a TIR may comprise a sequence TGTNNNNN and/or a reverse complement of TGTNNNNN, optionally wherein the TIR at the 3′ end of the second transposon end sequence is the reverse complement of the TIR at the 5′ end of the first transposon end sequence (e.g., the TIR of the first transposon end sequence may comprise a sequence of TGTNNNNN and the TIR of the second transposon end sequence may comprise a sequence that is the reverse complement of TGTNNNNN (i.e., NNNNNACA)). In some embodiments, the TIR may comprise a sequence of TGTTGAAA, TGTTGATA, TGTTGATC, TGTCGTTT, TGTCGCTT, TGTCGCAA, TGTCGCTG, TGTGGCTG, or TGTGGCTA, or the reverse complement thereof.

In some embodiments, (a) the TIR of the first transposon end sequence comprises a sequence of TGTTGAAA and the TIR of the second transposon end sequence comprises the reverse complement of TGTTGATA (TATCAACA), (b) the TIR of the first transposon end sequence comprises a sequence of TGTTGAAA and the TIR of the second transposon end sequence comprises the reverse complement of TGTTGAAA (TTTCAACA), (c) the TIR of the first transposon end sequence comprises a sequence of TGTTGATC and the TIR of the second transposon end sequence comprises the reverse complement of TGTTGATC (GATCAACA), (d) the TIR of the first transposon end sequence comprises a sequence of TGTCGTTT and the TIR of the second transposon end sequence comprises the reverse complement of TGTCGTTT (AAACGACA), (e) the TIR of the first transposon end sequence comprises a sequence of TGTCGTTT and the TIR of the second transposon end sequence comprises the reverse complement of TGTCGCTT (AAACGACA); (f) the TIR of the first transposon end sequence comprises a sequence of TGTCGCAA and the TIR of the second transposon end sequence comprises the reverse complement of TGTCGCTG (AAGCGACA), (g) the TIR of the first transposon end sequence comprises a sequence of TGTGGCTG and the TIR of the second transposon end sequence comprises the reverse complement of TGTGGCTA (TAGCCACA), and/or (h) the TIR of the first transposon end sequence comprises a sequence of TGTCGCTG and the TIR of the second transposon end sequence comprises the reverse complement of TGTCGCTG (CAGCGACA).

In some embodiments, for a system of the invention derived from: (a) Aliiglaciecola sp., the TIR of the first transposon end sequence comprises a sequence of TGTTGATC and the TIR of the second transposon end sequence comprises the reverse complement of TTGTTGATC (GATCAACA), (b) Vibrio sp., the TIR of the first transposon end sequence comprises a sequence of TGTGGCTG and the TIR of the second transposon end sequence comprises the reverse complement of TGTGGCTA (TAGCCACA), (c) H. titanicae, the TIR of the first transposon end sequence comprises a sequence of TGTCGTTT and the TIR of the second transposon end sequence comprises the reverse complement of TGTCGTTT (AAACGACA), (d) Photobacterium aquae, the TIR of the first transposon end sequence comprises a sequence of TGTCGCAA and the TIR of the second transposon end sequence comprises the reverse complement of TGTCGCTG (AAGCGACA), (e) Photobacterium iliopiscarium, the TIR of the first transposon end sequence comprises a sequence of TGTTGAAA and the TIR of the second transposon end sequence comprises the reverse complement of TGTTGATA (TATCAACA), (f) Photobacterium piscicola, the TIR of the first transposon end sequence comprises a sequence of TGTTGAAA and the TIR of the second transposon end sequence comprises the reverse complement of TGTTGAAA (TTTCAACA), (g) Psychromonas sp., the TIR of the first transposon end sequence comprises a sequence of TGTCGCTG and the TIR of the second transposon end sequence comprises the reverse complement of TGTCGCTG (CAGCGACA), (h) Klebsiella oxytoca, the TIR of the first transposon end sequence comprises a sequence of TGTCGTTT and the TIR of the second transposon end sequence comprises the reverse complement of TGTCGCTT (AAACGACA), (i) Colwellia polaris, the TIR of the first transposon end sequence comprises a sequence of TGTCGCTG and the TIR of the second transposon end sequence comprises the reverse complement of TGTCGCTG (CAGCGACA), and/or (j) Neptunomonas qingdaonensis, the TIR of the first transposon end sequence comprises a sequence of TGTCGTTT and the TIR of the second transposon end sequence comprises the reverse complement of TGTCGTTT (AAACGACA).

In some embodiments, when the system of the invention is derived from (A) Aliiglaciecola sp., the first transposon end sequence and the second transposon end sequence comprise a nucleotide sequence of SEQ ID NO:59 and SEQ ID NO:60, respectively; (B) Vibrio sp., the first transposon end sequence and the second transposon end sequence comprise a nucleotide sequence of SEQ ID NO: 156 and SEQ ID NO: 157, respectively; (C) Halomonas titanicae, the first transposon end sequence and the second transposon end sequence comprise a nucleotide sequence of SEQ ID NO:79 and SEQ ID NO:80, respectively; (D) Photobacterium aquae, the first transposon end sequence and the second transposon end sequence comprise a nucleotide sequence of SEQ ID NO:138 and SEQ ID NO: 139, respectively; (E) Photobacterium iliopiscarium, the first transposon end sequence and the second transposon end sequence comprise a nucleotide sequence of SEQ ID NO:20 and SEQ ID NO: 21, respectively; (F) Photobacterium piscicola, the first transposon end sequence and the second transposon end sequence comprise a nucleotide sequence of SEQ ID NO:38 and SEQ ID NO:39, respectively; (G) Psychromonas sp., the first transposon end sequence and the second transposon end sequence comprise a nucleotide sequence of SEQ ID NO:175 and SEQ ID NO:176, respectively; (H) Klebsiella oxytoca, the first transposon end sequence and the second transposon end sequence comprise a nucleotide sequence of SEQ ID NO:118 and SEQ ID NO:119, respectively; (I) Colwellia polaris, the first transposon end sequence and the second transposon end sequence comprise a nucleotide sequence of SEQ ID NO:193 and SEQ ID NO: 194, respectively; and/or (J) Neptunomonas qingdaonensis, the first transposon end sequence and the second transposon end sequence comprise a nucleotide sequence of SEQ ID NO: 101 and SEQ ID NO:102, respectively.

In some embodiments, the CRISPR-Cas system and the Tn7-like transposon system may be on the same vector. In some embodiments, the guide nucleic acid may be on the same vector as the CRISPR-Cas system and the Tn7-like transposon system. In some embodiments, the CRISPR-Cas system, the Tn7-like transposon system, and the guide nucleic acid may be on two or more separate vectors in any combination. In some embodiments, the CRISPR-Cas system, the Tn7-like transposon system, the guide nucleic acid and the donor DNA may be on the same vector or they may be on two or more separate vectors in any combination.

A system of the invention that is directed to RNA-guided DNA integration (e.g., an engineered CRISPR-Cas system and an engineered Tn7-like transposon system as described herein) may be comprised on one or more (e.g., 1, 2, 3, 4, 5 or 6 or more) vectors. In some embodiments, the one or more vectors may be viral vectors, plasmid vectors, phage vectors, phagemid vectors, cosmid vectors, fosmid vectors, bacteriophage, artificial chromosome, and/or Agrobacterium binary vectors, in any combination. In some embodiments, the one or more vectors may be one or more plasmid(s).

The present invention further provides a ribonucleoprotein (RNP) comprising (a) one or more of a Cas6 polypeptide, a Cas7 polypeptide, and/or a Cas8 polypeptide, a Transposon 7 protein A (TnsA), a Transposon 7 protein B (Tns B), a Transposon 7 protein C (Tns C), and a transposition of integron protein Q (TniQ), and (b) a Clustered Regularly Interspaced Short Palindromic Repeats (CRISPR) array comprising two or more repeat sequences and one or more spacer sequence(s), wherein each of the one or more spacer sequences is linked at its 5′ end and at its 3′ end to a repeat sequence, and each of the one or more spacer sequences is complementary to one or more target sequences in a target DNA of a target organism.

The present invention further provides recombinant cells comprising the system of the invention and or an RNP of the invention and/or recombinant organisms comprising one or more cells comprising the system of the invention and or an RNP of the invention. A cell from any organism (e.g., a target organism, host organism, or organism of interest) may be transformed/transduced with the system of the invention and or an RNP of the invention to produce recombinant cells comprising the system of the invention and or an RNP of the invention.

The compositions of the present invention (e.g., systems, recombinant nucleic acid constructs) may be used in methods for RNA-directed DNA integration, methods for modifying nucleic acids such as modifying the genome of a target organism or a cell thereof. In some embodiments, the nucleic acid modification may be carried out in a cell free system. In some embodiments, the nucleic acid or genome modification may be directed to targeted gene silencing, repression of expression and/or modulation of the repression of expression in an organism of interest or cell thereof or in a cell free system. Other methods include selection of or screening for variants in a population of cells or selected killing of cells in a population.

Accordingly for use in such methods, the recombinant nucleic acid constructs of the invention may be introduced into a target organism and/or a cell of a target organism. In some embodiments, the recombinant nucleic acid constructs of the invention may be contacted with a target nucleic acid in a cell free system. In some embodiments, the recombinant nucleic acid constructs of the invention may be stably or transiently introduced into a target organism and/or a cell of a target organism.

“Introducing,” “introduce,” “introduced” (and grammatical variations thereof) in the context of a polynucleotide of interest and a cell of an organism means presenting the polynucleotide of interest to the host organism or cell of the organism (e.g., host cell) in such a manner that the nucleotide sequence gains access to the interior of a cell and includes such terms as transformation,” “transfection,” and/or “transduction.” Where more than one nucleotide sequence is to be introduced these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides can be introduced into cells in a single transformation event, in separate transformation events, or, for example, they can be incorporated into an organism by conventional breeding protocols. Thus, in some aspects of the present invention one or more recombinant nucleic acid constructs of this invention may be introduced into a host organism or a cell of the host organism.

The terms “transformation,” “transfection,” and “transduction” as used herein refer to the introduction of a heterologous nucleic acid into a cell. Such introduction into a cell may be stable or transient. Thus, in some embodiments, a host cell or host organism is stably transformed with a nucleic acid construct of the invention. In other embodiments, a host cell or host organism is transiently transformed with a recombinant nucleic acid construct of the invention.

As used herein, the term “stably introduced” means that the introduced polynucleotide is stably incorporated into the genome of the cell, and thus the cell is stably transformed with the polynucleotide. When a nucleic acid construct is stably transformed and therefore integrated into a cell, the integrated nucleic acid construct is capable of being inherited by the progeny thereof, more particularly, by the progeny of multiple successive generations.

“Transient transformation” in the context of a polynucleotide means that a polynucleotide is introduced into the cell and does not integrate into the genome of the cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., an animal, a mammal, an insect, a fish, a plant, a bacterium, and the like). Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, the nucleotide sequences, constructs, expression cassettes may be expressed transiently and/or they may be stably incorporated into the genome of a target organism. In some embodiments, when transient transformation is desired, the loss of the plasmids and the recombinant nucleic acids comprised therein may achieved by removal of selective pressure for plasmid maintenance.

A recombinant nucleic acid construct of the invention (e.g., one or more vectors comprising a system of the invention) can be introduced into a cell by any method known to those of skill in the art. Exemplary methods of transformation or transfection include biological methods using viruses and bacteria (e.g., Agrobacterium), physicochemical methods such as electroporation, floral dip methods, particle or ballistic bombardment, microinjection, whiskers technology, pollen tube transformation, calcium-phosphate-mediated transformation, nanoparticle-mediated transformation, polymer-mediated transformation including cyclodextrin-mediated and polyethylene glycol-mediated transformation, sonication, infiltration, as well as any other electrical, chemical, physical (mechanical) and/or biological mechanism that results in the introduction of nucleic acid into a cell, including any combination thereof.

In some embodiments of the invention, transformation of a cell comprises nuclear transformation. In other embodiments, transformation of a cell comprises plastid transformation (e.g., chloroplast transformation). In still further embodiments, the recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. 2013. Nature Protocols 8:2281-2308)

A nucleic acid therefore can be introduced into a target organism or its cell in any number of ways that are well known in the art. The methods of the invention do not depend on a particular method for introducing one or more nucleotide sequences into the organism, only that they gain access to the interior of at least one cell of the organism. Where more than one polynucleotide is to be introduced, they can be assembled as part of a single nucleic acid construct, or as separate nucleic acid constructs, and can be located on the same or different nucleic acid constructs. Accordingly, the polynucleotides can be introduced into the cell of interest in a single transformation event, or in separate transformation events, or, alternatively, where relevant, a nucleotide sequence can be incorporated as part of a breeding protocol.

Accordingly, a method for RNA-guided DNA integration is provided, the method comprising, introducing into a cell a system of the invention, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid.

In some embodiments, a method of modifying the genome of a target organism is provided, the method comprising introducing into a cell a system of the invention, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the genome of the target organism.

In some embodiments, a method of modifying (e.g., editing) a nucleic acid (e.g., target region; target DNA) in the genome of a cell is provided, the method comprising introducing into a cell a system of the invention, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the nucleic acid in the genome of the cell.

In some embodiments, a method of killing a cell is provided, the method comprising introducing into a cell a system of the invention, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into an essential gene in the nucleic acid, thereby killing the cell.

In some embodiments, a method of killing a prokaryotic cell is provided, the method comprising introducing into a prokaryotic cell a system of the invention, a donor DNA, and a guide nucleic acid comprising at least one spacer having complementarity to a target site present in a nucleic acid present in the prokaryotic cell, wherein the engineered CRISPR-Cas system binds to the target site and the engineered transposon system integrates the donor sequence into an essential gene in the nucleic acid, thereby killing the prokaryotic cell.

In some embodiments, a method for screening for non-essential genes in a cell is provided, the method comprising introducing into a cell a system of the invention, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into a gene in the nucleic acid, wherein when the cell survives, the gene is non-essential, thereby screening for non-essential genes in the cell.

In some embodiments, a method for screening for non-essential genes in a prokaryotic cell is provided, the method comprising introducing into a prokaryotic cell a system of the invention, a donor DNA, and a guide nucleic acid comprising at least one spacer having complementarity to a target site present in a nucleic acid present in the prokaryotic cell, wherein the engineered CRISPR-Cas system binds to the target site and the engineered transposon system integrates the donor sequence into a gene in the nucleic acid, wherein when the prokaryotic cell survives, the gene is non-essential, thereby screening for non-essential genes in the prokaryotic cell.

In some embodiments, a method of altering the expression of a gene in a cell, the method comprising introducing into a cell a system of the invention, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into a regulator region of a gene in the nucleic acid, thereby altering the expression of a gene in a cell.

In some embodiments, a method of altering the expression of a gene in a prokaryotic cell, the method comprising introducing into a prokaryotic cell a system of the invention, a guide nucleic acid comprising at least one spacer having complementarity to a target site present in a nucleic acid present in the prokaryotic cell, and a donor DNA, wherein the engineered CRISPR-Cas system binds to the target site and the engineered transposon system integrates the donor sequence into a regulator region of a gene in the nucleic acid, thereby altering the expression of a gene in a prokaryotic cell.

Any cell or organism (e.g., target cell, target organism, host organism, or organism of interest) may be used with the methods of the invention, including, but not limited to, eukaryotic organisms, prokaryotic organisms and/or viruses. One or more cells from a target organism may be transformed/transduced with the system of the invention and or an RNP of the invention to produce recombinant cells comprising the system of the invention and or an RNP of the invention. A target organism may be, for example, a eukaryotic organism or a prokaryotic organism or a virus. Non-limiting examples of a prokaryote are bacteria, cyanobacteria, or archaea. A eukaryote can include, but is not limited to, an animal, a mammal, an insect, a plant, a fungus, a nematode, an insect, a bird, a fish, an amphibian, a reptile, or a cnidarian. In some embodiments, a mammal can include, but is not limited to, a rodent, a horse, a dog a cat, a human, a non-human primate (e.g., monkeys, baboons, and chimpanzees), a goat, a pig, a cow (e.g., cattle), a sheep, laboratory animals (e.g., rats, rabbits, mice, gerbils, hamsters, and the like) and the like. Non-limiting examples of birds useful with this invention include chickens, ducks, turkeys, geese, quails and birds kept as pets (e.g., parakeets, parrots, macaws, and the like). Additional embodiments can include, for example, mammalian and insect cell lines. Non-limiting examples of mammalian and insect cell lines include HEK293 cells, HeLa cells, CHO cells, MEF cells, 3T3 cells, Hi-5 cells, and Sf21 cells.

Suitable target organisms can include both males and females and subjects of all ages including embryonic (e.g., in utero or in ovo), infant, juvenile, adolescent, adult and geriatric subjects. In embodiments of the invention, the target organism is not a human embryonic subject.

In some embodiments, non-limiting examples of bacteria useful with this invention can include Escherichia spp., Salmonella spp., Bacillus spp., Corynebacterium Clostridium spp., Clostridium spp., Psuedomonas spp., Clostridium spp., Lactococcus spp. Acinetobacter spp., Mycobacterium spp., Myxococcus spp., Staphylococcus spp., Streptococcus spp., or cyanobacteria. In some embodiments, non-limiting examples of bacterial species useful with this invention include Escherichia coli, Salmonella enterica, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, or Streptococcus pyogenes. Further non-limiting examples of bacteria useful with this invention include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals.

Non-limiting examples of such archaea include Pyrococcus furiosus, Thermus aquaticus, Sulfolobus sulfataricus, or haloarchaea including but not limited to Haladaptatus (e.g., Haladaptatus paucihalophilus), Halalkalicoccus (e.g., Halalkalicoccus tibetensis), Halobaculum (e.g., Halobaculum gomorrense), Halobellus (e.g., Halobellus clavatus), Halomicrobium (e.g., Halomicrobium mukohataei), Natrialba (e.g., Natrialba asiatica), Natrinema (e.g., Natrinema pellirubrum), Natronorubrum (e.g., Natronorubrum bangense), or Salarchaeum (e.g., Salarchaeum japonicum)

A plant, plant part and/or plant cell useful with this invention can include, but is not limited to, plants from the genera of Camelina, Glycine, Sorghum, Brassica, Allium, Armoracia, Poa, Agrostis, Lolium, Festuca, Calamogrostis, Deschampsia, Spinacia, Beta, Pisum, Chenopodium, Helianthus, Pastinaca, Daucus, Petroselium, Populus, Prunus, Castanea, Eucalyptus, Acer, Quercus, Salix, Juglans, Picea, Pinus, Abies, Lemna, Wolffia, Spirodela, Oryza, Zea or Gossypium. In some embodiments, the plant, plant part and/or plant cell can include, but is not limited to, the plant species of Camelina alyssum (Mill.) Thell., Camelina microcarpa Andrz. ex DC., Camelina rumelica Velen., Camelina sativa (L.) Crantz, Sorghum bicolor (e.g., Sorghum bicolor L. Moench), Gossypium hirsutum, Glycine max, Zea mays, Brassica oleracea, Brassica rapa, Brassica napus, Raphanus sativus, Armoracia rusticana, Allium sative, Allium cepa, Populus grandidentata, Populus tremula, Populus tremuloides, Prunus serotina, Prunus pensylvanica, Castanea dentate, Populus balsamifer, Populus deltoids, Acer Saccharum, Acer nigrum, Acer negundo, Acer rubrum, Acer saccharinum, Acer pseudoplatanus or Oryza sativa. In some embodiments, a plant, plant part and/or plant cell can be, but is not limited to, wheat, barley, oats, turfgrass (bluegrass, bentgrass, ryegrass, fescue), feather reed grass, tufted hair grass, spinach, beets, chard, quinoa, sugar beets, lettuce, sunflower (Helianthus annuus), peas (Pisum sativum), parsnips (Pastinaca sativa), carrots (Daucus carota), parsley (Petroselinum crispum), duckweed, pine, spruce, fir, eucalyptus, oak, walnut, or willow. In some embodiments, the plant, plant part and/or plant cell can be Arabidopsis thaliana. In some embodiments, the plant and/or plant cell can be camelina, wheat, rice, corn, rape, canola, soybean, sorghum, tomato, bamboo, or cotton.

In some embodiments, a plant and/or plant cell may be an algae or algae cell including, but not limited to, a Bacillariophyceae (diatoms), Haptophyceae, Phaeophyceae (brown algae), Rhodophyceae (red algae) or Glaucophyceae (red algae). In still other embodiments, non-limiting examples of an algae or algae cell include Achnanthidium, Actinella, Nitzschia, Nupela, Geissleria, Gomphonema, Planothidium, Halamphora, Psammothidium, Navicula, Eunotia, Stauroneis, Chlamydomonas, Dunaliella, Nannochloris, Nannochloropsis, Scenedesmus, Chlorella, Cyclotella, Amphora, Thalassiosira, Phaeodactylum, Chrysochromulina, Prymnesium, Thalassiosira, Phaeodactylum, Glaucocystis, Cyanophora, Galdieria, or Porphyridium.

Non-limiting examples of fungi useful with this invention include Candida spp., Fusarium spp., Aspergillus spp., Cryptococcus spp., Coccidioides spp., Tinea spp., Sporothrix spp., Blastomyces spp., Histoplasma spp., Pneumocystis spp, Saccharomyces spp., Saccharomycodes spp., Kluyveromyces spp., Pichia spp., Candida spp., Zygosaccharomyces spp. or Hanseniaspora spp. In some embodiments, a fungal species useful with the invention can include, but is not limited to, Saccharomyces cerevisiae, S. uvarum (carlsbergensis), S. diastaticus, Saccharomycodes ludwigii, Kluyveromyces marxianus, Pichia pastoris, Candida stellata, C. pulcherrima, Zygosaccharomyces fermentati, Hanseniaspora uvarum, Aspergillus fumigatus, Aspergillus flavus, Aspergillus niger, Aspergillus terreus, Aspergillus nidulans, Candida albicans, Coccidioides immitis, Cryptococcus neoformans, Fusarium solani, Fusarium culmorum, Tinea unguium, Tinea corporis, Tinea cruris, Sporothrix schenckii, Blastomyces dermatitidis, Histoplasma capsulatum, Pneumocystis carinii, or Histoplasma duboisii.

A CasTns system of the invention useful with the methods of the invention are as described herein and comprises (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), or comprising: (a) Transposon 7 protein AB (TnsAB), (b) Transposon 7 protein C (TnsC), and (c) transposition of integron protein Q (TniQ). In some embodiments, the CasTns system may be derived from

In some embodiments, the CasTns system may be derived from Aliiglaciecola sp., Vibrio sp., Halomonas titanicae, Photobacterium aquae, Photobacterium iliopiscarium, Photobacterium piscicola, Psychromonas sp., Klebsiella oxytoca, Colwellia polaris and/or Neptunomonas qingdaonensis, optionally from Aliiglaciecola sp. strain M165, Vibrio sp. strain EJY3, H. titanicae strain BH1, P. aquae strain CGMCC 1.12159, P. iliopiscarium strain ATCC 51760, P. piscicola sp. strain NCCB 100098, Psychromonas sp. strain RZ5, K. oxytoca strain 67, C. polaris strain MCCC 1C00015; and/or N. qingdaonensis strain CGMCC 1.10971.

In some embodiments, when the CasTns system is derived from: (A) Aliiglaciecola sp., Cas6 is encoded by a nucleotide sequence having at least 90% (e.g., 90%, 91%, 92%, 93%, 94%, 95%, 96%, 97%, 98%, 99%, and/or 100%) sequence identity to SEQ ID NO:43 and/or comprises an amino acid sequence having at least 90% (e.g., 90%, 91%, 92%, 93%, 94%, 95%, 96%, 97%, 98%, 99%, and/or 100%) sequence identity to SEQ ID NO:50, Cas7 is encoded by a nucleotide sequence having at least 90% sequence identity to SEQ ID NO:42 and/or comprises an amino acid sequence having at least 90% sequence identity to SEQ ID NO: 49, Cas8 is encoded by a nucleotide sequence having at least 90% sequence identity to SEQ ID NO:41 and/or comprises an amino acid sequence having at least 90% sequence identity to SEQ ID NO:48, TnsA is encoded by a nucleotide sequence having at least 90% sequence identity to SEQ ID NO:44 and/or comprises an amino acid sequence having at least 90% sequence identity to SEQ ID NO:51, TnsB is encoded by a nucleotide sequence having at least 90% sequence identity to SEQ ID NO:45 and/or comprises an amino acid sequence having at least 90% sequence identity to SEQ ID NO:52, TnsC is encoded by a nucleotide sequence having at least 90% sequence identity to SEQ ID NO:46 and/or comprises an amino acid sequence having at least 90% sequence identity to SEQ ID NO:53, and/or TniQ is encoded by a nucleotide sequence having at least 90% sequence identity to SEQ ID NO:40 and/or comprises an amino acid sequence having at least 90% sequence identity to SEQ ID NO: 47; (B) Vibrio sp., Cas6 is encoded by a nucleotide sequence having at least 90% sequence identity to SEQ ID NO:143 and/or comprises an amino acid sequence having at least 90% sequence identity to SEQ ID NO: 150, Cas7 is encoded by a nucleotide sequence having at least 90% sequence identity to SEQ ID NO:142 and/or comprises an amino acid sequence having at least 90% sequence identity to SEQ ID NO:149, Cas8 is encoded by a nucleotide sequence having at least 90% sequence identity to SEQ ID NO:141 and/or comprises an amino acid sequence having at least 90% sequence identity to SEQ ID NO: 148, TnsA is encoded by a nucleotide sequence having at least 90% sequence identity to SEQ ID NO: 144 and/or comprises an amino acid sequence having at least 90% sequence identity to SEQ ID NO:151, TnsB is encoded by a nucleotide sequence having at least 90% sequence identity to SEQ ID NO: 145 and/or comprises an amino acid sequence having at least 90% sequence identity to SEQ ID NO:152, TnsC is encoded by a nucleotide sequence having at least 90% sequence identity to SEQ ID NO: 146 and/or comprises an amino acid sequence having at least 90% sequence identity to SEQ ID NO:153, and/or TniQ is encoded by a nucleotide sequence having at least 90% sequence identity to SEQ ID NO:140 and/or comprises an amino acid sequence having at least 90% sequence identity to SEQ ID NO: 147; (C) H. titanicae, Cas6 is encoded by a nucleotide sequence having at least 90% sequence identity to SEQ ID NO:64 and/or comprises an amino acid sequence having at least 90% sequence identity to SEQ ID NO:71, Cas7 is encoded by a nucleotide sequence having at least 90% sequence identity to SEQ ID NO: 63 and/or comprises an amino acid sequence having at least 90% sequence identity to SEQ ID NO:70, Cas8 is encoded by a nucleotide sequence having at least 90% sequence identity to SEQ ID NO:62 and/or comprises an amino acid sequence having at least 90% sequence identity to SEQ ID NO:69, TnsA is encoded by a nucleotide sequence having at least 90% sequence identity to SEQ ID NO:65 and/or comprises an amino acid sequence having at least 90% sequence identity to SEQ ID NO:72, TnsB is encoded by a nucleotide sequence having at least 90% sequence identity to SEQ ID NO:66 and/or comprises an amino acid sequence having at least 90% sequence identity to SEQ ID NO:73, TnsC is encoded by a nucleotide sequence having at least 90% sequence identity to SEQ ID NO:67 and/or comprises an amino acid sequence having at least 90% sequence identity to SEQ ID NO:74, and/or TniQ is encoded by a nucleotide sequence having at least 90% sequence identity to SEQ ID NO:61 and/or comprises an amino acid sequence having at least 90% sequence identity to SEQ ID NO: 68; (D) P. aquae, Cas6 is encoded by a nucleotide sequence having at least 90% sequence identity to SEQ ID NO:123 and/or comprises an amino acid sequence having at least 90% sequence identity to SEQ ID NO:130, Cas7 is encoded by a nucleotide sequence having at least 90% sequence identity to SEQ ID NO:122 and/or comprises an amino acid sequence having at least 90% sequence identity to SEQ ID NO:129, Cas8 is encoded by a nucleotide sequence having at least 90% sequence identity to SEQ ID NO: 121 and/or comprises an amino acid sequence having at least 90% sequence identity to SEQ ID NO: 128, TnsA is encoded by a nucleotide sequence having at least 90% sequence identity to SEQ ID NO: 124 and/or comprises an amino acid sequence having at least 90% sequence identity to SEQ ID NO:131, TnsB is encoded by a nucleotide sequence having at least 90% sequence identity to SEQ ID NO: 125 and/or comprises an amino acid sequence having at least 90% sequence identity to SEQ ID NO: 132, TnsC is encoded by a nucleotide sequence having at least 90% sequence identity to SEQ ID NO: 126 and/or comprises an amino acid sequence having at least 90% sequence identity to SEQ ID NO:133, and/or TniQ is encoded by a nucleotide sequence having at least 90% sequence identity to SEQ ID NO:120 and/or comprises an amino acid sequence having at least 90% sequence identity to SEQ ID NO:127; (E) P. iliopiscarium, Cas6 is encoded by a nucleotide sequence having at least 90% sequence identity to SEQ ID NO:4 and/or comprises an amino acid sequence having at least 90% sequence identity to SEQ ID NO:11, Cas7 is encoded by a nucleotide sequence having at least 90% sequence identity to SEQ ID NO: 3 and/or comprises an amino acid sequence having at least 90% sequence identity to SEQ ID NO: 10, Cas8 is encoded by a nucleotide sequence having at least 90% sequence identity to SEQ ID NO:2 and/or comprises an amino acid sequence having at least 90% sequence identity to SEQ ID NO:9, TnsA is encoded by a nucleotide sequence having at least 90% sequence identity to SEQ ID NO:5 and/or comprises an amino acid sequence having at least 90% sequence identity to SEQ ID NO:12, TnsB is encoded by a nucleotide sequence having at least 90% sequence identity to SEQ ID NO:6 and/or comprises an amino acid sequence having at least 90% sequence identity to SEQ ID NO:13, TnsC is encoded by a nucleotide sequence having at least 90% sequence identity to SEQ ID NO:7 and/or comprises an amino acid sequence having at least 90% sequence identity to SEQ ID NO: 14, and/or TniQ is encoded by a nucleotide sequence having at least 90% sequence identity to SEQ ID NO:1 and/or comprises an amino acid sequence having at least 90% sequence identity to SEQ ID NO:8; (F) P. piscicola, Cas6 is encoded by a nucleotide sequence having at least 90% sequence identity to SEQ ID NO:25 and/or comprises an amino acid sequence having at least 90% sequence identity to SEQ ID NO:32, Cas7 is encoded by a nucleotide sequence having at least 90% sequence identity to SEQ ID NO:24 and/or comprises an amino acid sequence having at least 90% sequence identity to SEQ ID NO:31, Cas8 is encoded by a nucleotide sequence having at least 90% sequence identity to SEQ ID NO:23 and/or comprises an amino acid sequence having at least 90% sequence identity to SEQ ID NO:30, TnsA is encoded by a nucleotide sequence having at least 90% sequence identity to SEQ ID NO:26 and/or comprises an amino acid sequence having at least 90% sequence identity to SEQ ID NO:33, TnsB is encoded by a nucleotide sequence having at least 90% sequence identity to SEQ ID NO:27 and/or comprises an amino acid sequence having at least 90% sequence identity to SEQ ID NO:34, TnsC is encoded by a nucleotide sequence having at least 90% sequence identity to SEQ ID NO:28 and/or comprises an amino acid sequence having at least 90% sequence identity to SEQ ID NO: 35, and/or TniQ is encoded by a nucleotide sequence having at least 90% sequence identity to SEQ ID NO:22 and/or comprises an amino acid sequence having at least 90% sequence identity to SEQ ID NO:29; (G) Psychromonas sp., Cas6 is encoded by a nucleotide sequence having at least 90% sequence identity to SEQ ID NO:161 and/or comprises an amino acid sequence having at least 90% sequence identity to SEQ ID NO: 168, Cas7 is encoded by a nucleotide sequence having at least 90% sequence identity to SEQ ID NO:160 and/or comprises an amino acid sequence having at least 90% sequence identity to SEQ ID NO:167, Cas8 is encoded by a nucleotide sequence having at least 90% sequence identity to SEQ ID NO: 159 and/or comprises an amino acid sequence having at least 90% sequence identity to SEQ ID NO: 166, TnsA is encoded by a nucleotide sequence having at least 90% sequence identity to SEQ ID NO: 162 and/or comprises an amino acid sequence having at least 90% sequence identity to SEQ ID NO: 169, TnsB is encoded by a nucleotide sequence having at least 90% sequence identity to SEQ ID NO:163 and/or comprises an amino acid sequence having at least 90% sequence identity to SEQ ID NO: 170, TnsC is encoded by a nucleotide sequence having at least 90% sequence identity to SEQ ID NO: 164 and/or comprises an amino acid sequence having at least 90% sequence identity to SEQ ID NO: 171, and/or TniQ is encoded by a nucleotide sequence having at least 90% sequence identity to SEQ ID NO: 158 and/or comprises an amino acid sequence having at least 90% sequence identity to SEQ ID NO: 165; (H) K. oxytoca, Cas6 is encoded by a nucleotide sequence having at least 90% sequence identity to SEQ ID NO: 106 and/or comprises an amino acid sequence having at least 90% sequence identity to SEQ ID NO:112, Cas7 is encoded by a nucleotide sequence having at least 90% sequence identity to SEQ ID NO: 105 and/or comprises an amino acid sequence having at least 90% sequence identity to SEQ ID NO: 111, Cas8 is encoded by a nucleotide sequence having at least 90% sequence identity to SEQ ID NO:104 and/or comprises an amino acid sequence having at least 90% sequence identity to SEQ ID NO: 110, TnsAB is encoded by a nucleotide sequence having at least 90% sequence identity to SEQ ID NO:107 and/or comprises an amino acid sequence having at least 90% sequence identity to SEQ ID NO:113, TnsC is encoded by a nucleotide sequence having at least 90% sequence identity to SEQ ID NO: 108 and/or comprises an amino acid sequence having at least 90% sequence identity to SEQ ID NO:114, and/or TniQ is encoded by a nucleotide sequence having at least 90% sequence identity to SEQ ID NO:103 and/or comprises an amino acid sequence having at least 90% sequence identity to SEQ ID NO:109; (I) C. polaris, Cas6 is encoded by a nucleotide sequence having at least 90% sequence identity to SEQ ID NO: 180 and/or comprises an amino acid sequence having at least 90% sequence identity to SEQ ID NO: 187, Cas7 is encoded by a nucleotide sequence having at least 90% sequence identity to SEQ ID NO:179 and/or comprises an amino acid sequence having at least 90% sequence identity to SEQ ID NO:186, Cas8 is encoded by a nucleotide sequence having at least 90% sequence identity to SEQ ID NO: 178 and/or comprises an amino acid sequence having at least 90% sequence identity to SEQ ID NO: 185, TnsA is encoded by a nucleotide sequence having at least 90% sequence identity to SEQ ID NO:181 and/or comprises an amino acid sequence having at least 90% sequence identity to SEQ ID NO: 188, TnsB is encoded by a nucleotide sequence having at least 90% sequence identity to SEQ ID NO: 182 and/or comprises an amino acid sequence having at least 90% sequence identity to SEQ ID NO: 189, TnsC is encoded by a nucleotide sequence having at least 90% sequence identity to SEQ ID NO: 183 and/or comprises an amino acid sequence having at least 90% sequence identity to SEQ ID NO: 190, and/or TniQ is encoded by a nucleotide sequence having at least 90% sequence identity to SEQ ID NO: 177 and/or comprises an amino acid sequence having at least 90% sequence identity to SEQ ID NO: 184; and/or (J) N. qingdaonensis, Cas6 is encoded by a nucleotide sequence having at least 90% sequence identity to SEQ ID NO:84 and/or comprises an amino acid sequence having at least 90% sequence identity to SEQ ID NO:91, Cas7 is encoded by a nucleotide sequence having at least 90% sequence identity to SEQ ID NO:83 and/or comprises an amino acid sequence having at least 90% sequence identity to SEQ ID NO: 90, Cas8 is encoded by a nucleotide sequence having at least 90% sequence identity to SEQ ID NO:82 and/or comprises an amino acid sequence having at least 90% sequence identity to SEQ ID NO:89, TnsA is encoded by a nucleotide sequence having at least 90% sequence identity to SEQ ID NO:85 and/or comprises an amino acid sequence having at least 90% sequence identity to SEQ ID NO: 92, TnsB is encoded by a nucleotide sequence having at least 90% sequence identity to SEQ ID NO:86 and/or comprises an amino acid sequence having at least 90% sequence identity to SEQ ID NO:93, TnsC is encoded by a nucleotide sequence having at least 90% sequence identity to SEQ ID NO:87 and/or comprises an amino acid sequence having at least 90% sequence identity to SEQ ID NO:94, and/or TniQ is encoded by a nucleotide sequence having at least 90% sequence identity to SEQ ID NO:81 and/or comprises an amino acid sequence having at least 90% sequence identity to SEQ ID NO:88.

In some embodiments, the CasTns system may be optimized for expression (e.g., enhanced or improved expression) in a species of interest. In some embodiments, a CasTns system is an optimized CasTns system with enhanced expression in a species of plant as compared to expression of the nonoptimized CasTns system (wild type) in the same species of plant. In some embodiments, the CasTns system that is optimized may be derived from Psychromonas sp. and the optimized sequences comprise: a Cas6 encoded by a nucleotide sequence having at least 90% sequence identity to SEQ ID NO:213 and/or comprising an amino acid sequence having at least 90% sequence identity to SEQ ID NO: 168, a Cas7 encoded by a nucleotide sequence having at least 90% sequence identity to SEQ ID NO:214 and/or comprising an amino acid sequence having at least 90% sequence identity to SEQ ID NO: 167, a Cas8 encoded by a nucleotide sequence having at least 90% sequence identity to SEQ ID NO:215 and/or comprising an amino acid sequence having at least 90% sequence identity to SEQ ID NO: 166, a TnsA encoded by a nucleotide sequence having at least 90% sequence identity to SEQ ID NO:217 and/or comprising an amino acid sequence having at least 90% sequence identity to SEQ ID NO: 169, a TnsB encoded by a nucleotide sequence having at least 90% sequence identity to SEQ ID NO:218 and/or comprising an amino acid sequence having at least 90% sequence identity to SEQ ID NO:170, a TnsC encoded by a nucleotide sequence having at least 90% sequence identity to SEQ ID NO:219 and/or comprising an amino acid sequence having at least 90% sequence identity to SEQ ID NO:171, and/or a TniQ encoded by a nucleotide sequence having at least 90% sequence identity to SEQ ID NO:216 and/or comprising an amino acid sequence having at least 90% sequence identity to SEQ ID NO: 165.

In some embodiments, the CasTns system may be optimized for expression (e.g., enhanced or improved expression) in maize. In some embodiments, a CasTns system optimized for maize may be derived from Psychromonas sp. and the optimized sequences comprise: a Cas7 encoded by a nucleotide sequence having at least 90% sequence identity to any one of the nucleotide sequences of SEQ ID NO:230-233 and/or comprising an amino acid sequence having at least 90% sequence identity to SEQ ID NO:167, and/or a TnsC encoded by a nucleotide sequence having at least 90% sequence identity to any one of the nucleotide sequences of SEQ ID NO:234-237 and/or comprising an amino acid sequence having at least 90% sequence identity to SEQ ID NO:171.

In some embodiments, the donor DNA that is introduced to carry out a method of this invention comprises a cargo nucleic acid sequence and a first transposon end sequence and a second transposon end sequence, wherein the cargo nucleic acid sequence is flanked by the first transposon end sequence and the second transposon end sequence, and wherein each of the first transposon end sequence and the second transposon end sequence comprises at least one TnsB binding site. In some embodiments, the first transposon end sequence and the second transposon end sequence each comprise at least two TnsB binding sites. In some embodiments, the at least two TnsB binding sites of a first transposon end sequence and a second transposon end sequence may be immediately adjacent to each other (e.g., no intervening nucleotides (no variable region)). In some embodiments, a variable region may be located between the at least two TnsB binding sites of the first transposon end sequence and the second transposon end sequence.

In some embodiments, a variable region that is located between two TnsB binding sites may have a length of about 1 nucleotide to about 80 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 nucleotides to about 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 70, 75, or 80 nucleotides), optionally a length of about 1 nucleotide to about 10 nucleotides (e.g., 1, 2, or 3 nucleotides to about 4, 5, 6, 7, 8, 9, or 10 nucleotides) and/or a length of about 1 nucleotide to about 3 nucleotides (e.g., 1, 2, or 3 nucleotides).

In some embodiments, the first transposon end sequence and/or the second transposon end sequence of the donor DNA that is introduced may comprise three or four TnsB binding sites, optionally, wherein at least two of the three or four TnsB binding sites are immediately adjacent to each other (e.g., no intervening nucleotides (no variable region) between the at least two of the three or four TnsB binding sites), and/or wherein a variable region of 1 nucleotide to about 80 nucleotides, optionally about 1 nucleotide to about 10 nucleotides and/or about 1 nucleotide to about 3 nucleotides, is located between at least two of the three to four TnsB binding sites.

In some embodiments, the 5′ end of a first transposon end sequence and the 3′ end of a second transposon end sequence useful with the invention comprise a terminal inverted repeat (TIR), optionally wherein the TIR for the second transposon end sequence may be the reverse complement of the TIR of the first transposon end sequence, optionally wherein the reverse complement of the second transposon end sequence may be a perfect reverse complement or wherein the reverse complement may have one or more mismatches as compared to the TIR of the first transposon end sequence. In some embodiments, a TIR of a first transposon end sequence and a TIR of a second transposon end sequence may each comprise a length of about 8 base pairs. In some embodiments, a TIR may comprise a sequence TGTNNNNN and/or a reverse complement of TGTNNNNN, optionally wherein the TIR at the 3′ end of the second transposon end sequence is the reverse complement of the TIR at the 5′ end of the first transposon end sequence (e.g., the TIR of the first transposon end sequence may comprise a sequence of TGTNNNNN and the TIR of the second transposon end sequence may comprise a sequence that is the reverse complement of TGTNNNNN (i.e., NNNNNACA)). In some embodiments, the TIR may comprise a sequence of TGTTGAAA, TGTTGATA, TGTTGATC, TGTCGTTT, TGTCGCTT, TGTCGCAA, TGTCGCTG, TGTGGCTG, or TGTGGCTA, or the reverse complement thereof.

In some embodiments, (a) the TIR of the first transposon end sequence comprises a sequence of TGTTGAAA and the TIR of the second transposon end sequence comprises the reverse complement of TGTTGATA (TATCAACA), (b) the TIR of the first transposon end sequence comprises a sequence of TGTTGAAA and the TIR of the second transposon end sequence comprises the reverse complement of TGTTGAAA (TTTCAACA), (c) the TIR of the first transposon end sequence comprises a sequence of TGTTGATC and the TIR of the second transposon end sequence comprises the reverse complement of TGTTGATC (GATCAACA), (d) the TIR of the first transposon end sequence comprises a sequence of TGTCGTTT and the TIR of the second transposon end sequence comprises the reverse complement of TGTCGTTT (AAACGACA), (e) the TIR of the first transposon end sequence comprises a sequence of TGTCGTTT and the TIR of the second transposon end sequence comprises the reverse complement of TGTCGCTT (AAACGACA); (f) the TIR of the first transposon end sequence comprises a sequence of TGTCGCAA and the TIR of the second transposon end sequence comprises the reverse complement of TGTCGCTG (AAGCGACA), (g) the TIR of the first transposon end sequence comprises a sequence of TGTGGCTG and the TIR of the second transposon end sequence comprises the reverse complement of TGTGGCTA (TAGCCACA), and/or (h) the TIR of the first transposon end sequence comprises a sequence of TGTCGCTG and the TIR of the second transposon end sequence comprises the reverse complement of TGTCGCTG (CAGCGACA).

In some embodiments, in a method of the invention when the system is derived from (a) Aliiglaciecola sp., the TIR of the first transposon end sequence comprises a sequence of TGTTGATC and the TIR of the second transposon end sequence comprises the reverse complement of TTGTTGATC (GATCAACA), (b) Vibrio sp., the TIR of the first transposon end sequence comprises a sequence of TGTGGCTG and the TIR of the second transposon end sequence comprises the reverse complement of TGTGGCTA (TAGCCACA), (c) H. titanicae, the TIR of the first transposon end sequence comprises a sequence of TGTCGTTT and the TIR of the second transposon end sequence comprises the reverse complement of TGTCGTTT (AAACGACA), (d) Photobacterium aquae, the TIR of the first transposon end sequence comprises a sequence of TGTCGCAA and the TIR of the second transposon end sequence comprises the reverse complement of TGTCGCTG (AAGCGACA), (e) Photobacterium iliopiscarium, the TIR of the first transposon end sequence comprises a sequence of TGTTGAAA and the TIR of the second transposon end sequence comprises the reverse complement of TGTTGATA (TATCAACA), (f) Photobacterium piscicola, the TIR of the first transposon end sequence comprises a sequence of TGTTGAAA and the TIR of the second transposon end sequence comprises the reverse complement of TGTTGAAA (TTTCAACA), (g) Psychromonas sp., the TIR of the first transposon end sequence comprises a sequence of TGTCGCTG and the TIR of the second transposon end sequence comprises the reverse complement of TGTCGCTG (CAGCGACA), (h) Klebsiella oxytoca, the TIR of the first transposon end sequence comprises a sequence of TGTCGTTT and the TIR of the second transposon end sequence comprises the reverse complement of TGTCGCTT (AAACGACA), (i) Colwellia polaris, the TIR of the first transposon end sequence comprises a sequence of TGTCGCTG and the TIR of the second transposon end sequence comprises the reverse complement of TGTCGCTG (CAGCGACA), and/or (j) Neptunomonas qingdaonensis, the TIR of the first transposon end sequence comprises a sequence of TGTCGTTT and the TIR of the second transposon end sequence comprises the reverse complement of TGTCGTTT (AAACGACA).

As described herein, a guide nucleic acid useful with the methods of the invention comprises at least one spacer having complementarity to a target site and at least two repeat sequences, wherein one of the two repeat sequences is linked to the 5′ end and the other of the two repeat sequences is linked to the 3′ end of the spacer.

In some embodiments, when CasTns system (e.g., the engineered CRISPR-Cas system and the engineered Tn7-like transposon system) is derived from Aliiglaciecola sp., the repeat sequence(s) may be any one of the nucleotide sequences of SEQ ID NOs: 54-58, in any combination, optionally, the repeat sequences may be SEQ ID NO:54 and/or SEQ ID NO:55. In some embodiments, when the system is derived from Vibrio sp., the repeat sequence(s) may be any one of the nucleotide sequences of SEQ ID NO: 154 and/or SEQ ID NO: 155. In some embodiments, when the system is derived from Halomonas titanicae, the repeat sequence(s) may be any one of the nucleotide sequences of SEQ ID NOs: 75-78, in any combination, optionally, the repeat sequences may be SEQ ID NO:75 and/or SEQ ID NO:76. In some embodiments, when the system is derived from Photobacterium aquae, the repeat sequence(s) may be any one of the nucleotide sequences of SEQ ID NOs: 134-137, in any combination, optionally, the repeat sequences may be SEQ ID NO:134 and/or SEQ ID NO: 135. In some embodiments, when the system is derived from Photobacterium iliopiscarium, the repeat sequence(s) may be any one of the nucleotide sequences of SEQ ID NOs: 15-19, in any combination, optionally, the repeat sequences may be SEQ ID NO:15 and/or SEQ ID NO: 16. In some embodiments, when the system is derived from Photobacterium piscicola, the repeat sequence(s) may be any one of the nucleotide sequences of SEQ ID NOs: 15, 16, 36 or 37, in any combination, optionally, the repeat sequences may be SEQ ID NO:15 and/or SEQ ID NO: 16. In some embodiments, when the system is derived from Psychromonas sp., the repeat sequence(s) may be any one of the nucleotide sequences of SEQ ID NOs: 172-174, in any combination, optionally, the repeat sequences may be SEQ ID NO: 172 and/or SEQ ID NO:173. In some embodiments, when the system is derived from Klebsiella oxytoca, the repeat sequence(s) may be any one of the nucleotide sequences of SEQ ID NOs: 115-117, in any combination, optionally, the repeat sequences may be SEQ ID NO:115 and/or SEQ ID NO:116. In some embodiments, when the system is derived from Colwellia polaris, the repeat sequence(s) may be any one of the nucleotide sequences of SEQ ID NO:191 and/or SEQ ID NO: 192. In some embodiments, when the system is derived from Neptunomonas qingdaonensis, the repeat sequence(s) may be any one of the nucleotide sequences of SEQ ID NOs: 95-100, in any combination, optionally, the repeat sequences may be SEQ ID NO:95, SEQ ID NO: 96, SEQ ID NO:97 and/or SEQ ID NO:98, in any combination.

In some embodiments, the target site is present in a nucleic acid present in the prokaryotic cell. In some embodiments, a target site having complementarity to a spacer of a guide nucleic acid is located immediately adjacent (3′) to a protospacer adjacent motif (PAM). The PAM requirements for the systems of this invention are more relaxed/flexible when compared to the PAM requirements of other CRISPR-Cas systems such as that for Streptococcus pyogenes Cas9, which typically requires a 5′-NGG-3′ PAM and near-complete spacer-target sequence complementary. Thus, in some embodiments, a target site having complementarity to a spacer of a guide nucleic acid may not be located adjacent to a PAM (i.e., the system for DNA integration does not require a PAM (e.g., a system derived from Psychromonas sp.; PAM requirement of 5′-NNNNN-3′, e.g., or any PAM)). In some embodiments, a system of the invention may function with highly variable PAM sequences or no PAM sequence. In some embodiments, the efficiency of integration of the donor DNA may be affected with less preferred PAMs.

Accordingly, a PAM useful with the systems and methods of the invention for RNA-guided DNA integration may vary with the CasTn system. In some embodiments, the PAM may comprise a nucleotide sequence of 5′-NNNNN-3′ that is immediately adjacent to and 5′ of the target site (protospacer). In some embodiments, a PAM useful with the systems and methods of the invention comprises a nucleotide sequence of 5′-NNNCN-3′ or 5′-NNNCC-3′ that is immediately adjacent to and 5′ of the target site.

Thus, for methods of this invention, when the CasTn system (e.g., an engineered CRISPR-Cas system, and an engineered Tn7-like transposon system as described herein) is derived from (a) Aliiglaciecola sp., optionally from Aliiglaciecola sp. strain M165, the PAM is 5′-NNNCN-3′, (b) Vibrio sp., optionally from Vibrio sp. strain EJY3, the PAM is 5′-NNNCC-3′, (c) H. titanicae, optionally from H. titanicae strain BH1, the PAM is 5′-NNNCN-3′, (d) P. aquae, optionally from P. aquae strain CGMCC 1.12159, the PAM is 5′-NNNCN-3′, (e) P. iliopiscarium, optionally from P. iliopiscarium strain ATCC 51760, the PAM is 5′-NNNCN-3′, (f) P. piscicola, optionally from P. piscicola sp. strain NCCB 100098, the PAM is 5′-NNNCN-3′, (g) Psychromonas sp., optionally from is Psychromonas sp. strain RZ5, the PAM is 5′-NNNNN-3′ (e.g., no PAM requirement), (h) K. oxytoca, optionally from K. oxytoca strain 67, the PAM is 5′-NNNCC-3′, (i) C. polaris, optionally from C. polaris strain MCCC 1C00015, the PAM is 5′-NNNCC-3′, and/or (j) N. qingdaonensis, optionally from N. qingdaonensis strain CGMCC 1.10971, the PAM is 5′-NNNCN-3′.

In some embodiments, when present, the PAM sequence may be oriented 5′-3′, resulting in the donor DNA being integrated downstream of the target site. Thus, for example, when the PAM sequence is in the 5′-3′ orientation, integration event results in 5′-[PAM][Target]-(about 25 bp to about 60 bp)-[Donor DNA in RL or LR orientation]-3′. In some embodiments, when the PAM sequence is oriented 3′-5′, then the donor DNA is integrated upstream of the target site. Thus, when the PAM sequence is in the 3′-5′ orientation, integration event results in 5′-[Donor DNA in RL or LR orientation]-(about 25 bp to about 60 bp)-[Target][PAM]-3′. In some embodiments, the cargo nucleic acid sequence may be integrated into the nucleic acid, 5′ to 3′, the first transposon end sequence, the cargo nucleic acid sequence, and the second transposon end sequence (e.g., in a right to left orientation). In some embodiments, the cargo nucleic acid sequence may be integrated into the nucleic acid, 5′ to 3′, the second transposon end sequence, the cargo nucleic acid sequence, and the first transposon end sequence (e.g., in a left to right orientation).

In some embodiments, a donor DNA may be introduced in a vector, such as, but not limited to, a plasmid. In some embodiments, a donor DNA may be linear, optionally wherein the donor DNA may be used in vitro or electroporated into a cell.

In some embodiments, the cargo nucleic acid sequence comprised in a donor DNA useful with a method of this invention may have a length of about 100 nucleotides to about 100,000 nucleotides (e.g., a length of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 100, 1500, 200, 2500, 3000, 3500, 4000, 4500, 5000, 6000, 7000, 8000, 9000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 nucleotides).

In some embodiments, the methods of the invention provide integration of the donor DNA at a location that is at about 25 consecutive nucleotides to about 60 consecutive nucleotides (i.e., integration distance) (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 consecutive nucleotides) from the target site, optionally about 35 consecutive nucleotides to about 55 consecutive nucleotides (e.g., about 35, 36, 37, 38, 39, 40, 41, 42, 43, 44, 45, 46, 47, 48, 49, 50, 51, 52, 53, 54, 55, 56, 57, 58, 59, or 60 consecutive nucleotides) from the target site, or about 45 consecutive nucleotides to about 55 consecutive nucleotides (e.g., about 45, 46, 47, 48, 49, 50, 51, 52, 53, 54, or 55 consecutive nucleotides) from the target site, or about 47, 48, 49, 50, 51, or 52 consecutive nucleotides from the target site. In some embodiments, the integration distance may be about 48, 49, or 50 consecutive nucleotides from the target site, optionally about 49 consecutive nucleotides from the target site.

In some embodiments, the CRISPR-Cas system and the Tn7-like transposon system may be comprised on the same vector, optionally wherein the CRISPR-Cas system, the Tn7-like transposon system and the guide nucleic acid are comprised on the same vector. In some embodiments, the CRISPR-Cas system, the Tn7-like transposon system, and the guide nucleic acid may be comprised on two or more different vectors in any combination, optionally, wherein the CRISPR-Cas system, the Tn7-like transposon system, the guide nucleic acid and the donor DNA may be on the same vector or may be on two or more different vectors in any combination.

In some embodiments, kits comprising one or more vectors encoding the CasIns systems and guide nucleic acids (CRISPR, crRNA) of this invention

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

The present invention is directed to reconstruction of type I-F3 systems for exogenous expression and RNA-directed DNA integration. In some embodiments, the type I-F3 systems may be expressed in the form of two plasmids: one plasmid (pEffector) encodes a CRISPR array and the Cascade and transposition machinery, while the other plasmid (pDonor) encodes a synthetic transposon comprised of “cargo” DNA flanked by the predicted terminal ends of the given transposon (donor DNA). A bacterial cell may be transformed with the pEffector and pDonor plasmids associated with a given type I-F3 system and targeting is directed to the site encoded by the spacer within the CRISPR array present in pEffector. Interactions between the TniQ-Cascade complex and the TnsABC machinery then facilitate integration of the pDonor transposon downstream of the target site. To test efficiency for integration for a given system at a specific target site, the appropriate pEffector and pDonor plasmids are used for transformation of E. coli, followed by incubation, DNA extraction, and integration efficiency calculation via qPCR. This manner of type I-F3 CRISPR-Cas system prediction, reconstruction, testing was performed for 10 type I-F3 transposon-associated CRISPR-Cas systems. The 10 systems are derived from a variety of species and possess diverse protein sequence composition, atypical CRISPR repeats, transposon ends (TnsB binding sites), and native transposon content and size. These ten type I-F3 CRISPR-Cas systems revealed diverse PAM preferences and varied integration distances.

### Example 1. CasTn Methods

Plasmid construction. The base pEffector expression vector was designed to express the CRISPR array and genes from a single T7 promoter and includes a lac operator and T7 terminator for gene expression and regulation. A multiple cloning site was included downstream of the lac operator for downstream cloning purposes. The base pEffector expression vector was created using the backbone of pSL0284 (Addgene #130635) and a gBlock containing the multiple cloning site. The pSL0284 backbone and multiple cloning site fragments were generated as PCR products using Q5 High-Fidelity 2X Master Mix (NEB #M0492) and assembled using NEBuilder® HiFi DNA Assembly Master Mix (NEB #E2621). Further pEffector derivatives were generated by restriction digestion of the pEffector plasmid followed by ligation of annealed oligos (IDT), containing either a CRISPR array or a single spacer sequence, with overhangs complementary to those generated by restriction digestion. pDonor constructs were made by amplifying a backbone from pSL0527 (Addgene #130634) and system-specific gBlocks comprised of a multiple cloning site flanked by the transposon ends. The pSL0527 backbone and system-specific fragments were generated as PCR products using Q5 High-Fidelity 2X Master Mix (NEB #M0492) and assembled using NEBuilder® HiFi DNA Assembly Master Mix (NEB #E2621). Further pDonor derivatives were generated by inserting additional DNA fragments within the multiple cloning site flanked by the transposon ends to generate pDonor constructs with transposons of approximately 1 kb or 10 kb in size.

Transposition. Transposition experiments were performed using chemically competent (NEB #C2527H) or electrocompetent (Millipore Sigma #CMC0016) E. coli BL21 (DE3) cells. For transposition experiments involving a pDonor construct with a donor transposon of approximately 1 kb in size, pEffector and pDonor constructs were co-transformed into chemically competent cells by using 50 ng of each plasmid. For transposition experiments involving pDonor constructs with a donor transposon of approximately 10 kb in size, 50 ng of each plasmid was co-transformed via electroporation into electrocompetent cells using the manufacturer's recommended settings. Cells were plated on double antibiotic (Spectinomycin 50 μg/ml, Carbenicillin 100 μg/ml) LB-agar plates with 0.1 mM IPTG (Thermo Scientific #R1171) and 40 μg/ml X-gal (Thermo Scientific #R0941). Incubations were performed at 30° C. for 30 hours or at 25° C. for 48 hours. Experiments pertaining to system orthogonality or involving pDonor constructs harboring a donor transposon approximately 10 kb in size were incubated at 25° C. Following incubation, sample lysates were generated similarly to previous work (Klompe et al. 2019. Nature 571:219-225). For each sample, colonies were scraped and resuspended in PBS, then OD600 measurements were taken and approximately 3.2×10⁸ cells were transferred to a 1.5 ml microtube and resuspended in PBS for a total volume of 200 μl for downstream processing. The resuspended cells were pelleted by centrifugation, supernatant was decanted, and the pellets were resuspended in 80 μl of nuclease-free H2O. The cells were then lysed on a dry block incubator at 95° C. for 10 min and cooled to room temperature. Cell debris pelleted by centrifugation and the lysate, containing genomic DNA, was diluted 20-fold in nuclease-free H2O in a fresh microtube for further use.

qPCR analysis. Three primer pairs were used for quantification of integration events within a sample. One primer pair served to generate a reference amplicon from the rssA gene in the E. coli BL21 (DE3) genome, while the other two primer pairs were designed to generate amplicons specific to integrated transposons in either the RL or LR orientation near the target site. The integration-specific primers were benchmarked against primers previously shown to accurately quantify integration activity for Tn6677 at the same loci (Klompe et al. 2019. Nature 571:219-225) and gDNA from clonal RL or LR integration events by individual systems was isolated, serially diluted, and tested to ensure adequate PCR efficiency between systems. Each qPCR reaction was 10 μl in total volume and consisted of: 5 μl of SsoAdvanced Universal SYBR® Green Supermix (Bio-Rad), 2 μl of 2.5 UM primer mix of two primers, 2 μl of a 20-fold dilution of sample lysate, and 1 μl of nuclease-free H2O. Reactions were loaded in 96-well qPCR plates (Thermo Scientific #AB3396) and run on a CFX Connect Real-Time PCR Detection System (Bio-Rad). The cycling conditions consisted of an initial denaturation step for 2.5 min at 98° C., followed by 40 amplification cycles of 10 sec at 98° C. and 1 min at 62° C., followed by melt curve production with 5 sec per 0.5° C. step for a range of 65-95° C. Samples were from three biological replicates, and three technical replicates were performed per sample, per primer pair. Integration efficiency was calculated using the 2^{Δq} method where, per sample, the Cq of the reference reaction is subtracted from the Cq value of either integration-specific reaction. The two calculated efficiencies are then combined to calculate the total integration efficiency. To determine RL:LR orientation bias, the calculated integration efficiency for the RL orientation was divided by the calculated integration efficiency for the LR orientation.

Plasmid library assay for PAM determination. The plasmid library with a randomized 5N region was created as previously described (Maxwell et al. 2018. Methods. 2018; 143:48-57). The plasmid library was co-transformed into electrocompetent E. coli BL21 (DE3) cells alongside pEffector and pDonor constructs by using 100 ng each of the plasmid library, pEffector, and pDonor constructs. Cells were plated on triple antibiotic (Spectinomycin 50 μg/ml, Carbenicillin 100 μg/ml, Kanamycin 50 μg/ml) LB-agar plates with 0.1 mM IPTG (Thermo Scientific #R1171) and incubated at 30° C. for 30 hours.

Following incubation, colonies were scraped, and plasmid DNA was extracted with a MidiPrep Kit (QIAGEN). PCR products were generated using extracted plasmid DNA as a template with Q5 High-Fidelity 2X Master Mix (NEB #M0492) and 0.5 UM of a primer pair designed to amplify across the 5N region of the plasmid library with a forward primer specific to the plasmid library and a reverse primer specific to transposon integration in the RL orientation. PCR thermocycling involved an annealing temperature of 67° C. and 25 cycles and PCR products were visualized on 1% agarose gel using SYBR® Safe (Thermo Scientific). PCR products were isolated and purified using Monarch DNA Gel Extraction and PCR & DNA Cleanup kits (NEB), and NGS libraries were prepared and sequenced on an Illumina platform for 2×250 bp paired-end reads (GENEWIZ® Amplicon-EZ). The resulting sequence data was analyzed using SeqKit (Shen et al. 2016. PLOS ONE 11(10):e0163962). Reads were filtered based on the presence of no mismatches, when compared to the base plasmid library, within the 20 bp upstream and 32 bp downstream (spacer sequence) from the 5N region. The 5N region was then extracted from these filtered reads and PAM frequencies were calculated and normalized based on PAM frequencies calculated for the base plasmid library: ((Count of PAM X)/(Total PAM count))/((Frequency of PAM X in base library)/(Expected frequency of PAM X in base library)). The top 10% of normalized PAM frequencies for each system were used as an input for making WebLogos (Crooks et al. 2004. Genome Research 14:1188-1190).

Integration distance analyses. For lacZ integration experiments, PCR products were generated using diluted sample lysates, Q5 High-Fidelity 2X Master Mix (NEB #M0492), 0.5 UM of primers specific to RL orientation transposon integration events. Thermocycling conditions involved an annealing temperature of 68° C. and 20 cycles and PCR products were visualized on 1% agarose gel using SYBR® Safe (Thermo Scientific). PCR products were isolated and purified using Monarch DNA Gel Extraction and PCR & DNA Cleanup kits (NEB), and NGS libraries were prepared and sequenced on an Illumina platform for 2×250 bp paired-end reads (GENEWIZ® Amplicon-EZ). The resulting sequence data was analyzed using SeqKit (v2.2.0, Shen et al. 2016. PLOS ONE 11(10): e0163962). Reads were filtered based on 12 perfectly matching bases specific to the right transposon end, followed by extracting the 20 bases immediately upstream of the 5′ end of the integrated right transposon end. After filtering for sequences only 20 bp in length, the remaining sequences were mapped back to lacZ using Bowtie (Langmead et al. 2009. Genome Biol. 10: R25) with no mismatches allowed. Positional data corresponding to the integration distance of the transposon from the target site was extracted from the resulting .sam files and integration distances comprising at least 1% of the data set were plotted.

### Example 2. System Characterization of CasTns

To understand the mechanistic diversity of type I-F3 CasTns, we selected ten diverse and previously uncharacterized systems from Gammaproteobacteria for functional characterization. Features necessary for transposition were predicted in silico, namely the genes tnsA, tnsB, tnsC, tniQ, cas8, cas7, and cas6, the CRISPR array and associated repeat sequences, and the transposon ends that define the transposon boundaries due to the presence of a terminal inverted repeat (TIR) and TnsB binding sites at each end. Each CasTn system was reconstituted as a two-plasmid system for testing in E. coli BL21 (DE3): a pEffector plasmid expresses the CRISPR array and all genes essential for transposition, while a pDonor plasmid encodes a donor transposon of approximately 1 kb in length, including transposon ends (referred to as “right” and “left” ends), unless noted otherwise. Transposition assays in E. coli BL21 (DE3) involved measuring transposition efficiency via qPCR by quantifying the amplification of a reference amplicon alongside amplicons specific to right-to-left (RL) and left-to-right (LR) transposition events at a given target site.

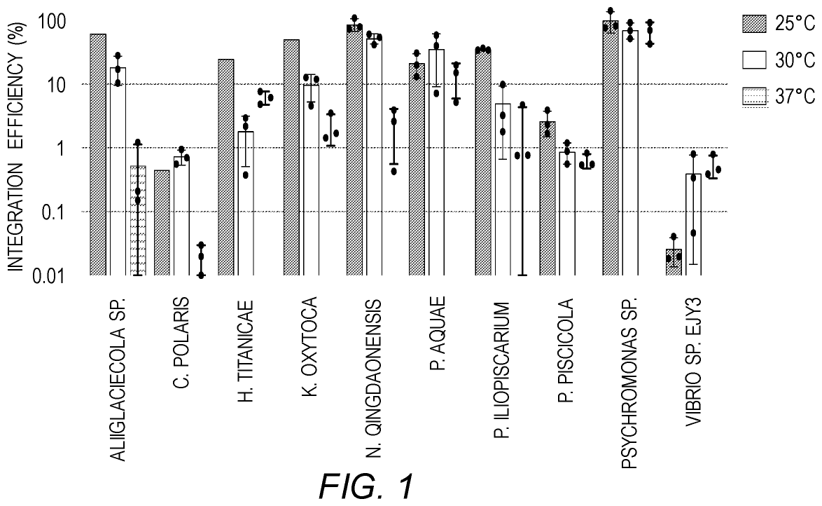

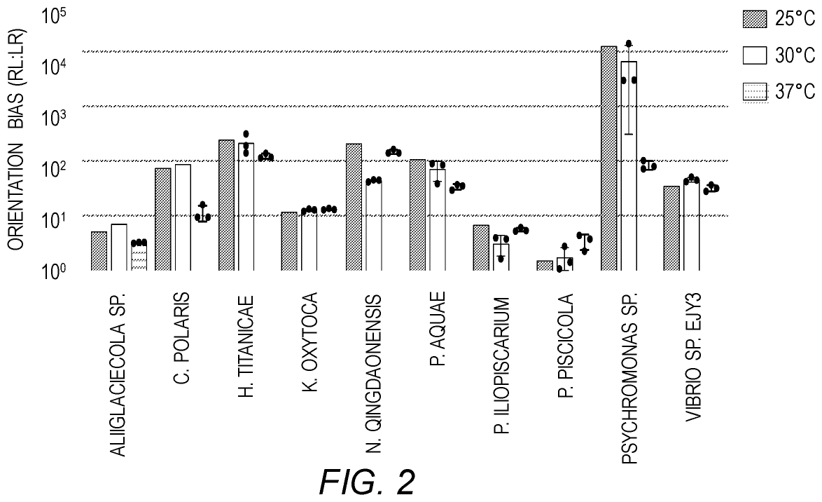

Initial characterization of each CasTn involved targeting an E. coli BL21 (DE3) lacZ locus which possesses 5′-CC-3′ PAM canonical for type I-F CRISPR-Cas systems. We tested our ten novel CasTns at this target site at 25° C., 30° C. and 37° C. incubation temperatures (FIG. 1). Interestingly, in these conditions, we found that transposition (or integration) efficiencies for these CasTns ranged from approximately 0.1% to 100%. Overall, transposition efficiencies generally increased or remained comparable, when the incubation temperature was reduced from 37° C. or 30° C. to 25° C. The integration efficiency of the CasTn originating from Vibrio sp. EJY3 appears reduced at 25° C., but we note that is difference is not significant and is near our defined limit of detection of 0.01% integration efficiency. As the total integration efficiency is the result of combining the quantification of right-to-left and left-to-right orientation transposition events, we can compare the quantification of each orientation to determine transposon orientation bias (FIG. 2). We found that, at a single target site with a 5′-CC-3′ PAM, these systems possess orientation biases universally skewed toward the RL orientation, with biases ranging from slightly greater than 1:1 (RL:LR orientation) to greater than 103:1, and that these biases remained generally consistent regardless of incubation temperature. Overall, we show that our ten novel CasIns are capable of RNA-guided DNA transposition, with transposition preference universally biased toward the RL orientation, at a single target site with a 5′-CC-3′ PAM.

### Example 3. Determination of PAM Preference

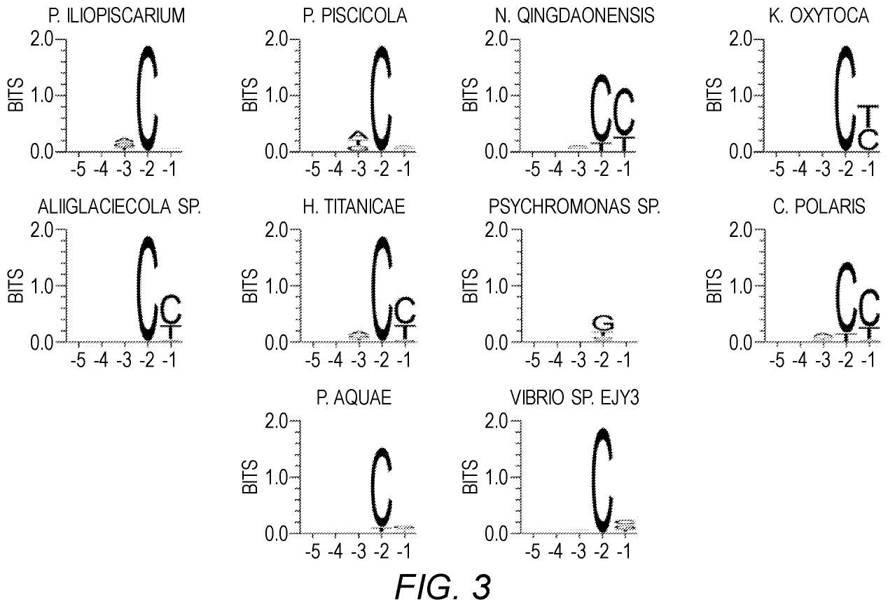

As a wide range of integration efficiency was observed across the CasTns for a target site with a 5′-CC-3′ PAM, we wanted to determine if PAM preference varies between our CasTns and if those variations in preference have an effect on integration activity. To determine PAM preference for each CasTn, we conducted a plasmid library-based assay in which the pEffector plasmid for a given system bears a spacer to direct integration within the plasmid library at a target site with a randomized five-nucleotide (5N) region upstream (5′ end) of the target site. For each CasTn, PCR was used to generate an amplicon specific to RL orientation integration events within the plasmid library, deep sequenced the amplicon, and analyzed the nucleotide frequencies within the 5N variable region to determine PAM preference (FIG. 3). For most systems, we found a strong preference for a C nucleotide in the −2 position, and we note that the −2 position is repeatedly the position displaying the most significant degree of preference within these PAM profiles. We also find that the canonical 5′-CC-3′ PAM (for positions −2 and −1) preference is only present within a few systems, and that the preference for a C in the −1 position is lesser than the preference for a C in the −2 position. Slight preferences for within the −3 position can be seen in some systems, such as those derived from P. piscicola and H. titanicae. A striking result from this assay was the discovery that the Psychromonas sp. CasTn possesses near PAM-less functionality, showing only a very slight preference in the −2 position for a G nucleotide, which may be attributed to the motif frequency within the base plasmid library.

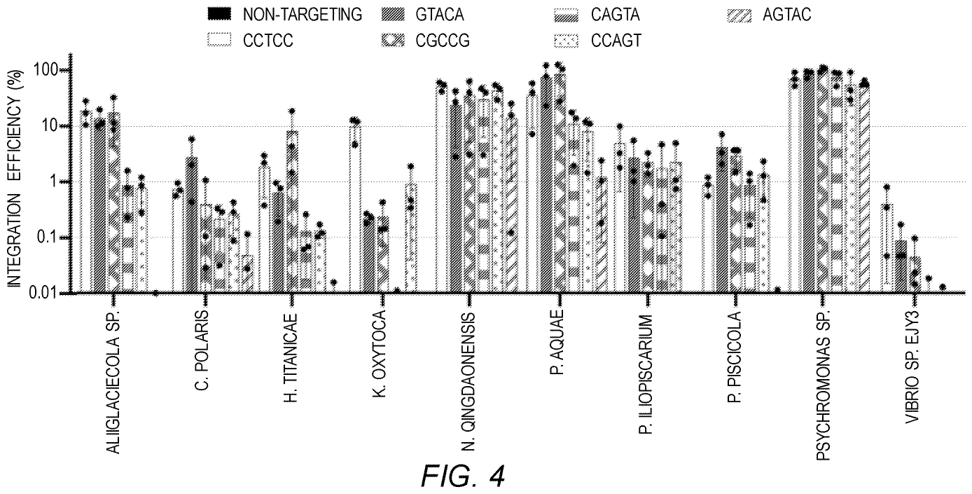

To validate these findings within the plasmid library assay, five additional target sites were selected within lacZ, each possessing a distinct PAM sequence (FIG. 4). Two of the additional targets retain a C in the −2 position of the PAM, while the remaining three targets instead have a T, G, or A at the −2 position. This set of target sites allowed us to evaluate the extent of preferences toward a canonical 5′-CC-3′ PAM or the less restrictive 5′-CN-3′ PAM that we found to be more applicable to most of our CasTns. We discovered that the differences in PAM flexibility initially seen in our plasmid library assay do translate to differences in integration efficiency when targeting different genomic loci. We found that only one system, the K. oxytoca CasTn, relies heavily on a C in the −1 position, as it integrates most effectively at the target site with a 5′-CC-3′ PAM and shows a significant drop in efficiency when the −2 position C is retained but the −1 position C is replaced with an A or a G. Additionally, integration with the K. oxytoca CasTn was nearly or entirely undetectable when the −2 position in the PAM contained a T or A. Other CasTns, such as those derived from Aliiglaciecola sp. and H. titanicae, have similar integration activity at all three 5′-CN-3′ PAM targets, but efficiency declined when the −2 C of the PAM was replaced with a T, G, or A. Interestingly, most of these systems show near or entirely undetectable integration activity for the target site possessing a 5′-AC-3′ PAM. Without wishing to be limited by any particular theory, this bias against a 5′-AC-3′ PAM may be to mitigate self-targeting activity within the CRISPR array due to the 5′-AN-3′ motif present at the 3′ end of the CRISPR repeats associated with these systems.

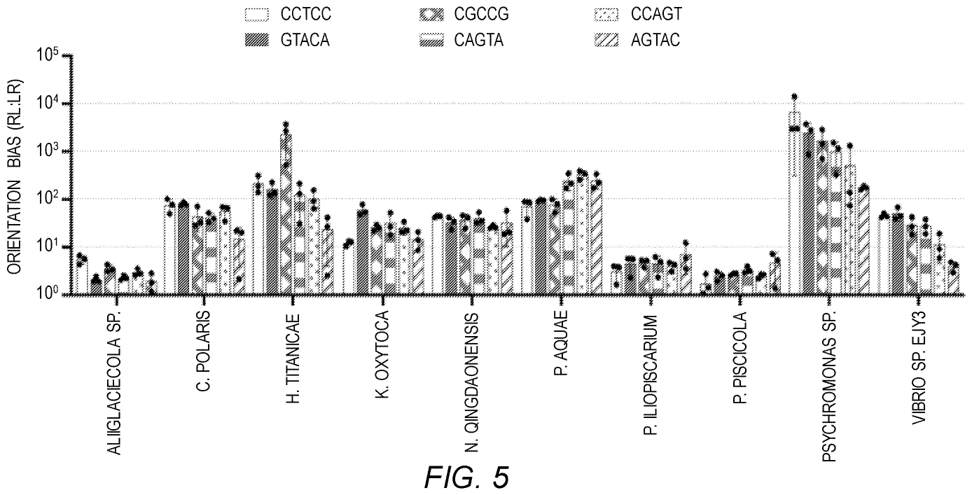

These experiments further validated the plasmid library assay results that demonstrated the Psychromonas sp. CasTn to be a near PAM-free system. Regardless of the target site, the Psychromonas sp. CasTn exhibited highly efficient integration activity. As noted above, we were able to obtain transposition orientation bias data from the sum integration efficiency data. For the additional integration at different targets within lacZ, we found that orientation bias is generally consistent between targets for any given system (FIG. 5). Our original bias data is supported here, as we found a range of bias from approximately 1:1-10:1 to greater than 103:1 for RL:LR orientation events.

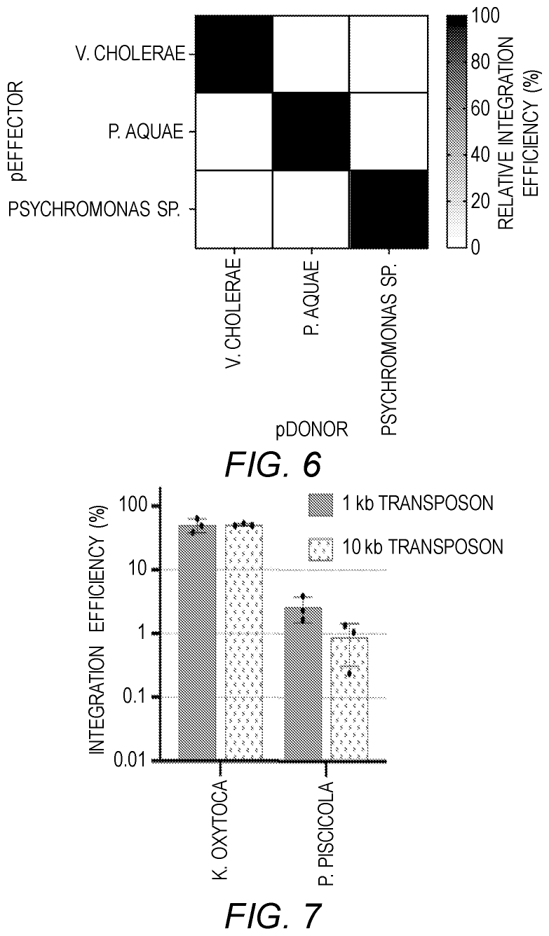

We determined the compatibility groups for two of our novel CasIns and performed orthogonality testing by using all nine pEffector-pDonor combinations for the three selected systems, including the V. cholerae Tn6677 system (FIG. 6). Integration activity was detected only when testing plasmid combinations involving pEffector-pDonor combinations where both plasmids were associated with the same CasTn, indicating that the CasTns from V. cholerae, P. aquae, and Psychromonas sp. are orthogonal and can be used for sequential integration events at the same or distance target sites to, respectively, circumvent issues arising from target site immunity and transposon remobilization.

### Example 4. Effect of Transposon Size

To determine the effect of transposon size (i.e., cargo DNA, donor DNA) on integration efficiency, we selected the CasTns derived from K. oxytoca, which possesses a natural TnsAB fusion, and P. piscicola, which had relatively low integration efficiencies in our initial tests. These were tested with pDonor plasmids containing a donor DNA approximately 10 kb in size. The same lacZ target site possessing a 5′-CC-3′ PAM was used as in our initial tests, and incubation occurred at 25° C. to alleviate potential cytotoxicity and maximize integration efficiency. We found that integration efficiencies were similar when comparing integration involving donor DNAs of approximately 1 kb and 10 kb in size (FIG. 7).

### Example 5. Integration Distance

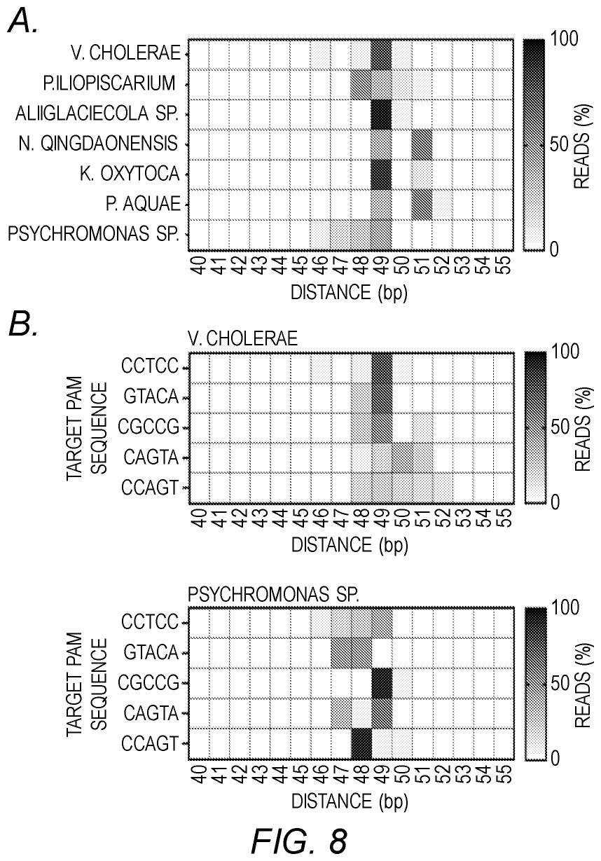

Integration distance was determined, where integration distance is defined as the number of nucleotides between the end of the target site (spacer) sequence and the beginning of the transposon end sequence. We generated PCR products specific to RL orientation integration events, deep sequenced the amplicons, and mapped the sequences to lacZ to determine integration distance (FIG. 8). For a single target site with a 5′-CC-3′ PAM, the integration distance was determined for seven systems, with data from V. cholerae Tn6677 shown for reference (FIG. 8, panel A). We found that the integration distance for each system was frequently about 49 bp, but the relative distance patterns vary among systems. To determine if these patterns would remain consistent across sites for multiple CasTns, we determined the integration distance patterns for V. cholerae and Psychromonas sp. at multiple target sites (FIG. 8, panel B). These studies too showed that the integration distance pattern remains about 49 bp for both systems, however the integration distance is somewhat unpredictable when looking at multiple targets for a single system or when comparing the two systems at a single target. However, we do note that the integration distance for V. cholerae tended to skew toward >49 bp for these sites, while the integration distance of the Psychromonas sp. CasTn tended to be <49 bp. Overall, we found that no system has a single, defined integration distance but that integration distance is typically within about a 5 bp window for a single target site. We also note that the PCR-based method used for detection may have missed distant integration events that occur outside of the range defined by the PCR primers used.

### Example 6. Self-Targeting Spacers

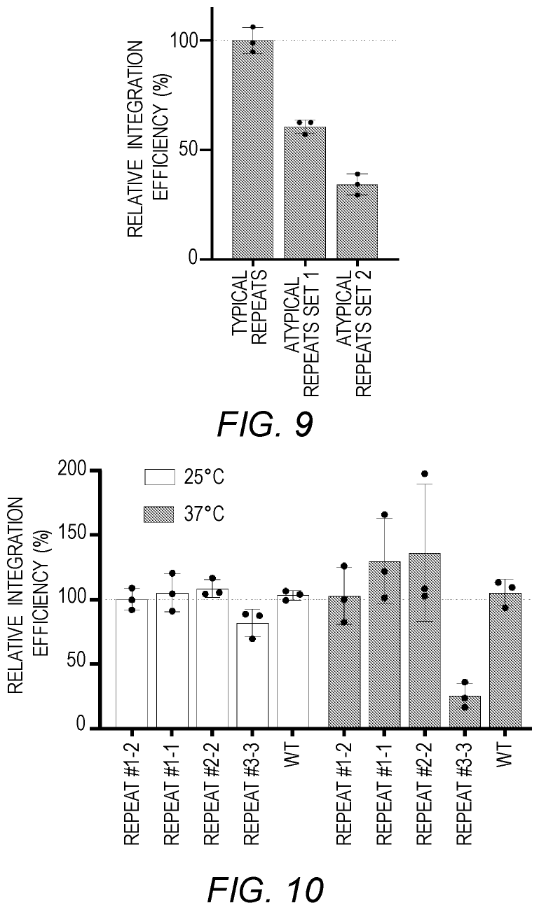

Alignment of CRISPR repeats from the N. qingdaonensis system revealed that the atypical repeats associated with the self-targeting spacers are distinct from one another in terms of sequence and putative crRNA structure. For instance, one atypical repeat (SEQ ID NO: 96) from N. qingdaonensis contains a shifted stem loop structure with a shorter 4-nt loop, while the other atypical repeats (SEQ ID NO:97 and SEQ ID NO:98) have highly disrupted putative stem-loop structures. Given the presence of multiple self-targeting spacers and their associated atypical repeats in N. qingdaonensis, we wanted to determine if one set of repeats enables more efficient RNA-guided DNA integration. Intriguingly, we discovered that the N. qingdaonensis system uses the first two associated repeats of its native CRISPR array, which flank a non-self-targeting spacer and possess expected crRNA stem loop structures, for the most efficient integration activity at a lacZ target site (FIG. 9).

### Example 7. Characterization of the Psychromonas sp. System

Similar to the analysis presented in Example 2, the Psychromonas sp. system was tested at two different temperatures, 30° C. to 25° C., for RNA-guided DNA transposition at the 5′-CCTCC-3′ PAM lacZ target site with different combinations of native CRISPR repeats. The results of this analysis (FIG. 10) indicate that when utilizing a CRISPR array, consisting of two repeats flanking one spacer, the use of two of the same repeat from Psychromonas sp. (“repeat #3”, SEQ ID NO: 173) results in lower rates of integration across both tested incubation temperatures when compared to use of other combinations of CRISPR repeats native to Psychromonas sp.

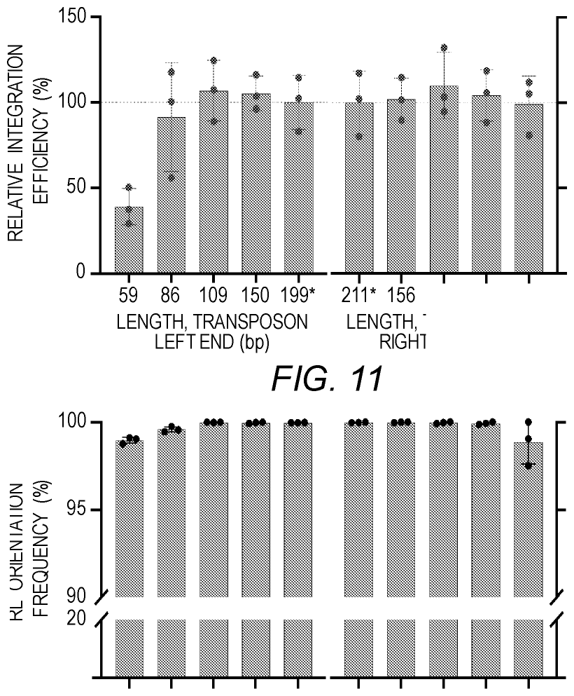

The relative integration efficiency for the Psychromonas sp. system was tested at 25° C. for RNA-guided DNA transposition at the 5′-CCTCC-3′ PAM lacZ target site with different lengths of the associated left and right transposon ends. The results of this analysis (FIG. 11) indicate that, at an incubation temperature of 25° C., integration efficiency remained comparable to the originally tested left transposon end length (199 bp) at lengths of 150 and 109 bp, while integration efficiency decreased at left transposon end lengths of 86 and 59 bp. All truncations of the right transposon end resulted in comparable integration efficiencies relative to the originally tested right transposon end length of 211 bp.

It was noted that the RL:LR orientation bias of this system heavily favors the RL orientation; we were unable to detect the LR orientation in some instances, meaning orientation bias (RL/LR) data was unable to be generated. Thus, RL orientation frequency or the frequency with which the transposon was integrated in the right-to-left orientation was determined. The results of this analysis (FIG. 12) indicate that slight decreases in RL orientation frequency were observed when the left transposon end was truncated to a length of 59 bp and when the right transposon end was truncated to a length of 50 bp. However, all observed RL orientation frequencies were above 95%, demonstrating the heavy bias of the Psychromonas sp. system for integration of the transposon in the right to left (RL) orientation.

### Example 8. Expression of Psychromonas sp. System in Maize

Without protein sequence modifications, each of the coding sequences of Cas6, Cas7, Cas8, TniQ, TnsA, TnsB, and TnsC isolated from Psychromonas sp. were optimized for expression in plants and cloned into a pUC57-based plant expression vector (Table 2). Gene expression was under the control of the maize Ubi1 promoter and the protein coding sequences were fused in-frame with an SV40 nuclear localization signal (NLS) at the N-terminus and ZsGreen (a human codon-optimized variant of the green fluorescent protein isolated from reef coral (Zoanthus sp.) at the C-terminus In addition, a Cas8 construct was generated with the NLS fused to the C-terminus and ZsGreen fused to the N-terminus (GFP_Cas6_pUC57)

TABLE 2
Expression constructs encoding components
of the Psychromonas sp. system

| | Optimized | | |
|---|---|---|---|
| | coding | | Expression |
| Psychromonas | sequence | Expression vector | Vector |
| Component | SEQ ID NO: | Designation | SEQ ID NO: |
| Cas6 | 213 | Cas6_GFP_pUC57 | 220 |
| Cas7 | 214 | Cas7_GFP_pUC57 | 221 |
| Cas8 | 215 | Cas8_GFP_pUC57 | 222 |
| | | GFP_Cas6_pUC57 | 223 |
| TiQ | 216 | TniQ_GFP_pUC57 | 224 |
| TnsA | 217 | TnsA_GFP_pUC57 | 225 |
| TnsB | 218 | TnsB_GFP_pUC57 | 226 |
| TnsC | 219 | TnsC_GFP_pUC57 | 227 |

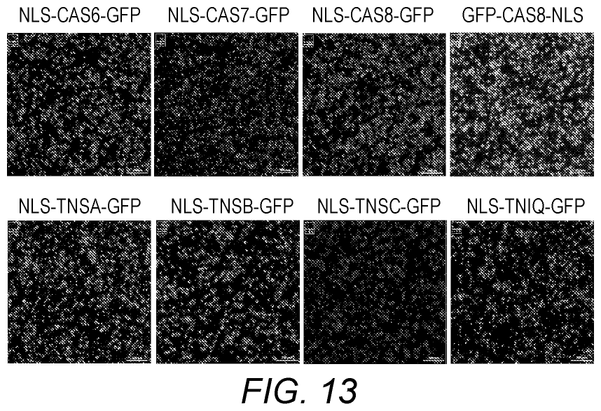

The expression vectors were introduced into maize protoplasts and expression was assessed by fluorescence. The results indicated that each of the components of the Psychromonas sp. system could be expressed in maize cells (FIG. 13). A difference in the fluorescence intensity was observed between the two Cas8 constructs, which differed by the location of the NLS and ZsGreen locations (i.e., N-terminus vs. C-terminus). To determine whether the difference was an artifact of the ZsGreen location or an actual difference in expression. Cas8 constructs without ZsGreen were generated. Expression vector constructs included the SV40 NLS at the N-terminus of Cas8 (n-NLS-Cas8; SEQ ID NO:228) or the SV40 NLS at the C-terminus of Cas8 (c-NLS-Cas8; SEQ ID NO:229). Expression of both constructs was under the control of the Ubi promoter. The constructs were introduced into maize protoplasts and expression was assessed by mass spectrometry. Expression data were normalized against a transfected red fluorescent protein control (a set amount of RFP control plasmid was added to each well). This analysis indicated that both Cas8 constructs were expressed at approximately the same level (Table 3).

TABLE 3
Percent relative expression of Cas8 constructs

| | Normalization Method | | | | |
|---|---|---|---|---|---|
| Construct | Peptides | Log2 | Median | Mean | VSN |
| c-NLS-Cas8 | 11 | 19.48041 | 19.49433 | 19.50603 | 19.5068 |
| Run 1 | | | | | |
| c-NLS-Cas8 | 8 | 18.89648 | 18.87883 | 18.89234 | 18.85072 |
| Run 2 | | | | | |
| Average* | | 19.18844 | 19.18658 | 19.19918 | 19.17876 |
| n-NLS-Cas8 | 10 | 19.21234 | 19.19598 | 19.19475 | 19.16613 |
| Run 1 | | | | | |
| n-NLS-Cas8 | 10 | 19.60249 | 19.60447 | 19.60953 | 19.61078 |
| Run 2 | | | | | |
| Average* | | 19.40742 | 19.40022 | 19.40214 | 19.38845 |

*The average normalized values are based upon the two independent runs above each average.

To further enhance the expression of TnsC and Cas7, a series of coding sequences were codon optimized for expression in maize (Table 4).

TABLE 4
Codon optimization of TnsC and Cas7 for expression in maize

| | Construct, | | Seq | | | | Expression |
|---|---|---|---|---|---|---|---|
| | SEQ ID | Construct | Coverage | Unique | RFP | Total | Vector, |
| Construct | NO: | Score | (%) | Pep | Score | Peptides | SEQ ID NO: |
| Cas7-n1 | 230 | 63 | 29 | 8 | 120 | 15.8K | 238 |
| Cas7-n2 | 231 | 42 | 35 | 10 | 48 | 16.2K | 239 |
| Cas7-n3 | 232 | 23 | 35 | 11 | 60 | 16.0K | 240 |
| Cas7-n4 | 233 | 102 | 40 | 11 | 112 | 15.4K | 241 |
| TnsC-n1 | 234 | 129 | 57 | 17 | 120 | 15.8K | 242 |
| TnsC-n2 | 235 | 90.9 | 57 | 18 | 48 | 16.2K | 243 |
| TnsC-n3 | 236 | 36 | 36 | 11 | 60 | 16.0K | 244 |
| TnsC-n4 | 237 | 76 | 36 | 11 | 112 | 15.4K | 245 |

The maize codon optimized nucleic acid sequences encoding TnsC and Cas7 were inserted into a maize expression vector harboring the maize Ubi1 promoter and SV40 NLS, introduced into maize protoplasts, and expression assessed by mass spectrometry. The results of this analysis indicated that all codon optimized nucleic acid sequences encoding TnsC and 5 Cas7 were expressed in maize protoplasts (Table 5).

TABLE 5
Percent relative expression of codon
optimized constructs of TnsC and Cas7

Normalization Method

| Construct | Log2 | Median | Mean | VSN |
|---|---|---|---|---|
| Cas7-n1 | 94.96 | 95.02 | 94.88 | 95.00 |
| Cas7-n2 | 95.96 | 95.76 | 95.66 | 95.67 |
| Cas7-n3 | 98.53 | 98.64 | 98.58 | 98.60 |
| Cas7-n4 | 100 | 100 | 100 | 100 |
| TnsC-n1 | 97.85 | 98.21 | 98.18 | 98.16 |
| TnsC-n2 | 100 | 100 | 100 | 100 |
| TnsC-n3 | 93.01 | 93.42 | 93.47 | 93.36 |
| TnsC-n4 | 93.42 | 93.85 | 93.95 | 93.83 |

1. A system for RNA-guided DNA integration, the system comprising:
(A) one or more vectors heterologous to Aliiglaciecola sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Aliiglaciecola sp.;
(B) one or more vectors heterologous to Vibrio sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (Tns B), (c) Transposon 7 protein C (TnsC), and (d) transposition of integron protein Q (TniQ), wherein the engineered Tn7-like transposon system is derived from Vibrio sp.;
(C) one or more vectors heterologous to Halomonas titanicae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from H. titanicae;
(D) one or more vectors heterologous to Photobacterium aquae encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. aquae;
(E) one or more vectors heterologous to Photobacterium iliopiscarium encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. iliopiscarium;
(F) one or more vectors heterologous to Photobacterium piscicola encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from P. piscicola;
(G) one or more vectors heterologous to Psychromonas sp. encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from Psychromonas sp.;
(H) one or more vectors heterologous to Klebsiella oxytoca encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein AB (TnsAB), (b) Transposon 7 protein C (TnsC), and (c) transposition of integron protein Q (TniQ), wherein the engineered Tn7-like transposon system is derived from K. oxytoca;
(I) one or more vectors heterologous to Colwellia polaris encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from C. polaris; or
(J) one or more vectors heterologous to Neptunomonas qingdaonensis encoding: (i) an engineered Clustered Regularly Interspaced Short Palindromic Repeats (CRISPR)-CRISPR associated (Cas) (CRISPR-Cas) system, the engineered CRISPR-Cas system comprising: Cas6, Cas7 and Cas8; and (ii) an engineered transposon 7-like (Tn7-like) transposon system, the engineered Tn7-like transposon system comprising: (a) Transposon 7 protein A (TnsA), (b) Transposon 7 protein B (TnsB), (c) Transposon 7 protein C (TnsC), and (d) transposition of integron protein Q (TniQ), wherein the engineered Tn7-like transposon system is derived from N. qingdaonensis.

2. The system of claim 1, wherein: (A) Aliiglaciecola sp. is Aliiglaciecola sp. strain M165; (B) Vibrio sp. is Vibrio sp. strain EJY3; (C) H. titanicae is H. titanicae strain BH1; (D) P. aquae is P. aquae strain CGMCC 1.12159; (E) P. iliopiscarium is P. iliopiscarium strain ATCC 51760; (F) P. piscicola is P. piscicola sp. strain NCCB 100098; (G) Psychromonas sp. is Psychromonas sp. strain RZ5; (H) K. oxytoca is K. oxytoca strain 67; (I) C. polaris is C. polaris strain MCCC 1C00015; and/or (J) N. qingdaonensis is N. qingdaonensis strain CGMCC 1.10971.

3. The system of claim 1, wherein for:
(A) Aliiglaciecola sp., Cas6 is encoded by a nucleotide sequence having at least 90% sequence identity to SEQ ID NO:43 and/or comprises an amino acid sequence having at least 90% sequence identity to SEQ ID NO:50, Cas7 is encoded by a nucleotide sequence having at least 90% sequence identity to SEQ ID NO:42 and/or comprises an amino acid sequence having at least 90% sequence identity to SEQ ID NO:49, Cas8 is encoded by a nucleotide sequence having at least 90% sequence identity to SEQ ID NO:41 and/or comprises an amino acid sequence having at least 90% sequence identity to SEQ ID NO:48, TnsA is encoded by a nucleotide sequence having at least 90% sequence identity to SEQ ID NO:44 and/or comprises an amino acid sequence having at least 90% sequence identity to SEQ ID NO:51, TnsB is encoded by a nucleotide sequence having at least 90% sequence identity to SEQ ID NO:45 and/or comprises an amino acid sequence having at least 90% sequence identity to SEQ ID NO: 52, TnsC is encoded by a nucleotide sequence having at least 90% sequence identity to SEQ ID NO: 46 and/or comprises an amino acid sequence having at least 90% sequence identity to SEQ ID NO:53, and/or TniQ is encoded by a nucleotide sequence having at least 90% sequence identity to SEQ ID NO:40 and/or comprises an amino acid sequence having at least 90% sequence identity to SEQ ID NO:47;
(B) Vibrio sp., Cas6 is encoded by a nucleotide sequence having at least 90% sequence identity to SEQ ID NO:143 and/or comprises an amino acid sequence having at least 90% sequence identity to SEQ ID NO:150, Cas7 is encoded by a nucleotide sequence having at least 90% sequence identity to SEQ ID NO: 142 and/or comprises an amino acid sequence having at least 90% sequence identity to SEQ ID NO:149, Cas8 is encoded by a nucleotide sequence having at least 90% sequence identity to SEQ ID NO:141 and/or comprises an amino acid sequence having at least 90% sequence identity to SEQ ID NO:148, TnsA is encoded by a nucleotide sequence having at least 90% sequence identity to SEQ ID NO:144 and/or comprises an amino acid sequence having at least 90% sequence identity to SEQ ID NO:151, TnsB is encoded by a nucleotide sequence having at least 90% sequence identity to SEQ ID NO:145 and/or comprises an amino acid sequence having at least 90% sequence identity to SEQ ID NO: 152, TnsC is encoded by a nucleotide sequence having at least 90% sequence identity to SEQ ID NO:146 and/or comprises an amino acid sequence having at least 90% sequence identity to SEQ ID NO: 153, and/or TniQ is encoded by a nucleotide sequence having at least 90% sequence identity to SEQ ID NO: 140 and/or comprises an amino acid sequence having at least 90% sequence identity to SEQ ID NO:147;
(C) H. titanicae, Cas6 is encoded by a nucleotide sequence having at least 90% sequence identity to SEQ ID NO:64 and/or comprises an amino acid sequence having at least 90% sequence identity to SEQ ID NO:71, Cas7 is encoded by a nucleotide sequence having at least 90% sequence identity to SEQ ID NO:63 and/or comprises an amino acid sequence having at least 90% sequence identity to SEQ ID NO:70, Cas8 is encoded by a nucleotide sequence having at least 90% sequence identity to SEQ ID NO:62 and/or comprises an amino acid sequence having at least 90% sequence identity to SEQ ID NO:69, TnsA is encoded by a nucleotide sequence having at least 90% sequence identity to SEQ ID NO:65 and/or comprises an amino acid sequence having at least 90% sequence identity to SEQ ID NO:72, TnsB is encoded by a nucleotide sequence having at least 90% sequence identity to SEQ ID NO:66 and/or comprises an amino acid sequence having at least 90% sequence identity to SEQ ID NO: 73, TnsC is encoded by a nucleotide sequence having at least 90% sequence identity to SEQ ID NO: 67 and/or comprises an amino acid sequence having at least 90% sequence identity to SEQ ID NO:74, and/or TniQ is encoded by a nucleotide sequence having at least 90% sequence identity to SEQ ID NO:61 and/or comprises an amino acid sequence having at least 90% sequence identity to SEQ ID NO:68;
(D) P. aquae, Cas6 is encoded by a nucleotide sequence having at least 90% sequence identity to SEQ ID NO:123 and/or comprises an amino acid sequence having at least 90% sequence identity to SEQ ID NO:130, Cas7 is encoded by a nucleotide sequence having at least 90% sequence identity to SEQ ID NO:122 and/or comprises an amino acid sequence having at least 90% sequence identity to SEQ ID NO:129, Cas8 is encoded by a nucleotide sequence having at least 90% sequence identity to SEQ ID NO: 121 and/or comprises an amino acid sequence having at least 90% sequence identity to SEQ ID NO:128, TnsA is encoded by a nucleotide sequence having at least 90% sequence identity to SEQ ID NO:124 and/or comprises an amino acid sequence having at least 90% sequence identity to SEQ ID NO:131, TnsB is encoded by a nucleotide sequence having at least 90% sequence identity to SEQ ID NO: 125 and/or comprises an amino acid sequence having at least 90% sequence identity to SEQ ID NO: 132, TnsC is encoded by a nucleotide sequence having at least 90% sequence identity to SEQ ID NO:126 and/or comprises an amino acid sequence having at least 90% sequence identity to SEQ ID NO:133, and/or TniQ is encoded by a nucleotide sequence having at least 90% sequence identity to SEQ ID NO:120 and/or comprises an amino acid sequence having at least 90% sequence identity to SEQ ID NO:127;
(E) P. iliopiscarium, Cas6 is encoded by a nucleotide sequence having at least 90% sequence identity to SEQ ID NO:4 and/or comprises an amino acid sequence having at least 90% sequence identity to SEQ ID NO:11, Cas7 is encoded by a nucleotide sequence having at least 90% sequence identity to SEQ ID NO:3 and/or comprises an amino acid sequence having at least 90% sequence identity to SEQ ID NO:10, Cas8 is encoded by a nucleotide sequence having at least 90% sequence identity to SEQ ID NO:2 and/or comprises an amino acid sequence having at least 90% sequence identity to SEQ ID NO:9, TnsA is encoded by a nucleotide sequence having at least 90% sequence identity to SEQ ID NO:5 and/or comprises an amino acid sequence having at least 90% sequence identity to SEQ ID NO:12, TnsB is encoded by a nucleotide sequence having at least 90% sequence identity to SEQ ID NO:6 and/or comprises an amino acid sequence having at least 90% sequence identity to SEQ ID NO:13, TnsC is encoded by a nucleotide sequence having at least 90% sequence identity to SEQ ID NO: 7 and/or comprises an amino acid sequence having at least 90% sequence identity to SEQ ID NO: 14, and/or TniQ is encoded by a nucleotide sequence having at least 90% sequence identity to SEQ ID NO: 1 and/or comprises an amino acid sequence having at least 90% sequence identity to SEQ ID NO:8;
(F) P. piscicola, Cas6 is encoded by a nucleotide sequence having at least 90% sequence identity to SEQ ID NO:25 and/or comprises an amino acid sequence having at least 90% sequence identity to SEQ ID NO:32, Cas7 is encoded by a nucleotide sequence having at least 90% sequence identity to SEQ ID NO:24 and/or comprises an amino acid sequence having at least 90% sequence identity to SEQ ID NO:31, Cas8 is encoded by a nucleotide sequence having at least 90% sequence identity to SEQ ID NO:23 and/or comprises an amino acid sequence having at least 90% sequence identity to SEQ ID NO:30, TnsA is encoded by a nucleotide sequence having at least 90% sequence identity to SEQ ID NO:26 and/or comprises an amino acid sequence having at least 90% sequence identity to SEQ ID NO:33, TnsB is encoded by a nucleotide sequence having at least 90% sequence identity to SEQ ID NO:27 and/or comprises an amino acid sequence having at least 90% sequence identity to SEQ ID NO: 34, TnsC is encoded by a nucleotide sequence having at least 90% sequence identity to SEQ ID NO: 28 and/or comprises an amino acid sequence having at least 90% sequence identity to SEQ ID NO:35, and/or TniQ is encoded by a nucleotide sequence having at least 90% sequence identity to SEQ ID NO:22 and/or comprises an amino acid sequence having at least 90% sequence identity to SEQ ID NO:29;
(G) Psychromonas sp., Cas6 is encoded by a nucleotide sequence having at least 90% sequence identity to SEQ ID NO:161 and/or comprises an amino acid sequence having at least 90% sequence identity to SEQ ID NO:168, Cas7 is encoded by a nucleotide sequence having at least 90% sequence identity to SEQ ID NO: 160 and/or comprises an amino acid sequence having at least 90% sequence identity to SEQ ID NO:167, Cas8 is encoded by a nucleotide sequence having at least 90% sequence identity to SEQ ID NO:159 and/or comprises an amino acid sequence having at least 90% sequence identity to SEQ ID NO:166, TnsA is encoded by a nucleotide sequence having at least 90% sequence identity to SEQ ID NO:162 and/or comprises an amino acid sequence having at least 90% sequence identity to SEQ ID NO:169, TnsB is encoded by a nucleotide sequence having at least 90% sequence identity to SEQ ID NO:163 and/or comprises an amino acid sequence having at least 90% sequence identity to SEQ ID NO: 170, TnsC is encoded by a nucleotide sequence having at least 90% sequence identity to SEQ ID NO:164 and/or comprises an amino acid sequence having at least 90% sequence identity to SEQ ID NO:171, and/or TniQ is encoded by a nucleotide sequence having at least 90% sequence identity to SEQ ID NO:158 and/or comprises an amino acid sequence having at least 90% sequence identity to SEQ ID NO:165;
(H) K. oxytoca, Cas6 is encoded by a nucleotide sequence having at least 90% sequence identity to SEQ ID NO:106 and/or comprises an amino acid sequence having at least 90% sequence identity to SEQ ID NO:112, Cas7 is encoded by a nucleotide sequence having at least 90% sequence identity to SEQ ID NO:105 and/or comprises an amino acid sequence having at least 90% sequence identity to SEQ ID NO:111, Cas8 is encoded by a nucleotide sequence having at least 90% sequence identity to SEQ ID NO:104 and/or comprises an amino acid sequence having at least 90% sequence identity to SEQ ID NO:110, TnsAB is encoded by a nucleotide sequence having at least 90% sequence identity to SEQ ID NO:107 and/or comprises an amino acid sequence having at least 90% sequence identity to SEQ ID NO:113, TnsC is encoded by a nucleotide sequence having at least 90% sequence identity to SEQ ID NO:108 and/or comprises an amino acid sequence having at least 90% sequence identity to SEQ ID NO: 114, and/or TniQ is encoded by a nucleotide sequence having at least 90% sequence identity to SEQ ID NO:103 and/or comprises an amino acid sequence having at least 90% sequence identity to SEQ ID NO: 109;
(I) C. polaris, Cas6 is encoded by a nucleotide sequence having at least 90% sequence identity to SEQ ID NO: 180 and/or comprises an amino acid sequence having at least 90% sequence identity to SEQ ID NO:187, Cas7 is encoded by a nucleotide sequence having at least 90% sequence identity to SEQ ID NO:179 and/or comprises an amino acid sequence having at least 90% sequence identity to SEQ ID NO:186, Cas8 is encoded by a nucleotide sequence having at least 90% sequence identity to SEQ ID NO: 178 and/or comprises an amino acid sequence having at least 90% sequence identity to SEQ ID NO:185, TnsA is encoded by a nucleotide sequence having at least 90% sequence identity to SEQ ID NO:181 and/or comprises an amino acid sequence having at least 90% sequence identity to SEQ ID NO:188, TnsB is encoded by a nucleotide sequence having at least 90% sequence identity to SEQ ID NO:182 and/or comprises an amino acid sequence having at least 90% sequence identity to SEQ ID NO: 189, TnsC is encoded by a nucleotide sequence having at least 90% sequence identity to SEQ ID NO:183 and/or comprises an amino acid sequence having at least 90% sequence identity to SEQ ID NO:190, and/or TniQ is encoded by a nucleotide sequence having at least 90% sequence identity to SEQ ID NO: 177 and/or comprises an amino acid sequence having at least 90% sequence identity to SEQ ID NO: 184; and/or
(J) N. qingdaonensis, Cas6 is encoded by a nucleotide sequence having at least 90% sequence identity to SEQ ID NO:84 and/or comprises an amino acid sequence having at least 90% sequence identity to SEQ ID NO:91, Cas7 is encoded by a nucleotide sequence having at least 90% sequence identity to SEQ ID NO:83 and/or comprises an amino acid sequence having at least 90% sequence identity to SEQ ID NO:90, Cas8 is encoded by a nucleotide sequence having at least 90% sequence identity to SEQ ID NO:82 and/or comprises an amino acid sequence having at least 90% sequence identity to SEQ ID NO:89, TnsA is encoded by a nucleotide sequence having at least 90% sequence identity to SEQ ID NO:85 and/or comprises an amino acid sequence having at least 90% sequence identity to SEQ ID NO:92, TnsB is encoded by a nucleotide sequence having at least 90% sequence identity to SEQ ID NO:86 and/or comprises an amino acid sequence having at least 90% sequence identity to SEQ ID NO: 93, TnsC is encoded by a nucleotide sequence having at least 90% sequence identity to SEQ ID NO: 87 and/or comprises an amino acid sequence having at least 90% sequence identity to SEQ ID NO:94, and/or TniQ is encoded by a nucleotide sequence having at least 90% sequence identity to SEQ ID NO:81 and/or comprises an amino acid sequence having at least 90% sequence identity to SEQ ID NO:88.

4. The system of claim 1, further comprising a guide nucleic acid (e.g., guide RNA, gRNA; e.g., CRISPR array (e.g., crRNA when processed)), wherein the guide nucleic acid comprises one or more (e.g., 1, 2, 3, 4, 5, 6 or more) spacer sequences having complementarity to a target site.

5. The system of claim 4, wherein the spacer sequence is oriented 5′-3′.

6. The system of claim 4, wherein the spacer sequence is oriented 3′-5′.

7. The system claim 1, wherein the target site is located immediately adjacent (3′) to a protospacer adjacent motif (PAM).

8. The system of claim 7, wherein the PAM comprises a nucleotide sequence of 5′-NNNNN-3′ that is immediately adjacent to and 5′ of the target site (protospacer), optionally wherein the PAM comprises a nucleotide sequence of 5′-NNNCN-3′ or 5′-NNNCC-3′ that is immediately adjacent to and 5′ of the target site.

9. The system of claim 7, wherein when the system is derived from (a) Aliiglaciecola sp., the PAM is 5′-NNNCN-3′, (b) Vibrio sp., the PAM is 5′-NNNCC-3′, (c) H. titanicae, the PAM is 5′-NNNCN-3′, (d) P. aquae, the PAM is 5′-NNNCN-3′, (e) P. iliopiscarium, the PAM is 5′-NNNCN-3′, (f) P. piscicola, the PAM is 5′-NNNCN-3′, (g) Psychromonas sp., the PAM is 5′-NNNNN-3′ (e.g., no PAM requirement), (h) K. oxytoca, the PAM is 5′-NNNCC-3′, (i) C. polaris, the PAM is 5′-NNNCC-3′, and/or (j) N. qingdaonensis, the PAM is 5′-NNNCN-3′.

10. The system of claim 3, wherein each of the one or more spacer sequences is linked at its 5′-end and at its 3′-end to a repeat sequence (e.g., repeat-spacer-repeat, repeat-spacer-repeat-spacer-repeat, repeat-spacer-repeat-spacer-repeat-spacer-repeat, and the like).

11. The system of claim 10, wherein the repeat sequence for a guide nucleic acid (e.g., CRISPR array) for: (A) Aliiglaciecola sp. is any one of the nucleotide sequences of SEQ ID NOs: 54-58, in any combination, optionally, SEQ ID NO:54 and/or SEQ ID NO:55; (B) Vibrio sp. is any one of the nucleotide sequences of SEQ ID NO: 154 and/or SEQ ID NO:155; (C) H. titanicae is any one of the nucleotide sequences of SEQ ID NOs: 75-78, in any combination, optionally, SEQ ID NO:75 and/or SEQ ID NO:76; (D) P. aquae is any one of the nucleotide sequences of SEQ ID NOs: 134-137, in any combination, optionally, SEQ ID NO: 134 and/or SEQ ID NO:135; (E) P. iliopiscarium is any one of the nucleotide sequences of SEQ ID NOs: 15-19, in any combination, optionally, SEQ ID NO:15 and/or SEQ ID NO:16; (F) P. piscicola is any one of the nucleotide sequences of SEQ ID NOs: 15, 16, 36 or 37, in any combination, optionally, SEQ ID NO:15 and/or SEQ ID NO:16; (G) Psychromonas sp. is any one of the nucleotide sequences of SEQ ID NOs: 172-174, in any combination, optionally, SEQ ID NO: 172 and/or SEQ ID NO:173; (H) K. oxytoca is any one of the nucleotide sequences of SEQ ID NOs: 115-117, in any combination, optionally, SEQ ID NO:115 and/or SEQ ID NO: 116; (I) C. polaris is any one of the nucleotide sequences of SEQ ID NO: 191 and/or SEQ ID NO: 192; and/or (J) N. qingdaonensis is any one of the nucleotide sequences of SEQ ID NOs: 95-100, in any combination, optionally, SEQ ID NO:95, SEQ ID NO:96, SEQ ID NO:97 and/or SEQ ID NO:98, in any combination.

12. The system of claim 1, further comprising a donor DNA to be integrated, wherein the donor DNA comprises a cargo nucleic acid sequence and a first transposon end sequence and a second transposon end sequence, wherein the cargo nucleic acid sequence is flanked by the first transposon end sequence and the second transposon end sequence, and wherein each of the first transposon end sequence and the second transposon end sequence comprises at least one TnsB binding site.

13. The system of claim 12, wherein the first transposon end sequence and the second transposon end sequence each comprise at least two TnsB binding sites.

14. The system of claim 13, wherein the at least two TnsB binding sites are immediately adjacent to each other (e.g., no intervening nucleotides (no variable region)).

15. The system of claim 12, wherein a variable region is located between the at least two TnsB binding sites.

16. The system of claim 15 wherein the variable region is a length of about 1 nucleotide to about 80 nucleotides, optionally a length of about 1 nucleotide to about 3 nucleotides and/or about 1 nucleotide to about 10 nucleotides.

17. The system of claim 12, wherein the first transposon end sequence and/or the second transposon end sequence comprise three or four TnsB binding sites, optionally,
wherein at least two of the three or four TnsB binding sites are immediately adjacent to each other (e.g., no intervening nucleotides (no variable region) between the at least two of the three or four TnsB binding sites), and/or
wherein a variable region of 1 nucleotide to about 80 nucleotides is located between at least two of the three to four TnsB binding sites.

18. The system of claim 12, wherein the 5′ end of the first transposon end sequence and the 3′ end of the second transposon end sequence comprise a terminal inverted repeat (TIR).

19. The system of claim 18, wherein the TIR comprises a length of about 8 base pairs.

20. The system of claim 19, wherein the TIR comprises a nucleotide sequence of TGTNNNNN and/or a reverse complement of TGTNNNNN.

21. The system of claim 19, wherein the second transposon end sequence comprises a sequence that is the reverse complement of the TIR of the first transposon end sequence.

22. The system of claim 19, wherein the TIR of the first transposon end sequence comprises a nucleotide sequence of TGTNNNNN and the TIR of the second transposon end sequence comprises a sequence that is the reverse complement of the nucleotide sequence of TGTNNNNN (i.e., NNNNNACA).

23. The system of claim 19, wherein the TIR comprises a nucleotide sequence of TGTTGAAA, TGTTGATA, TGTTGATC, TGTCGTTT, TGTCGCTT, TGTCGCAA, TGTCGCTG, TGTGGCTG, or TGTGGCTA, or the reverse complement thereof.

24. The system of claim 19, wherein
(a) the TIR of the first transposon end sequence comprises a sequence of TGTTGAAA and the TIR of the second transposon end sequence comprises the reverse complement of TGTTGATA (TATCAACA), (b) the TIR of the first transposon end sequence comprises a sequence of TGTTGAAA and the TIR of the second transposon end sequence comprises the reverse complement of TGTTGAAA (TTTCAACA), (c) the TIR of the first transposon end sequence comprises a sequence of TGTTGATC and the TIR of the second transposon end sequence comprises the reverse complement of TGTTGATC (GATCAACA), (d) the TIR of the first transposon end sequence comprises a sequence of TGTCGTTT and the TIR of the second transposon end sequence comprises the reverse complement of TGTCGTTT (AAACGACA), (e) the TIR of the first transposon end sequence comprises a sequence of TGTCGTTT and the TIR of the second transposon end sequence comprises the reverse complement of TGTCGCTT (AAACGACA); (f) the TIR of the first transposon end sequence comprises a sequence of TGTCGCAA and the TIR of the second transposon end sequence comprises the reverse complement of TGTCGCTG (AAGCGACA), (g) the TIR of the first transposon end sequence comprises a sequence of TGTGGCTG and the TIR of the second transposon end sequence comprises the reverse complement of TGTGGCTA (TAGCCACA), and/or (h) the TIR of the first transposon end sequence comprises a sequence of TGTCGCTG and the TIR of the second transposon end sequence comprises the reverse complement of TGTCGCTG (CAGCGACA).

25. The system of claim 19, wherein (a) for Aliiglaciecola sp., the TIR of the first transposon end sequence comprises a nucleotide sequence of TGTTGATC and the TIR of the second transposon end sequence comprises the reverse complement of the nucleotide sequence of TTGTTGATC (GATCAACA), (b) for Vibrio sp., the TIR of the first transposon end sequence comprises a nucleotide sequence of TGTGGCTG and the TIR of the second transposon end sequence comprises the reverse complement of the nucleotide sequence of TGTGGCTA (TAGCCACA), (c) for H. titanicae, the TIR of the first transposon end sequence comprises a nucleotide sequence of TGTCGTTT and the TIR of the second transposon end sequence comprises the reverse complement of the nucleotide sequence of TGTCGTTT (AAACGACA), (d) for P. aquae, the TIR of the first transposon end sequence comprises a nucleotide sequence of TGTCGCAA and the TIR of the second transposon end sequence comprises the reverse complement of nucleotide sequence of TGTCGCTG (AAGCGACA), (e) for P. iliopiscarium, the TIR of the first transposon end sequence comprises a nucleotide sequence of TGTTGAAA and the TIR of the second transposon end sequence comprises the reverse complement of the nucleotide sequence of TGTTGATA (TATCAACA), (f) for P. piscicola, the TIR of the first transposon end sequence comprises a nucleotide sequence of TGTTGAAA and the TIR of the second transposon end sequence comprises the reverse complement of the nucleotide sequence of TGTTGAAA (TTTCAACA), (g) for Psychromonas sp., the TIR of the first transposon end sequence comprises a nucleotide sequence of TGTCGCTG and the TIR of the second transposon end sequence comprises the reverse complement of the nucleotide sequence of TGTCGCTG (CAGCGACA), (h) for K. oxytoca, the TIR of the first transposon end sequence comprises a nucleotide sequence of TGTCGTTT and the TIR of the second transposon end sequence comprises the reverse complement of the nucleotide sequence of TGTCGCTT (AAACGACA), (i) for C. polaris, the TIR of the first transposon end sequence comprises a nucleotide sequence of TGTCGCTG and the TIR of the second transposon end sequence comprises the reverse complement of the nucleotide sequence of TGTCGCTG (CAGCGACA), and/or (j) for N. qingdaonensis, the TIR of the first transposon end sequence comprises a nucleotide sequence of TGTCGTTT and the TIR of the second transposon end sequence comprises the reverse complement of the nucleotide sequence of TGTCGCTT (AAACGACA).

26. The system of claim 13, wherein the first transposon end sequence and the second transposon end sequence are Tn7-like transposon end sequences.

27. The system of claim 12, wherein for: (a) Aliiglaciecola sp., the first transposon end sequence and the second transposon end sequence comprise a nucleotide sequence of SEQ ID NO:59 and SEQ ID NO:60, respectively; (b) Vibrio sp., the first transposon end sequence and the second transposon end sequence comprise a nucleotide sequence of SEQ ID NO:156 and SEQ ID NO:157, respectively; (c) H. titanicae, the first transposon end sequence and the second transposon end sequence comprise a nucleotide sequence of SEQ ID NO:79 and SEQ ID NO:80, respectively; (d) P. aquae, the first transposon end sequence and the second transposon end sequence comprise a nucleotide sequence of SEQ ID NO: 138 and SEQ ID NO:139, respectively; (e) P. iliopiscarium, the first transposon end sequence and the second transposon end sequence comprise a nucleotide sequence of SEQ ID NO: 20 and SEQ ID NO:21, respectively; (f) P. piscicola, the first transposon end sequence and the second transposon end sequence comprise a nucleotide sequence of SEQ ID NO:38 and SEQ ID NO:39, respectively; (g) Psychromonas sp., the first transposon end sequence and the second transposon end sequence comprise a nucleotide sequence of SEQ ID NO:175 and SEQ ID NO: 176, respectively; (h) K. oxytoca, the first transposon end sequence and the second transposon end sequence comprise a nucleotide sequence of SEQ ID NO:118 and SEQ ID NO: 119, respectively; (i) C. polaris, the first transposon end sequence and the second transposon end sequence comprise a nucleotide sequence of SEQ ID NO: 193 and SEQ ID NO:194, respectively; and/or (j) N. qingdaonensis, the first transposon end sequence and the second transposon end sequence comprise a nucleotide sequence of SEQ ID NO:101 and SEQ ID NO: 102, respectively.

28. The system of claim 1, wherein the CRISPR-Cas system and the Tn7-like transposon system are on the same vector.

29. The system of claim 5, wherein the CRISPR-Cas system, the Tn7-like transposon system and the guide nucleic acid are on the same vector.

30. The system of claim 4, wherein the CRISPR-Cas system, the Tn7-like transposon system, and the guide nucleic acid are on two or more different vectors in any combination.

31. The system of claim 12, wherein the CRISPR-Cas system, the Tn7-like transposon system, the guide nucleic acid and the donor DNA are on the same vector or on two or more different vectors in any combination.

32. The system of claim 1, wherein the one or more vectors are plasmids.

33. The system of claim 12, wherein the cargo nucleic acid sequence is about 100 nucleotides to about 100,000 nucleotides in length.

34. The system of claim 12, wherein the donor DNA is linear.

35. A recombinant cell comprising the system of claim 1.

36. The recombinant cell of claim 35, wherein the cell is from eukaryotic organism or a prokaryotic organism, optionally wherein the prokaryote is a bacterium, a cyanobacterium, or an archaeon and/or the eukaryote is a plant or an animal.

37. A method for RNA-guided DNA integration comprising,
introducing into a cell the system of claim 1, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid.

38. A method of modifying the genome of a target organism, comprising
introducing into a cell the system of claim 1, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the genome of the target organism.

39. A method of modifying (e.g., editing) a nucleic acid (e.g., target region; target DNA) in the genome of a cell, comprising
introducing into a cell the system of claim 1, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into the nucleic acid, thereby modifying the nucleic acid in the genome of the cell.

40. A method of killing a cell, comprising
introducing into a cell the system of claim 1, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into an essential gene in the nucleic acid, thereby killing the cell.

41. A method of screening for non-essential genes in a cell, comprising
introducing into a cell the system of claim 1, a donor DNA, and a guide nucleic acid comprising at least one spacer having complementarity to a target site present in a nucleic acid present in the cell, wherein the engineered CRISPR-Cas system binds to the target site and the engineered transposon system integrates the donor sequence into a gene in the nucleic acid, wherein when the cell survives, the gene is non-essential, thereby screening for non-essential genes in the cell.

42. A method of altering the expression of a gene in a cell, comprising
introducing into a cell the system of claim 1, a guide nucleic acid comprising at least one spacer having complementarity to a target site present in a nucleic acid present in the cell, and a donor DNA, wherein the engineered CRISPR-Cas system binds to the target site and the engineered transposon system integrates the donor sequence into a regulator region of a gene in the nucleic acid, thereby altering the expression of a gene in a cell.

43. The method of claim 37, wherein the donor DNA comprises a cargo nucleic acid sequence and a first transposon end sequence and a second transposon end sequence, wherein the cargo nucleic acid sequence is flanked by the first transposon end sequence and the second transposon end sequence, and wherein each of the first transposon end sequence and the second transposon end sequence comprises at least one TnsB binding site.

44. The method of claim 43, wherein the first transposon end sequence and the second transposon end sequence each comprise at least two TnsB binding sites.

45. The method of claim 43, wherein the at least two TnsB binding sites are immediately adjacent to each other (e.g., no intervening nucleotides (no variable region)).

46. The method of claim 44 wherein a variable region is located between the at least two TnsB binding sites.

47. The method of claim 46, wherein the variable region is a length of about 1 nucleotide to about 80 nucleotides, optionally a length of about 1 nucleotide to about 3 nucleotides and/or about 1 nucleotide to about 10 nucleotides.

48. The method of claim 43, wherein the first transposon end sequence and/or the second transposon end sequence comprise three or four TnsB binding sites, optionally, wherein at least two of the three or four TnsB binding sites are immediately adjacent to each other (e.g., no intervening nucleotides (no variable region) between the at least two of the three or four TnsB binding sites), and/or wherein a variable region of 1 nucleotide to about 80 nucleotides is located between at least two of the three to four TnsB binding sites.

49. The method of claim 43, wherein the 5′ end of the first transposon end sequence and the 3′ end of the second transposon end sequence comprise a terminal inverted repeat (TIR).

50. The method of claim 49, wherein the TIR comprises a length of about 8 base pairs.

51. The method of claim 50, wherein the TIR comprises a nucleotide sequence of TGTNNNNN and/or a reverse complement of TGTNNNNN.

52. The method of claim 49, wherein the second transposon end sequence comprises a nucleotide sequence that is the reverse complement of the TIR of the first transposon end sequence.

53. The method of claim 49, wherein the TIR of the first transposon end sequence comprises a nucleotide sequence of TGTNNNNN and the TIR of the second transposon end sequence comprises a nucleotide sequence that is the reverse complement of TGTNNNNN (i.e., NNNNNACA).

54. The method of claim 49, wherein the TIR comprises a nucleotide sequence of TGTTGAAA, TGTTGATA, TGTTGATC, TGTCGTTT, TGTCGCTT, TGTCGCAA, TGTCGCTG, TGTGGCTG, or TGTGGCTA, or the reverse complement thereof.

55. The method of claim 49, wherein
(a) the TIR of the first transposon end sequence comprises a sequence of TGTTGAAA and the TIR of the second transposon end sequence comprises the reverse complement of TGTTGATA (TATCAACA), (b) the TIR of the first transposon end sequence comprises a sequence of TGTTGAAA and the TIR of the second transposon end sequence comprises the reverse complement of TGTTGAAA (TTTCAACA), (c) the TIR of the first transposon end sequence comprises a sequence of TGTTGATC and the TIR of the second transposon end sequence comprises the reverse complement of TGTTGATC (GATCAACA), (d) the TIR of the first transposon end sequence comprises a sequence of TGTCGTTT and the TIR of the second transposon end sequence comprises the reverse complement of TGTCGTTT (AAACGACA), (e) the TIR of the first transposon end sequence comprises a sequence of TGTCGTTT and the TIR of the second transposon end sequence comprises the reverse complement of TGTCGCTT (AAACGACA); (f) the TIR of the first transposon end sequence comprises a sequence of TGTCGCAA and the TIR of the second transposon end sequence comprises the reverse complement of TGTCGCTG (AAGCGACA), (g) the TIR of the first transposon end sequence comprises a sequence of TGTGGCTG and the TIR of the second transposon end sequence comprises the reverse complement of TGTGGCTA (TAGCCACA), and/or (h) the TIR of the first transposon end sequence comprises a sequence of TGTCGCTG and the TIR of the second transposon end sequence comprises the reverse complement of TGTCGCTG (CAGCGACA).

56. The method of claim 49, wherein (a) for Aliiglaciecola sp., the TIR of the first transposon end sequence comprises a nucleotide sequence of TGTTGATC and the TIR of the second transposon end sequence comprises the reverse complement of the nucleotide sequence of TTGTTGATC (GATCAACA), (b) for Vibrio sp., the TIR of the first transposon end sequence comprises a nucleotide sequence of TGTGGCTG and the TIR of the second transposon end sequence comprises the reverse complement of the nucleotide sequence of TGTGGCTA (TAGCCACA), (c) for H. titanicae, the TIR of the first transposon end sequence comprises a nucleotide sequence of TGTCGTTT and the TIR of the second transposon end sequence comprises the reverse complement of the nucleotide sequence of TGTCGTTT (AAACGACA), (d) for P. aquae, the TIR of the first transposon end sequence comprises a nucleotide sequence of TGTCGCAA and the TIR of the second transposon end sequence comprises the reverse complement of nucleotide sequence of TGTCGCTG (AAGCGACA), (e) for P. iliopiscarium, the TIR of the first transposon end sequence comprises a nucleotide sequence of TGTTGAAA and the TIR of the second transposon end sequence comprises the reverse complement of the nucleotide sequence of TGTTGATA (TATCAACA), (f) for P. piscicola, the TIR of the first transposon end sequence comprises a nucleotide sequence of TGTTGAAA and the TIR of the second transposon end sequence comprises the reverse complement of the nucleotide sequence of TGTTGAAA (TTTCAACA), (g) for Psychromonas sp., the TIR of the first transposon end sequence comprises a nucleotide sequence of TGTCGCTG and the TIR of the second transposon end sequence comprises the reverse complement of the nucleotide sequence of TGTCGCTG (CAGCGACA), (h) for K. oxytoca, the TIR of the first transposon end sequence comprises a nucleotide sequence of TGTCGTTT and the TIR of the second transposon end sequence comprises the reverse complement of the nucleotide sequence of TGTCGCTT (AAACGACA), (i) for C. polaris, the TIR of the first transposon end sequence comprises a nucleotide sequence of TGTCGCTG and the TIR of the second transposon end sequence comprises the reverse complement of the nucleotide sequence of TGTCGCTG (CAGCGACA), and/or (j) for N. qingdaonensis, the TIR of the first transposon end sequence comprises a nucleotide sequence of TGTCGTTT and the TIR of the second transposon end sequence comprises the reverse complement of the nucleotide sequence of TGTCGCTT (AAACGACA).

57. The method of claim 43, wherein the cargo nucleic acid sequence is about 100 nucleotides to about 100,000 nucleotides in length.

58. The method of claim 37, wherein the donor DNA integrates at about 25 nucleotides to about 60 nucleotides from the target site, optionally at about 35 nucleotides to about 55 nucleotides from the target site or at about 45 nucleotides to about 55 nucleotides from the target site.

59. The method of claim 37, wherein the target site is located immediately adjacent (3′) to a protospacer adjacent motif (PAM).

60. The method of claim 59, wherein the PAM comprises a nucleotide sequence of 5′-NNNNN-3′ that is immediately adjacent to and 5′ of the target site (protospacer), optionally wherein the PAM comprises a nucleotide sequence of 5′-NNNCN-3′ or 5′-NNNCC-3′ that is immediately adjacent to and 5′ of the target site.

61. The method of claim 59, wherein when the PAM sequence is oriented 5′-3′, then the donor DNA is integrated downstream of the target site.

62. The method of claim 59, wherein when the PAM sequence is oriented 3′-5′, then the donor DNA is integrated upstream of the target site.

63. The method of claim 59, wherein the target site is located immediately adjacent (3′) to a protospacer adjacent motif (PAM).

64. The method of claim 59, wherein the PAM comprises a nucleotide sequence of 5′-NNNNN-3′ that is immediately adjacent to and 5′ of the target site (protospacer), optionally wherein the PAM comprises a nucleotide sequence of 5′-NNNCN-3′ or 5′-NNNCC-3′ that is immediately adjacent to and 5′ of the target site.

65. The method of claim 59, wherein when the system is derived from (a) Aliiglaciecola sp., the PAM is 5′-NNNCN-3′, (b) Vibrio sp., the PAM is 5′-NNNCC-3′, (c) H. titanicae, the PAM is 5′-NNNCN-3′, (d) P. aquae, the PAM is 5′-NNNCN-3′, (e) P. iliopiscarium, the PAM is 5′-NNNCN-3′, (f) P. piscicola, the PAM is 5′-NNNCN-3′, (g) Psychromonas sp., the PAM is 5′-NNNNN-3′ (e.g., no PAM requirement), (h) K. oxytoca, the PAM is 5′-NNNCC-3′, (i) C. polaris, the PAM is 5′-NNNCC-3′, and/or (j) N. qingdaonensis, the PAM is 5′-NNNCN-3′.

66. The method of claim 37, wherein the donor DNA is linear.

67. The method of claim 37, wherein the CRISPR-Cas system and the Tn7-like transposon system are on the same vector.

68. The method of claim 37, wherein the CRISPR-Cas system, the Tn7-like transposon system and the guide nucleic acid are on the same vector.

69. The method of claim 37, wherein the CRISPR-Cas system, the Tn7-like transposon system, and the guide nucleic acid are on two or more different vectors in any combination.

70. The method of claim 37, wherein the CRISPR-Cas system, the Tn7-like transposon system, the guide nucleic acid and the donor DNA are on the same vector or on two or more different vectors in any combination.

71. The method of claim 43, wherein the cargo nucleic acid sequence is integrated into the nucleic acid, 5′ to 3′, the first transposon end sequence, the cargo nucleic acid sequence, and the second transposon end sequence (e.g., in a right to left orientation).

72. The method of claim 37, wherein the cargo nucleic acid sequence is integrated into the nucleic acid, 5′ to 3′, the second transposon end sequence, the cargo nucleic acid sequence, and the first transposon end sequence (e.g., in a left to right orientation).

72. The method of claim 37, wherein the cell is from eukaryotic organism or a prokaryotic organism, optionally wherein the prokaryote is a bacterium, a cyanobacterium, or an archaeon and/or the eukaryote is a plant or an animal.
